(19) United States
(12) Patent Application Publication
(10) Pub. No.: US 2021/0308270 A1
(43) Pub. Date: Oct. 7, 2021

(54) SYSTEMS AND METHODS FOR INTRACELLULAR DELIVERY VIA NON-CHARGED SEQUENCE-DEFINED CELL-PENETRATING OLIGOMERS

(71) Applicant: Cornell University, Ithaca, NY (US)

(72) Inventors: Christopher Akinleye ALABI, Ithaca, NY (US); Ngoc Nhu PHAN, Ithaca, NY (US)

(21) Appl. No.: 17/055,960

(22) PCT Filed: May 16, 2019
(86) PCT No.: PCT/US2019/032700

§ 371 Date: Nov. 16, 2020

Related U.S. Application Data
(60) Provisional application No. 62/672,454, filed on May 16, 2018.

(51) Int. Cl. A61K 47/59; A61K 47/54; A61P 31/04

(52) U.S. Cl. CPC A61K 47/59; A61P 31/04; A61K 47/54

(57) ABSTRACT

The present disclosure provides oligoTEAs and methods of using the oligoTEAs. The oligoTEAs may be functionalized with one or more cargo group. The oligoTEAs may be made by iterative thiol-ene and Michael reactions. The oligoTEAs functionalized with one or more cargo group may be used to treat bacterial infections, cancers, viral infections, urinary tract infections, skin infections, cystic fibrosis, sepsis, fungal infections, or a combination thereof.

### CROSS REFERENCE TO RELATED APPLICATIONS

This application claims priority to U.S. Provisional Application No. 62/672,454, filed on May 16, 2018, the disclosure of which is hereby incorporated by reference.

### STATEMENT REGARDING FEDERALLY SPONSORED RESEARCH OR DEVELOPMENT

This invention was made with government support under Grant No. CHE-1554046 awarded by the National Science Foundation. The government has certain rights in the invention.

### FIELD OF DISCLOSURE

The disclosure generally relates to oligoTEAs and synthesis and uses thereof. More particularly, the disclosure relates to oligoTEAs having cargo molecules for intracellular delivery.

### BACKGROUND OF THE DISCLOSURE

The plasma membrane of eukaryotic cells is a tightly controlled barrier used to protect intracellular components from the external environment and help regulate intracellular transport. The cell membrane is selectively permeable to ions and small organic molecules, making it difficult for exogenous large molecules such as therapeutic agents to gain access to their intracellular target. As such, drug delivery research is focused on the design of macromolecular transporters that can facilitate the transport of bioactive molecules across the cell membrane. The discovery of cell penetrating peptides (CPPs) about three decades ago ushered in a new mode for the intracellular transport of a wide variety of cargoes, ranging from DNA and proteins to small molecule drugs. The majority of CPPs are cationic or amphipathic peptides composed of 10-30 amino acids with a high percentage of basic amino acids, leading to a net positive charge. One of the first CPPs to be discovered was the HIV-1 Tat49-57 9-mer basic domain (RKKRRQRRR) (SEQ ID NO:2) (“Tat”). This peptide was shown to engage cell membrane phospholipids via electrostatic and hydrogen bonding interactions. This study subsequently led to the design of numerous arginine-rich peptides, most of which have been shown to efficiently translocate across the cell membrane and outperform Tat.

Despite their promising ability to facilitate intracellular delivery, CPPs have some drawbacks that hinder their potential for in vivo applications. These drawbacks include rapid metabolic degradation by proteases and low efficacy upon exposure to extracellular matrix (ECM) components such as heparan sulfate proteoglycans. Additional clinical disadvantages include a high propensity for triggering an immune response and possible kidney accumulation due to positive charge accumulation at the anionic glomerular filtration membrane. These shortcomings, mostly centered on their cationic charge and proteolytic susceptibility, motivate the development of synthetic alternatives that can overcome these limitations.

Intracellular drug delivery systems are often limited by their poor serum stability and delivery efficiency. The discovery of cell-penetrating peptides (CPPs) over three decades ago uncovered a novel method for transporting a variety of cargoes into cells, ranging from DNA and polymers to nanoparticles. Although promising, CPPs have several drawbacks that hinder their use for in vivo therapeutic applications such as rapid metabolic degradation by proteases, undesired interactions with the biological milieu, and a propensity for mounting an immune response. These issues highlight the need for inexpensive synthetic alternatives that are proteolytically stable and yet easy to assemble at scale with high structural diversity.

Based on the foregoing, there is an ongoing and unmet need for improved intracellular drug delivery systems.

### SUMMARY OF THE DISCLOSURE

In an aspect, the present disclosure provides compounds. The compounds are oligothioetheramides (oligoTEAs). The compounds may comprise a cargo group. The compounds may be charged or uncharged.

In an example, a compound has the following structure:

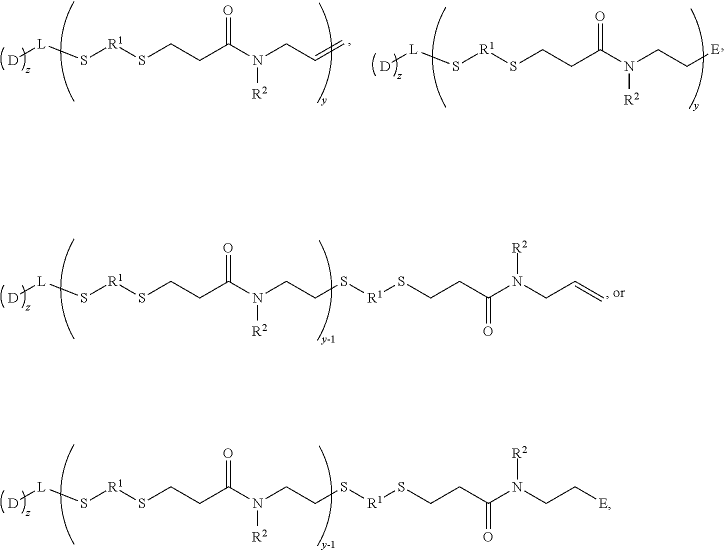

where L is chosen from a linking group, NH, N, O, and S. D is a cargo group. R¹ is independently at each occurrence in the compound chosen from straight chain or branched C₂ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)ₐ—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8); diol groups having the structure —CH₂—CHOH—(CH₂)ₑ—CHOH—CH₂—, where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like). R² is independently at each occurrence in the compound chosen from cationic groups (e.g., alkyl amine groups, alkyl guanidinium groups, and the like), aliphatic electrophilic groups, aliphatic nucleophilic groups, straight chain or branched C₁ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)ₐ—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8); diol groups having the structure —CH₂—CHOH—(CH₂)ₑ—CHOH—CH₂—; where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like). E is an end group chosen from ═CH₂, D, and L-(D)_{z}. y is 1 to 12 (e.g., 1, 2, 3, 4, 5, 6, 7, 8, 9, 10, 11, 12). z is 1 to 5 (e.g., 1, 2, 3, 4, 5). In an example, a compound may have one or more R² group(s) different than one or more R¹ group(s).

In an aspect, the disclosure provides methods of making compounds of the present disclosure. The compounds may be synthesized by iterative thiol-ene reactions and Michael reactions. The methods use a monomer having two or more functional groups (the monomers may have additional functional groups that do not react during the polymerization reactions) that react with a co-monomer under orthogonal conditions (i.e., a monomer with orthogonal functional groups).

In an aspect, the present disclosure provides compositions comprising compounds of the present disclosure. The compositions also comprise one or more pharmaceutically acceptable carrier.

In an aspect, the disclosure provides kits. A kit may comprise pharmaceutical preparations containing any one or any combination of compounds and printed material. In an example, a kit comprises a closed or sealed package that contains the pharmaceutical preparation. In various examples, the package comprises one or more closed or sealed vials, bottles, blister (bubble) packs, or any other suitable packaging for the sale, or distribution, or use of the compounds and compositions comprising compounds of the present disclosure. The printed material may include printed information.

In an aspect, the present disclosure provides methods of using one or more compound or composition thereof. The method may comprise intracellular delivery of one or more cargo group of the compound. Upon delivery, the cargo is delivered (e.g., released) in its effective form.

The compounds may be suitable in methods to treat cancers (e.g., leukemia, lung cancer (e.g., non-small cell lung cancer), dermatological cancer, premalignant lesions of the upper digestive tract, malignancies of the prostate, malignancies of the brain, malignancies of the breast, and the like, and combinations thereof), bacterial infections, viral infections, urinary tract infections, skin infections, cystic fibrosis, sepsis, fungal infections, and the like, and combinations thereof. Compounds of the present disclosure may also be fluorescent probes.

### BRIEF DESCRIPTION OF THE FIGURES

For a fuller understanding of the nature and objects of the disclosure, reference may be made to the following detailed description taken in conjunction with the accompanying figures.

FIG. 1 shows (A) cellular uptake of fluorescein cargo by oligoTEAs in HeLa cells measured by flow cytometry. Cells were treated with 5 μM of fluorescein-oligoTEA conjugates for 1 hour. (B) Live-cell confocal microscopy of fluorescein cargo uptake by PEO²-B and R9 in HeLa cells. Images are the merged green fluorescence of the fluorescein-oligoTEA conjugates and the bright field image of the cell at 63× magnification. (C) Cellular uptake of PEO²-B in HeLa, SKOV-3 and HEK293 cells measured by flow cytometry. All cells were treated with 5 μM of PEO²-B-fluorescein conjugates for 1 hour. (D) Dose-dependent uptake of PEO²-B in HeLa cells.

FIG. 2 shows (A) cellular uptake of PEO²-B and R9 conjugates in HeLa cells with and without serum. (B) Cellular uptake of PEO²-B and R9 in HeLa cells with and without heparin sulfate pretreatment at the indicated doses. (C) Temperature-dependent cellular uptake of PEO²-B in HeLa, SKOV-3 and HEK293 cells. (D) Effect of endocytosis inhibitors on cellular uptake of PEO²-B in HeLa, SKOV-3 and HEK293 cells. Cells were pre-treated with chlorpromazine to inhibit clathrin, filipin III to inhibit caveolae, cytochalasin D to inhibit macropinocytosis, and NaN3/2-deoxy-D-glucose to block ATP synthesis. Order of the bars are “no inhibitor”, “(−) Clathrin”, “(−) Caveolae”, (−) Macropin, (−) ATP.

FIG. 3 shows membrane fluidity reflected by the Laurdan GP values of HeLa, SKOV-3 and HEK293 cells.

FIG. 4 shows confocal microscopy of fluorescein-PEO²-B (green) co-delivered with (A) Dextran-AlexaFluor 647, and (B) Transferrin-AlexaFluor 647. Live cell confocal images were acquired at 63× magnification. Red—Dextran-AlexaFluor 647, Green—fluorescein-PEO²-B.

FIG. 5 shows (A) HPLC traces showing the retention times of all purified oligoTEAs used in this study. (B) Solubility testing of BDT-B, PEO²-B and PEO⁴-B.

FIG. 6 shows positive mode LCMS of DTT-G with the corresponding mass spectra; HPLC trace indicating purity is shown in FIG. 5. Calculated mass [M+H] 1458.65, observed mass [M+H] 1458.48; [M+2H] 730.07; [M+3H] 486.99; [M+4H] 365.58.

FIG. 7 shows positive mode LCMS of DTT-B with the corresponding mass spectra; HPLC trace indicating purity is shown in FIG. 5. Calculated mass [M+H] 1342.64, observed mass [M+H] 1342.19; [M+2H] 671.72.

FIG. 8 shows positive mode LCMS of BDT-B with the corresponding mass spectra; HPLC trace indicating purity is shown in FIG. 5. Calculated mass [M+H] 1214.68, observed mass [M+H] 1214.55; [M+2H] 607.90.

FIG. 9 shows positive mode LCMS of PEO¹-B with the corresponding mass spectra; HPLC trace indicating purity is shown in FIG. 5. Calculated mass [M+H] 1274.70, observed mass [M+H] 1274.44; [M+2H] 637.80.

FIG. 10 shows positive mode LCMS of PEO²-B with the corresponding mass spectra; HPLC trace indicating purity is shown in FIG. 5. Calculated mass [M+H] 1334.72, observed mass [M+H] 1334.60; [M+2H] 668.13.

FIG. 11 shows positive mode LCMS of PEO³-B with the corresponding mass spectra; HPLC trace indicating purity is shown in FIG. 5. Calculated mass [M+H] 1394.74, observed mass [M+H] 1394.50; [M+2H] 697.95.

FIG. 12 shows positive mode LCMS of PEO⁴-B with the corresponding mass spectra; HPLC trace indicating purity is shown in FIG. 5. Calculated mass [M+H] 1454.77, observed mass [M+H] 1454.50; [M+2H] 727.84.

FIG. 13 shows positive mode LCMS of R9-fluorescein with the absorbance at 230 nm (top) and the corresponding mass spectra (bottom). Calculated mass [M+H] 1951.10, observed mass [M+2H] 976.13; [M+3H] 651.12; [M+4H] 488.61; [M+5H] 391.11; [M+6H] 326.12.

FIG. 14 shows positive mode LCMS of fluorescein-DTT-G with the absorbance at 230 nm (top) and the corresponding mass spectra (bottom). Calculated mass [M+H] 1816.69, observed mass [M+2H] 908.80; [M+3H] 606.13; [M+4H] 454.87.

FIG. 15 shows positive mode LCMS of fluorescein-DTT-B with the absorbance at 230 nm (top) and the corresponding mass spectra (bottom). Calculated mass [M+H] 1700.69, observed mass [M+2H] 850.50; [M+3H] 567.60.

FIG. 16 shows positive mode LCMS of fluorescein-BDT-B with the absorbance at 230 nm (top) and the corresponding mass spectra (bottom). Calculated mass [M+H] 1572.73, observed mass [M+H] 1572.40; [M+2H] 787.03.

FIG. 17 shows positive mode LCMS of fluorescein-PEO¹-B with the absorbance at 230 nm (top) and the corresponding mass spectra (bottom). Calculated mass [M+H] 1632.75, observed mass [M+H] 1632.60; [M+2H] 816.90.

FIG. 18 shows positive mode LCMS of fluorescein-PEO²-B with the absorbance at 230 nm (top) and the corresponding mass spectra (bottom). Calculated mass [M+H] 1692.77, observed mass [M+2H] 846.70; [M+3H] 564.90.

FIG. 19 shows positive mode LCMS of fluorescein-PEO³-B with the absorbance at 230 nm (top) and the corresponding mass spectra (bottom). Calculated mass [M+H] 1752.85, observed mass [M+2H] 876.87; [M+3H] 584.95.

FIG. 20 shows positive mode LCMS of fluorescein-PEO⁴-B with the absorbance at 230 nm (top) and the corresponding mass spectra (bottom). Calculated mass [M+H] 1812.81, observed mass [M+2H] 906.90.

FIG. 21 shows trypan blue quenching of external fluorescence. Cells were treated for 1 hour with fluorescein conjugates (100 nM of Transferrin-Alexa Fluor 488 was used), washed 3-times with PBS and treated with a trypan blue solution at room temperature for 10 minutes prior to reading (reading done in the presence of trypan blue). In this figure 2PEG is another name for PEO²-B.

FIG. 22 shows Cellular uptake of R9-fluorescein and fluorescein-PEO²-B in HeLa, SKOV-3 and HEK293 cells, measured by flow cytometry. All cells were treated with 5 μM of fluorescein conjugates for 1 hour. PEO²-B left bars, R9 right bars.

FIG. 23 shows cytotoxicity of oligoTEAs in (A) HeLa cells via MTS assay and (B) red blood cells via hemolysis assay. Cells were treated with 1-40 μM of purified oligoTEAs for 1 hour at 37° C. In this figure 1PEG is another name for PEO¹-B; 2PEG is another name for PEO²-B; 3PEG is another name for PEO³-B; 4PEG is another name for PEO⁴-B.

FIG. 24 shows dose dependent uptake of PEO⁴-B (0.5-5 μM) in HeLa cells.

FIG. 25 shows uptake kinetics of fluorescein-PEO²-B at 2.5 μM in HeLa cells via flow cytometry. Cells were treated with the compound for 15-120 mins at 37° C.

FIG. 26 shows temperature-dependent uptake of R9-fluorescein in HeLa, SKOV-3 and HEK293 cells.

FIG. 27 shows effect of endocytosis inhibitors on cellular uptake of R9-fluorescein in HeLa, SKOV-3 and HEK293 cells. Cells were pre-treated with chloropromazine to inhibit clathrin, filipin III to inhibit caveolae, cytochalasin D to inhibit macropinocytosis and NaN3/2-deoxy-D-glucose to block ATP synthesis.

FIG. 28 shows ¹H NMR (600 MHz, CDCl₃) of the polyethylene glycol monomer.

FIG. 29 shows positive mode LCMS of the polyethylene glycol monomer with the TIC (top) and the corresponding mass spectra (bottom). Calculated mass [M+H] 228.16, observed mass [M+H] 228.20; [M+Na] 250.10.

FIG. 30 shows positive mode LCMS of BDT-P with the TIC (top) and the corresponding mass spectra (bottom). Calculated mass [M+H] 1454.77, observed mass [M+H] 1454.60; [M+2H] 727.80.

FIG. 31 shows positive mode LCMS of PDT-P with the TIC (top) and the corresponding mass spectra (bottom). Calculated mass [M+H] 1398.70, observed mass [M+H]; [M+2H].

FIG. 32 shows positive mode LCMS of DTT-P with the absorbance at 210 nm (top) and the corresponding mass spectra (bottom). Calculated mass [M+H] 1582.73, observed mass [M+H] 1583.30; [M+2H] 792.30; [M+3H] 528.60.

FIG. 33 shows positive mode LCMS of fluorescein-BDT-P with the absorbance at 230 nm (top) and the corresponding mass spectra (bottom). Calculated mass [M+H] 1812.81, observed mass [M+2H] 906.90.

FIG. 34 shows positive mode LCMS of fluorescein-PDT-P with the absorbance at 230 nm (top) and the corresponding mass spectra (bottom). Calculated mass [M+H] 1756.75, observed mass [M+2H] 878.90.

FIG. 35 shows positive mode LCMS of fluorescein-DTT-P with the absorbance at 230 nm (top) and the corresponding mass spectra (bottom). Calculated mass [M+H] 1940.77, observed mass [M+2H] 970.90; [M+3H] 647.90.

FIG. 36 shows negative mode MALDI of BODIPY-Vancomycin-BDT-P. Calculated mass [M+H] 3158.29, observed mass [M+H] 3158.34.

FIG. 37 shows HPLC traces showing the retention times of purified BDT-P, PDT-P, DTT-P, and PEO⁴-B. Peaks from left to right: DTT-P; PDT-P; BDT-P; PEO⁴-B; PEO²-B.

FIG. 38 shows solubility testing of BDT-P, PEO²-B, and PEO⁴-B.

FIG. 39 shows cellular uptake of fluorescein-BDT-P, fluorescein-PEO²-B, and fluorescein-PEO⁴-B in HeLa cells, measured by flow cytometry. All cells were treated with 5 μM of fluorescein conjugates for 1 hour and measured at voltage 400.

FIG. 40 shows cytotoxicity of R9, BDT-P, and fluorescein-BDT-P in (A) HeLa and SKOV-3 cells via MTS assay and (B) red blood cells via hemolysis assay. Cells were treated with 1-160 μM of R9 and BDT-P or 1-20 μM of fluorescein-BDT-P for 1 hour at 37° C.

FIG. 41 shows dose-dependent uptake of fluorescein-BDT-P in HeLa and SKOV-3 cells. All cells were treated with fluorescein conjugates for 1 hour and measured at voltage 400.

FIG. 42 shows cellular uptake of BODIPY-Vancomycin and BODIPY-Vancomycin-BDT-P in HeLa cells, measured by flow cytometry. All cells were treated with 1 μM of BODIPY conjugates for 1 hour and measured at voltage 450. Peaks from left to right: Cells only; BODIPY-Vancomycin; BODIPY-Vancomycin-BDT-P.

FIG. 43 shows cellular uptake of BODIPY-Vancomycin and BODIPY-Vancomycin-BDT-P in HeLa cells, measured by flow cytometry. All cells were treated with 0.5 or 2.5 μM of BODIPY conjugates for 1 hour and measured at voltage 450. Peaks from left to right: Cells only; 0.5 μM BODIPY-Vancomycin; 2.5 μM BODIPY-Vancomycin; 0.5 μM BODIPY-Vancomycin-BDT-P; 2.5 μM BODIPY-Vancomycin-BDT-P.

FIG. 44 shows cellular uptake in HeLa cells at various time points post treatment with R9-fluorescein and fluorescein-BDT-P, measured by flow cytometry All cells were treated with 5 μM of fluorescein conjugates for 1 hour and measured at voltage 400.

FIG. 45 shows cellular uptake of fluorescein-BDT-P, fluorescein-PDT-P, fluorescein-DTT-P, and fluorescein-PEO⁴-B in HeLa cells, measured by flow cytometry. All cells were treated with 5 μM of fluorescein conjugates for 1 hour and measured at voltage 400.

FIG. 46 shows positive ion mode LCMS of (PEG-BDT)₂-4Bu with the corresponding mass spectra; HPLC trace indicating purity is shown in FIG. 51. Calculated mass [M+H] 1334.72, observed mass [M+H] 1334.60; [M+2H] 668.13.

FIG. 47 shows positive ion mode LCMS of (PEG-Bu)₄ with the corresponding mass spectra; HPLC trace indicating purity is shown in FIG. 51. Calculated mass [M+H] 1454.77, observed mass [M+H] 1454.50; [M+2H] 727.84.

FIG. 48 shows positive ion mode LCMS of (BDT-PEG)₄ with the TIC (top) and the corresponding mass spectra (bottom); HPLC trace indicating purity is shown in FIG. 51. Calculated mass [M+H] 1454.77, observed mass [M+H] 1454.60; [M+2H] 727.80.

FIG. 49 shows positive ion mode LCMS of (PEG-PEG)₄ with the TIC (top) and the corresponding mass spectra (bottom); HPLC trace indicating purity is shown in FIG. 51. Calculated mass [M+H] 1694.85, observed mass [M+2H] 848.13.

FIG. 50 shows positive ion mode LCMS of (BDT-PEG₄)₄ with the TIC (top) and the corresponding mass spectra (bottom); HPLC trace indicating purity is shown in FIG. 51. Calculated mass [M+H] 1750.91, observed mass [M+2H] 876.83.

FIG. 51 shows HPLC traces showing the retention times of purified oligoTEAs.

FIG. 52 shows solubility of selective oligoTEAs in 1×PBS at pH 7.4. The hazy point is the point at which a faint cloudiness is observed and corresponds to an A600 of ˜0.05.

FIG. 53 shows positive ion mode LCMS of fluorescein-(PEG-BDT)₂-4Bu with the absorbance at 230 nm (top) and the corresponding mass spectra (bottom). Calculated mass [M+H] 1692.77, observed mass [M+2H] 846.70; [M+3H] 564.90.

FIG. 54 shows positive ion mode LCMS of fluorescein-(PEG-Bu)₄ ((PEG-Bu)₄ may be referred to as PEO⁴-B) with the absorbance at 230 nm (top) and the corresponding mass spectra (bottom). Calculated mass [M+H] 1812.81, observed mass [M+2H] 906.90.

FIG. 55 shows positive ion mode LCMS of fluorescein-(BDT-PEG)₄ with the absorbance at 230 nm (top) and the corresponding mass spectra (bottom). Calculated mass [M+H] 1812.81, observed mass [M+2H] 906.90; [M+3H] 605.00.

FIG. 56 shows positive ion mode LCMS of fluorescein-(PEG-PEG)₄ with the absorbance at 230 nm (top) and the corresponding mass spectra (bottom). Calculated mass [M+H] 2052.90, observed mass [M+2H] 1027.60; [M+3H] 685.90.

FIG. 57 shows positive ion mode LCMS of fluorescein-(BDT-PEG₄)₄ with the absorbance at 230 nm (top) and the corresponding mass spectra (bottom). Calculated mass [M+H] 2108.96, observed mass [M+2H] 1055.50; [M+3H] 704.3.

FIG. 58 shows HPLC purification of BODIPY-Vancomycin and (BDT-PEG)₄ reaction. Two peaks, denoted as P1 and P2, were collected and checked by MALDI-MS.

FIG. 59 shows MALD-MS spectrum of BODIPY-Vancomycin-(BDT-PEG)₄ P1 in negative ion mode.

FIG. 60 shows MALDI-MS spectrum of BODIPY-Vancomycin-(BDT-PEG)₄ P2 in negative ion mode.

FIG. 61 shows HPLC trace of the reaction between Vancomycin-HCl and Linker-(PEG-Bu)₄.

FIG. 62 shows MALDI-MS of Vancomycin-SS-(PEG-Bu)₄ P1 collected from HPLC in positive ion mode.

FIG. 63 shows MALDI-MS of Vancomycin-SS-(PEG-Bu)₄ P2 collected from HPLC in positive ion mode.

FIG. 64 shows cellular uptake of fluorescein cargo by oligoTEAs in J774 cells measured by flow cytometry. Cells were treated with 5 μM of fluorescein-oligoTEA conjugates for 1 hr.

FIG. 65 shows cellular uptake of fluorescein cargo by oligoTEAs in MC-3T3-E1 cells measured by flow cytometry. Cells were treated with 5 μM of fluorescein-oligoTEA conjugates for 1 hr.

FIG. 66 shows cellular uptake of fluorescein cargo by oligoTEAs in A549 cells measured by flow cytometry. Cells were treated with 5 μM of fluorescein-oligoTEA conjugates for 1 hr.

FIG. 67 shows cellular uptake of BODIPY-Vancomycin-(BDT-PEG)₄ P1 and P2 in HeLa cells measured by flow cytometry. Cells were treated with 1 μM of fluorescein-oligoTEA conjugates for 1 hr.

FIG. 68 shows cellular uptake of BODIPY-Vancomycin-(BDT-PEG)₄ P1 in HeLa cells measured by flow cytometry. Cells were treated with 0.5 and 2.5 μM of fluorescein-oligoTEA conjugates for 1 hr.

FIG. 69 shows cellular uptake of BODIPY-Vancomycin-(BDT-PEG)₄ P2 in HeLa cells measured by flow cytometry. Cells were treated with 0.5 μM of fluorescein-oligoTEA conjugates for 1 hr.

FIG. 70 shows cytotoxicity of vancomycin-SS-oligoTEA conjugates in J774 cells via MTS assay. Cells were treated with 10-50 μM of compounds for 4 hours at 37° C.

FIG. 71 shows cytotoxicity of vancomycin-SS-oligoTEA conjugates in J774 cells via MTS assay. Cells were treated with 15-120 μM of compounds for 4 hours at 37° C.

FIG. 72 shows (A) 14^{th} hr data points of the cell growth kinetics obtained from the in vitro intracellular infection assays and (B) Cell growth curves of all samples. J774 cells infected with Listeria monocytogenes DP-L1942 (MOI=2) were treated with all compounds for 4 hrs at 37° C. Vancomycin and ciprofloxacin were tested at 30 μM.

FIG. 73 shows percentage of reduction in bacteria growth. Data were taken at the 14^{th} hour time point from the bacteria growth curve and normalized to infected cells with no treatment (100%) and uninfected cells (0%).

FIG. 74 shows EICs of vancomycin, vancomycin-SH, and the full conjugates of P1 and P2 from the LCMS spectra at each time point of the cleavage using DL-DTT at 37° C. in 1×PBS at pH 7.4.

FIG. 75 shows mean count rate as a function of sample concentration (0.25 to 15 μM) of vancomycin-SS-(PEG-Bu)₄ P1 in water. Attenuator was fixed at 8.

FIG. 76 shows mean count rate as a function of sample concentration (0.25 to 15 μM) of vancomycin-SS-(PEG-Bu)₄ P2 in water. Attenuator was fixed at 8.

FIG. 77 shows synthetic scheme for the assembly of peptide-PEG₄-oligoTEA conjugates. Reaction conditions: (i) 5 eq Mal-PEG₄-NHS, 10 eq triethylamine, 1 h, r.t., (ii) 2 eq Mal-PEG₄-oligoTEA, 10 eq N,N-Diisopropylethylamine, 9 mM in 1:1 DMSO:DMF, 24 h, 37° C.

FIG. 78 shows confocal microscopic images of fixed HeLa cells treated with 5 μM peptide-(PEG-Bu)₄, and peptide-(Bu-PEG)₄ conjugates. Images were taken with a 40× water objective on the Zeiss inverted 880 microscope.

FIG. 79 shows RP-HPLC trace of the reaction between HA peptide and Mal-PEG₄-(PEO⁴-B). The product is highlighted by the two gray lines.

FIG. 80 shows LC-MS spectrum of peptide-PEG₄-(PEG-Bu)₄.

FIG. 81 shows RP-HPLC trace of the reaction between HA peptide and Mal-PEG₄-(BDT⁴-P). The product is highlighted by the two gray lines.

FIG. 82 shows LC-MS spectrum of Peptide-PEG₄-(Bu-PEG)₄.

FIG. 83 shows synthetic methodology utilized for the assembly of sequence-defined oligoTEAs. OligoTEAs are assembled with a series of iterative thiol-ene and thiol-Michael additions interspersed with fluorous solid phase purifications. DTT: dithiothreitol, BDT: 1,4-butane-dithiol, PDT: 1,3-propane-dithiol, PEO: polyethylene oxide/polyethylene glycol. Monomer shown containing PEO is 3,6-dioxa-1,8-octanedithiol. DTT-G: polymer with DTT incorporated into R group with guanidinium side chains.

FIG. 84 shows cleavage of a cleavable linking group.

FIG. 85 shows non-limiting examples of compounds of the present disclosure.

FIG. 85 shows a synthetic scheme to synthesize a compound of the present disclosure having two cargo groups.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out an example of a lower limit value and an example of an upper limit value. Unless otherwise stated, the ranges include all values to the magnitude of the smallest value (either lower limit value or upper limit value) and ranges between the values of the stated range.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). Illustrative examples of groups include:

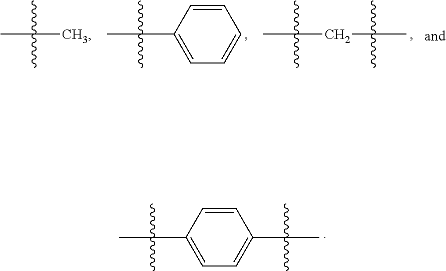

As used herein, unless otherwise indicated, the term “aliphatic” refers to branched or unbranched hydrocarbon groups that, optionally, contain one or more degrees of unsaturation. Degrees of unsaturation include, but are not limited to, alkenyl groups, alkynyl groups, and aliphatic cyclic groups. For example, the aliphatic groups are a C₁ to C₂₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). The aliphatic group may be unsubstituted or substituted with one or more substituent. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof. Groups that are aliphatic may be alkyl groups, alkenyl groups, alkynyl groups, or carbocyclic groups, and the like.

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group is C₁ to C₂₀, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). The alkyl group may be unsubstituted or substituted with one or more substituent. Examples of substituents include, but are not limited to, various substituents such as, for example, halogens (—F, —Cl, —Br, and —I), aliphatic groups (e.g., alkyl groups, alkenyl groups, alkynyl groups, and the like), aryl groups, alkoxide groups, carboxylate groups, carboxylic acids, ether groups, amine groups, and the like, and combinations thereof.

As used herein, unless otherwise indicated, the term “alkenyl group” refers to branched or unbranched unsaturated hydrocarbon groups comprising at least one carbon-carbon double bond. Examples of alkenyl groups include, but are not limited to, ethylene groups, propenyl groups, butenyl groups, isopropenyl groups, tert-butenyl groups, and the like. For example, the alkenyl group is C₁ to C₂₀, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). The alkenyl group may be unsubstituted or substituted with one or more substituent. Examples of substituents include, but are not limited to, various substituents such as, for example, halogens (—F, —Cl, —Br, and —I), aliphatic groups (e.g., alkyl groups, alkenyl groups, alkynyl groups, and the like), aryl groups, alkoxide groups, carboxylate groups, carboxylic acids, ether groups, amine groups, and the like, and combinations thereof.

As used herein, unless otherwise indicated, the term “alkynyl group” refers to branched or unbranched unsaturated hydrocarbon groups comprising at least one carbon-carbon triple bond. Examples of alkynyl groups include, but are not limited to, groups, ethynyl groups, propynyl groups, butynyl groups, and the like. For example, the alkynyl group is C₁ to C₂₀, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). The alkynyl group may be unsubstituted or substituted with one or more substituent. Examples of substituents include, but are not limited to, various substituents such as, for example, halogens (—F, —Cl, —Br, and —I), aliphatic groups (e.g., alkyl groups, alkenyl groups, alkynyl groups, and the like), aryl groups, alkoxide groups, carboxylate groups, carboxylic acids, ether groups, amine groups, and the like, and combinations thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused ring, biaryl groups, or a combination thereof. The aryl group may be unsubstituted or substituted with one or more substituent. Examples of substituents include, but are not limited to, substituents such as, for example, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Aryl groups may contain hetero atoms, such as, for example, nitrogen (e.g., pyridinyl groups and the like). Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, the term “aliphatic cyclic group” refers to a cyclic compound having a ring where all of the atoms forming the ring are carbon atoms. The aliphatic cyclic group ring may be aromatic or nonaromatic, and include compounds that are saturated and partially unsaturated, and fully unsaturated. The aliphatic cyclic groups may be terminal aliphatic cyclic groups or aliphatic cyclic groups covalently bonded to two functional groups. Examples of such groups include cyclobutyl, cyclopentanyl, cyclopentenyl, cyclohexyl, cyclohexenyl, cyclohexanonyl, cyclopentanonyl, cyclopentanolyl, indanyl, indanonyl, phenyl, naphthyl and the like. For example, the aliphatic cyclic group ring is a C₄ to C₈ carbocyclic ring, including all integer numbers of carbons and ranges of numbers of carbons therebetween (e.g., C₄, C₅, C₆, C₇, C₈). The aliphatic cyclic group may be unsubstituted or substituted with groups such as, for example, alkyl chain(s), alkenyl chain(s), alkynyl chain(s), carbonyl group(s), halogen(s), and the like, and combinations thereof.

The present disclosure provides oligothioetheramides (oligoTEAs) that may undergo, for example, cellular entry across different cell lines with low cytotoxicity. OligoTEAs of the present disclosure may outperform a widely used CPP, R9 peptide. The oligoTEAs may be distinct from other CPPs and may be used for delivery of therapeutics (e.g., intracellular delivery). Also provided are uses of the oligoTEAs.

In an aspect, the present disclosure provides compounds. The compounds are oligothioetheramides (oligoTEAs). The compounds may comprise a cargo group. The compounds may be charged or uncharged.

In an example, a compound has the following structure:

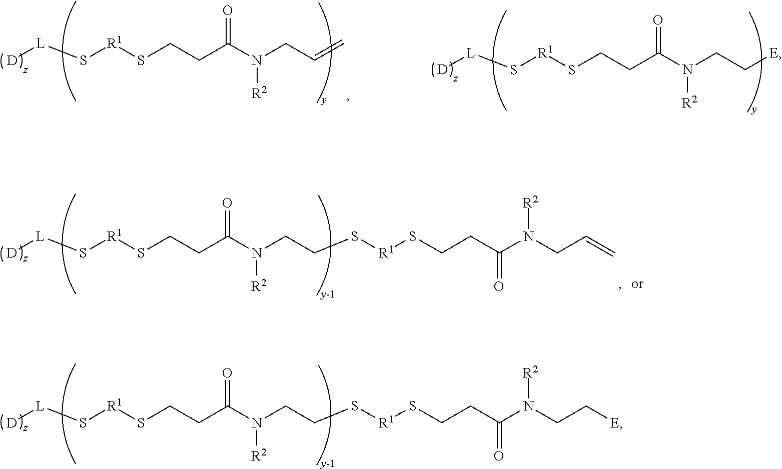

where L is chosen from a linking group, NH, N, O, and S. D is a cargo group. R1 is independently at each occurrence in the compound chosen from straight chain or branched C₂ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)ₐ—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8); diol groups having the structure —CH₂—CHOH—(CH₂)ₑ—CHOH—CH₂—, where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like). R² is independently at each occurrence in the compound chosen from cationic groups (e.g., alkyl amine groups, alkyl guanidinium groups, and the like), aliphatic electrophilic groups, aliphatic nucleophilic groups, straight chain or branched C₁ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)ₐ—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8); diol groups having the structure —CH₂—CHOH—(CH₂)ₑ—CHOH—CH₂—; where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like). E is an end group chosen from ═CH₂, D, and L-(D)_{z}. y is 1 to 12 (e.g., 1, 2, 3, 4, 5, 6, 7, 8, 9, 10, 11, 12). z is 1 to 5 (e.g., 1, 2, 3, 4, 5). In an example, a compound comprises at least two structurally distinct R¹ and/or at least two structurally distinct R².

In an example, a compound has the following structure:

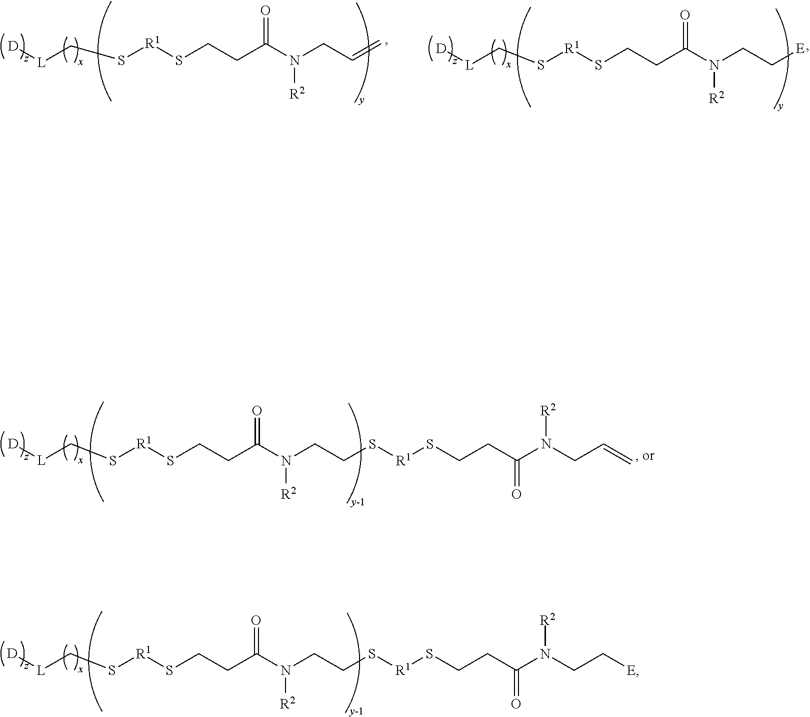

where L is chosen from a linking group, NH, N, O, and S. D is a cargo group. R¹ is independently at each occurrence in the compound chosen from straight chain or branched C₂ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)ₐ—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8); diol groups having the structure —CH₂—CHOH—(CH₂)ₑ—CHOH—CH₂—, where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like). R² is independently at each occurrence in the compound chosen from cationic groups (e.g., alkyl amine groups, alkyl guanidinium groups, and the like), aliphatic electrophilic groups, aliphatic nucleophilic groups, straight chain or branched C₁ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)ₐ—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8), diol groups having the structure —CH₂—CHOH—(CH₂)ₑ—CHOH—CH₂—; where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like). E is an end group chosen from ═CH₂, D, and L-(D)_{z}. x is 1 to 8 (e.g., 1, 2, 3, 4, 5, 6, 7, 8). y is 1 to 12 (e.g., 1, 2, 3, 4, 5, 6, 7, 8, 9, 10, 11, 12). z is 1 to 5 (e.g., 1, 2, 3, 4, 5). In an example, a compound comprises at least two structurally distinct R¹ and/or at least two structurally distinct R².

In an illustrative example, in a case where y=4, the first repeat unit y₁ may or may not equal the second repeat unit y₂, the third repeat unit y₃ and the fourth repeat unit y₄; and y₂ may or may not equal the third repeat unit y₃ and the fourth repeat unit y₄; and y₃ may or may not equal the fourth repeat unit y₄.

A compound may comprise various cargo groups. A cargo group may be formed from a cargo molecule. In an illustrative example, a cargo group may be formed from the reaction of a cargo molecule and a linking group, where the cargo molecule and linking group undergo conjugation chemistry (e.g., nucleophilic substitution), where, for example, the cargo molecule has a nucleophilic group/atom (e.g., a thiol group, an amine group, a hydroxyl group, sulfur atom, nitrogen atom, or oxygen atom) and the linking group has an electrophilic group (e.g., a carboxylic acid, ester, activated ester, and the like) or vice versa. For example, the nucleophilic group/atom undergoes a reaction with an electrophilic group, thus covalently bonding the cargo group to the linking group (e.g., an amine of vancomycin undergoes nucleophilic substitution with a carboxylic acid or activated ester of a linking group). Examples of conjugation chemistry (e.g., click chemistry, nucleophilic substitution, and the like) are known in the art. In an illustrative example, a cargo group may be formed from, for example, vancomycin. Non-limiting examples of a cargo groups include chemotherapeutic groups, antibiotic groups, fluorescent groups (e.g., fluorophore groups), peptide groups, protein groups, nucleic acid groups, kinase inhibitor groups (e.g., cobimetinib, erdafitinib, dasatinib, and the like), antibody groups, enzyme inhibitor groups, small molecule drug groups, sugar/glycan groups, and the like, and combinations thereof. In an example, the compound has a more than one cargo group. Non-limiting examples of cargo groups include a non-functionalized vancomycin group, a fluorophore-modified vancomycin group (e.g., BODIPY-functionalized vancomycin), a fluorescein group, an Atto 488 group

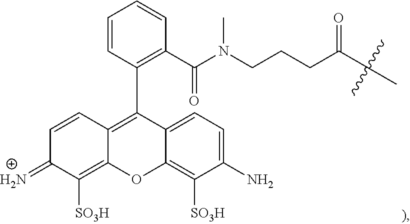 [SMILES: *C(=O)CCCN(C)C(=O)c1ccccc1-c1c2ccc(=[NH2+])c(S(=O)(=O)O)c-2oc2c(S(=O)(=O)O)c(N)ccc12]

a peptide group having the sequence: KADNAAIESIRNGTYDHDVYRDEALNNRFQIKGVELKSGYKDW (SEQ ID NO:1), and the like, and combinations thereof.

Examples of antibiotics from which antibiotic groups may be formed include, but are not limited to:

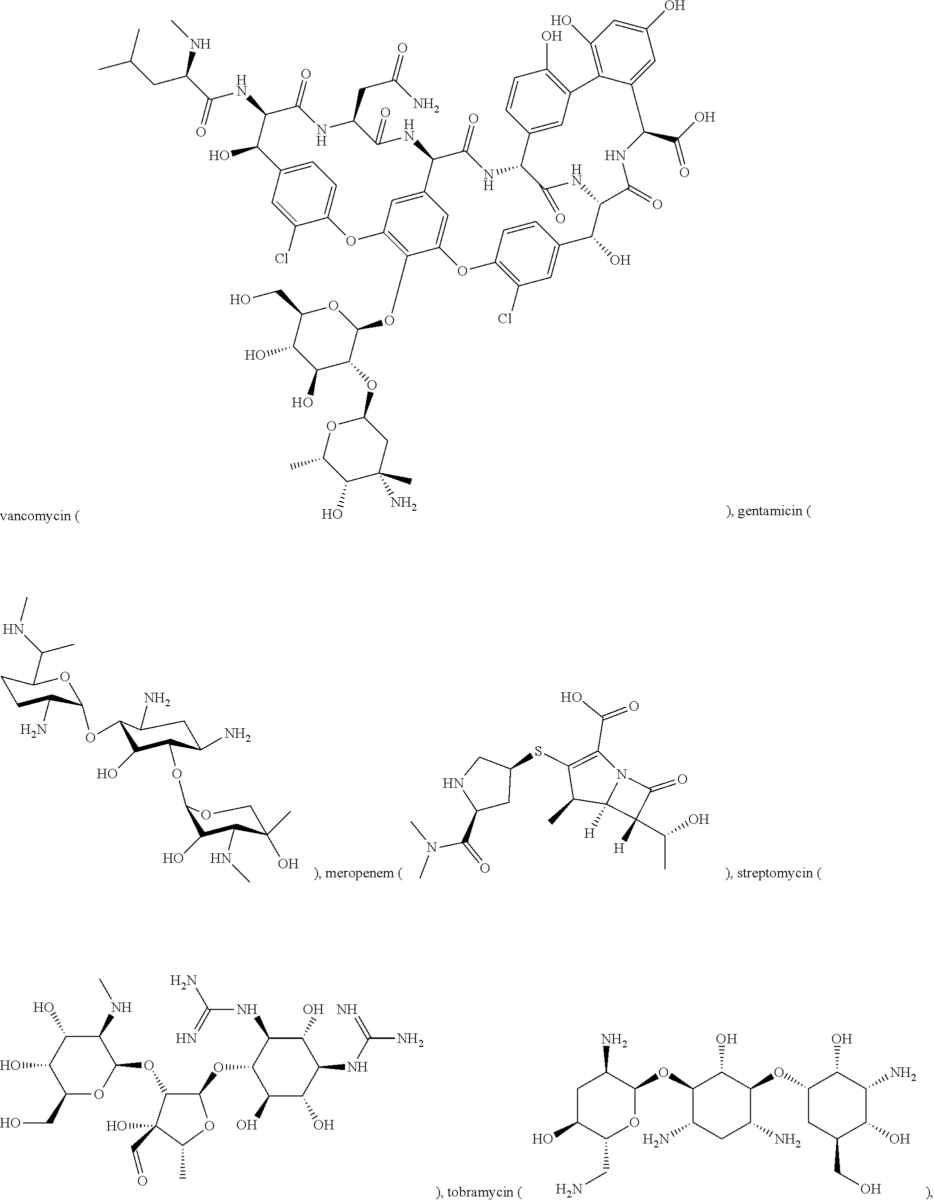

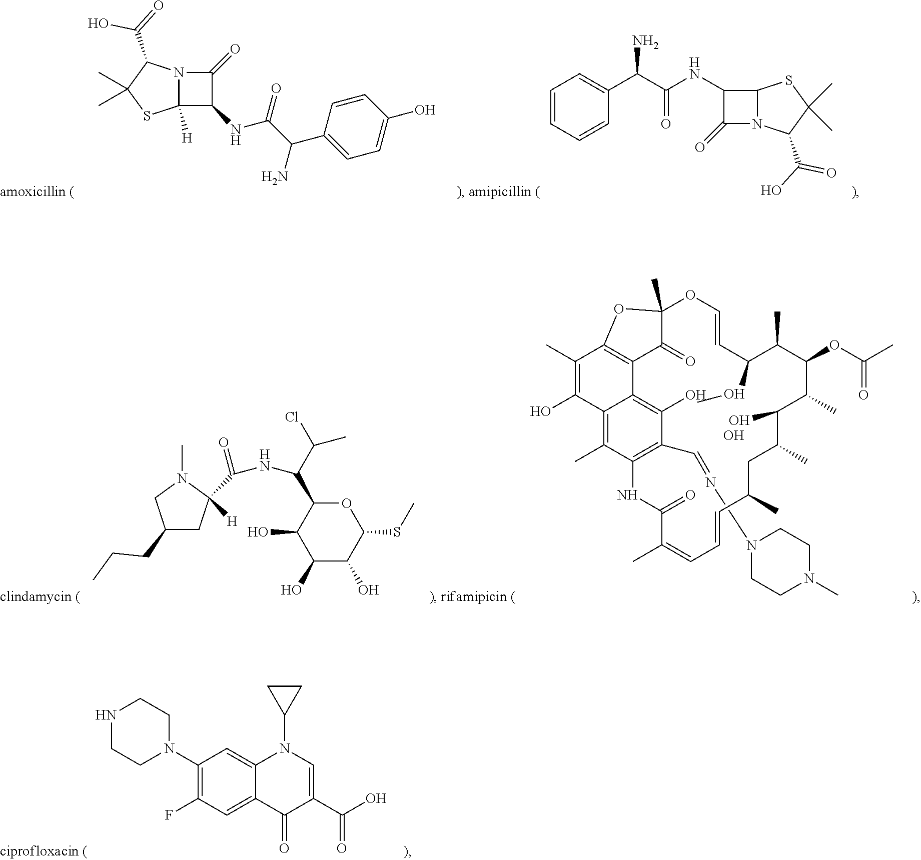

and the like.

Examples of chemotherapeutics from which chemotherapeutic groups can be formed include, but are not limited to:

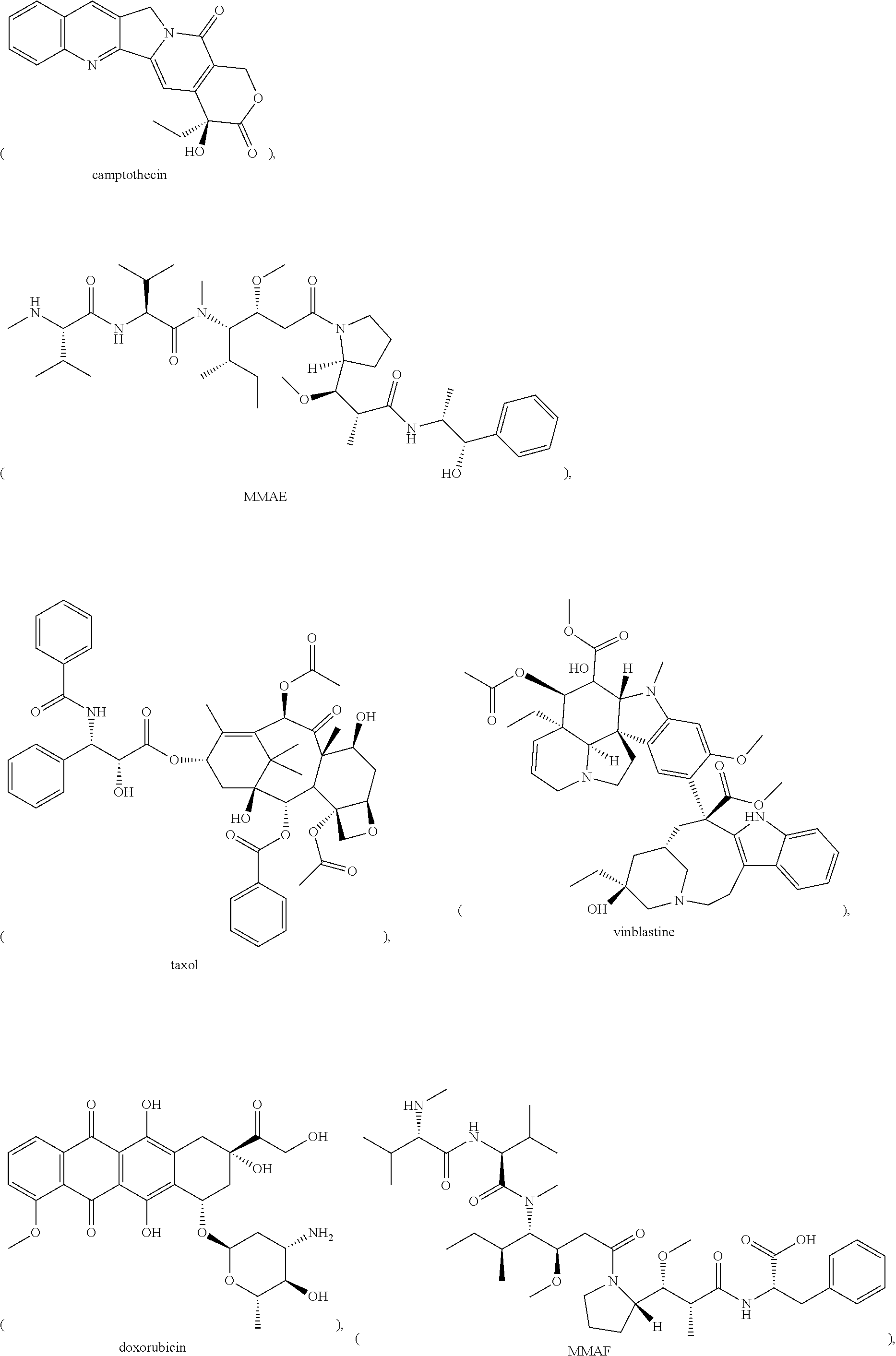

and the like.

Examples of antifungals from which antifungal groups can be formed include, but are not limited to:

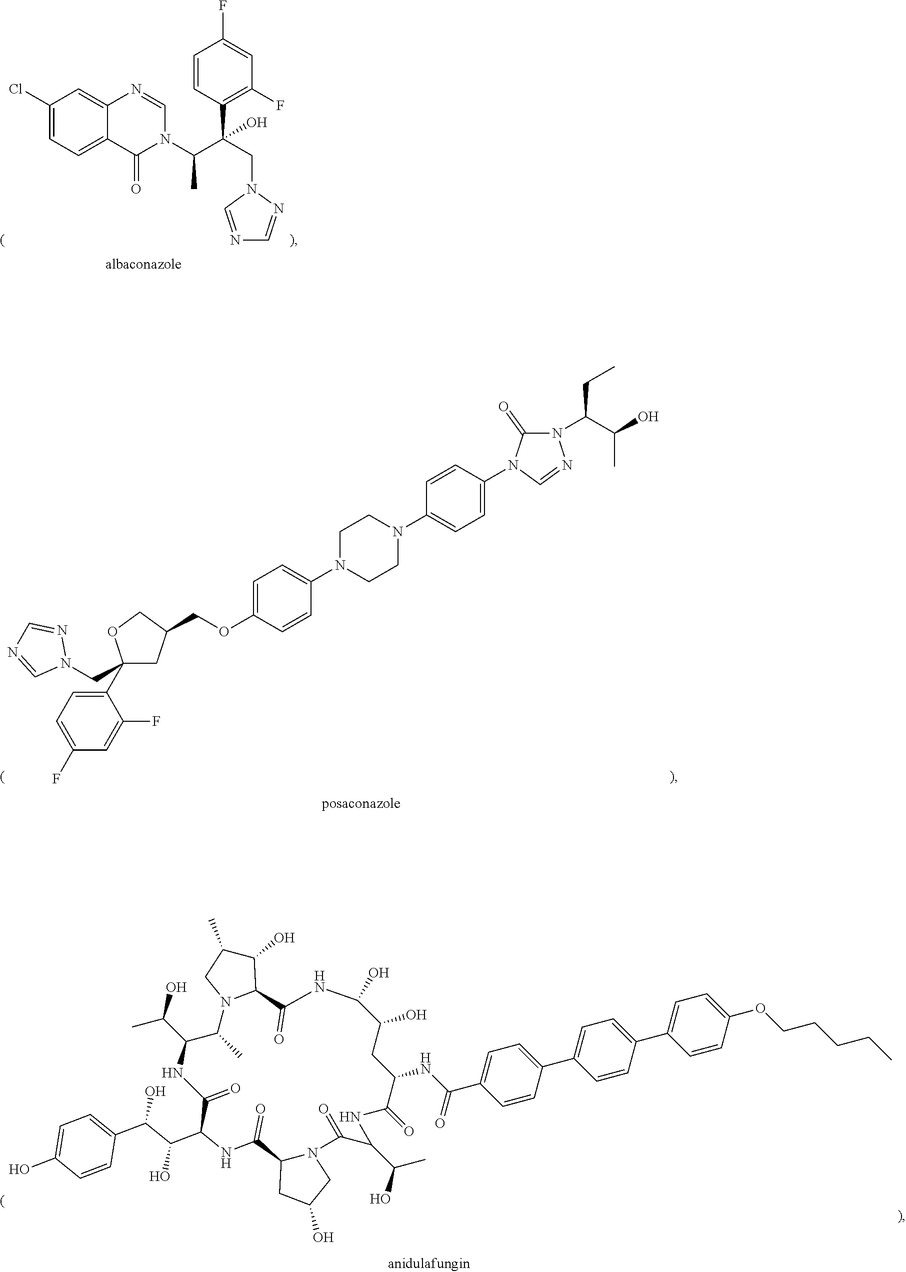 [SMILES: C[C@@H](n1cnc2cc(Cl)ccc2c1=O)[C@](O)(Cn1cncn1)c1ccc(F)cc1F ; CC[C@@H]([C@H](C)O)n1ncn(-c2ccc(N3CCN(c4ccc(OC[C@@H]5CO[C@@](Cn6cncn6)(c6ccc(F)cc6F)C5)cc4)CC3)cc2)c1=O ; CCCCCOc1ccc(-c2ccc(-c3ccc(C(=O)N[C@H]4C[C@@H](O)[C@@H](O)NC(=O)[C@@H]5[C@@H](O)[C@@H](C)CN5[C@H](C)[C@H]([C@@H](C)O)NC(=O)[C@H]([C@H](O)[C@@H](O)c5ccc(O)cc5)NC(=O)[C@@H]5C[C@@H](O)CN5C(=O)C([C@@H](C)O)NC4=O)cc3)cc2)cc1]

and the like.

Examples of kinase inhibitors from which kinase inhibitor groups may be formed from include, but are not limited to:

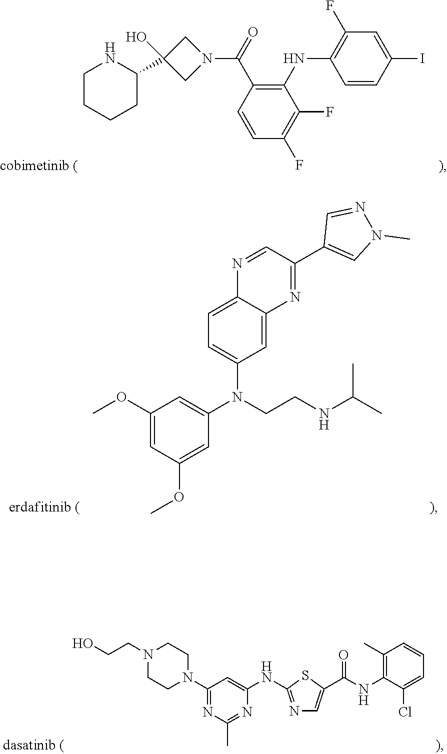

and the like.

A compound may comprise various R¹ groups. R¹ groups may be independently at each occurrence in the compound chosen from straight chain or branched C₂ to C₂₀ alkyl groups; straight chain or branched C₂ to C₂₀ alkenyl groups; straight chain or branched C₂ to C₂₀ alkynyl groups; polyether groups (which may be referred to as PEG or PEO groups) having the structure —(CH₂)_{b}—[—O—CH₂—CH₂—]ₐ—O—(CH₂)ₐ—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8); diol groups having the structure —CH₂—CHOH—(CH₂)ₑ—CHOH—CH₂—, where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like). Each R¹ group may be independently at each occurrence in the compound chosen from substituted or unsubstituted propyl groups, substituted or unsubstituted butyl groups,

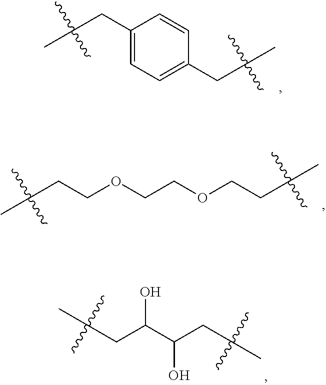 [SMILES: *Cc1ccc(C*)cc1 ; *CCOCCOCC* ; *CC(O)C(O)C*]

and the like, and combinations thereof.

A compound may comprise various R² groups. R² groups may be independently at each occurrence in the compound chosen from cationic groups (e.g., alkyl amine groups, alkyl guanidinium groups, and the like), aliphatic electrophilic groups, aliphatic nucleophilic groups, straight chain or branched C₁ to C₂₀ alkyl groups; straight chain or branched C₂ to C₂₀ alkenyl groups; straight chain or branched C₂ to C₂₀ alkynyl groups; polyether groups (which may be referred to as PEG or PEO groups) having the structure —(CH₂)_{b}—[—O—CH₂—CH₂—]ₐ—O—(CH₂)ₐ—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8), diol groups having the structure —CH₂—CHOH—(CH₂)ₑ—CHOH—CH₂—; where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like). R² groups may comprise a nucleophilic group (e.g., an amine, thiol, and the like) or electrophilic group (e.g., an activated ester, carboxylic acid, and the like) that may be used to form a cargo group from a cargo molecule. Each R² may be independently at each occurrence in the compound chosen from substituted or unsubstituted butyl groups, substituted or unsubstituted benzyl groups,

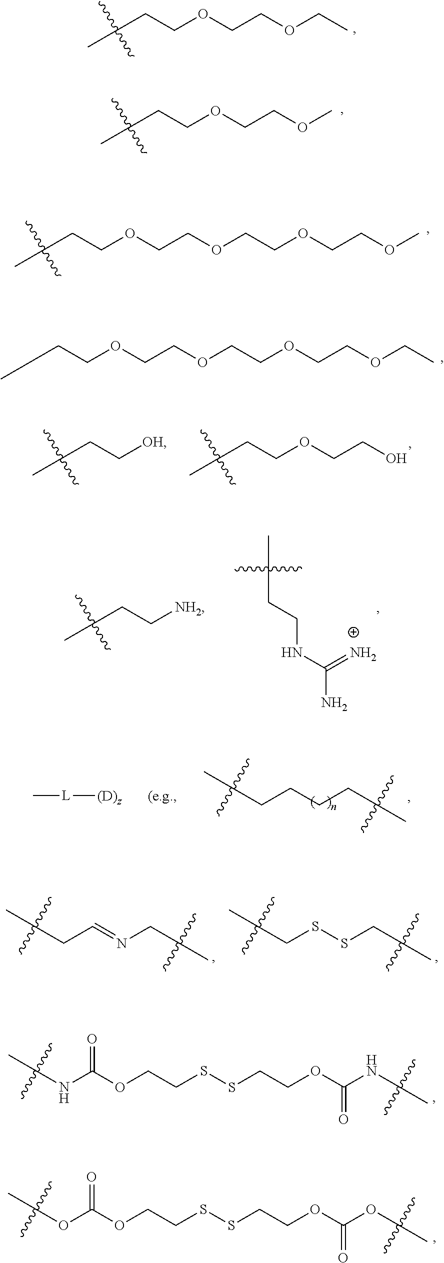

and the like, and combinations thereof, where a termini of R² is bonded to cargo group (D)).

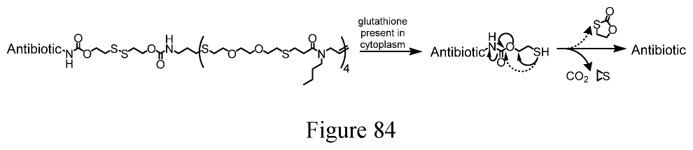

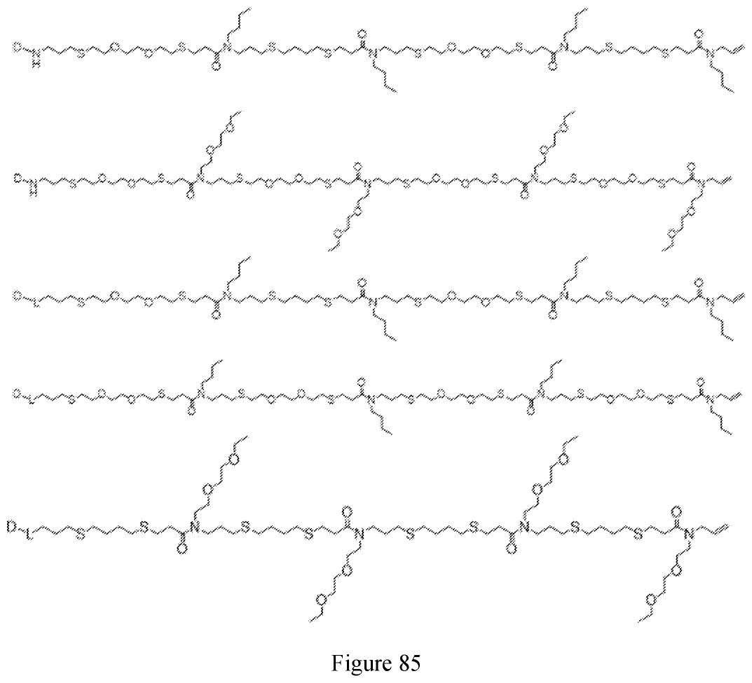

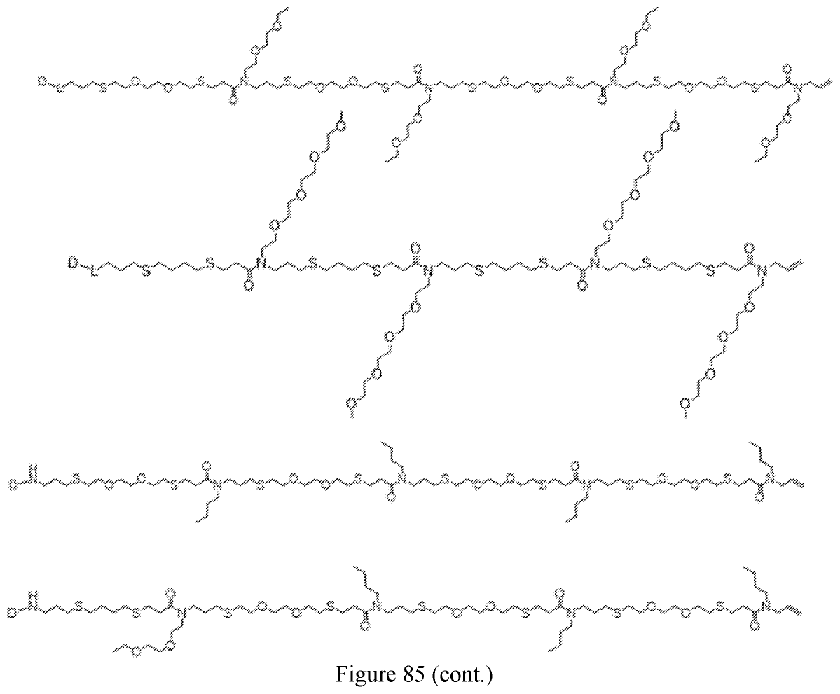

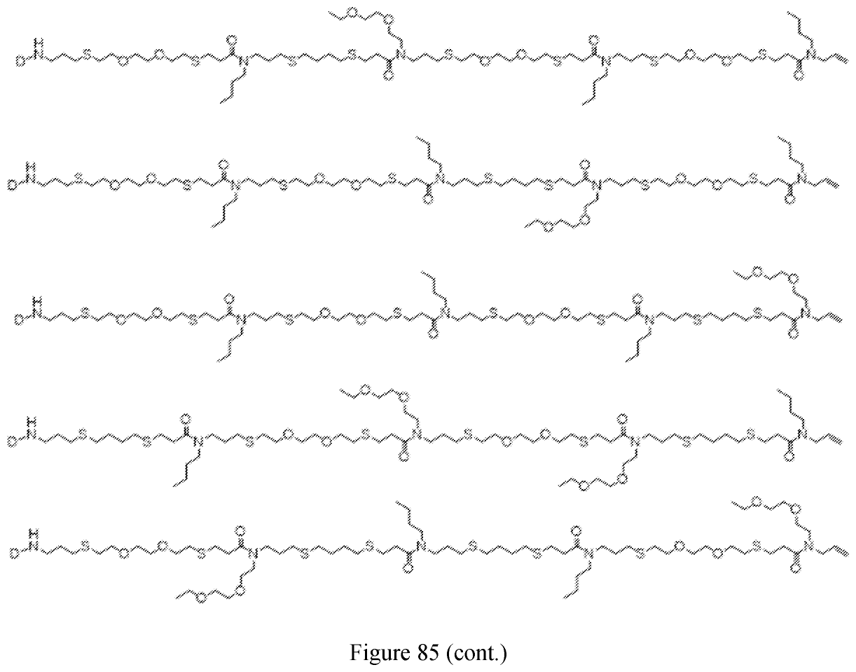

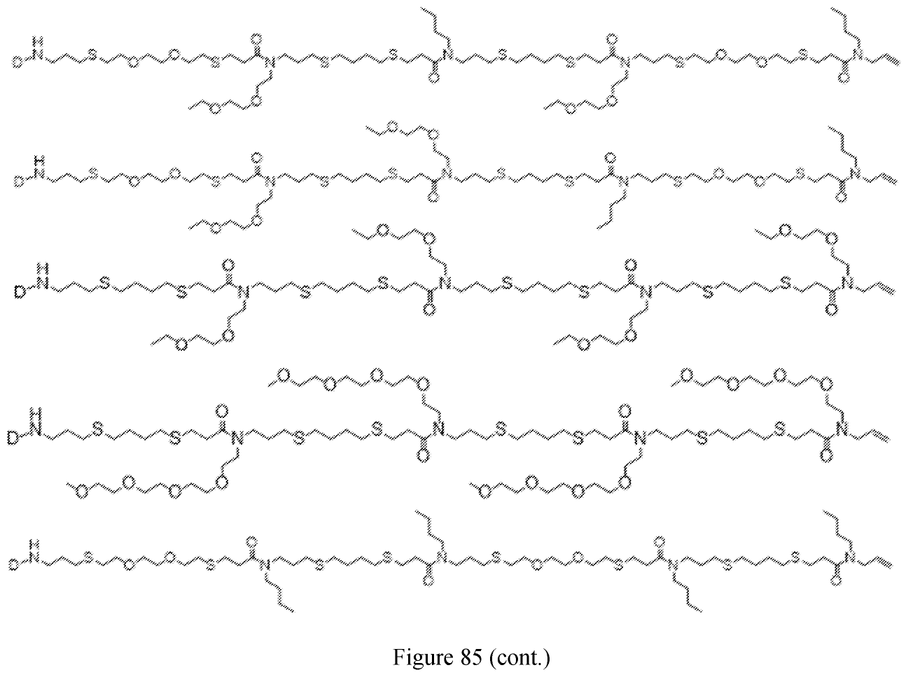

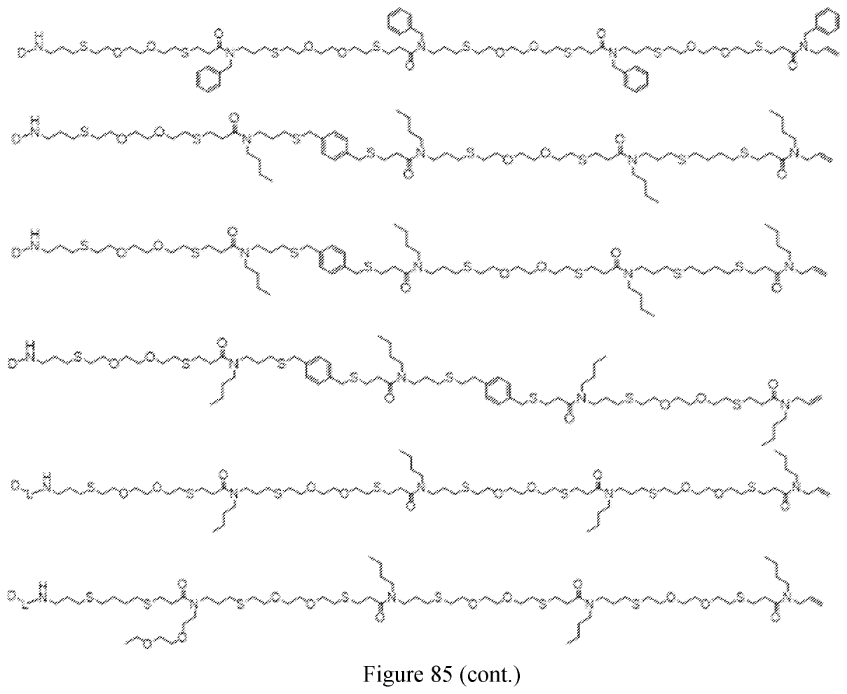

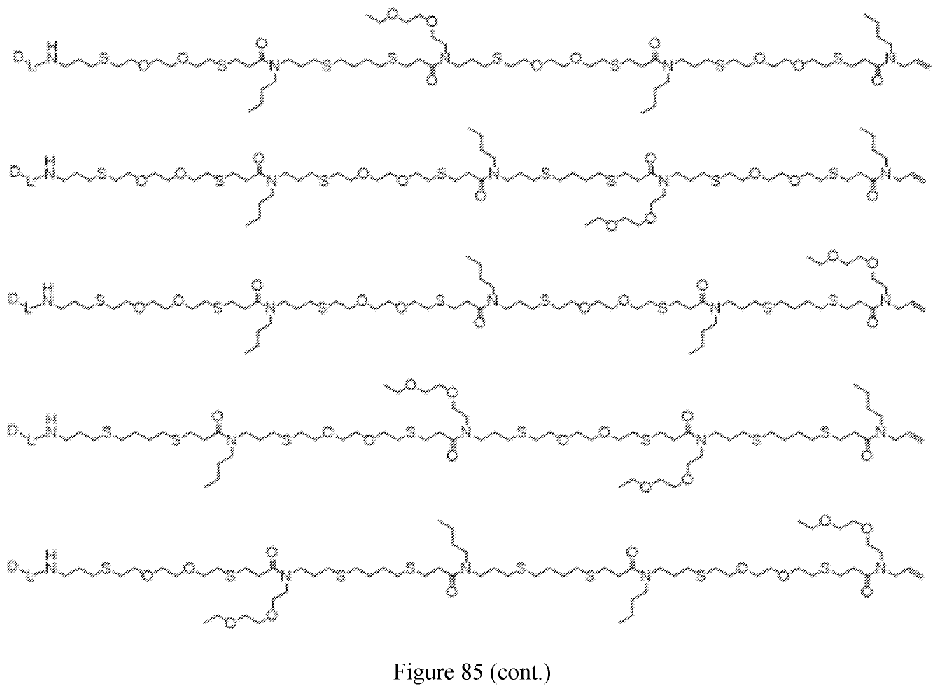

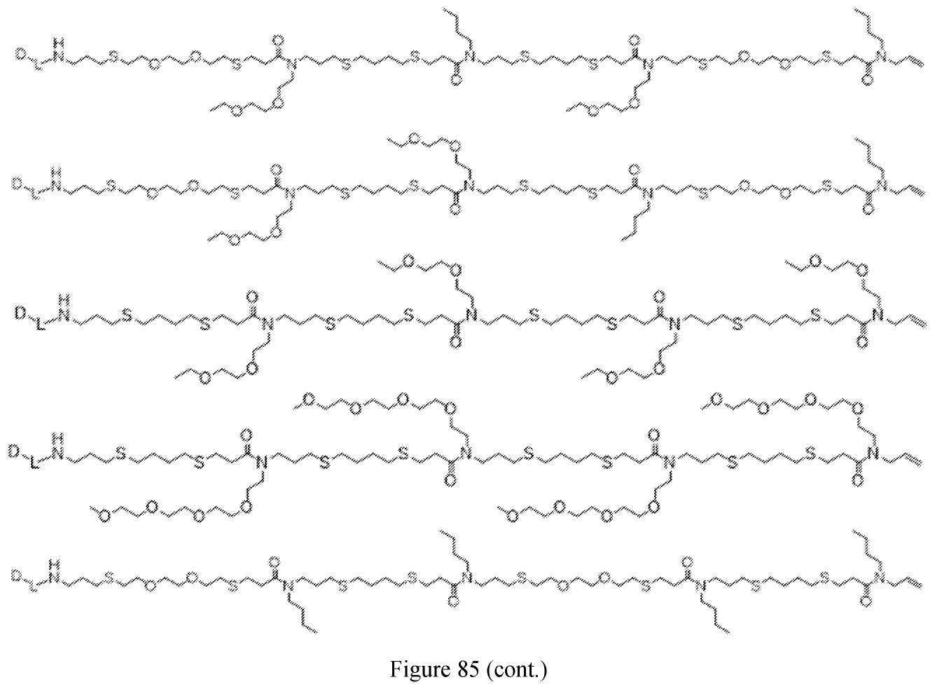

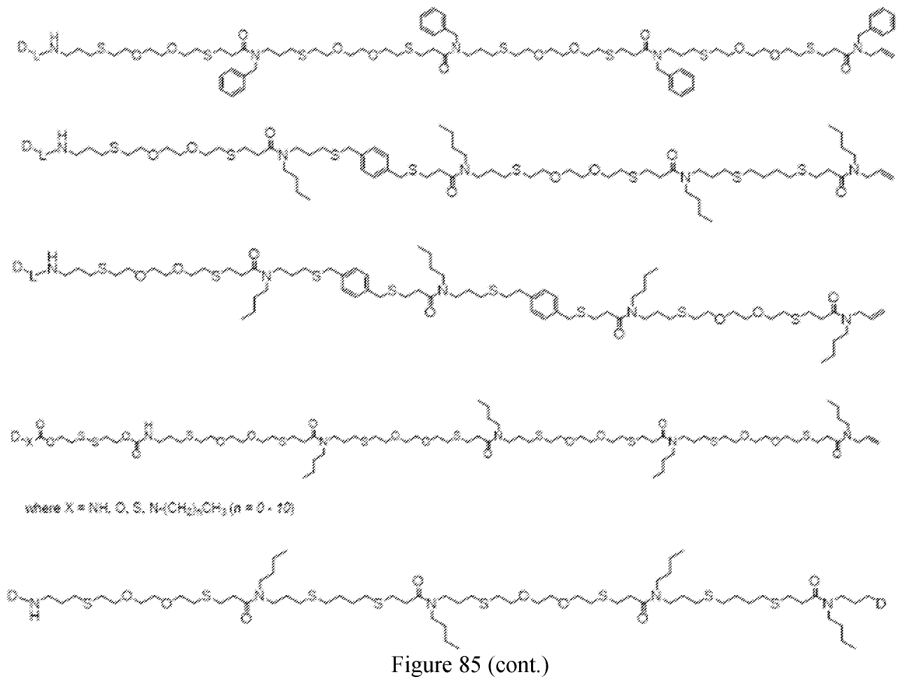

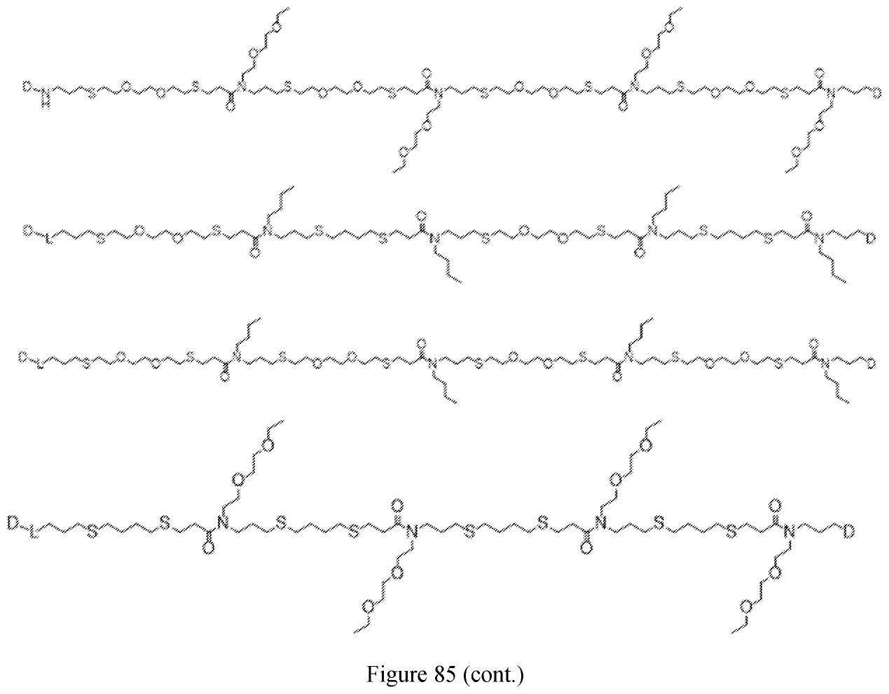

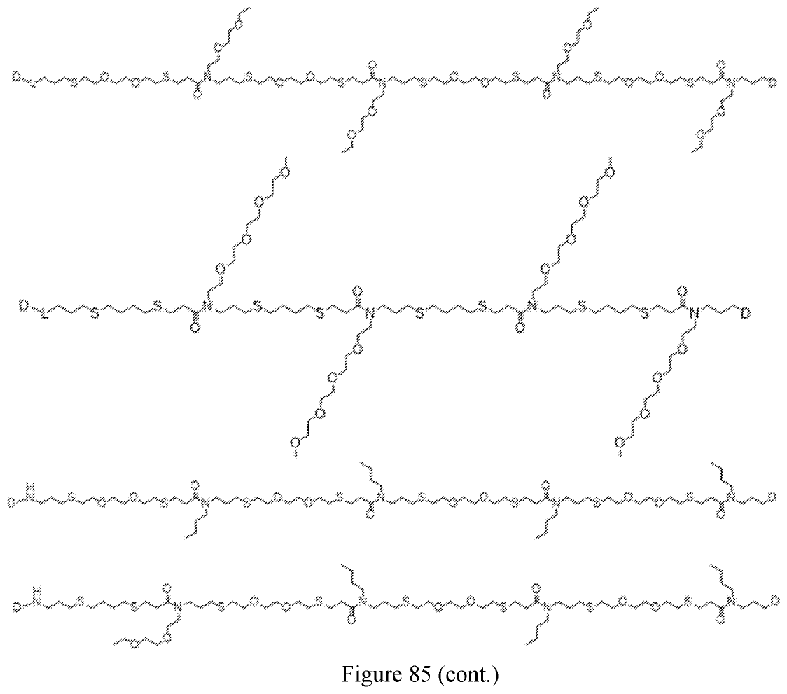

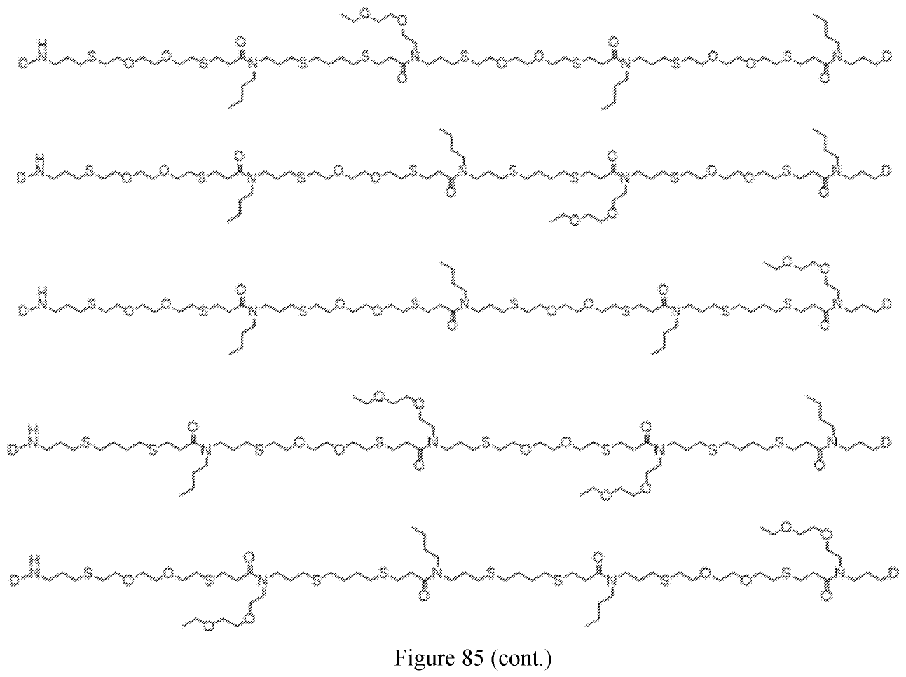

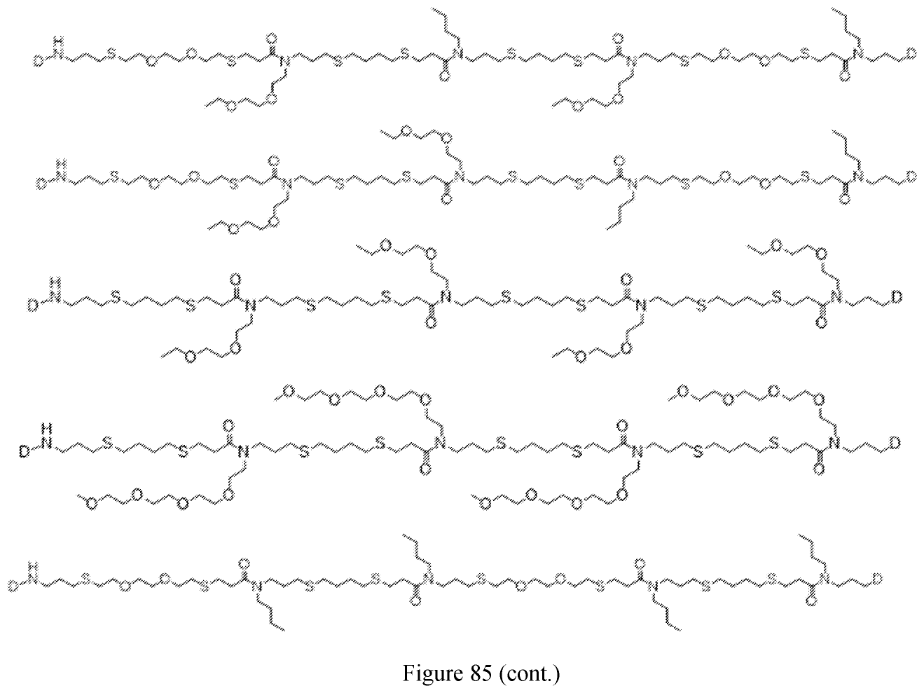

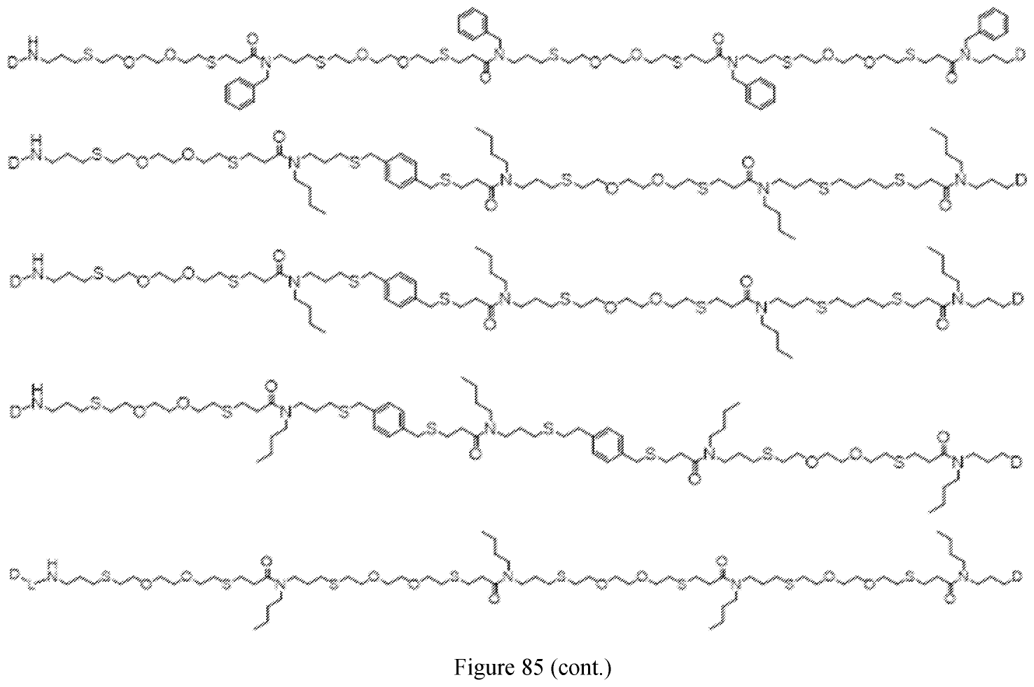

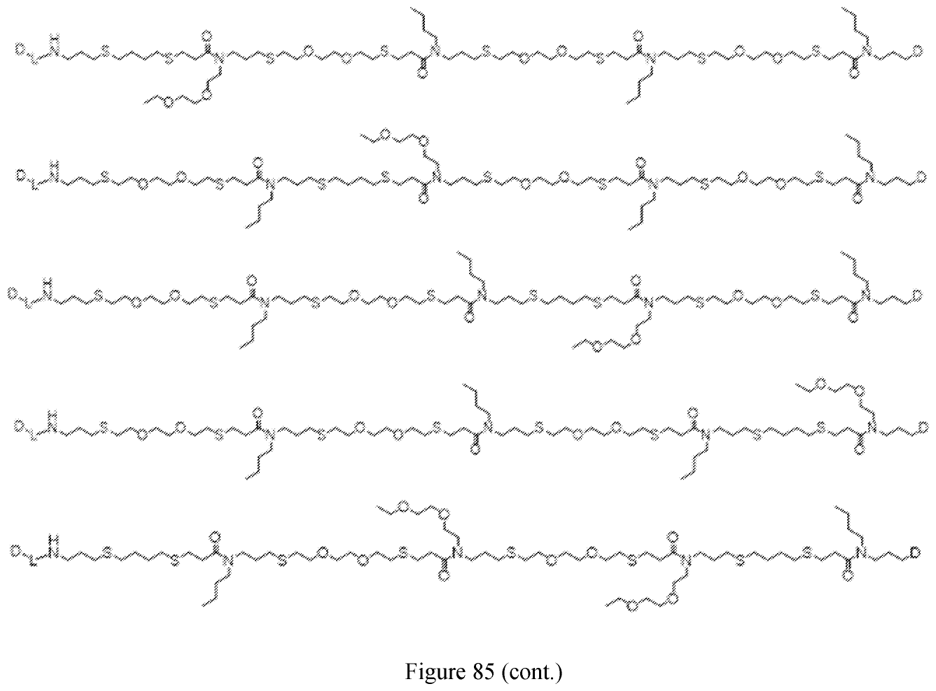

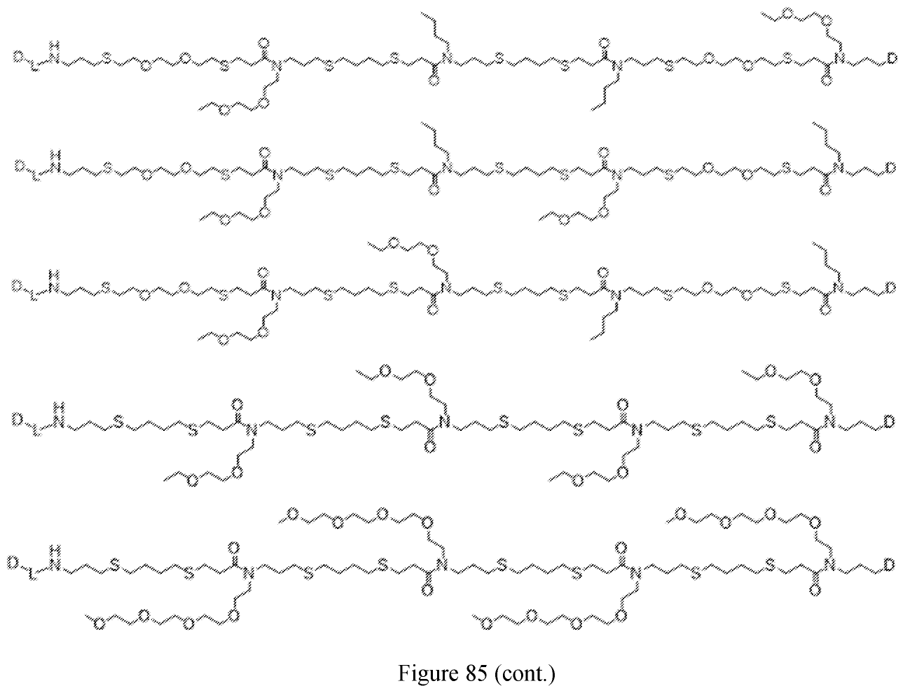

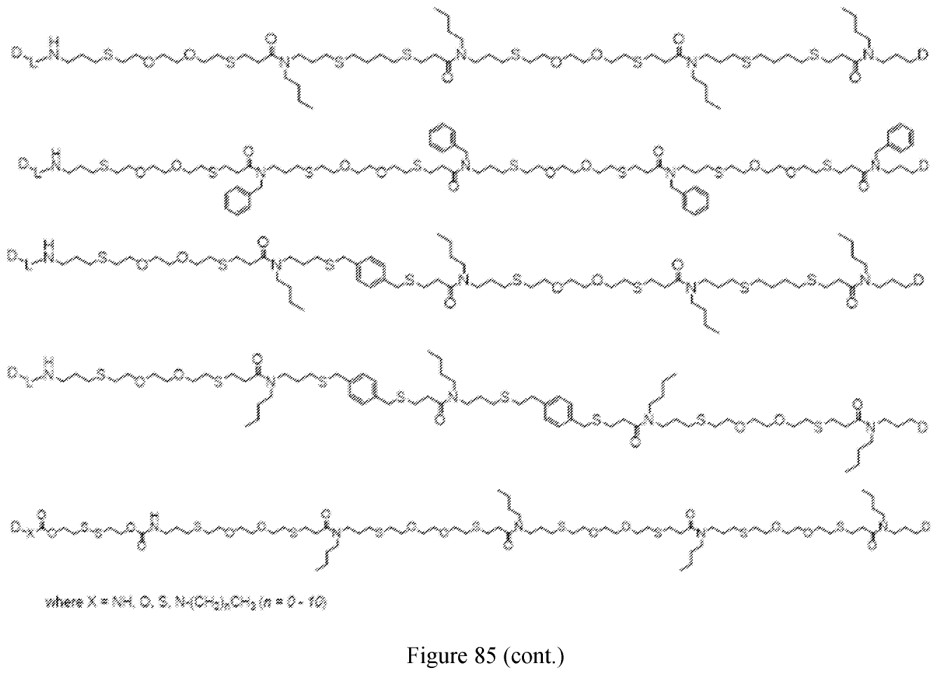

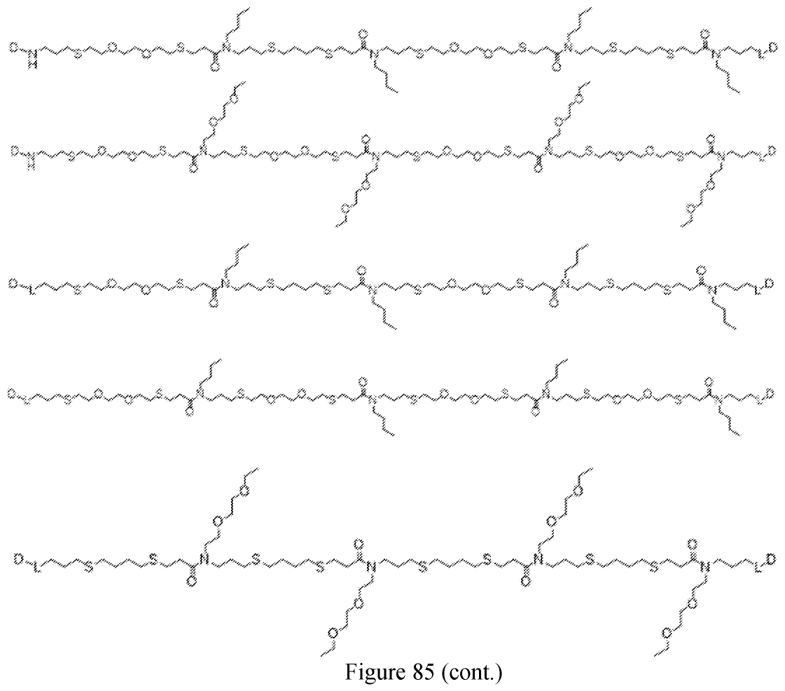

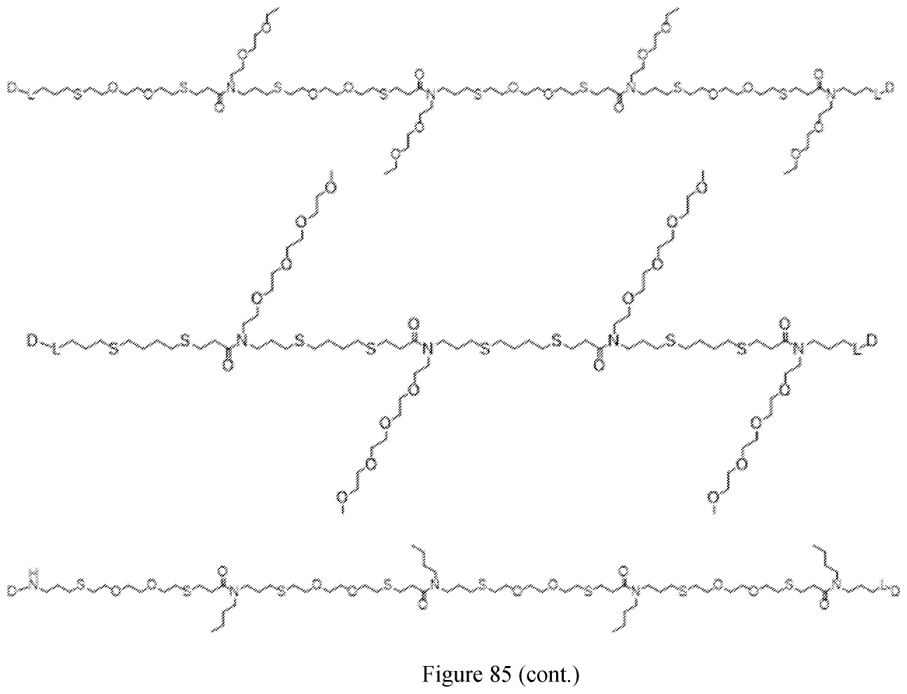

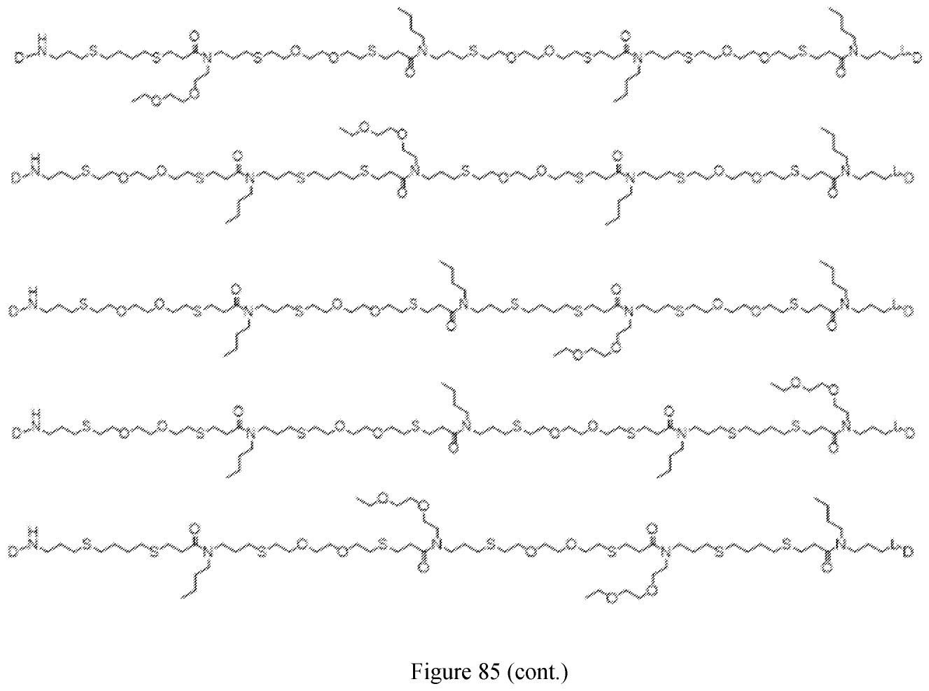

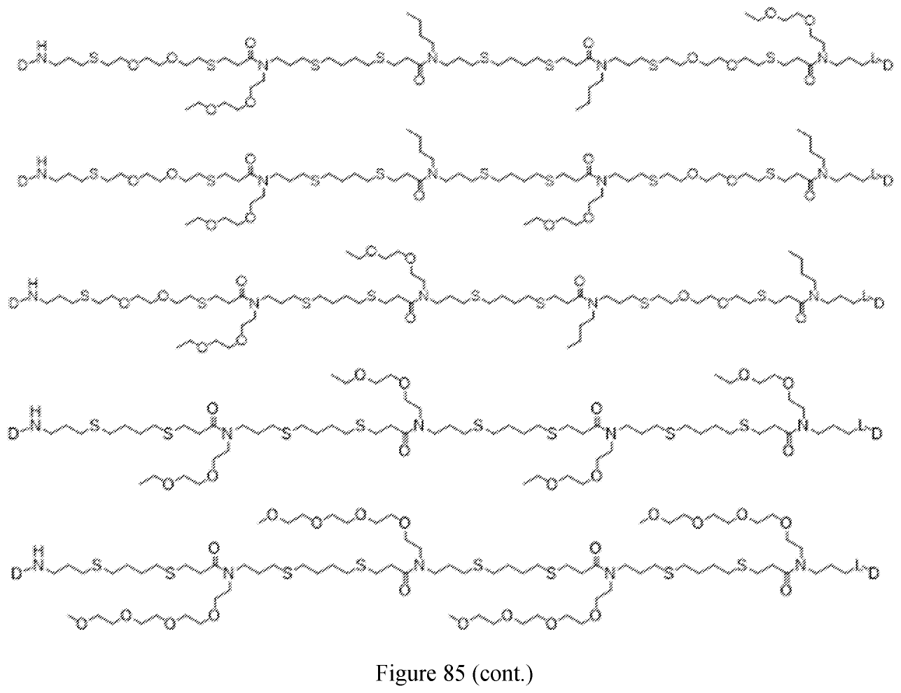

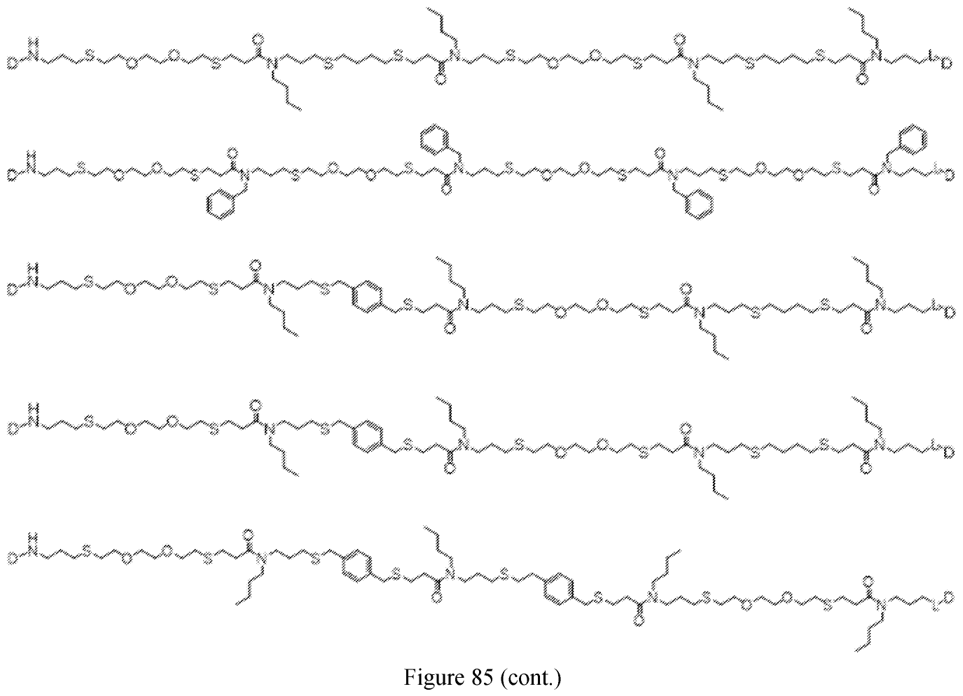

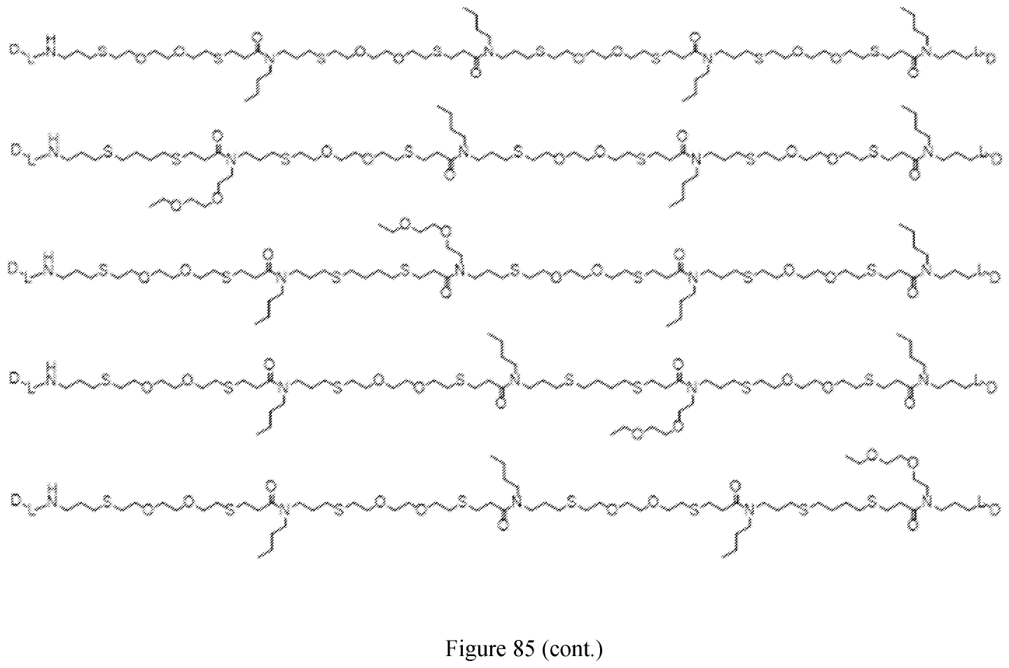

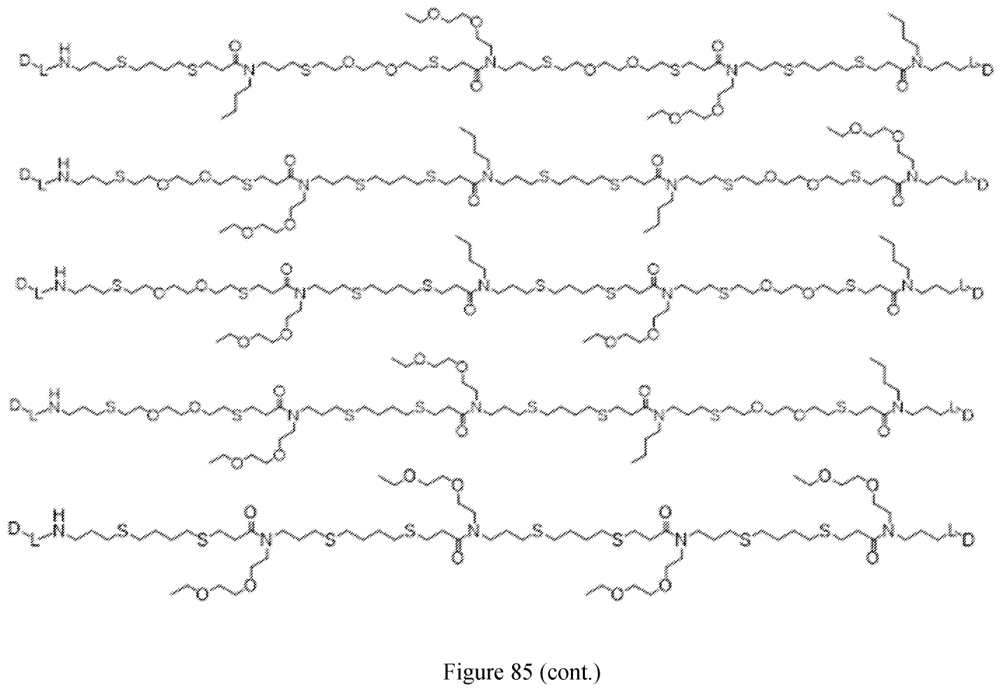

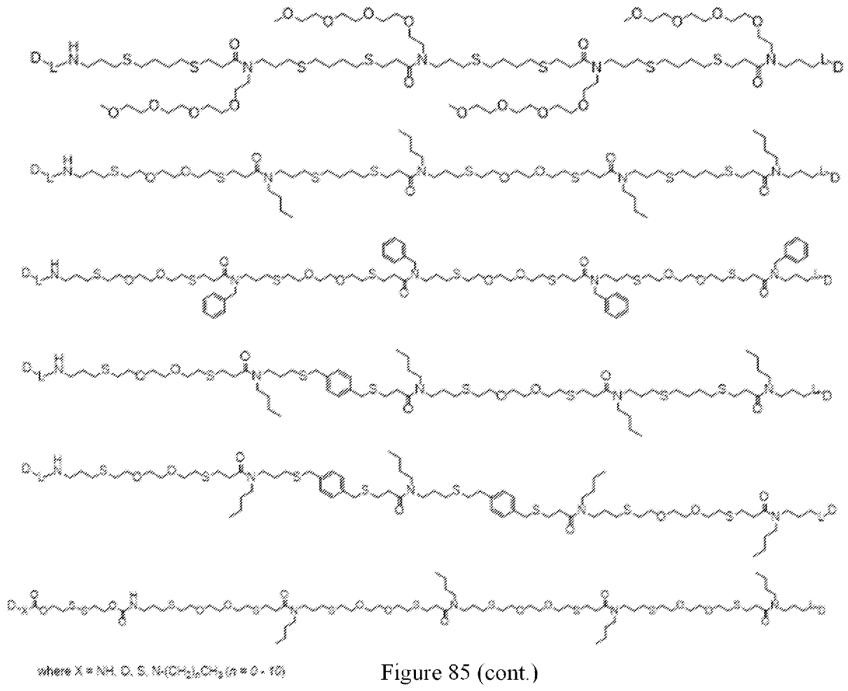

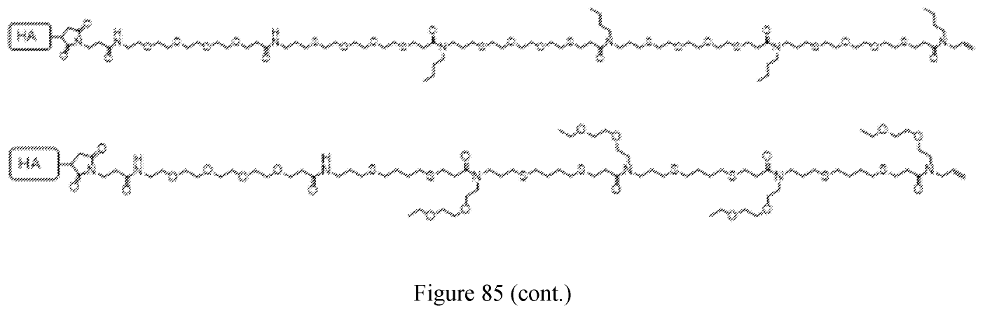

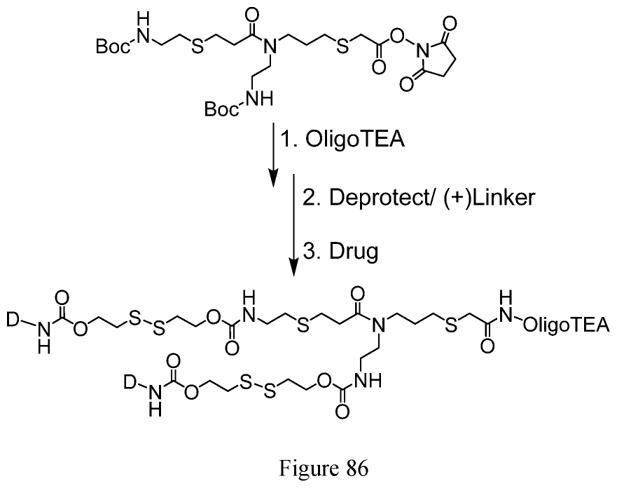

The compound may comprise various linking groups. A linking group may be cleavable in a biological environment (e.g., the reductive environment of a cell or through an enzyme, such as, for example, a protease), for example see FIG. 84. A linking group may be covalently bonded to one or more cargo groups. Non-limiting examples of linking groups include:

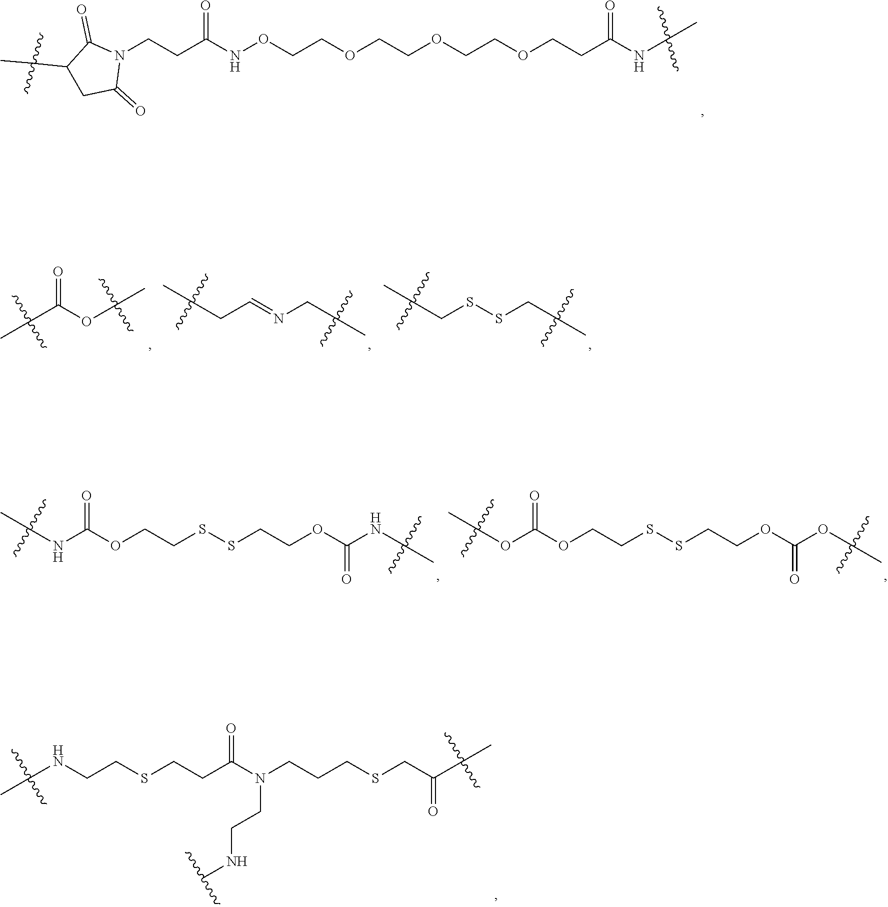

a dipeptide (e.g., -Val-citrulline-) and other cleavable peptides, and the like. The following linking groups may further comprise aliphatic groups (e.g., C₁ to C₂₀ aliphatic groups), cyclic aliphatic groups (e.g., C₁ to C₂₀ cyclic aliphatic groups), or aryl groups (e.g., C₅ to C₂₀ aryl groups) on one or both termini (e.g., an alkyl group on one or both termini):

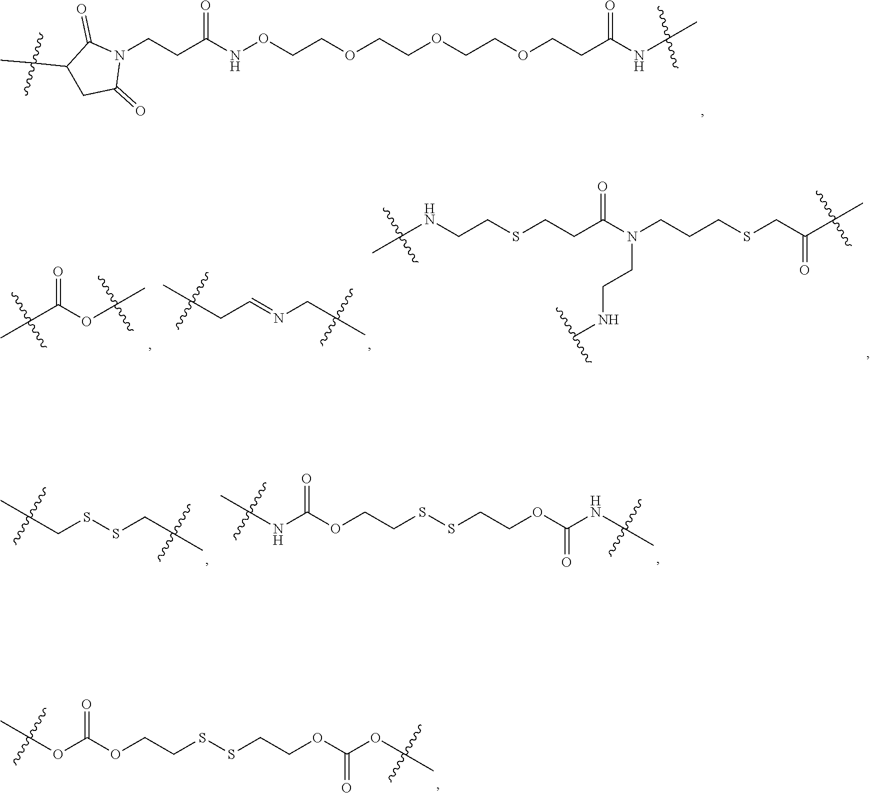

a dipeptide (e.g., -Val-citrulline-) and other cleavable peptides, and the like. Additional examples of linking groups include oxygen functionalized aliphatic groups (e.g., —O-alkyl), nitrogen functionalized aliphatic groups (e.g., —NH-alkyl or —N=alkyl), and sulfur functionalized aliphatic groups (e.g., —S-alkyl).

Non-limiting examples of compounds of the present disclosure include:

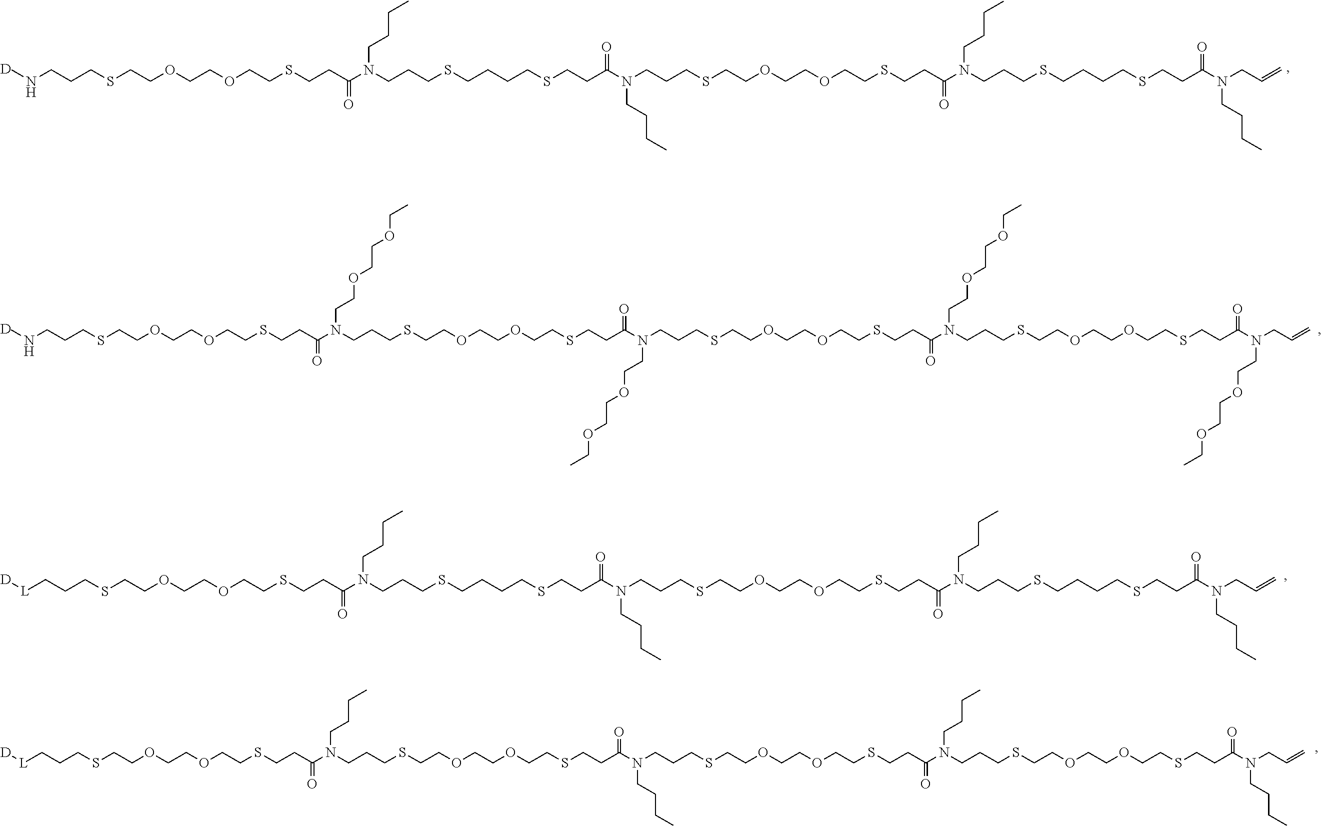

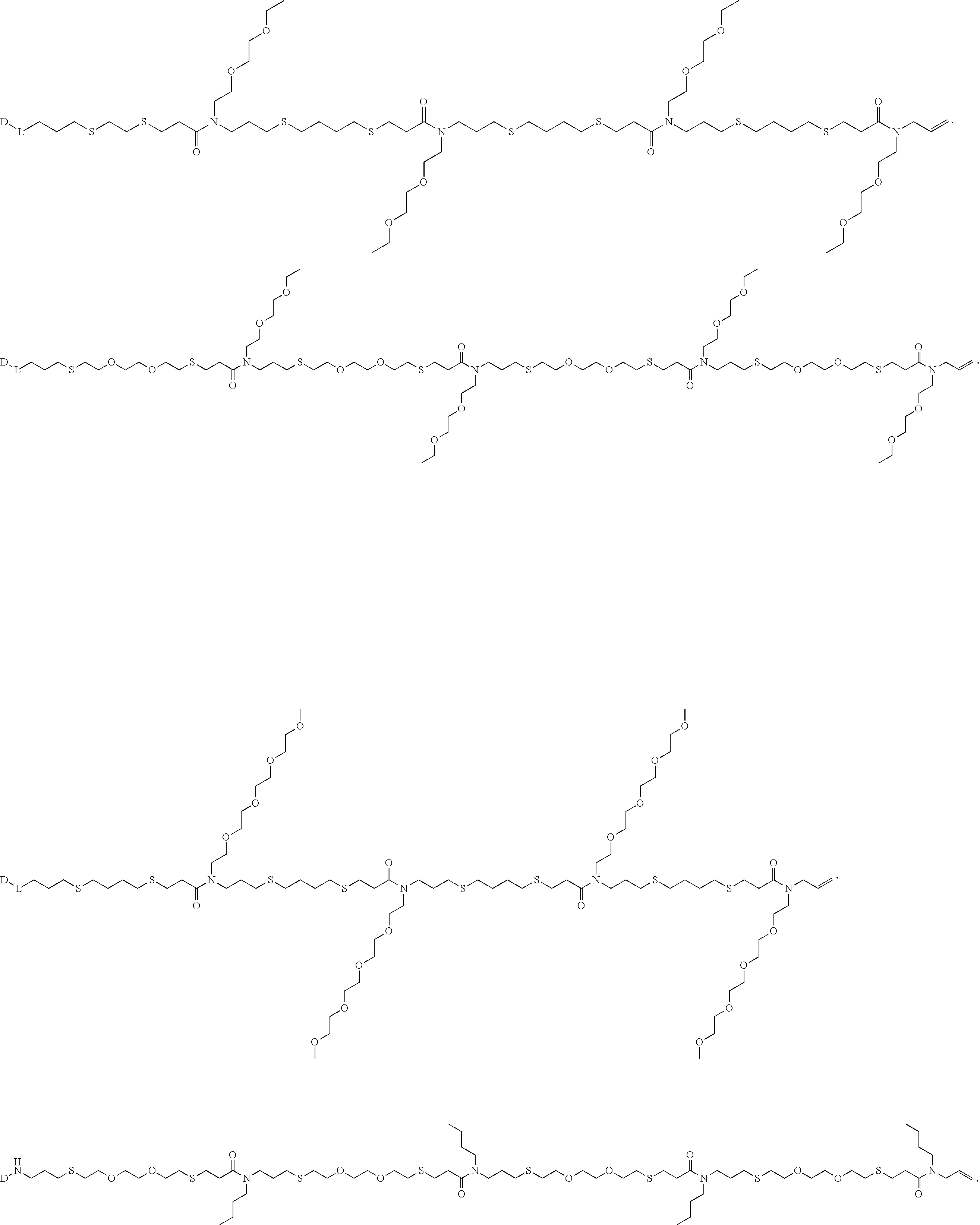

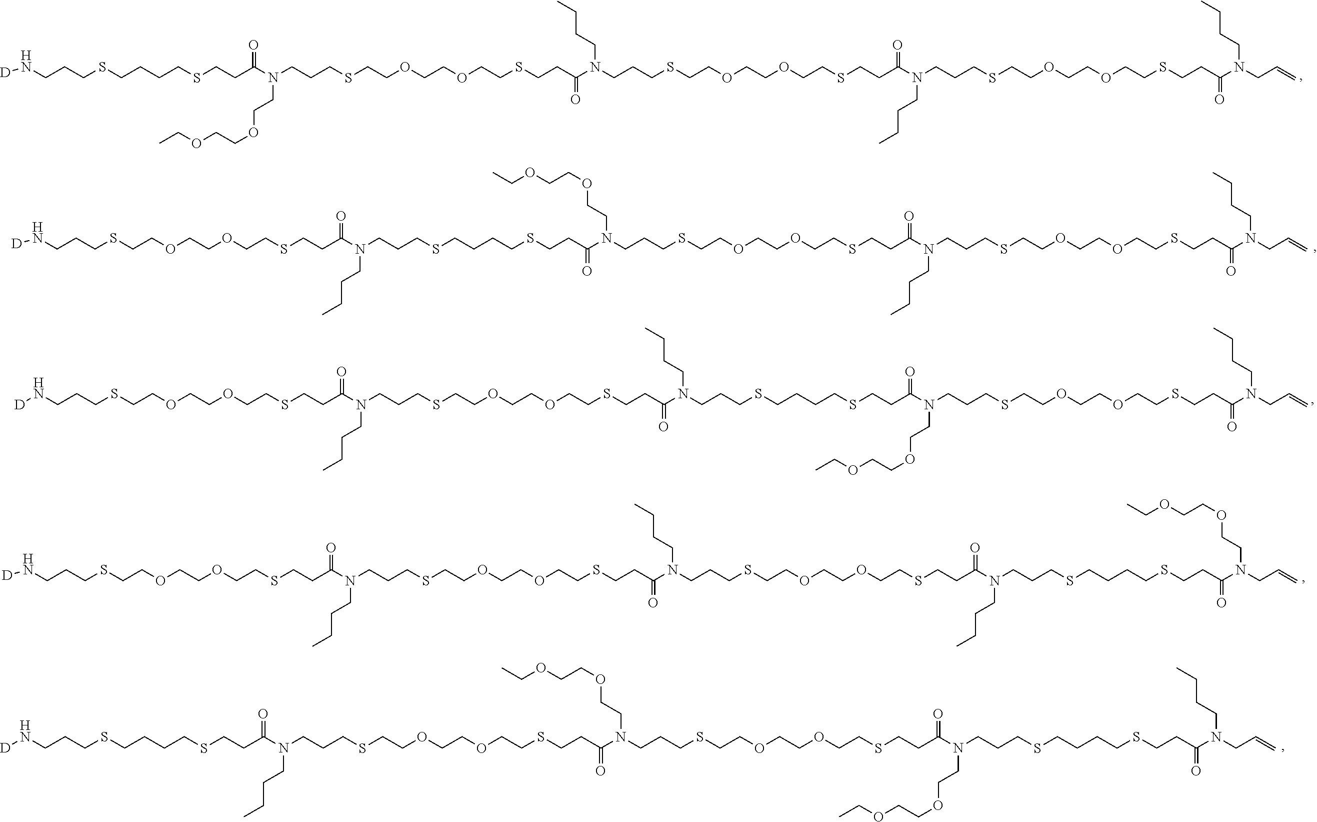

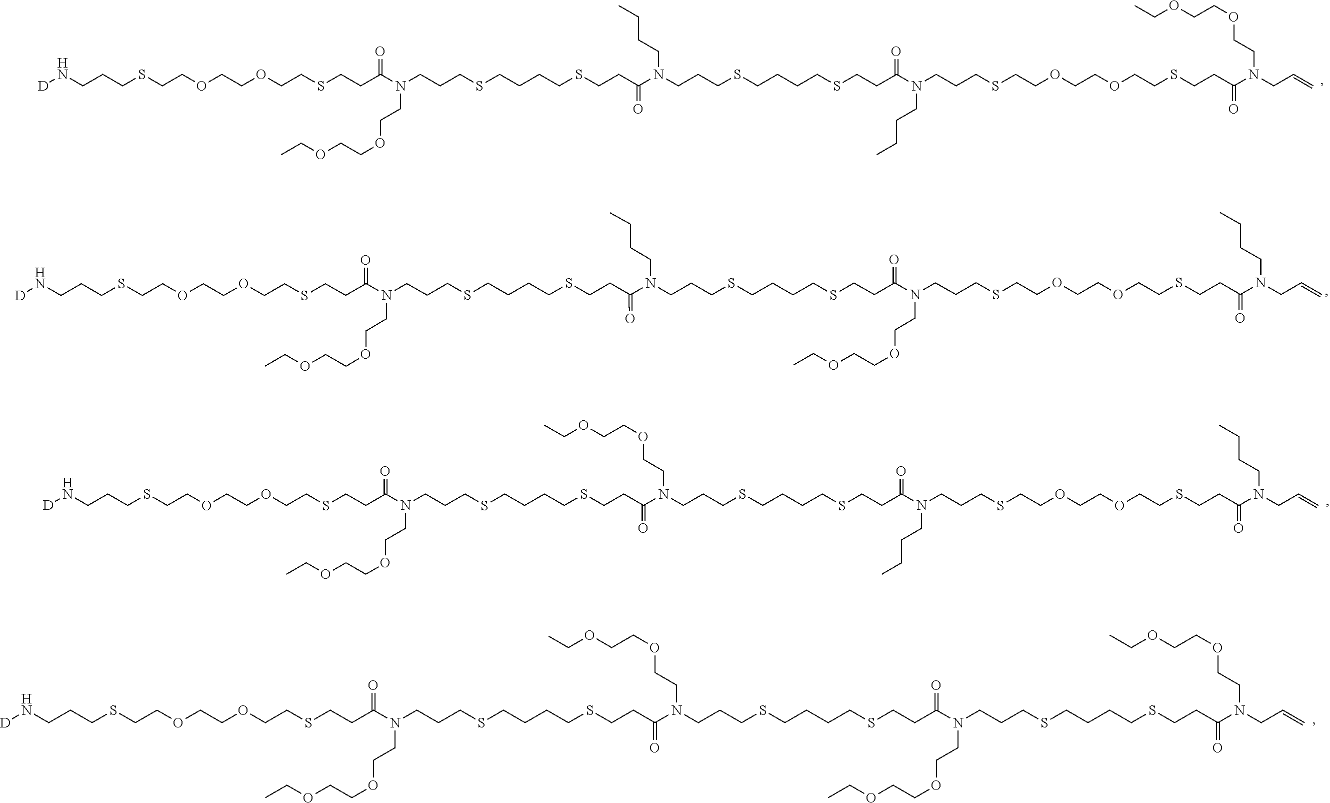

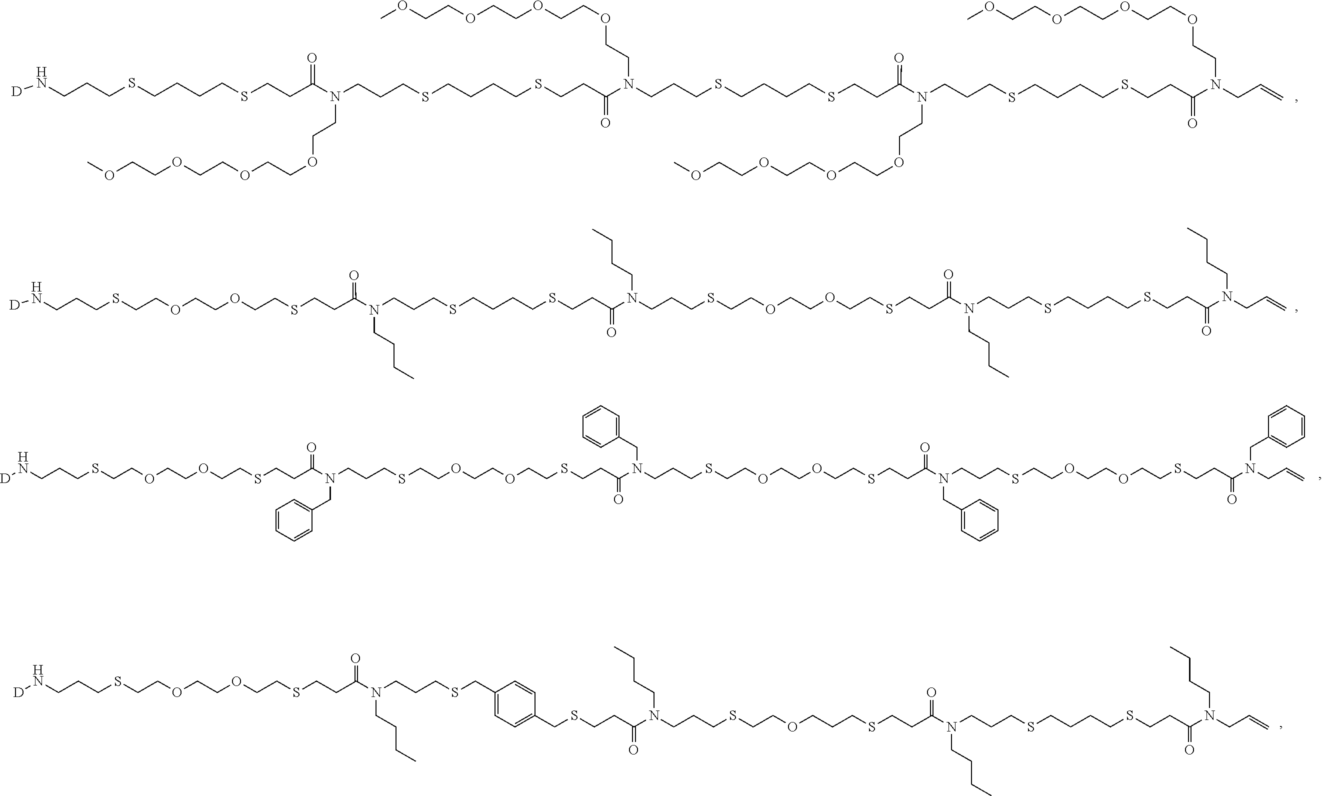

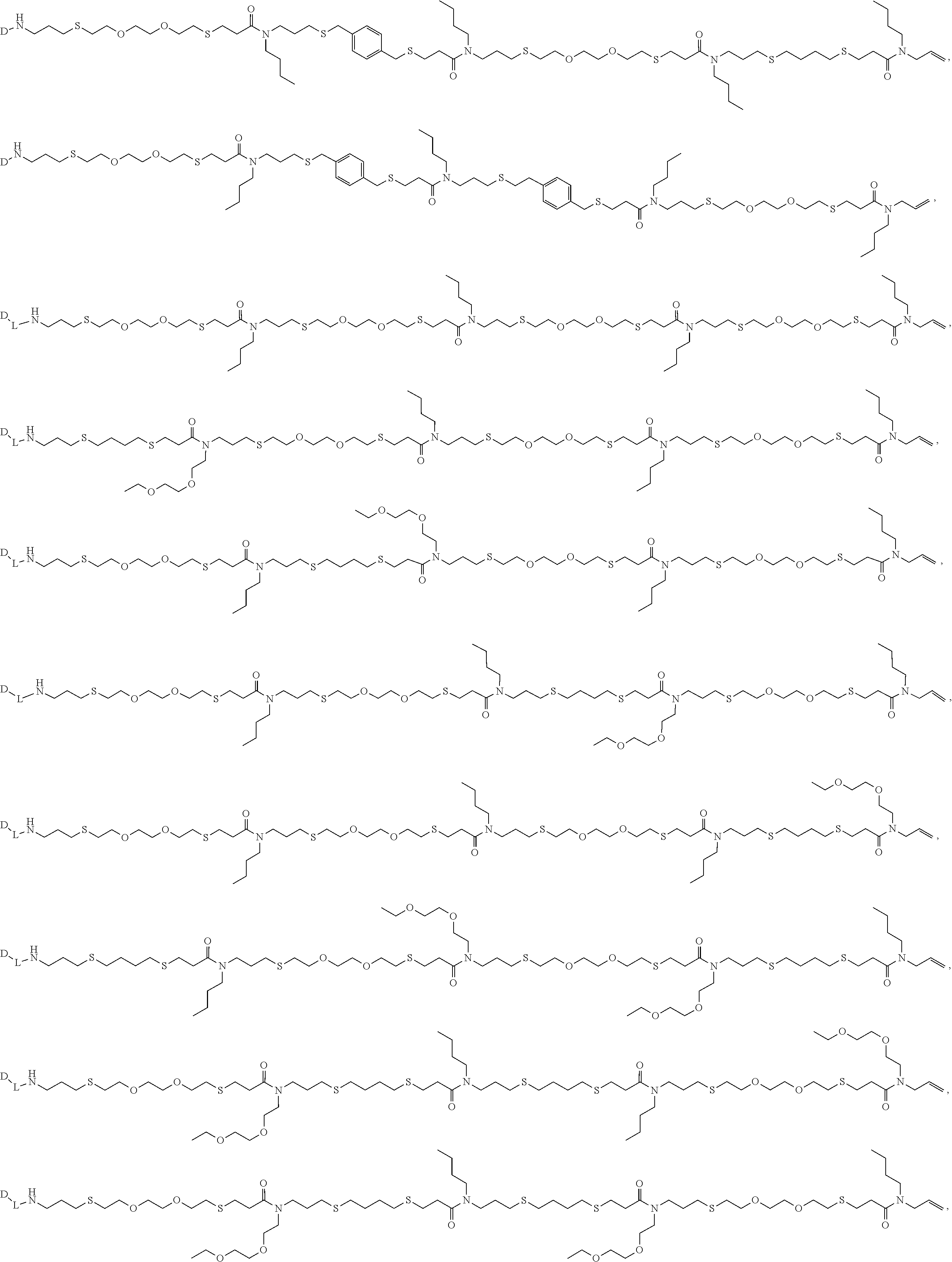

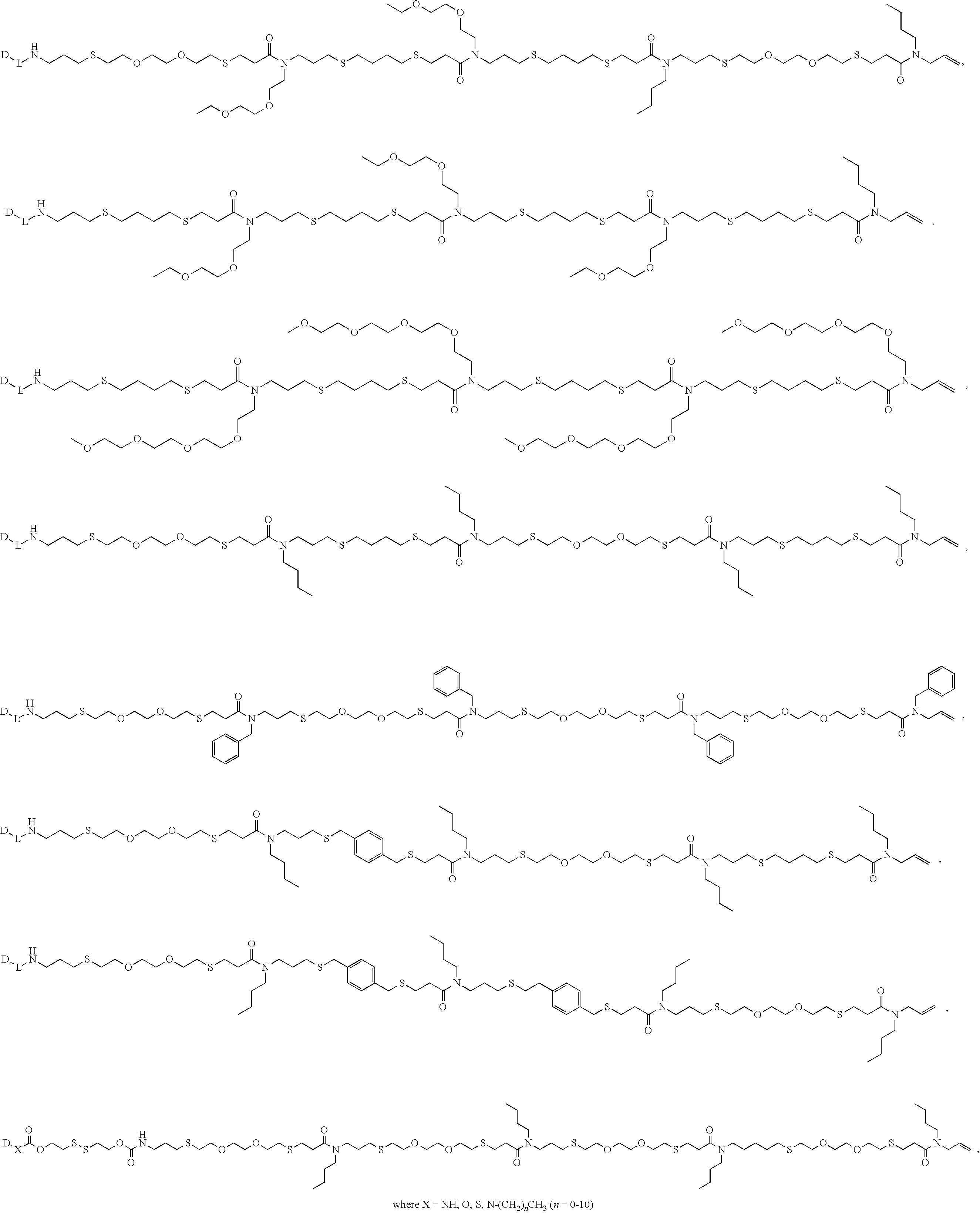

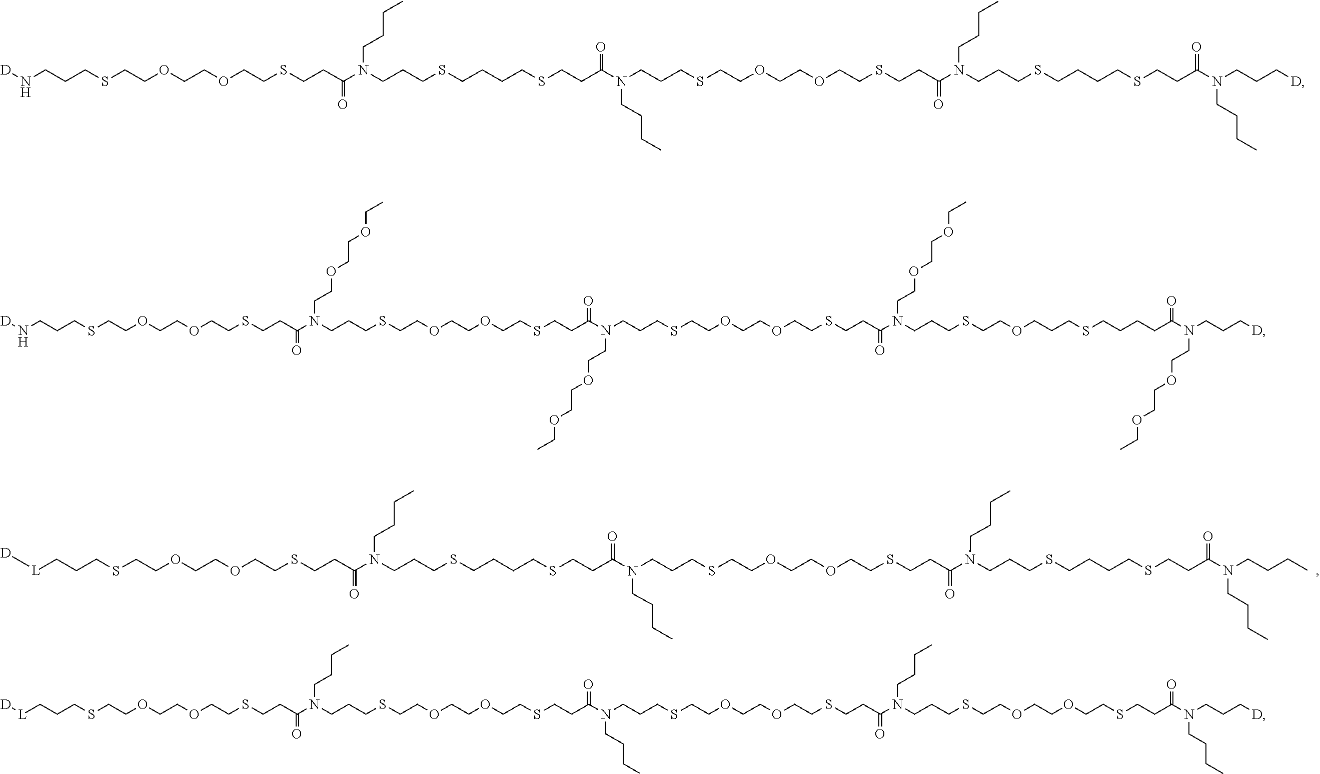

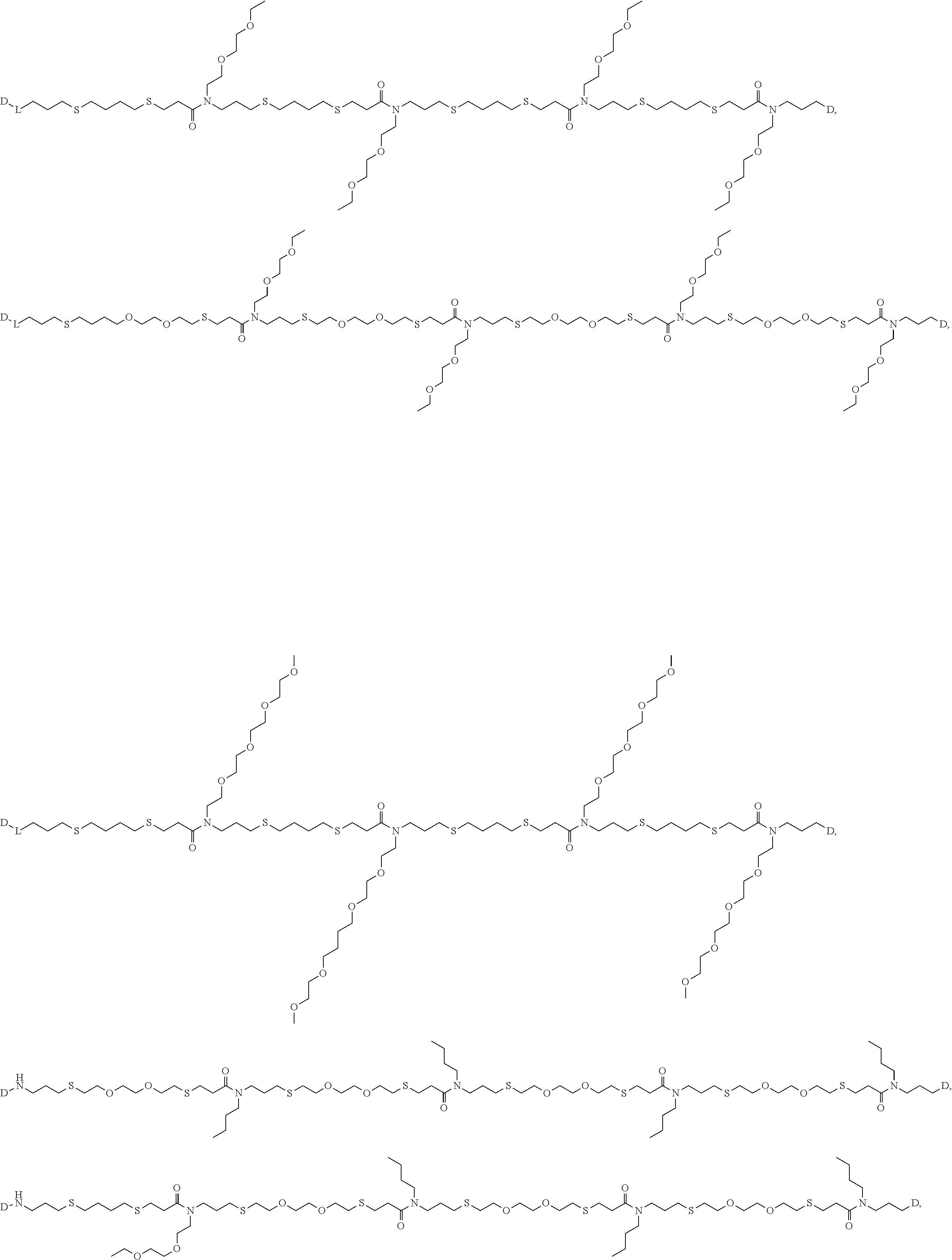

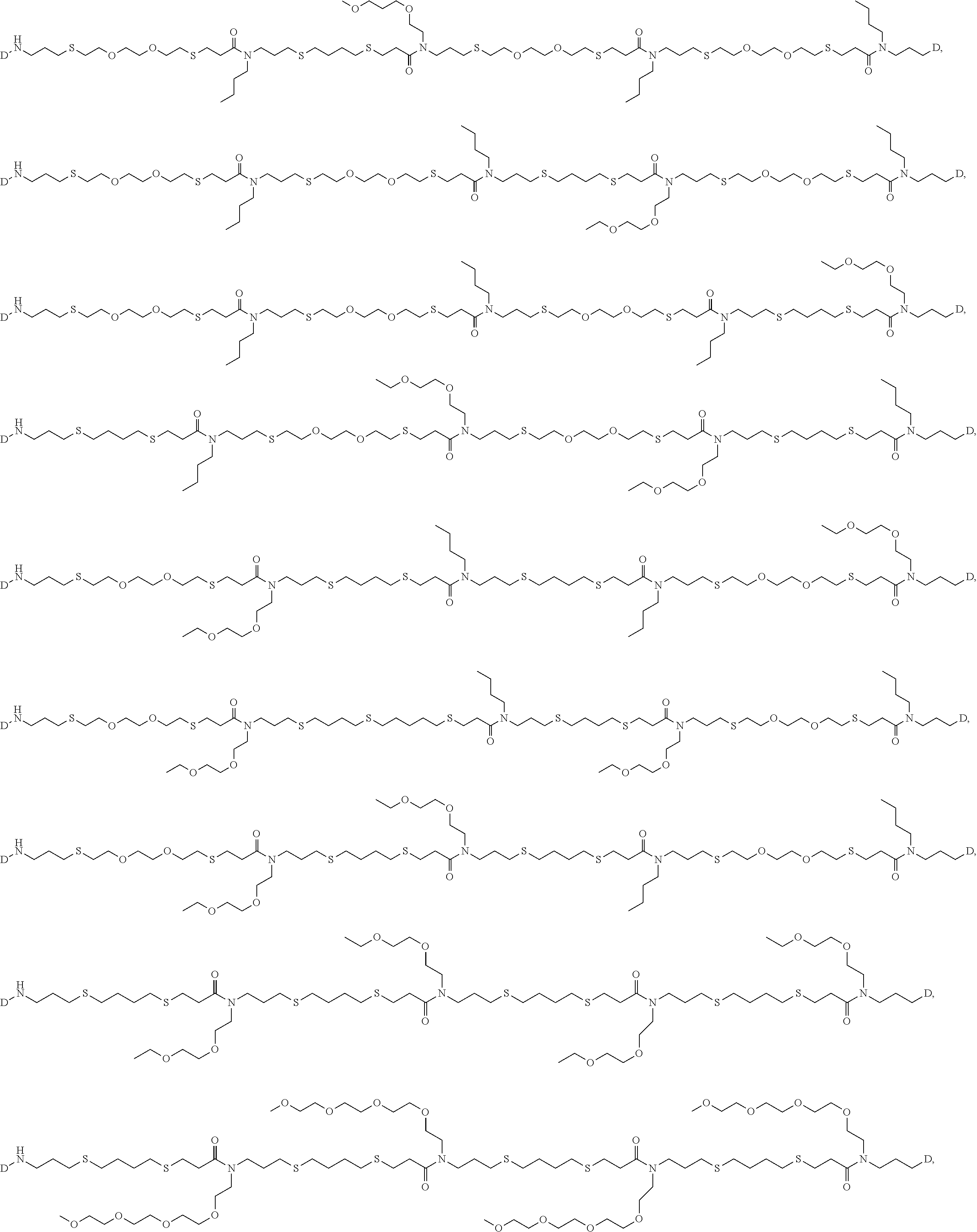

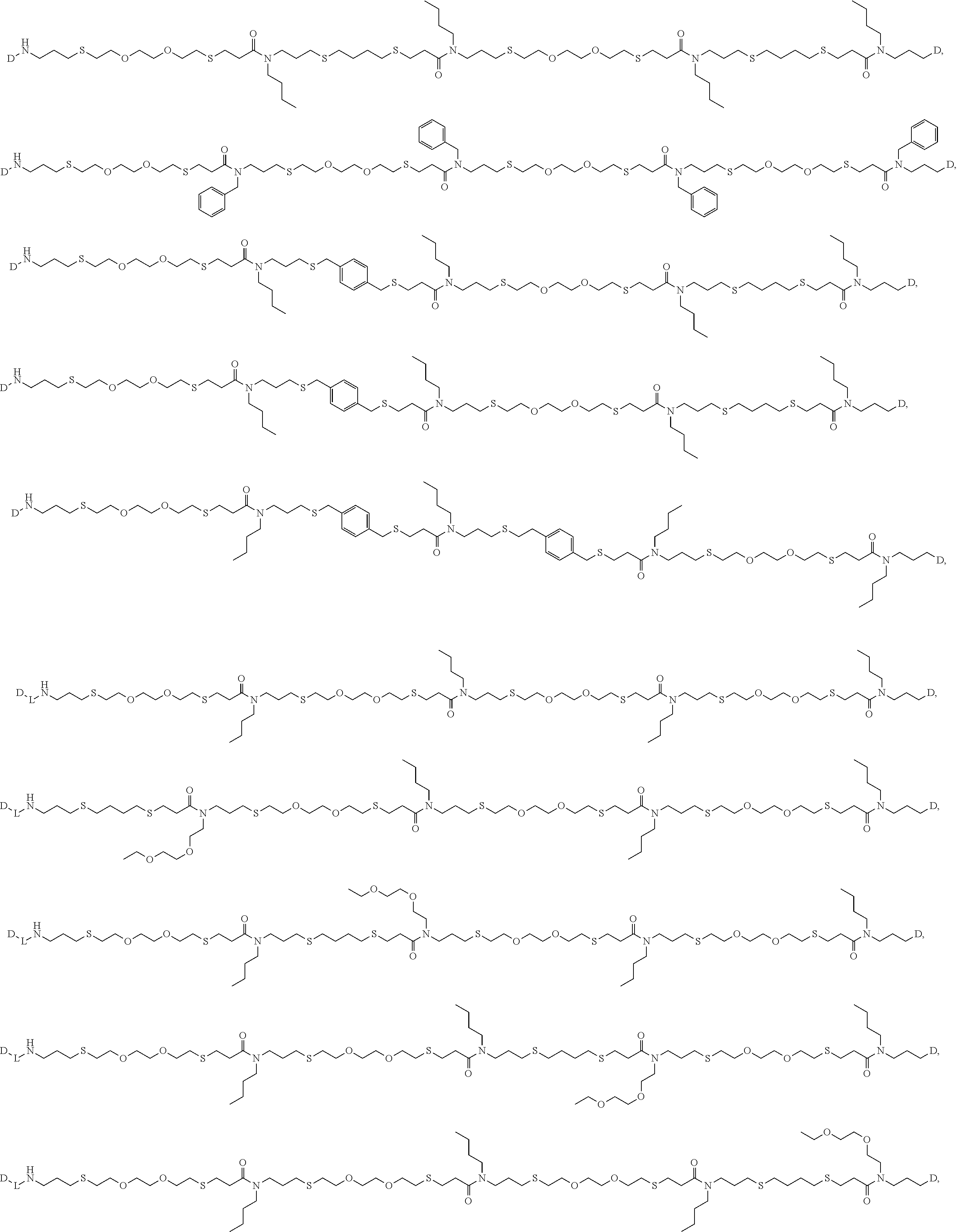

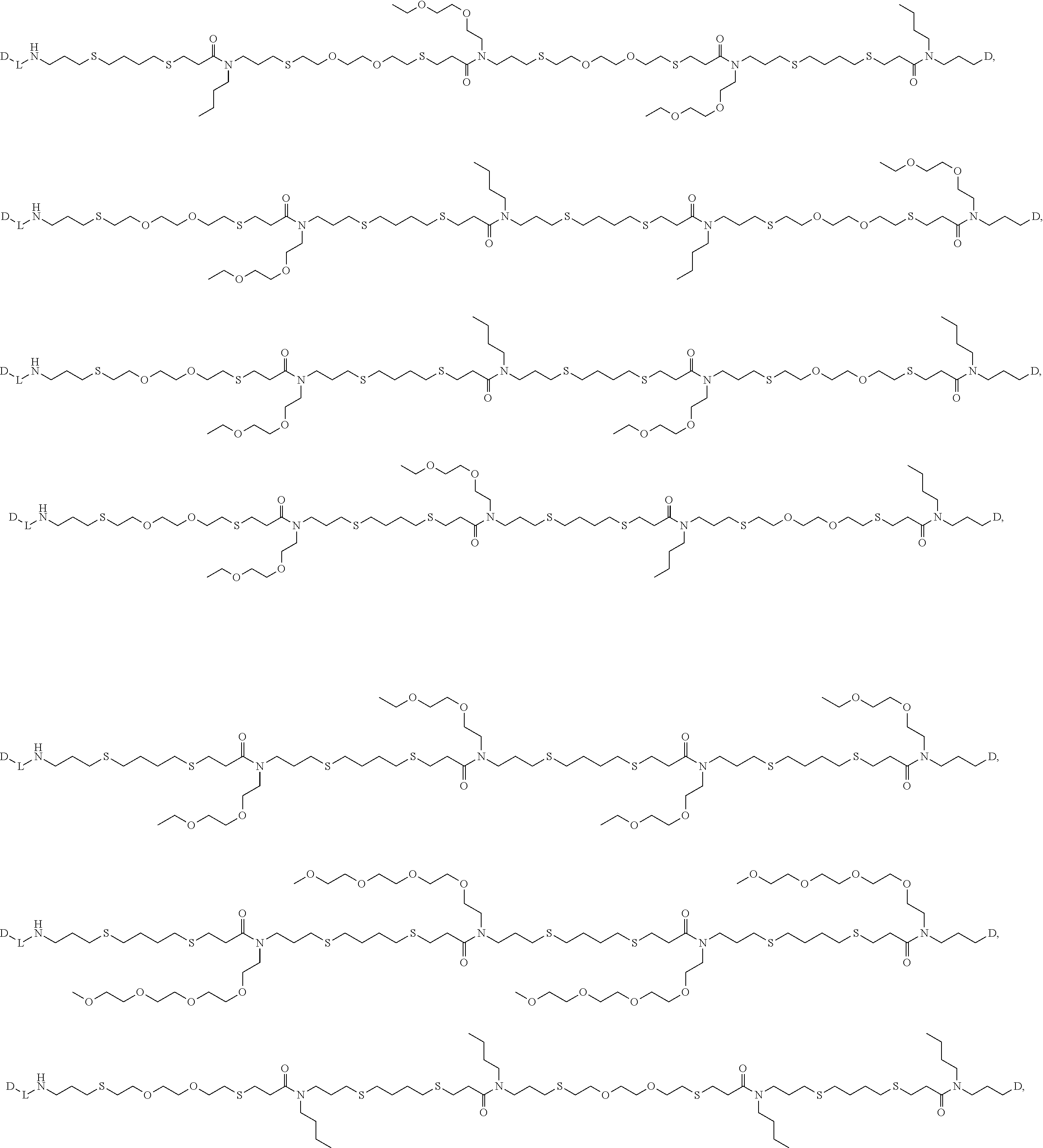

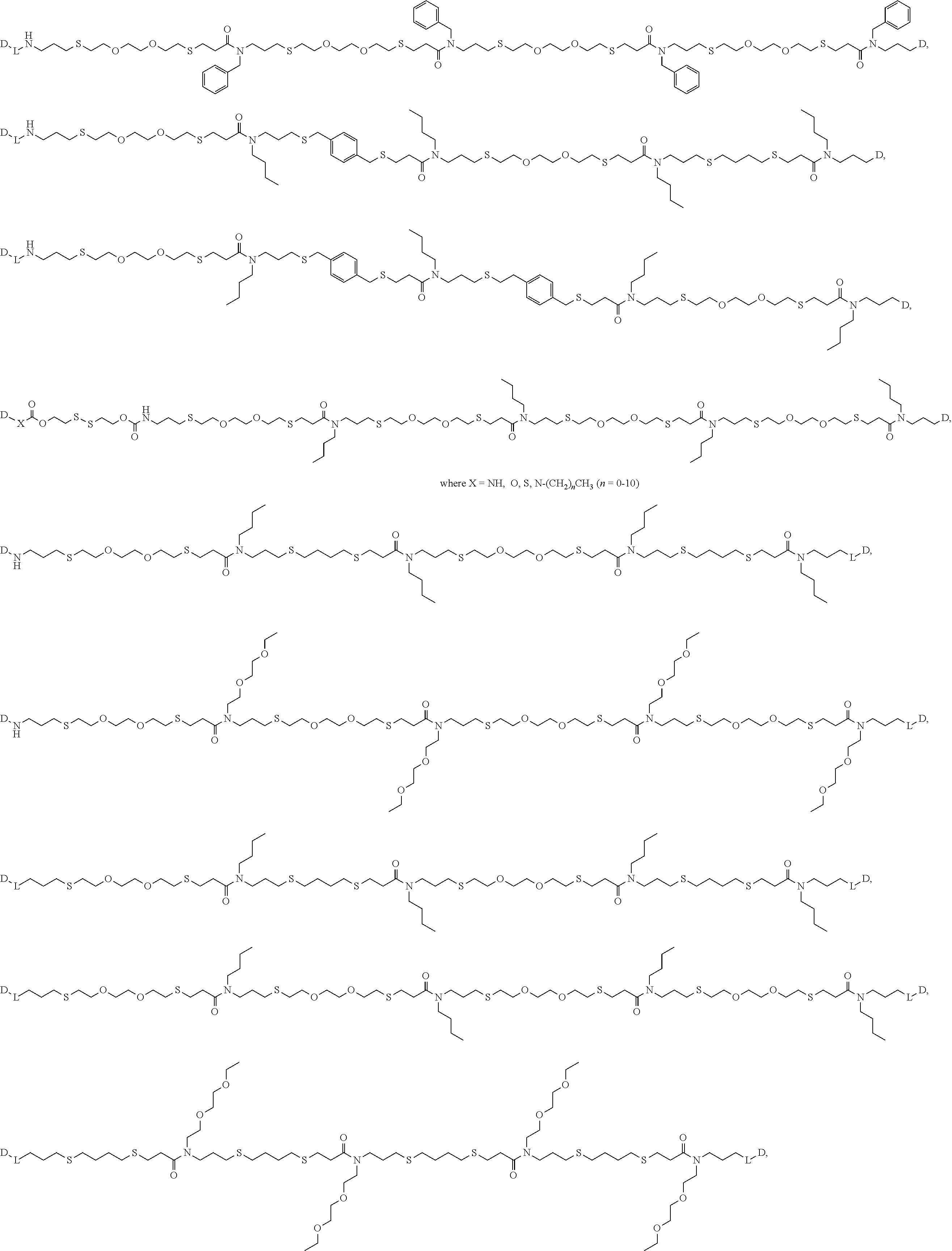

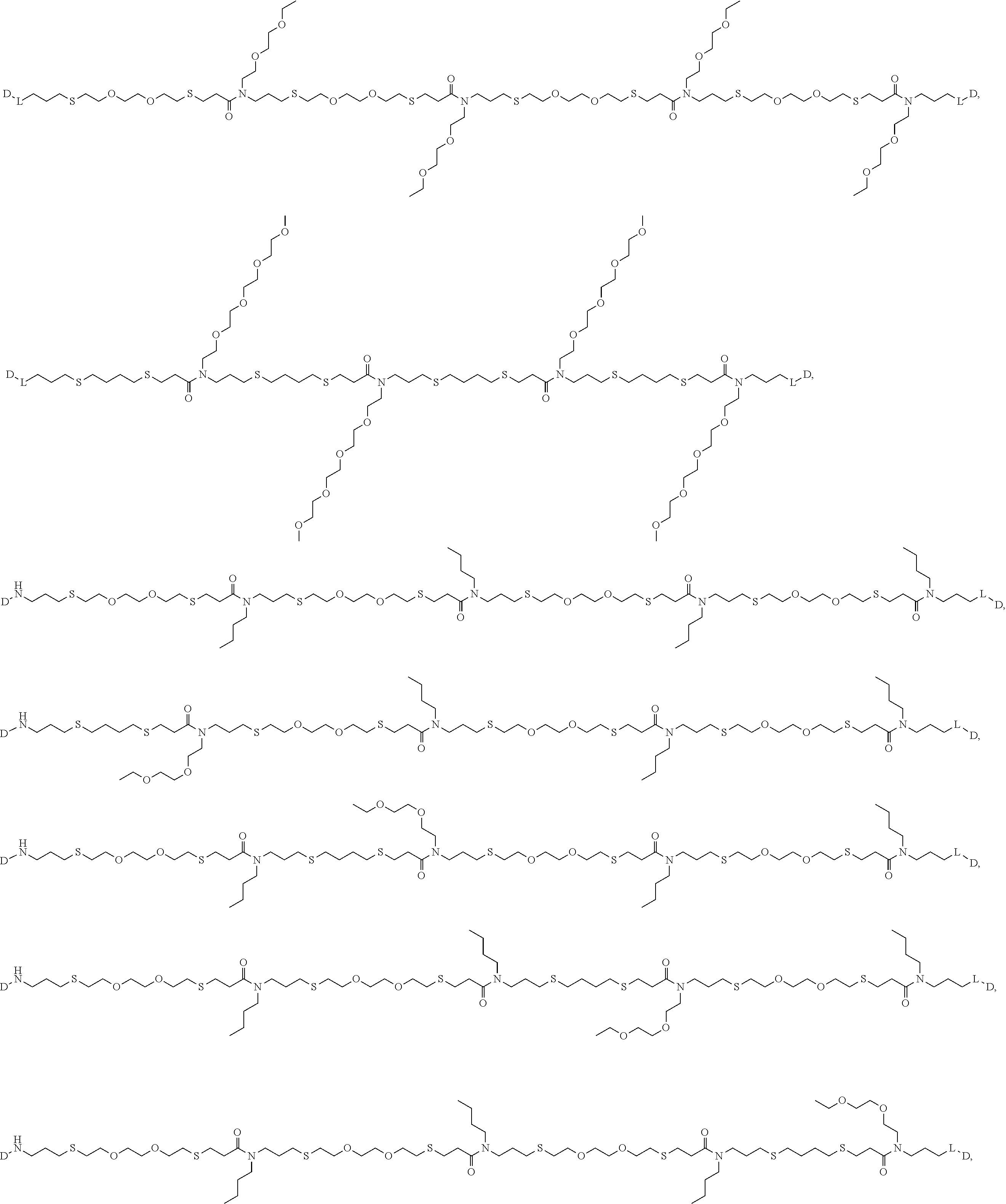

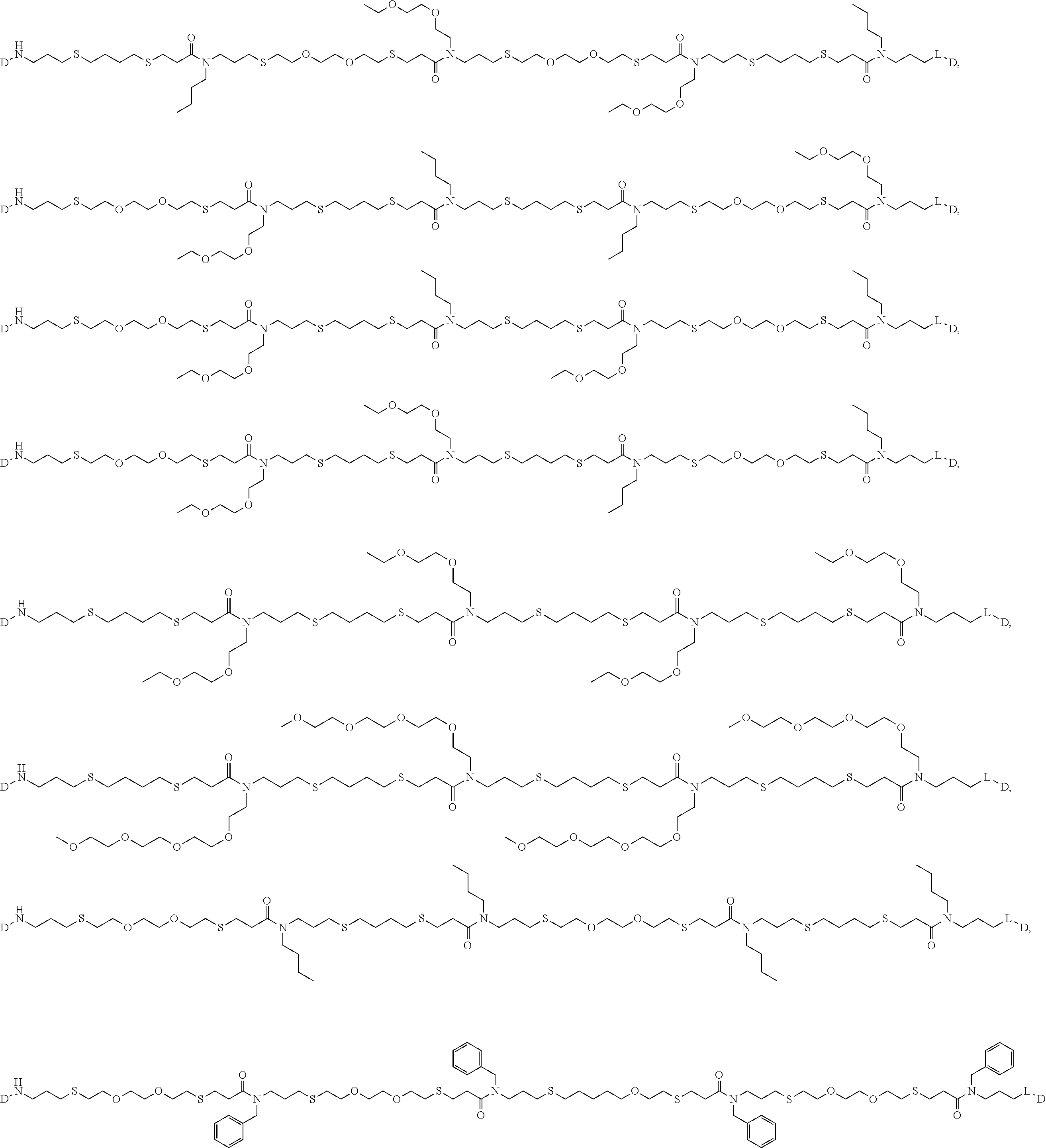

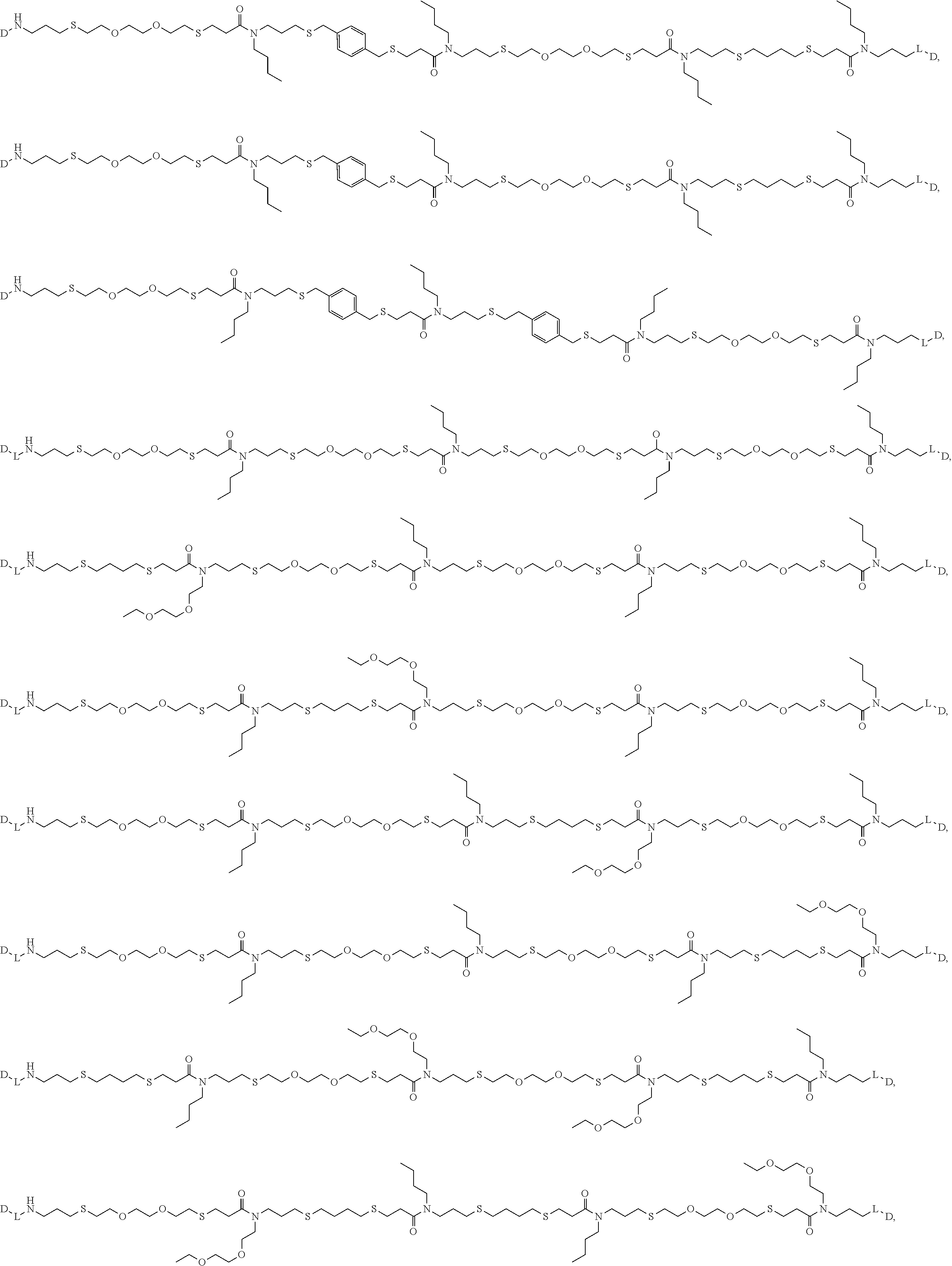

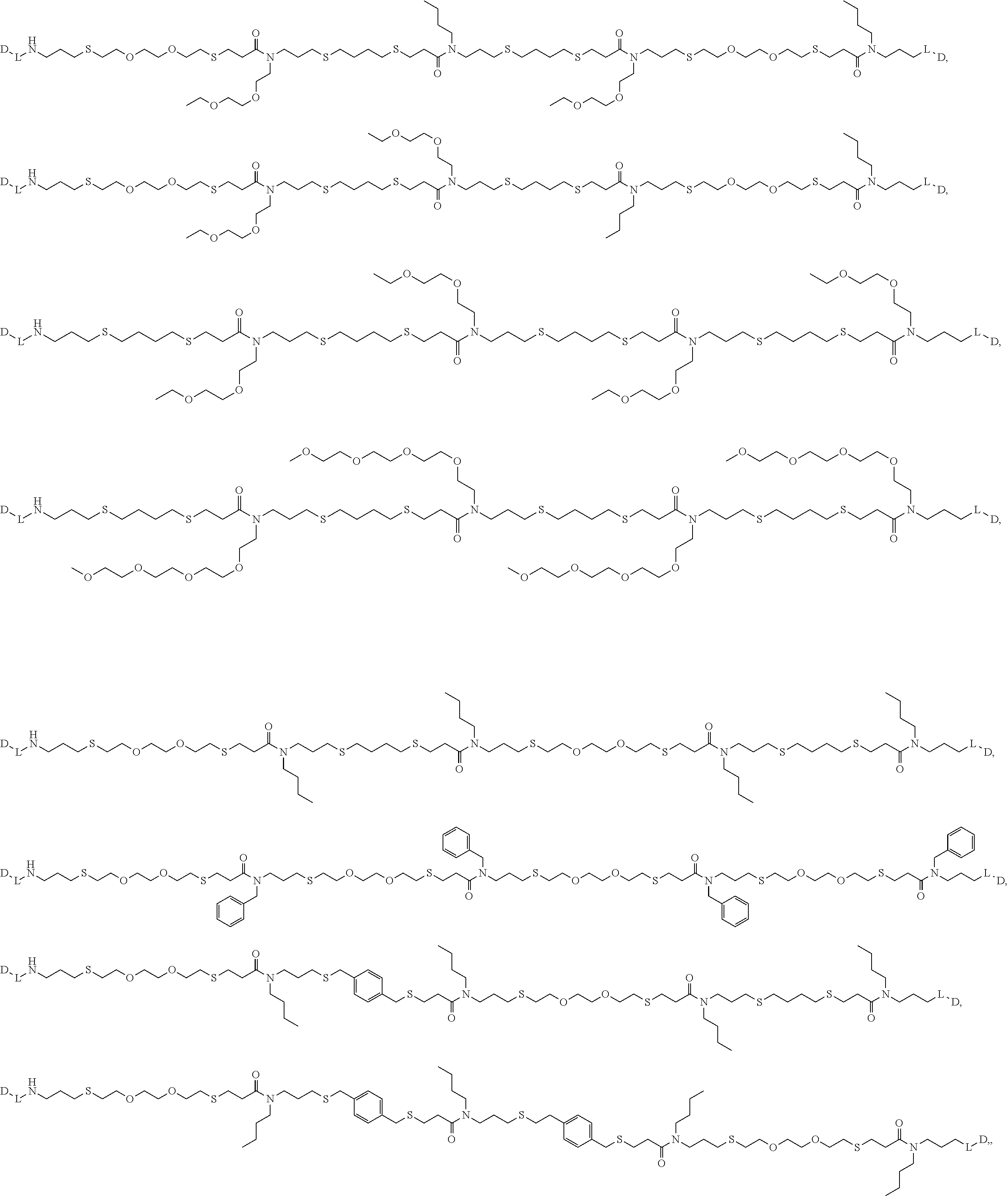

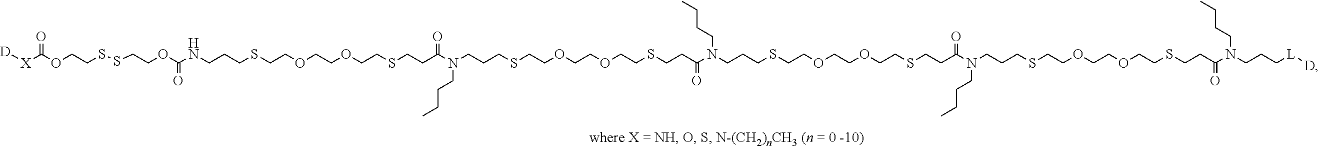

and isomers thereof, where Lis chosen from a linking group, NH, N, O, and S, and Dis one or more cargo group. In various examples, at least two cargo groups are attached to a linking group.

In various examples, compounds of the present disclosure has various isomers. In an illustrative example, the following compounds are isomers:

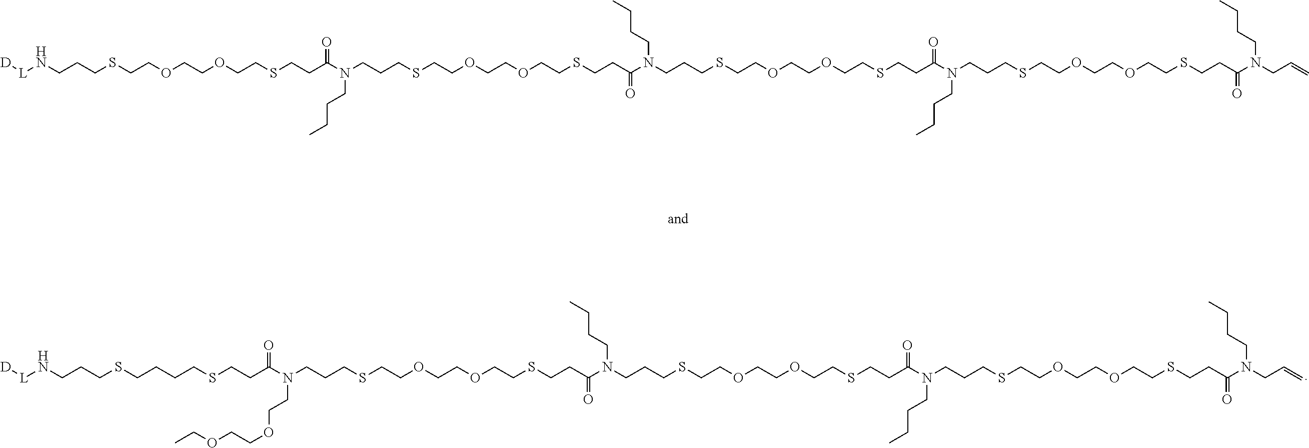

A compound of the present disclosure may have the following structure:

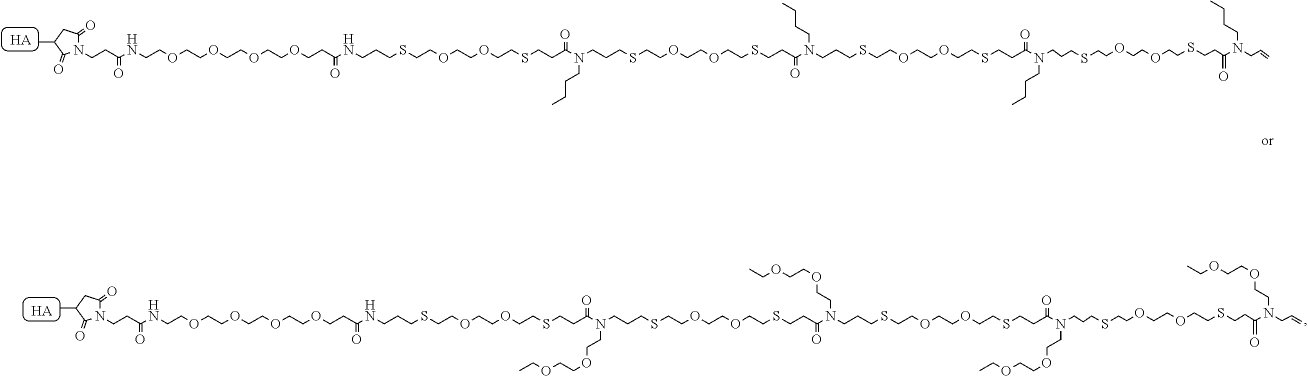

where HA is KADNAAIESIRNGTYDHDVYRDEALNNRFQIKGVELKSGYKDW-S- (SEQ ID NO:1) and the underlined S is a sulfur atom.

A compound of the present disclosure may have the following structure:

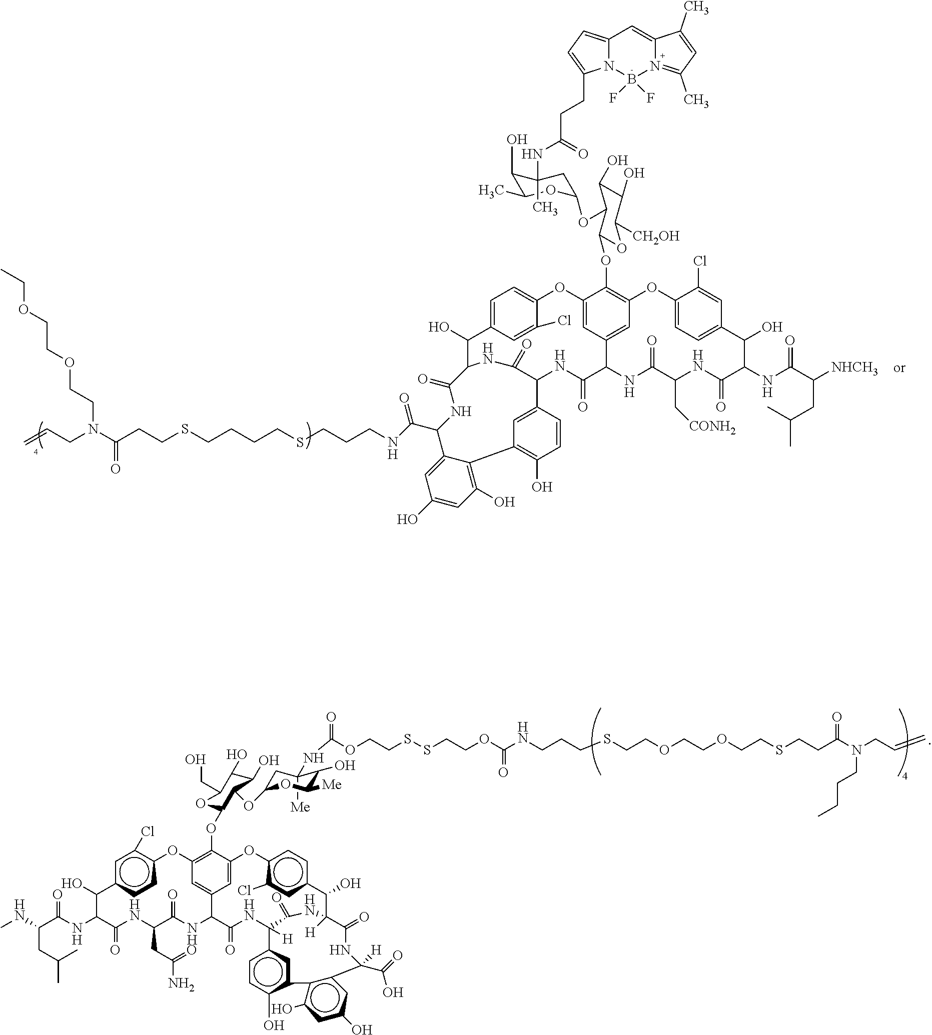

In an aspect, the disclosure provides methods of making compounds of the present disclosure. The compounds may be synthesized by iterative thiol-ene reactions and Michael reactions. The methods use a monomer having two or more functional groups (the monomers may have additional functional groups that do not react during the polymerization reactions) that react with a co-monomer under orthogonal conditions (i.e., a monomer with orthogonal functional groups). By “orthogonal conditions” it is meant that two functional groups on the monomer (a first functional group and a second functional group) react under conditions such that the first functional group reacts without any detectible reaction (such as, for example, by ¹H NMR or the like) of the second functional group and the second functional group reacts without any detectible reaction of the first functional group (such as, for example, by ¹H NMR, or the like).

In an example, a method of making a compound of the present disclosure comprises: a) contacting a first monomer having a free allyl group or free acrylamide group (e.g., either a free allyl group or free acrylamide group) and a first co-monomer having two thiol groups capable of reacting with the allyl group and/or the acrylamide group of the first monomer under conditions such that the allyl group or acrylamide group reacts with one of the thiol groups on the co-monomer to form a first reaction product; b) contacting the first reaction product with a second monomer having an allyl group and acrylamide group such that i) the acrylamide group of the second monomer reacts with a thiol group of the first reaction product without substantial reaction of the allyl group of the second monomer or ii) the allyl group of the second monomer reacts with the unreacted thiol group of the first reaction product without substantial reaction of the acrylamide group of the second monomer to form a second reaction product; c) optionally, contacting the second reaction product with a second co-monomer having two thiol groups such that i) if the allyl group of the second monomer reacted in b), the acrylamide group of the second product reacts with one of the thiol groups of the second co-monomer or ii) if the acrylamide group of the second monomer reacted in b), the allyl group of the second product reacts with one of the thiol groups of the second co-monomer to form a third reaction product; d) optionally, contacting the third reaction product with a third monomer having an allyl group and acrylamide group such that i) the acrylamide group of the third monomer reacts with the unreacted thiol group of the third reaction product without substantial reaction of the allyl group of the third monomer or ii) the allyl group of the third monomer reacts with the unreacted thiol group of the third reaction product without substantial reaction of the acrylamide group of the third monomer to form a fourth reaction product; and e) optionally, repeating c) and d) from 1 to 23 times such that a compound having 3 to 24 or 3 to 10 or 3 to 8 monomer units is formed, where a reaction product is an oligoTEA. In an example, the first monomer and/or second monomer and/or third monomer has one or two allyl groups and one acrylamide group and at least one of the groups (e.g., an allyl group or acrylamide group is free); f) optionally, contacting oligoTEA with a linking group such that the oligoTEA reacts with the linking group resulting in a reaction product comprising an oligoTEA comprising a linking group; g) contacting the oligoTEA (which may comprise a linking group) with a cargo molecule such that the cargo molecule reacts the oligoTEA (which may comprise a linking group) resulting in a reaction product comprising an oligoTEA comprising a cargo group, optionally further comprising a linking group.

In an example, a method of making a compound comprises: a) contacting a first monomer having a free allyl group and a first co-monomer having two thiol groups capable of reacting with the allyl group of the first monomer under conditions such that allyl group reacts with one of the thiol groups on the co-monomer to form a first reaction product; b) contacting the first reaction product with a second monomer having an allyl group and acrylamide group such that the acrylamide group of the second monomer reacts with the unreacted thiol group of the first reaction product without substantial (less than 50%) reaction of the allyl group of the second monomer to form a second reaction product; c) optionally, contacting the second reaction product with a second co-monomer having two thiol groups such that the allyl group of the second product reacts with one of the thiol groups of the second co-monomer, without substantial reaction of the acrylamide group to form a third reaction product; d) optionally, contacting the third reaction product with a third monomer having an allyl group and acrylamide group such that the acrylamide group of the third monomer reacts with the unreacted thiol group of the third reaction product without substantial reaction of the allyl group of the third monomer to form a fourth reaction product; and e) optionally, repeating c) and d) from 1 to 23 times such that a compound having 3 to 24 or 3 to 10 or 3 to 8 monomer units is formed, where a reaction product is an oligoTEA. In an example, the first monomer and/or second monomer and/or third monomer has one or two allyl groups and one acrylamide group and at least one of the groups (e.g., an allyl group or acrylamide group is free); f) optionally, contacting oligoTEA with a linking group such that the oligoTEA reacts with the linking group resulting in a reaction product comprising an oligoTEA comprising a linking group; g) contacting the oligoTEA (which may comprise a linking group) with a cargo molecule such that the cargo molecule reacts the oligoTEA (which may comprise a linking group) resulting in a reaction product comprising an oligoTEA comprising a cargo group, optionally further comprising a linking group.

The monomer has at least two functional groups that react under orthogonal conditions (e.g., a monomer with orthogonal functional groups). In the case where the monomer has two functional groups (e.g., an allyl group and an acrylamide group), the two groups react under orthogonal conditions. The first monomer used may only one functional group that can react under one of the orthogonal polymerization conditions (e.g., the other functional group is blocked (e.g., reacted to form a functional group that is not reactive under one of the orthogonal polymerization conditions) or tagged (e.g., tagged with a fluorous tag)). The monomers may have additional functional groups that do not react during the polymerization reactions.

By “orthogonal conditions,” it is meant that one (or one group) of functional groups of the monomer reacts without substantial reaction of the other functional groups of the monomer. By “substantial reaction” it is meant that 5% or less of the other functional groups react in the reaction one (or one group) of functional groups of the monomer. In various examples, 4% or less, 3% or less, 2% or less, 1% or less of the other functional groups react in the reaction one (or one group) of functional groups of the monomer. In an examples, there is no detectible reaction of the other functional groups in the reaction one (or one group) of functional groups of the monomer. The reaction of the one (or one group) of the functional groups of the monomer or other functional groups of the monomer can be detected by methods known in the art. For example, the reaction of these functional groups are detected by NMR spectroscopy (e.g., ¹H and/or ¹³C NMR).

Examples of functional groups that can react under orthogonal conditions include, but are not limited to, allyl and acrylamide groups, allyl and methacrylamide groups, methacrylamide and alkyne groups, allyl and vinylsulfone groups, vinylsulfone and acrylamide groups, vinylsulfones and methacrylamides, and the like. In an example, the monomer has an allyl group and an acrylamide group.

Monomers having three or more functional groups that react under orthogonal conditions may be used. Monomers that have one or more functional groups may react two or more times (e.g., an alkyne group) may be used. Use of these monomers may result in formation of branched compounds.

Monomers and co-monomers may be selected to provide a desired compound. The monomers may be selected to provide a desired structural element (derived from a monomer or co-monomer) at desired positions in the compound. Various combinations of monomers may be used to provide a desired structural element at desired positions in the compounds.

In an example, the monomer has the following structure:

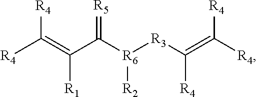

where [X] is any halogen, [A] is any atom except a hydrogen, [Q] is any atom except carbon or hydrogen [Ak] is any aliphatic chain, [Cy] is a cycle ([Cy] includes [Cb] and [Hy]), [Cb] is a carbocycle, and [Hy] is a heterocycle. R₁ is selected from [Ak], [Cy], and hydrogen atom. R₂ is selected from [Ak], [Cy], [X], and hydrogen atom when R₆ is a nitrogen atom. When R₆ is not a nitrogen atom then R₂ is absent. R₃ is selected from [Ak] and [Cy]. R₄ is independently selected from [Ak], [Cy], and hydrogen atom. R₅ is selected from an oxygen, sulfur, and nitrogen atom. R₆ is selected from an oxygen, sulfur, and nitrogen atom. In various embodiments, R₁ is a hydrogen atom or a methyl group (—CH₃), R₄ is a hydrogen atom, R₅ is an oxygen atom, and/or R₆ is a nitrogen atom.

In an example, the monomer has the following structure:

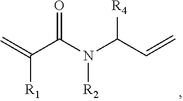

where R₁ is selected from [Ak], [Cy] or hydrogen atom. R₂ is selected from [Ak], [Cy], [X] or hydrogen. R₄ is selected from [Ak], [Cy], or hydrogen atom. For example, R₁ is a hydrogen atom or a methyl group (—CH₃). In another example, R₁ is a hydrogen atom or a methyl group, R₂ is a hydrogen atom, [Ak], or [Cy], and R₄ is a hydrogen atom or [Ak]. In yet another example, R₂ contains one or more alkenyl groups.

In an example, the monomer has the following structure:

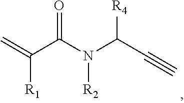

where R₁ is selected [Ak], [Cy] or hydrogen atom. R₂ is selected from [Ak], [Cy], [X] or hydrogen. R₄ is selected from [Ak], [Cy], or hydrogen atom. For example, R₁ is a hydrogen atom or a methyl group (—CH₃), R₂ is a hydrogen atom, and R₄ is a hydrogen atom or [Ak].

In an example, the monomer is an allyl acrylamide. Suitable allyl acrylamides are known in the art.

The co-monomer has two functional groups that react with the orthogonal functional groups of the monomer under orthogonal conditions. The co-monomers may have additional functional groups that do not react during the polymerization reactions.

Examples of co-monomer functional groups include thiols and secondary amines. In an embodiment, the co-monomer has two thiol groups or a thiol group and a secondary amine functional group.

In an example, the co-monomer has the following structure:

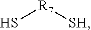

where [A] is any atom except a hydrogen, [Ak] is any aliphatic chain, and [Cy] is any cycle, and where R₇ is independently selected from any [A], [Ak] or [Cy]. In an example, the co-monomer is alkyl dithiol, where the alkyl chain of the alkyl dithiol has 1 to 20 carbons.

In an example, the co-monomer is an aliphatic dithiol. The aliphatic chain (e.g., R₇) can have 1 to 20 carbons, including all integer number of carbons and ranges therebetween. The aliphatic group can be substituted or unsubstituted and/or branched or linear. Examples of suitable aliphatic dithiols include: ethane dithiol, DTT, PEG dithiol and

| | | Exact |
|---|---|---|
| Structure | IUPAC Name | rmass |
| 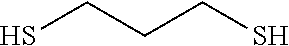 | 1,3-propanedithiol | 108.01 |
| 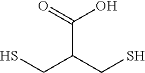 | 3-mercapto-2-(mercaptomethyl) propanoic acid | 152 |

In an example, the orthogonal reactions are a thiol-ene reaction (e.g., a photo-initiated thiol-ene reaction) and a Michael addition reaction (a phosphine catalyzed Michael addition). In an example, the monomer is an allyl acrylamide and the co-monomer is an alkyl dithiol.

In an example, the co-monomer has the following structure:

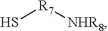

where [A] is any atom except a hydrogen, [Ak] is any aliphatic chain, and [Cy] is any cycle, and where R₇ is independently selected from [A], [Ak] or [Cy] and R₈ is an alkyl chain.

In an example, the co-monomer is an aminothiol. The alkyl chain of the aminothiol (e.g., R₇) can have 1 to 20 carbons, including all integer number of carbons and ranges therebetween. The alkyl chain that is a terminal substituent of the amine moiety (e.g., R₈) can have 1 to 20 carbons, including all integer number of carbons and ranges therebetween. The alkyl moieties, independently, can be substituted or unsubstituted and/or branched or linear.

It is desirable that the polymerization reactions have fast kinetics. In an example, each of the polymerization reactions is complete in 600 seconds or less. In various examples, each of the polymerization reactions is complete in 300 seconds or less or 100 seconds or less. In an example, the each of the polymerization reactions is complete in 1 to 600 seconds, including all integer second values and ranges therebetween. In other examples, the each of the polymerization reactions is complete in 1 to 300, or 1 to 100 seconds. By complete it is meant that the limiting reagent (the monomer, co-monomer, or reaction product) is not detectible by, for example, NMR spectroscopy.

A low ratio of monomer to co-monomer or intermediate (e.g., first reaction product, a second reaction product, and so on) to monomer or co-monomer monomer may be used. In an example, the ratio of monomer to co-monomer or intermediate (e.g., first reaction product, a second reaction product, and so on) to monomer or co-monomer is 1:0.5 to 1:10, including all values to 0.1 and ranges therebetween or 0.5:1 to 10:1, including all values to 0.1 and ranges therebetween. In another example, the ratio of monomer to co-monomer or intermediate (e.g., first reaction product, a second reaction product, and so on) to monomer or co-monomer is 1:0.5 to 1:5 or 0.5:1 to 5:1.

Determination of the reaction conditions (e.g., reaction time and temperature) required to make a desired compound are within the purview of one having skill in the art.

Suitable reaction times for a thiol-ene reaction may be 90-300 seconds. Suitable reaction times for a Michael addition may be 5-60 minutes. For a thiol-ene and/or Michael addition, suitable reaction temperatures may be room temperature to 60° C. and suitable catalyst concentration may be 0.1-20 mol % catalyst.

Each reaction (e.g., addition of monomer or co-monomer) can be carried out at high yield. For example, at 4 mg reaction scale the yield (including purification) of each step is greater than 86% and at 20 mg scale the yield (including purification) of each step is greater than 97%.

During polymerization, the product of each monomer and/or co-monomer addition may be purified. To facilitate such purification, a monomer conjugated to a solid support or a monomer having a fluorous tag may be used.

In an aspect, the present disclosure provides compositions comprising compounds of the present disclosure. The compositions also comprise one or more pharmaceutically acceptable carrier.

The compositions may include one or more standard pharmaceutically acceptable carriers. Non-limiting examples of compositions include solutions, suspensions, emulsions, solid injectable compositions that are dissolved or suspended in a solvent before use, and the like. The compositions may be prepared by dissolving, suspending, or emulsifying one or more of the active ingredients in a diluent. Non-limiting examples of diluents are distilled water (e.g., distilled water for injection), physiological saline, vegetable oil, alcohol, and a combination thereof. Further, the injections may contain stabilizers, solubilizers, suspending agents, emulsifiers, soothing agents, buffers, preservatives, and the like. The compositions may be sterilized in the final formulation step or prepared by sterile procedure. The composition of the disclosure may also be formulated into a sterile solid preparation, for example, by freeze-drying, and may be used after sterilized or dissolved in sterile water (e.g., sterile water suitable for injection) or other sterile diluent(s) immediately before use. Non-limiting examples of pharmaceutically acceptable carriers can be found in: Remington: The Science and Practice of Pharmacy (2005) 21st Edition, Philadelphia, Pa. Lippincott Williams & Wilkins.

In an aspect, the disclosure provides kits. A kit may comprise pharmaceutical preparations containing any one or any combination of compounds and printed material. In an example, a kit comprises a closed or sealed package that contains the pharmaceutical preparation. In various examples, the package comprises one or more closed or sealed vials, bottles, blister (bubble) packs, or any other suitable packaging for the sale, or distribution, or use of the compounds and compositions comprising compounds of the present disclosure. The printed material may include printed information. The printed information may be provided on a label, or on a paper insert, or printed on the packaging material itself. The printed information may include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material may include an indication that the pharmaceutical composition and/or any other agent provided with it is for treatment of a subject having cancer and/or other diseases and/or any disorder associated with cancer and/or other diseases. In various examples, the product includes a label describing the contents of the container and providing indications and/or instructions regarding use of the contents of the container to treat a subject having any cancer and/or other diseases. A kit may comprise a single dose or multiple doses.

In an aspect, the present disclosure provides methods of using one or more compound or composition thereof. The method may comprise intracellular delivery of one or more cargo group of the compound. Upon delivery, the cargo is delivered (e.g., released) in its effective form.

The compounds may be suitable in methods to treat cancers (e.g., leukemia, lung cancer (e.g., non-small cell lung cancer), dermatological cancer, premalignant lesions of the upper digestive tract, malignancies of the prostate, malignancies of the brain, malignancies of the breast, and the like, and combinations thereof), bacterial infections, viral infections, urinary tract infections, skin infections, cystic fibrosis, sepsis, fungal infections, and the like, and combinations thereof. Compounds of the present disclosure may also be fluorescent probes. For example, one or more compounds of the present disclosure can be used to treat, for example, cancer, bacterial infections, viral infections, urinary tract infections, skin infections, cystic fibrosis, sepsis, fungal infections, and the like, and combinations thereof. The method may further comprise imaging (e.g., fluorescent imaging, molecular imaging, and the like) when a compound comprises a fluorescent group as a cargo group. A method can be carried out in combination with one or more known therapies.

In various examples, a compound and/or composition of the present disclosure is used to treat a bacterial infection caused by one or more bacteria. Non-limiting examples of bacteria include Listeria monocytogenes, Staphylococcus aureus, Pseudomonas aeruginosa, Tuberculosis, Salmonella enterica, Francisella tularensis, and the like, and combinations thereof.

In an example, one or more compound and/or one or more composition comprising one or more compound described herein are be administered to a subject in need of treatment using any known method and/or route, including oral, parenteral, subcutaneous, intraperitoneal, intrapulmonary, intranasal and intracranial injections. Parenteral infusions include intramuscular, intravenous, intraarterial, intraperitoneal, and subcutaneous administration. The present disclosure also provides topical and/or transdermal administration.

A method can be carried out in a subject in need of treatment who has been diagnosed with or is suspected of having cancer, bacterial infections, viral infections, urinary tract infections, skin infections, cystic fibrosis, sepsis, fungal infections, and the like, and combinations thereof (e.g., therapeutic use). A method can also be carried out in a subject who have a relapse or a high risk of relapse after being treated for cancer, bacterial infections, viral infections, urinary tract infections, skin infections, cystic fibrosis, sepsis, fungal infections, and the like, and combinations thereof.

A subject in need of treatment may be a human or non-human mammal. Non-limiting examples of non-human mammals include cows, pigs, mice, rats, rabbits, cats, dogs, other agricultural animal, pet, service animals, and the like.

In an example, a compound is used to inhibit cancer growth, kill bacteria, treat fungal infections, and the like. In an example, a method comprising administering to an individual in need of treatment with a compound or composition in an amount (e.g., 0.1 nM to 1 mM) and time sufficient to inhibit cancer growth, kill bacteria, treat fungal infections, and the like, and combinations thereof.

In an example a method of the present disclosure for treating cancer and/or a disease comprises: i) administering to a subject in need of treatment a composition of the present disclosure, where the compound delivers a cargo.

In an example, the compounds and compositions are suitable in methods using imaging (e.g., fluorescence microscopy). Methods may further comprise fluorescence microscopy. Methods comprising fluorescence microscopy may be combined with other techniques, such as, for example, flow cytometry. Techniques for fluorescence microscopy are known in the art.

The steps of any of the methods described in the various embodiments and examples disclosed herein are sufficient to carry out the methods and produce the compositions of the present disclosure. Thus, in an embodiment, the method consists essentially of a combination of the steps of the methods disclosed herein. In another embodiment, the method consists of such steps.

In the following Statements, various examples of the methods and compositions of the present disclosure are described:

Statement 1. A compound having the structure:

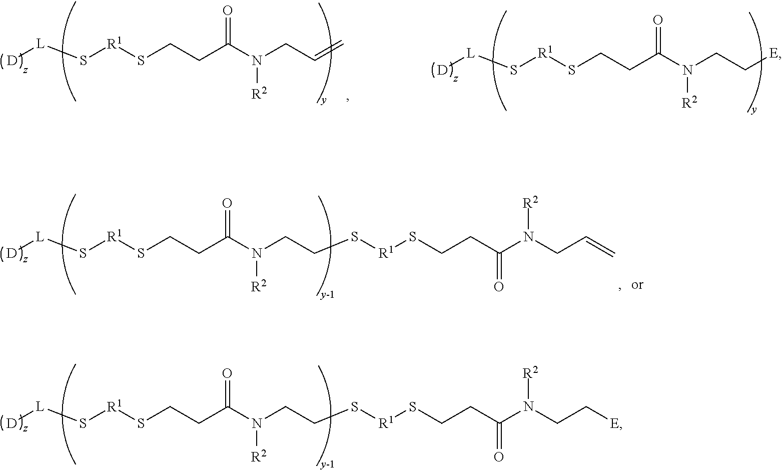

where L is chosen from a linking group, NH, N, O, and S; D is a cargo group; R′ is independently at each occurrence in the compound chosen from straight chain or branched C₂ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)_{d}—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8); diol groups having the structure —CH₂—CHOH—(CH₂)ₑ—CHOH—CH₂—, where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like); R² is independently at each occurrence in the compound chosen from cationic groups (e.g., alkyl amine groups, alkyl guanidinium groups, and the like), aliphatic electrophilic groups, aliphatic nucleophilic groups, straight chain or branched C₁ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)_{d}—, where a is 1 to 10 (e.g., 1, 2, 3, 4, 5, 6, 7, 8, 9, 10), b is 0 to 8 (e.g., 1, 2, 3, 4, 5, 6, 7, 8), and d is 0 to 8 (e.g., 1, 2, 3, 4, 5, 6, 7, 8); diol groups having the structure —CH₂—CHOH—(CH₂)ₑ—CHOH—CH₂—; where e is 0 to 10 (e.g., 1, 2, 3, 4, 5, 6, 7, 8, 9, 10); substituted or unsubstituted C₅ to C₁₀ aryl groups (e.g., phenyl groups, napthyl groups, hydroxybenzyl groups, tolyl groups, xylyl groups, furanyl groups, benzofuranyl groups, indolyl groups, imidazolyl groups, benzimidazolyl groups, pyridinyl groups, and the like); and substituted or unsubstituted C₃ to C₈ aliphatic cyclic groups (e.g., cyclobutyl groups, cyclopentyl groups, cyclohexyl groups, cycloheptyl groups, cyclooctyl groups, and the like); E is an end group chosen from ═CH₂, D, and L-(D)_{z}; y is 1 to 12; and z is 1 to 5.
Statement 2. The compound according to Statement 1, where the compound has the following structure:

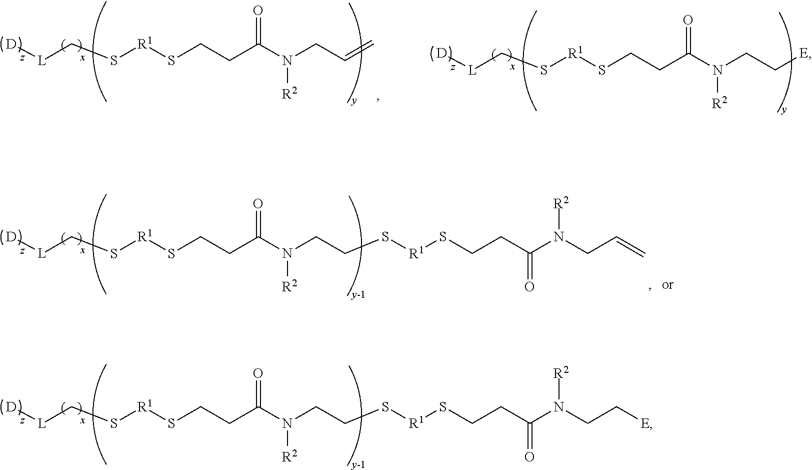

wherein x is 1 to 8.
Statement 3. The compound according to any one of the preceding Statements, where the cargo group is chosen from chemotherapeutic groups, antibiotic groups, fluorophore groups, peptide groups, protein groups, nucleic acid groups, kinase inhibitor groups, antibody groups, enzyme inhibitor groups, small molecule drug groups, sugars/glycan groups, and combinations thereof. Cargo groups may be formed from any of the following non-limiting examples:

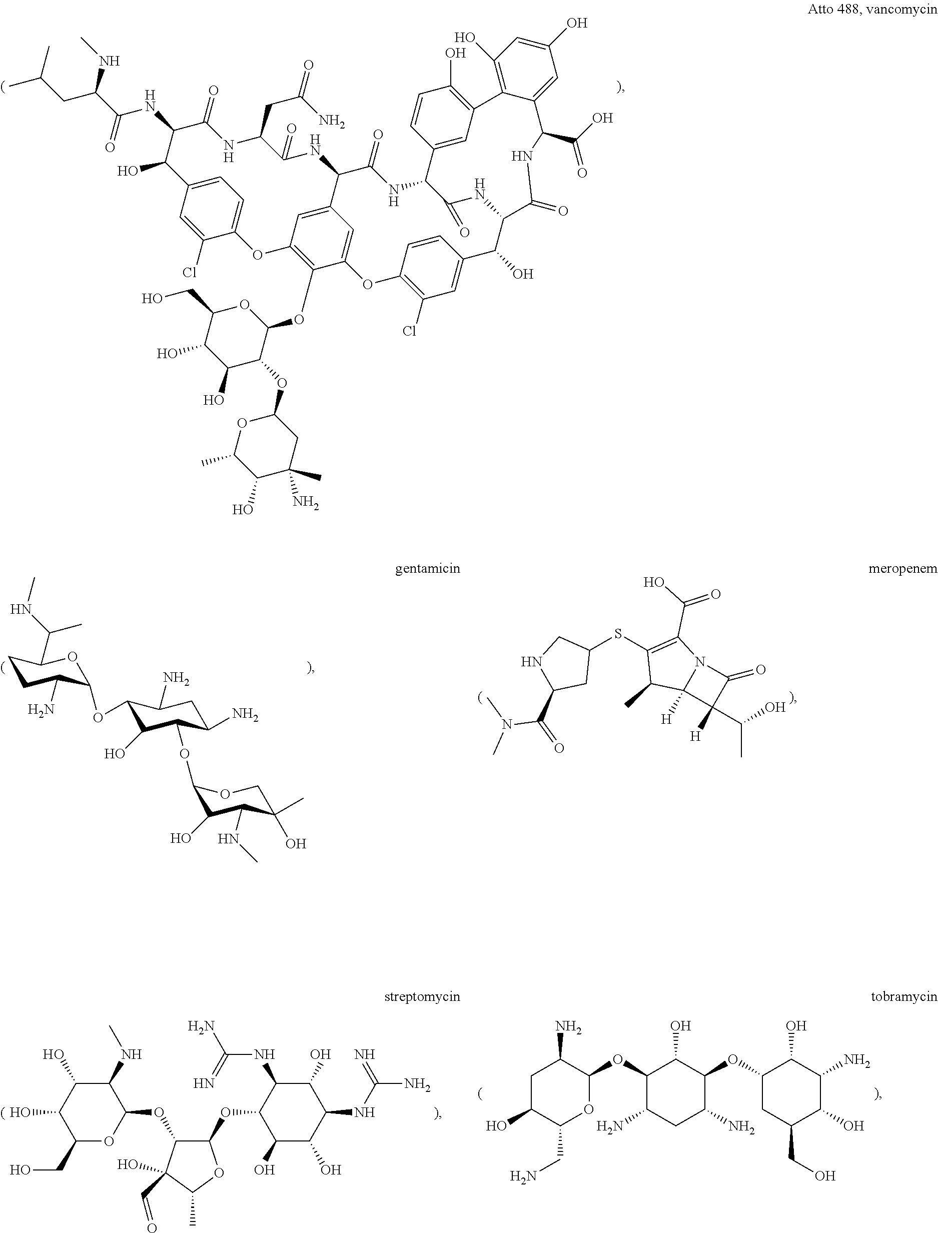

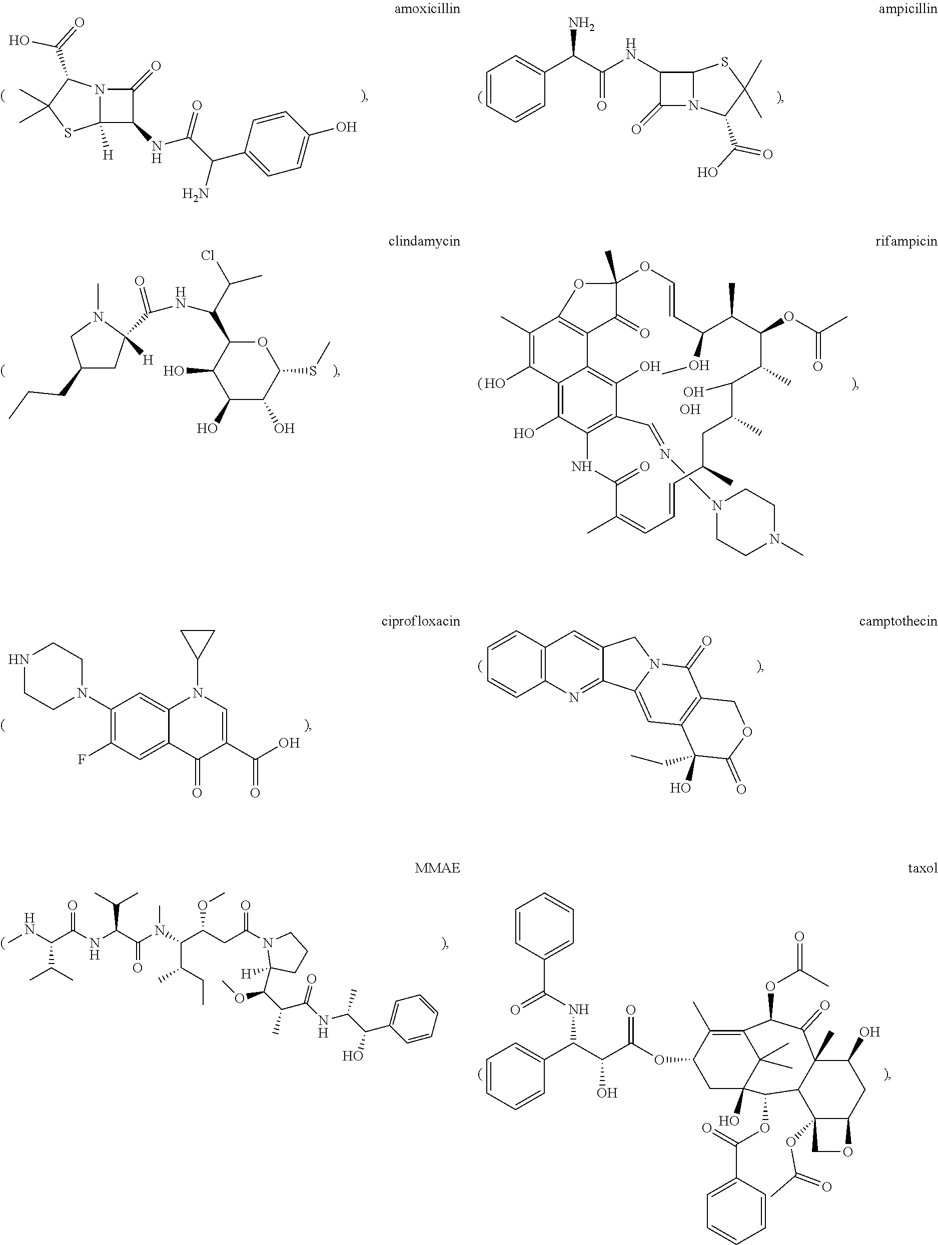

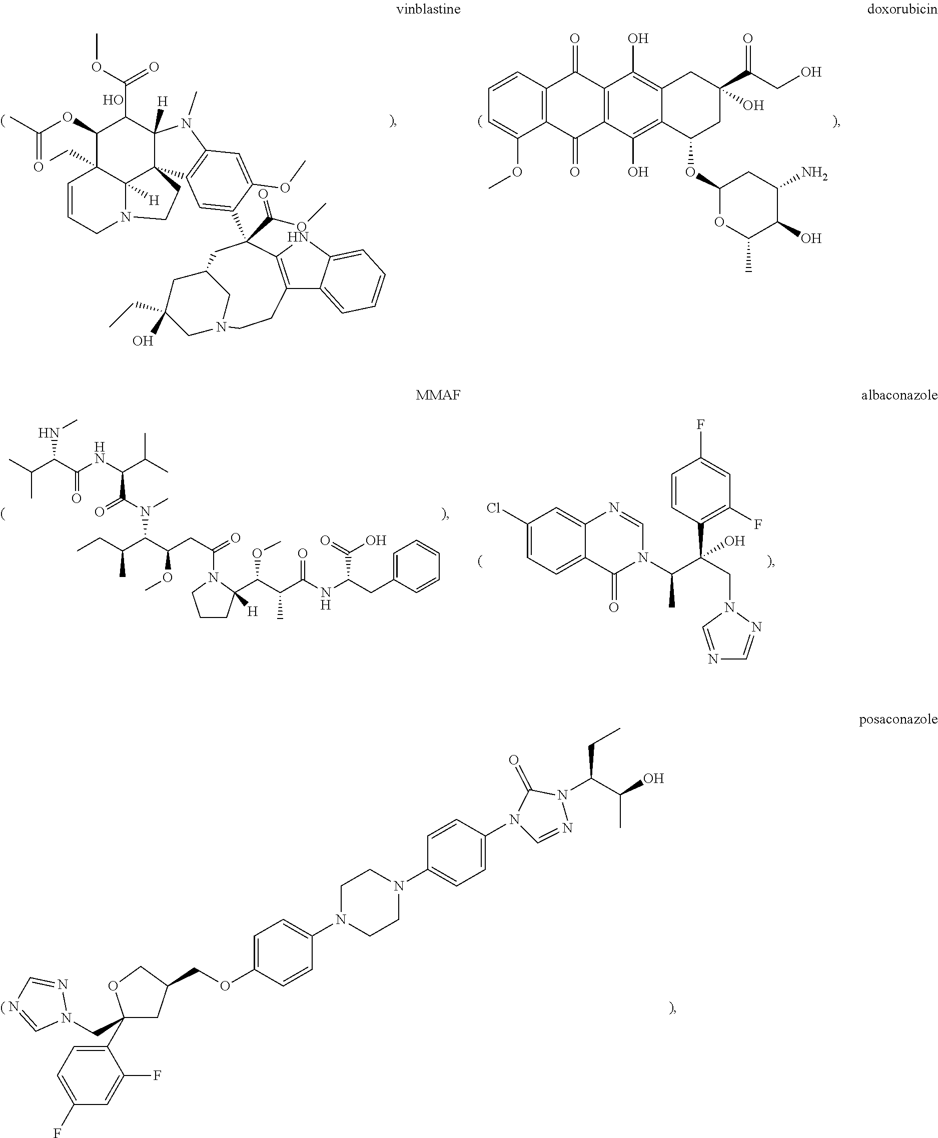

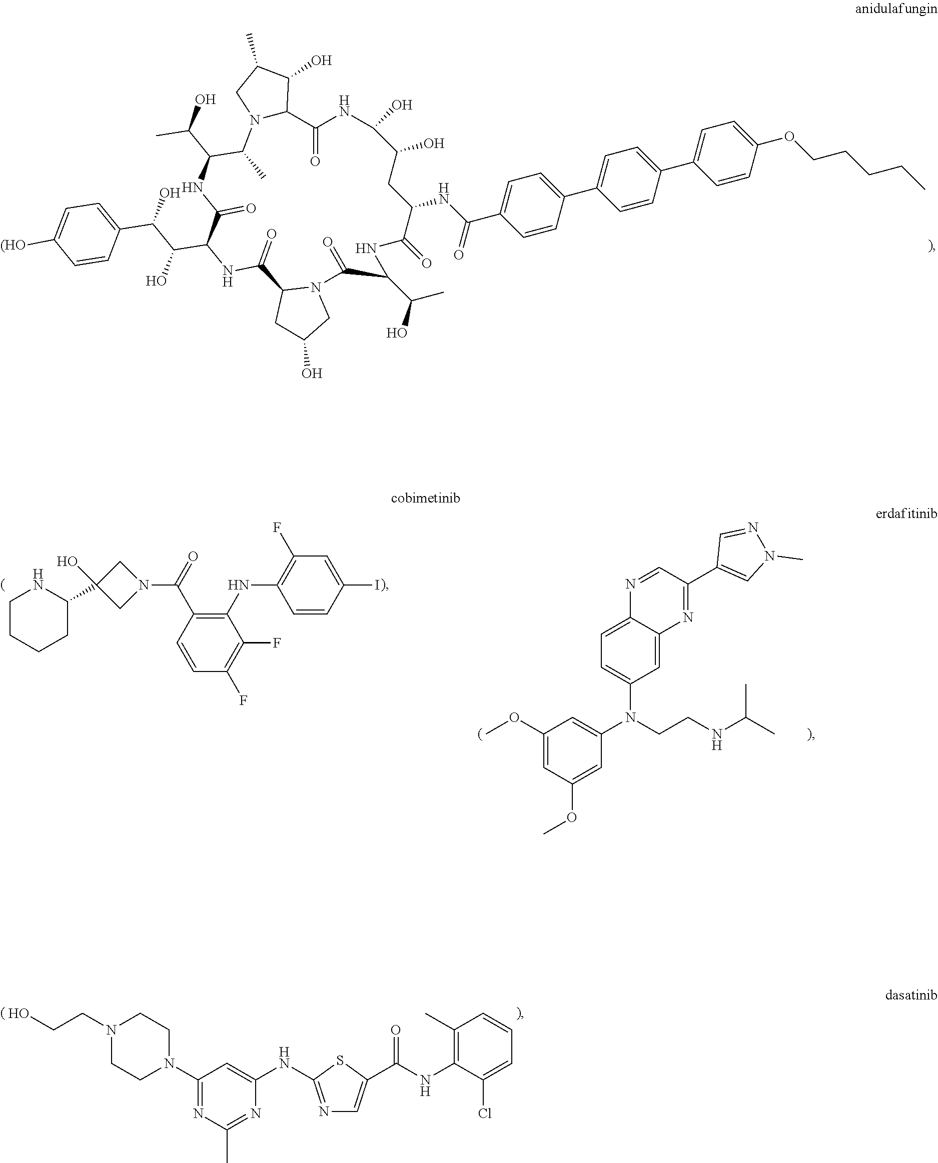

and the like.
Statement 4. The compound according to any one of the preceding Statements, wherein the cargo group is chosen from a non-functionalized vancomycin group, a fluorophore-modified vancomycin group, a fluorescein group, an Atto 488 group, and a peptide group having the sequence KADNAAIESIRNGTYDHIDVYRDEALNNRFQIKGVELKSGYKDW (SEQ ID NO: 1), and combinations thereof.
Statement 5. The compound according to any one of the preceding Statements, wherein R is independently at each occurrence in the compound chosen from substituted or unsubstituted propyl groups, substituted or unsubstituted butyl groups,

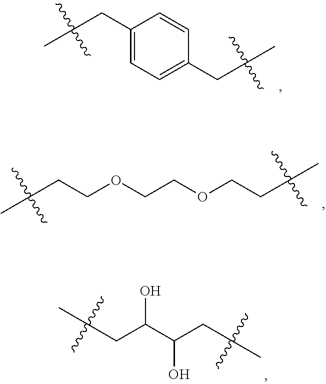 [SMILES: *Cc1ccc(C*)cc1 ; *CCOCCOCC* ; *CC(O)C(O)C*]

and combinations thereof.
Statement 6. The compound of any one of the preceding Statements, wherein R² is independently at each occurrence in the compound chosen from substituted or unsubstituted butyl groups, substituted or unsubstituted benzyl groups,

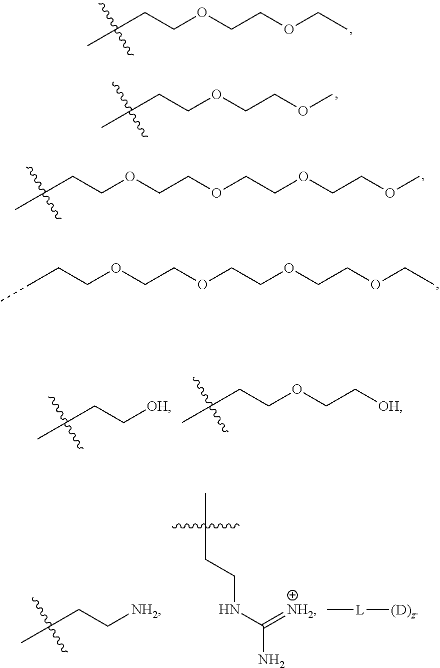

Statement 7. The compound according to any one of the preceding Statements, wherein the linking group is chosen from

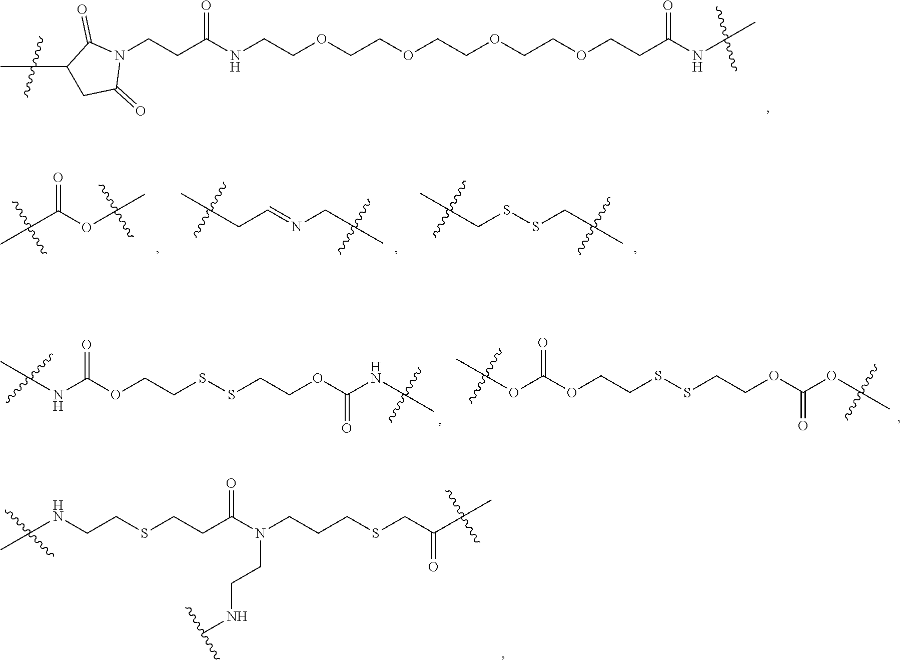

-Val-citrulline-, and combinations thereof.
Statement 8. The compound any one of the preceding Statements, wherein the compound has the following structure:

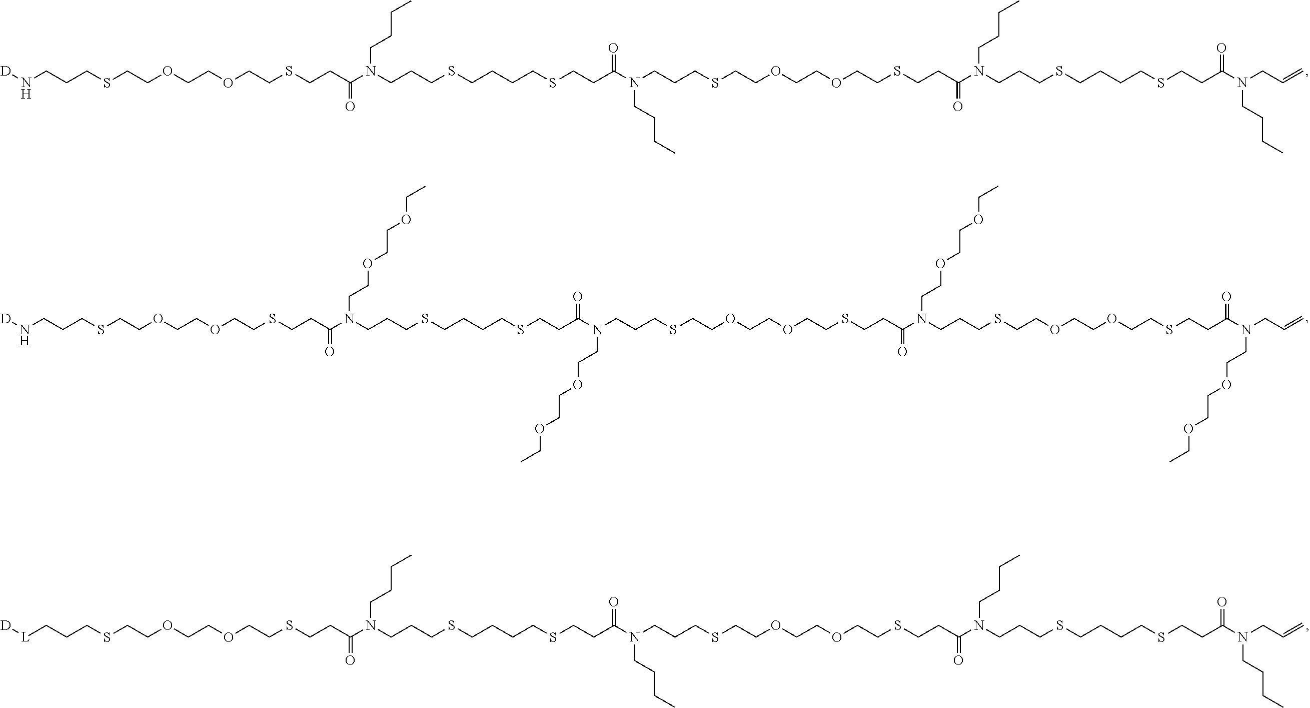

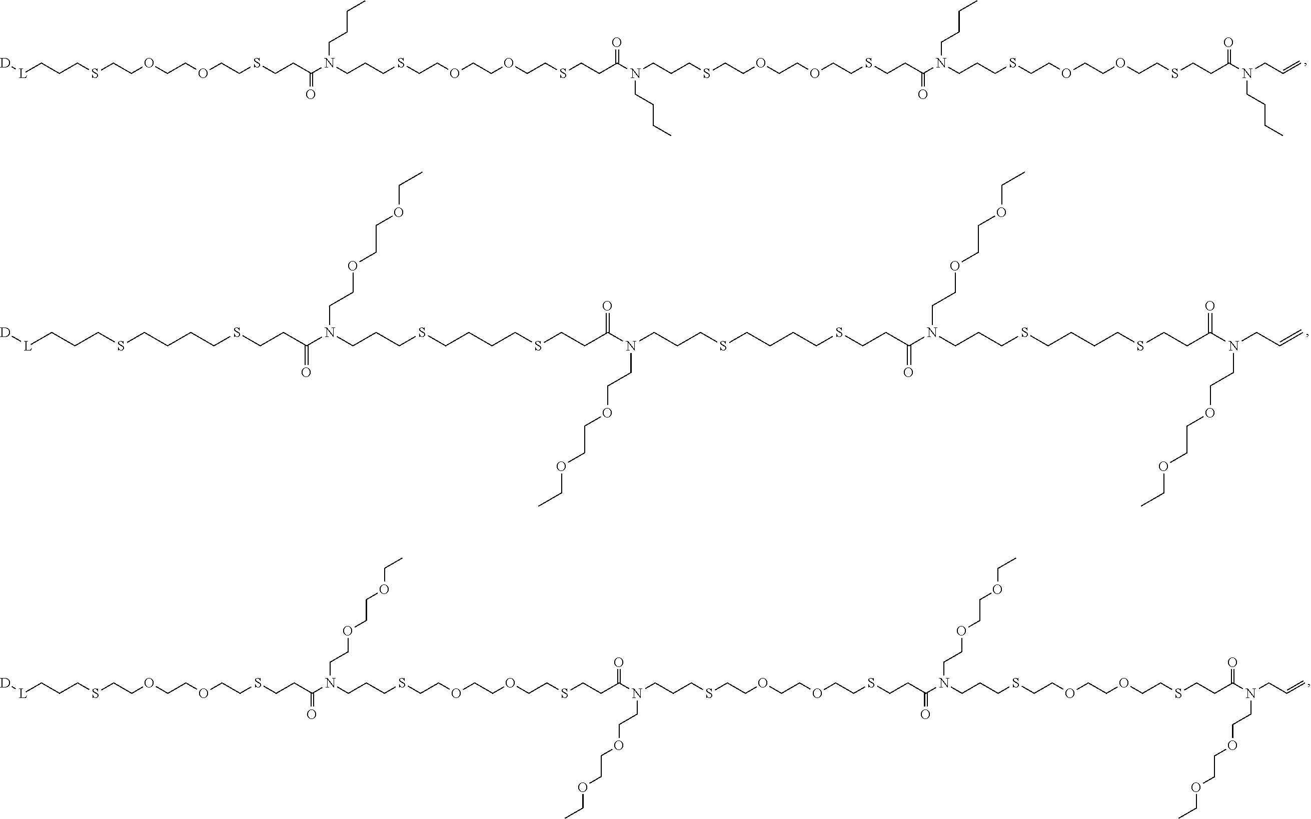

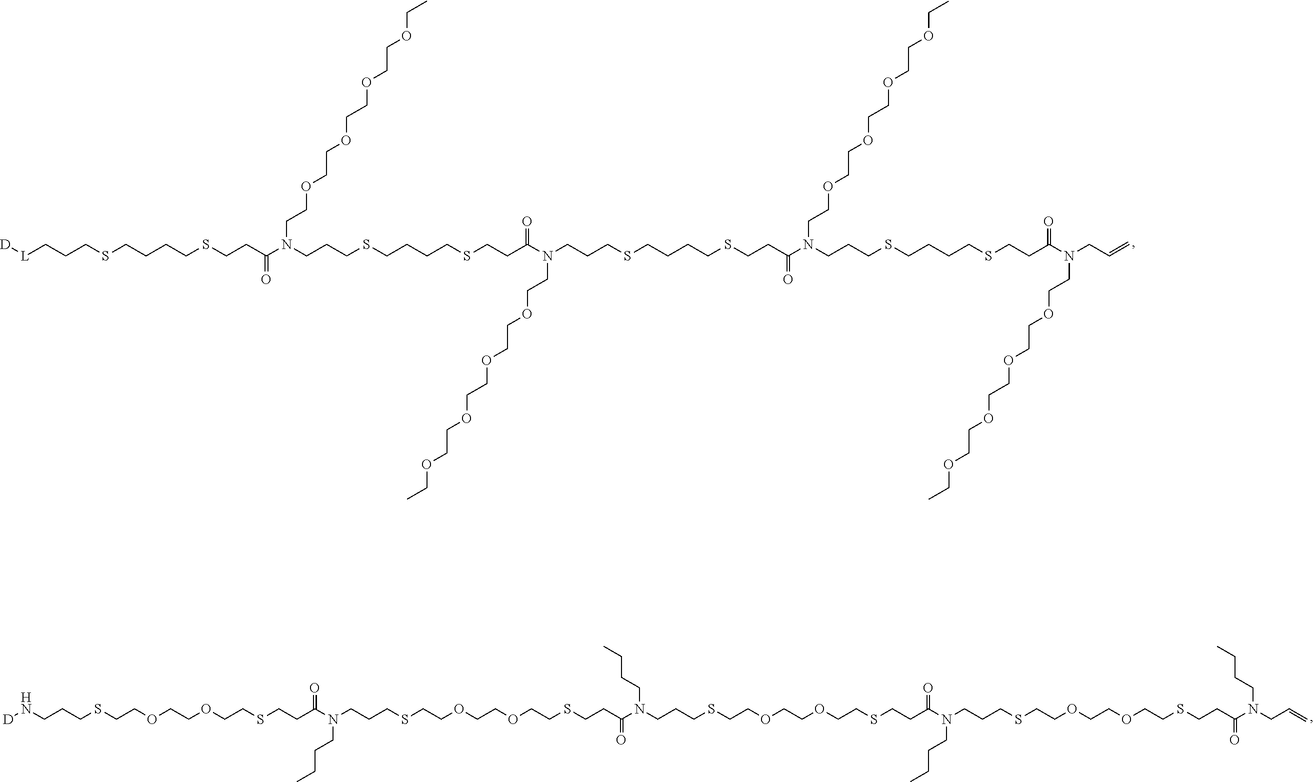

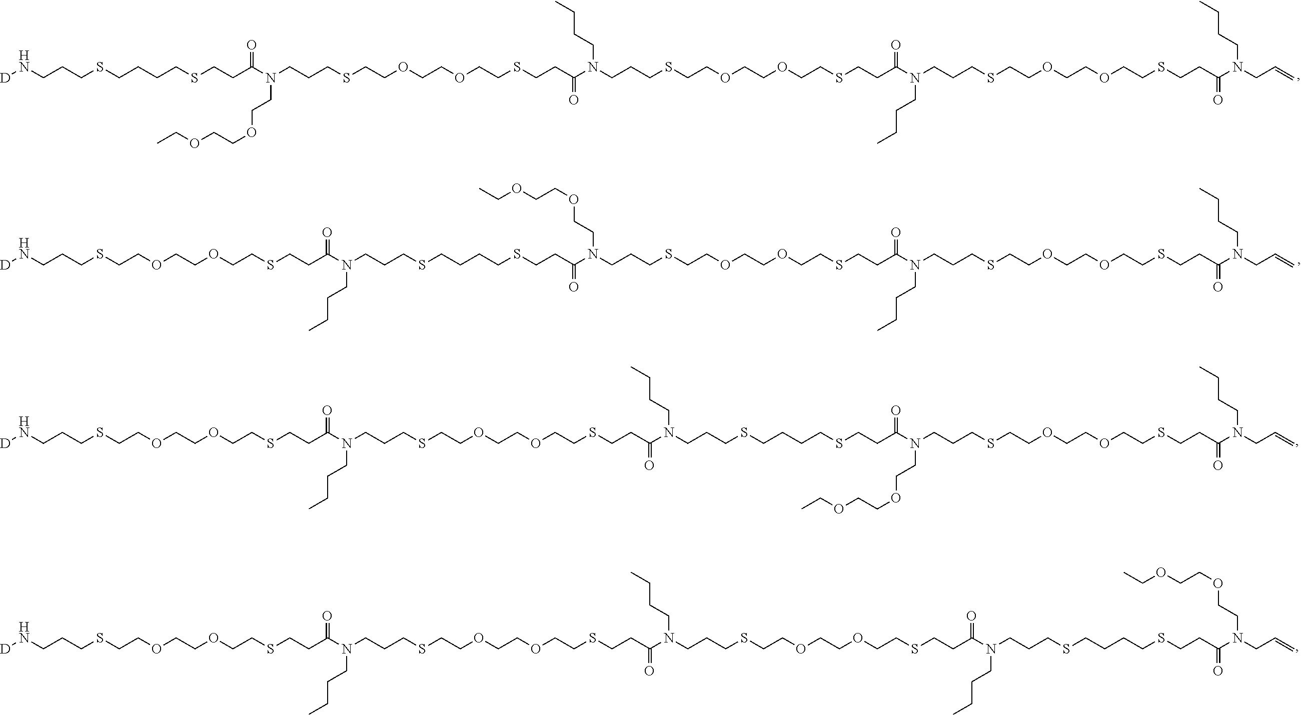

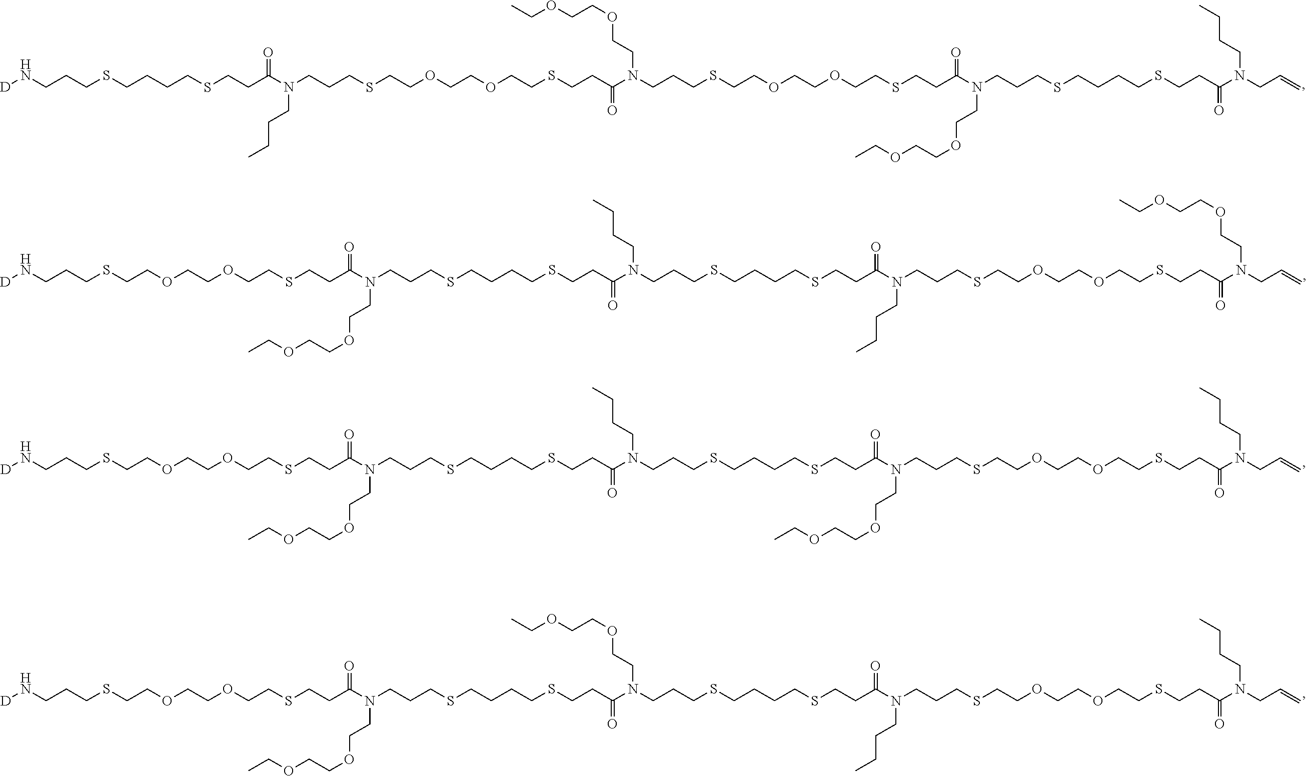

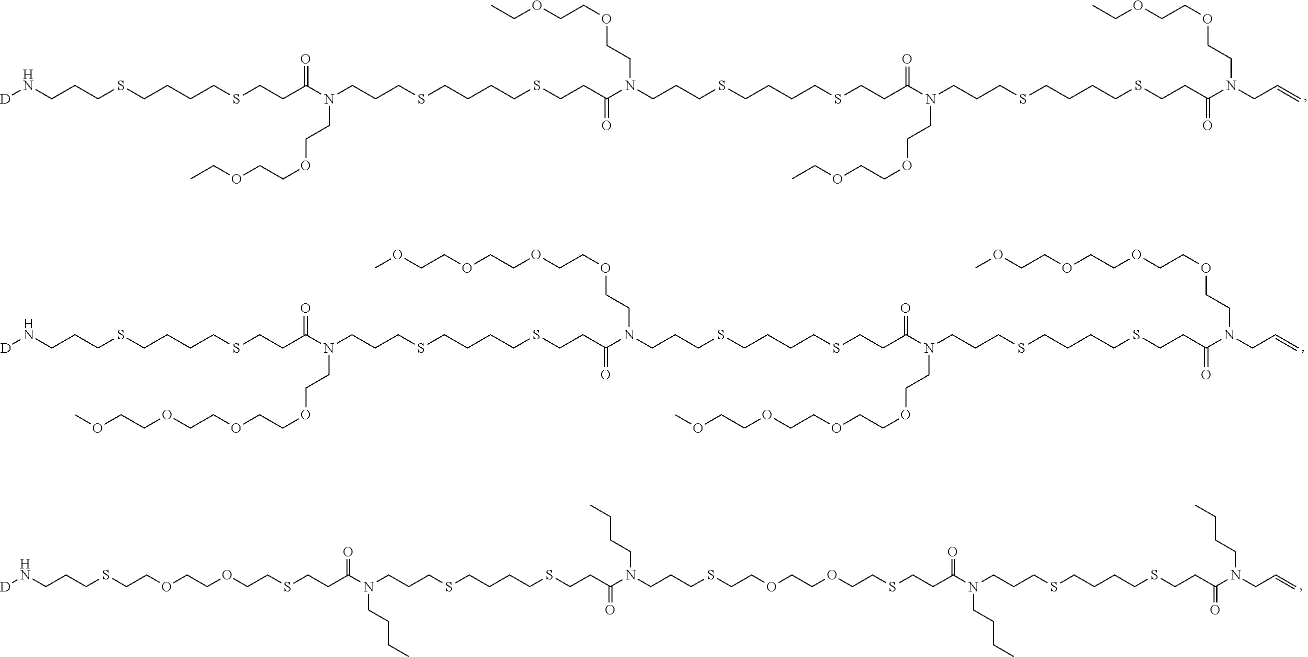

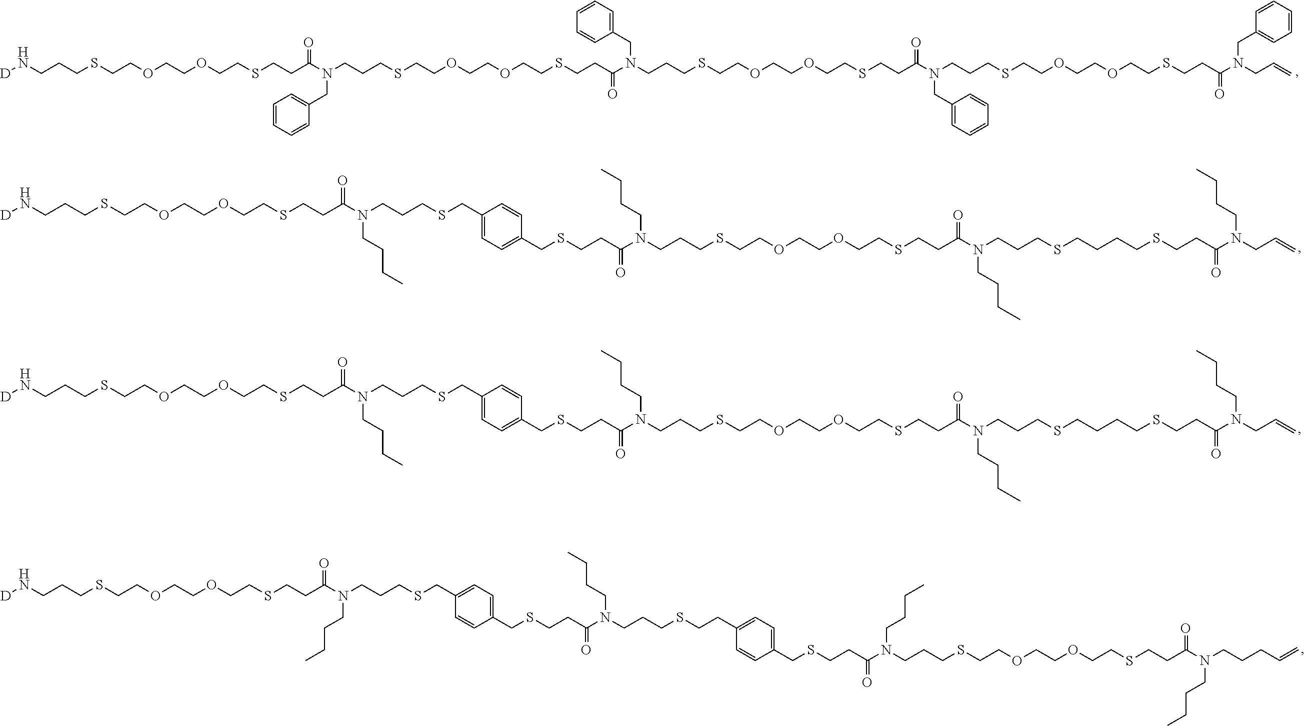

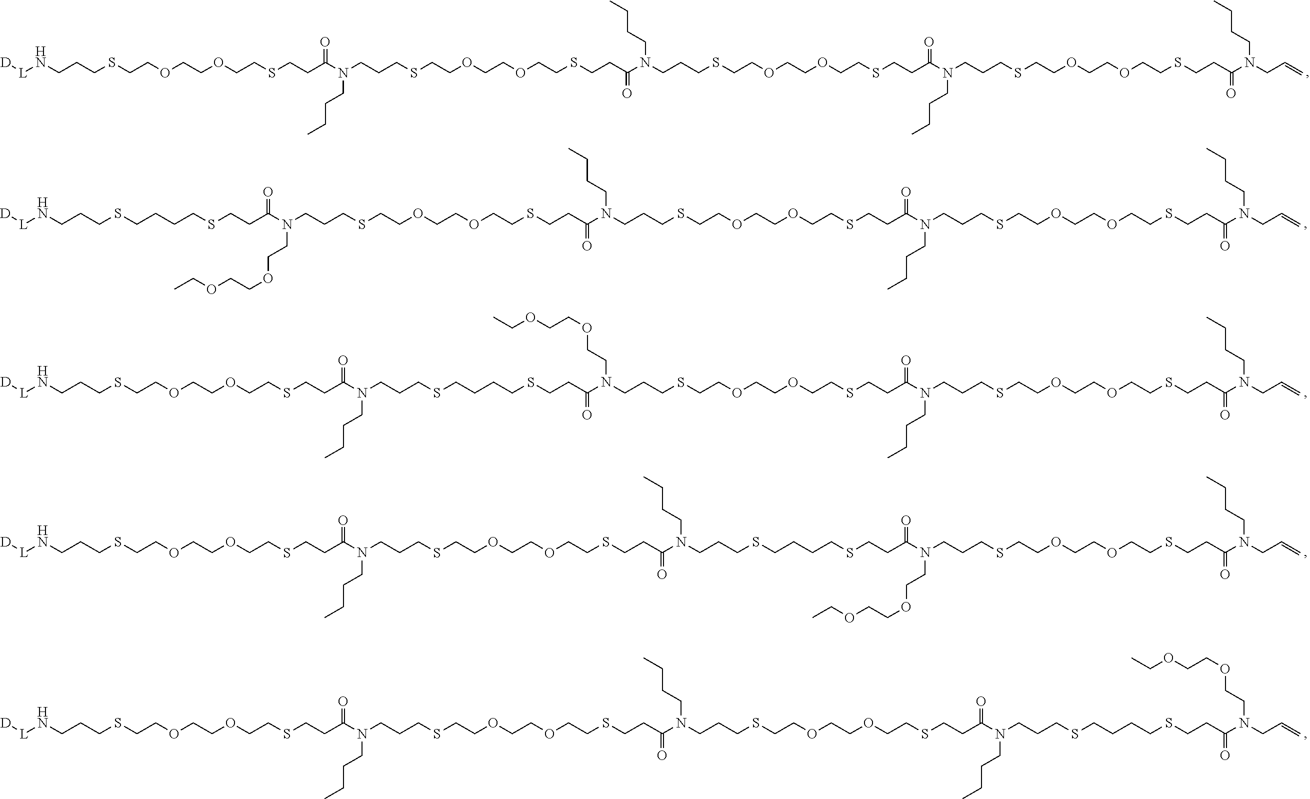

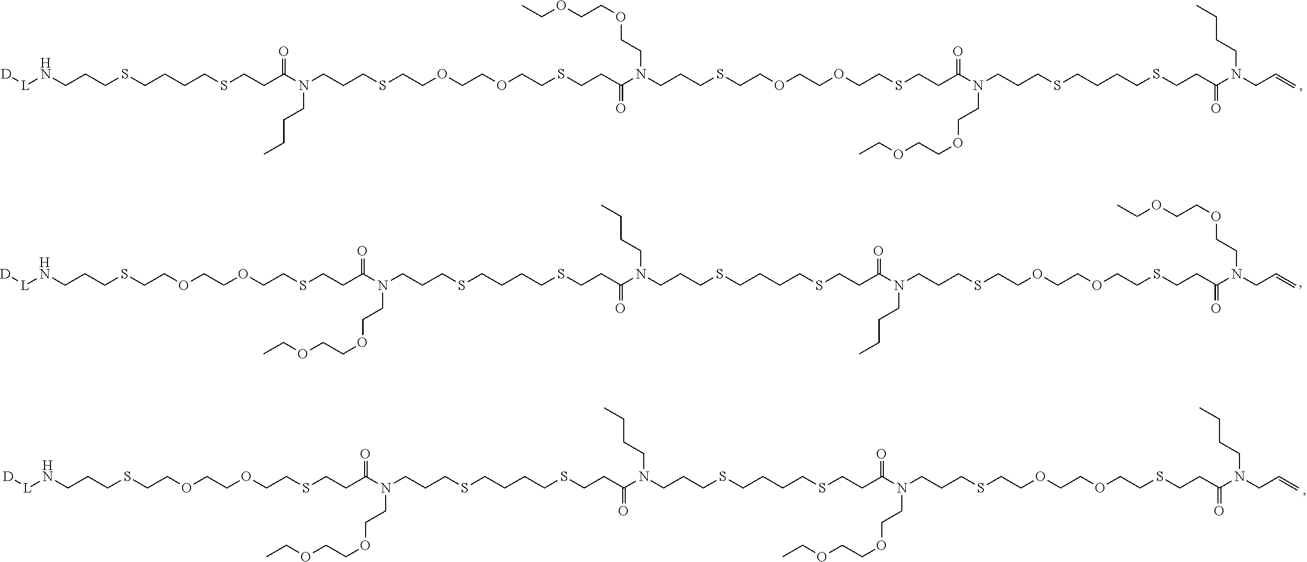

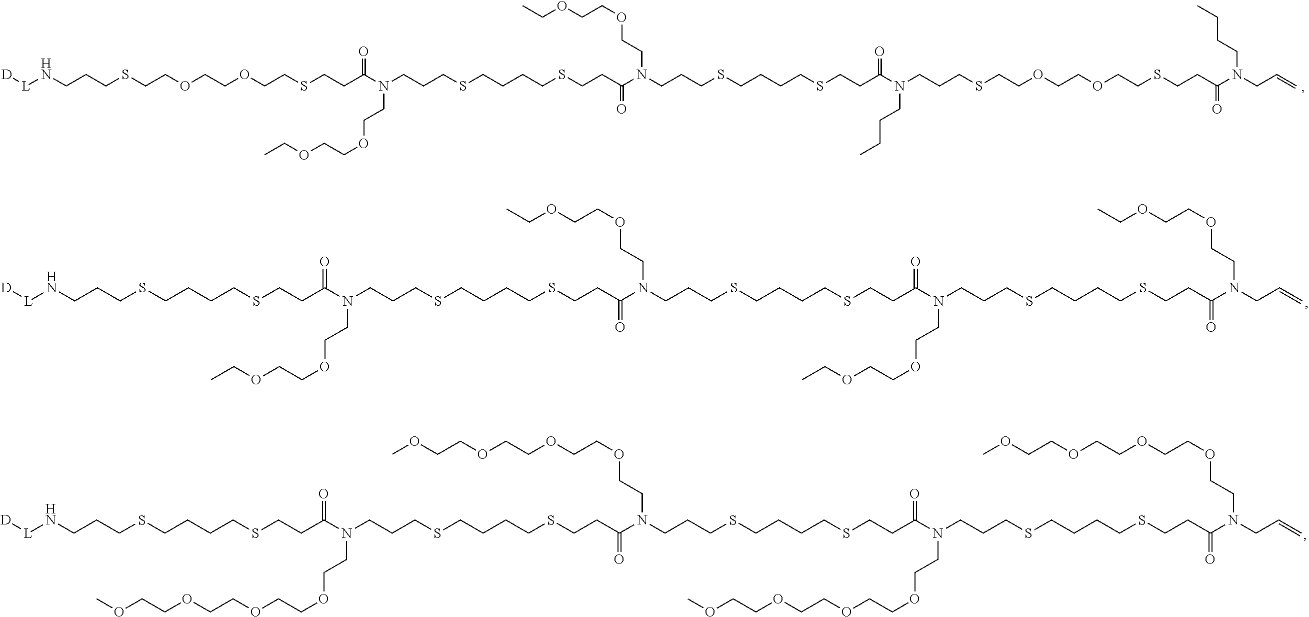

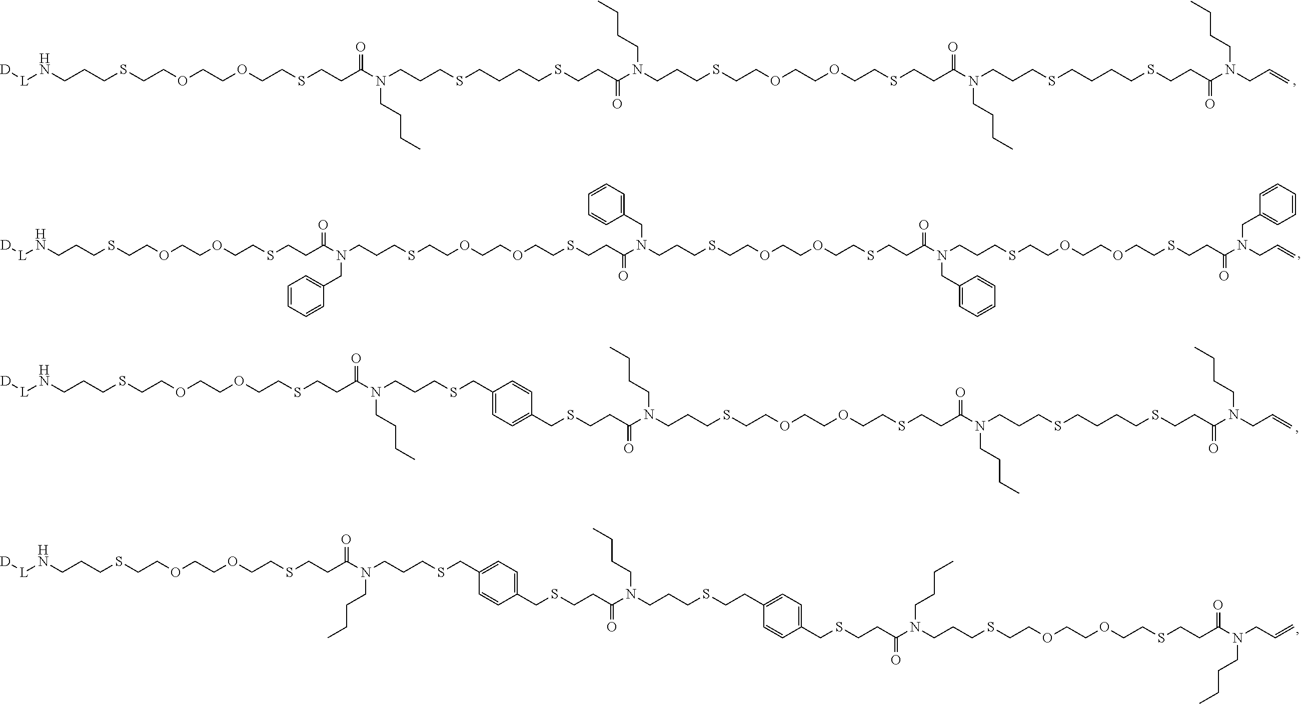

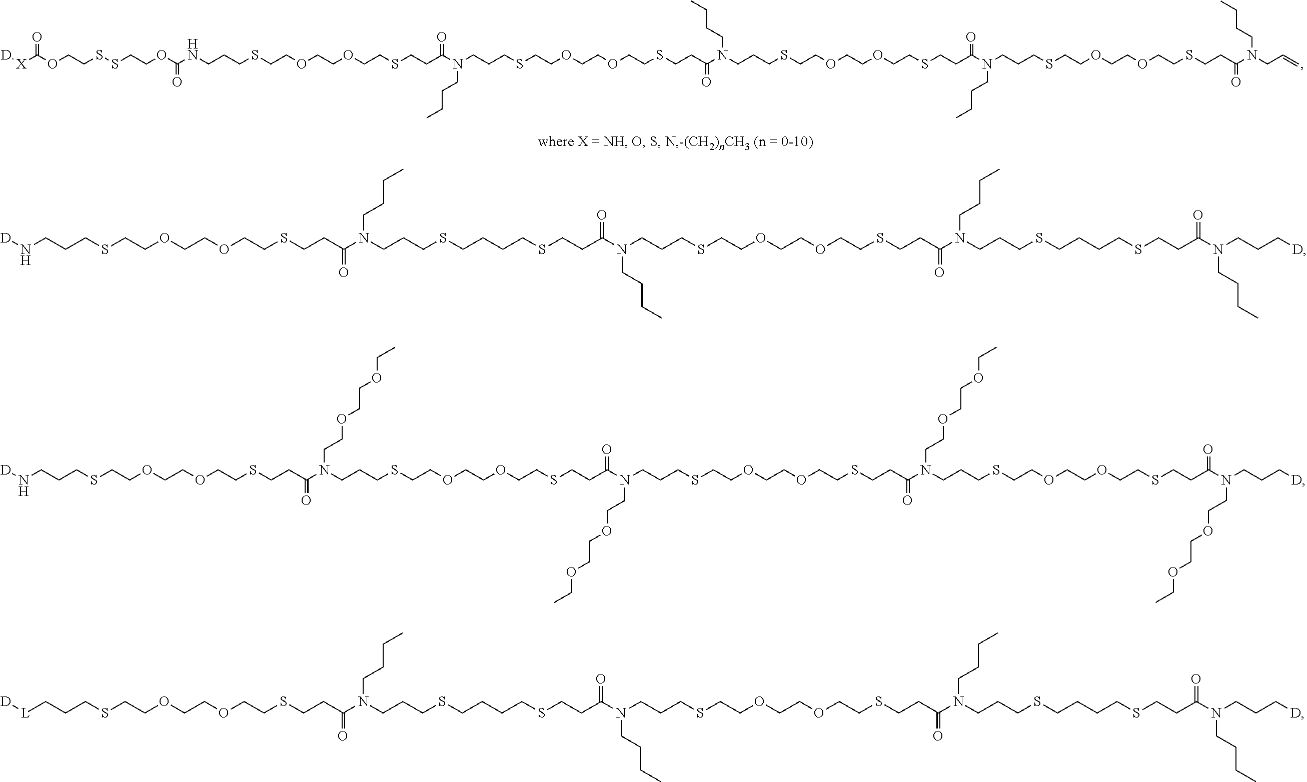

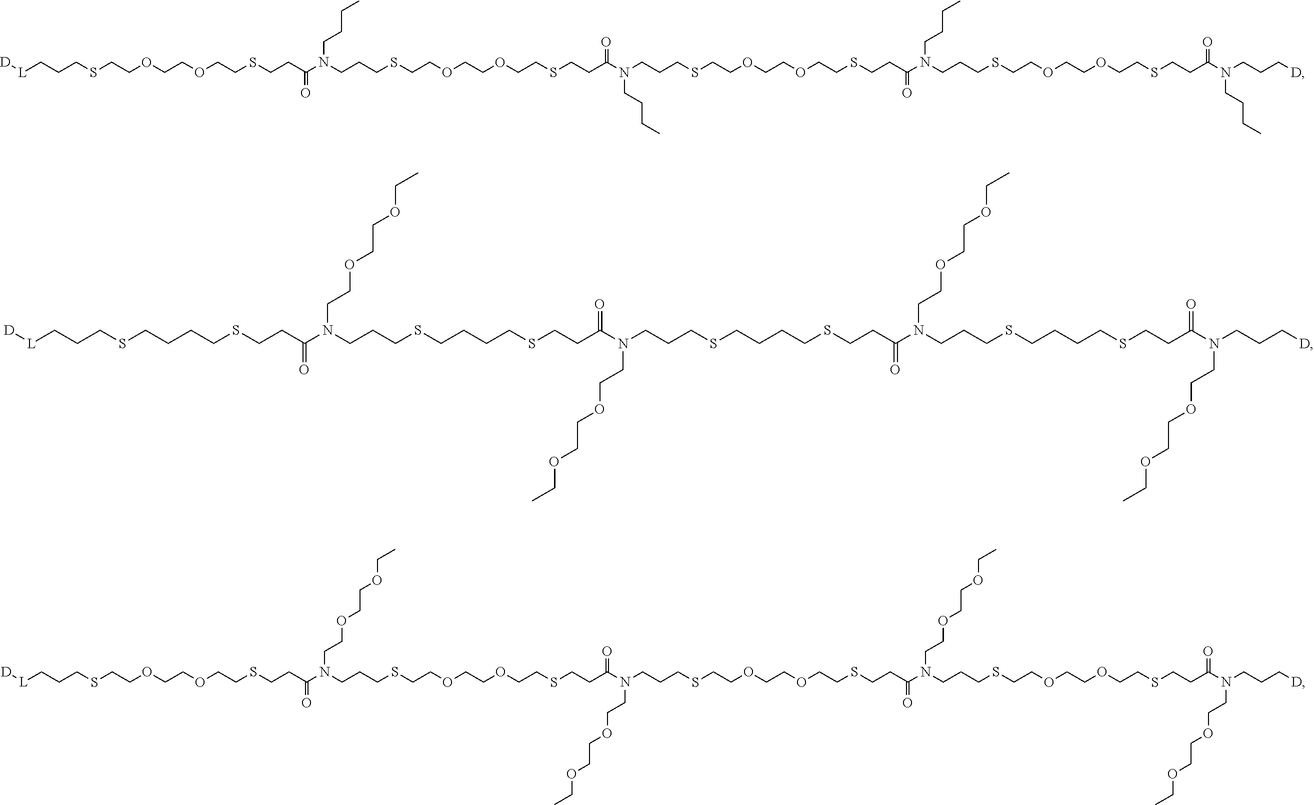

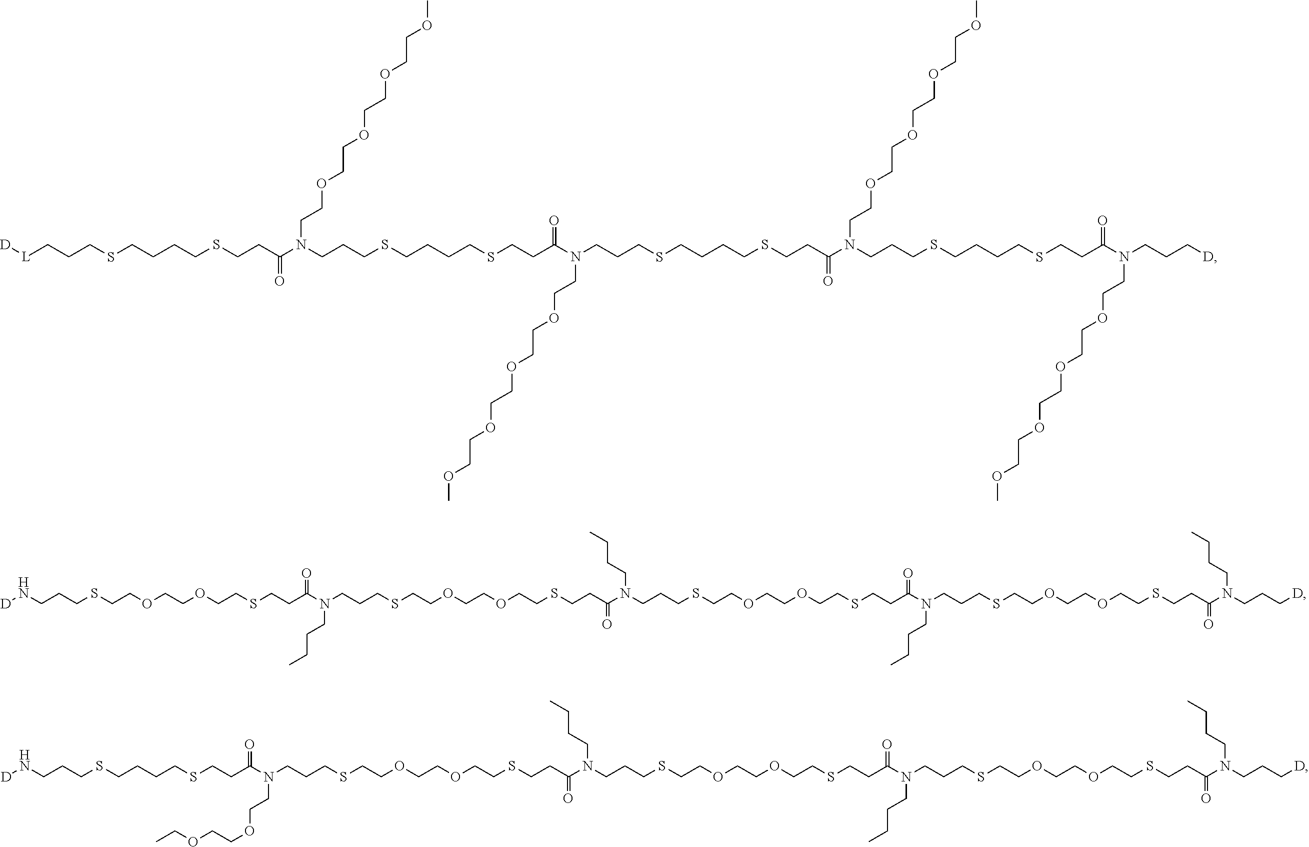

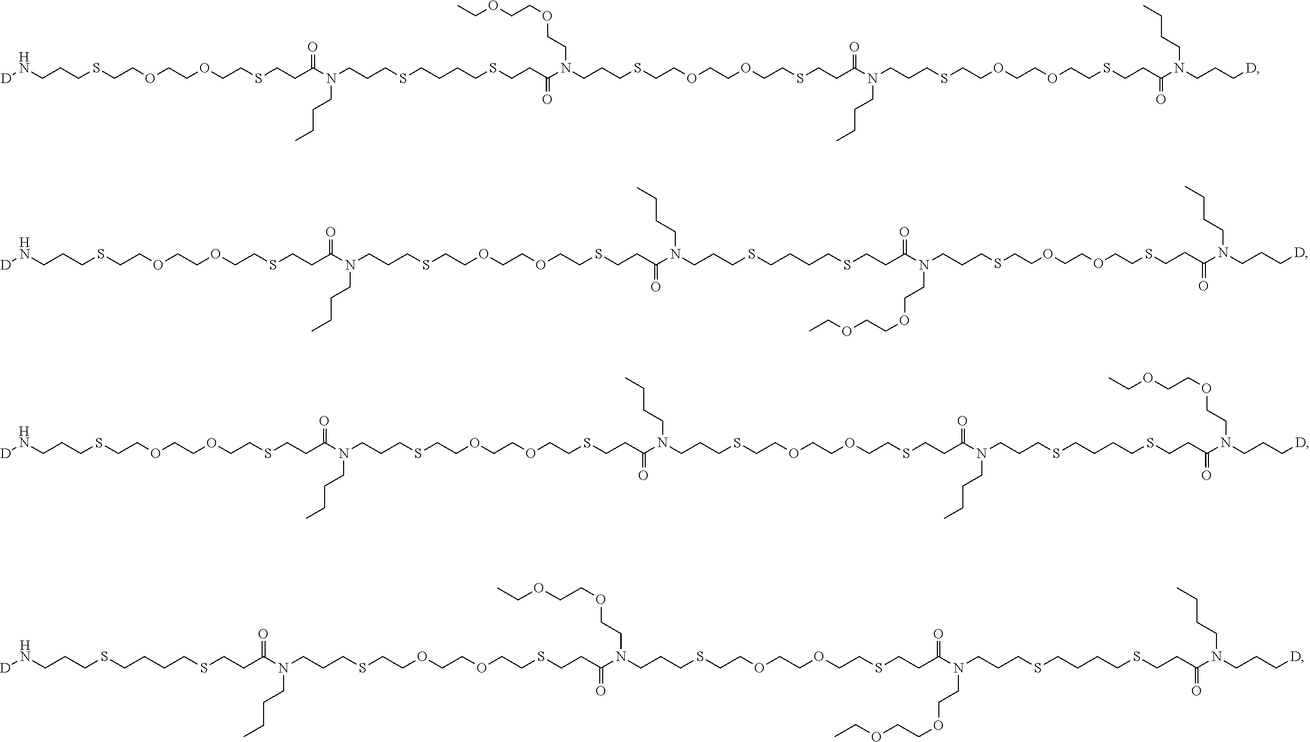

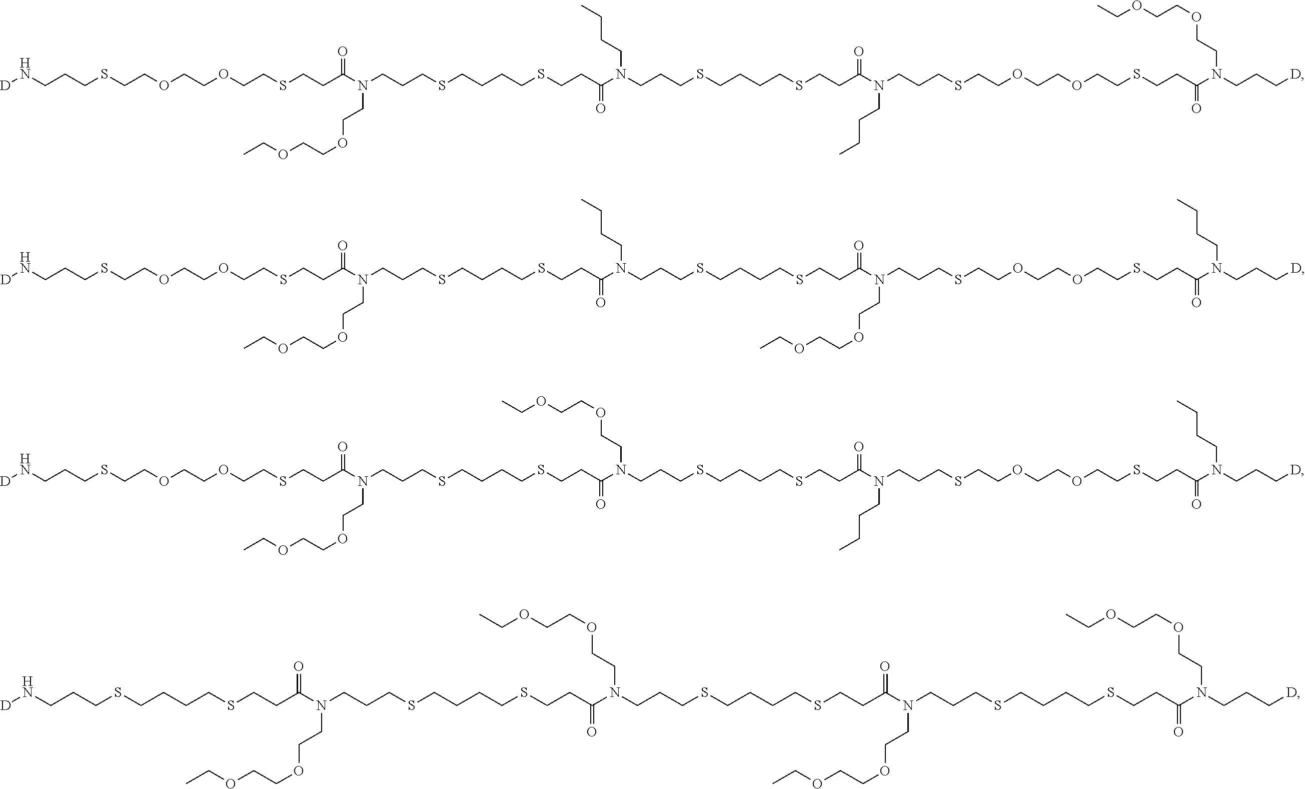

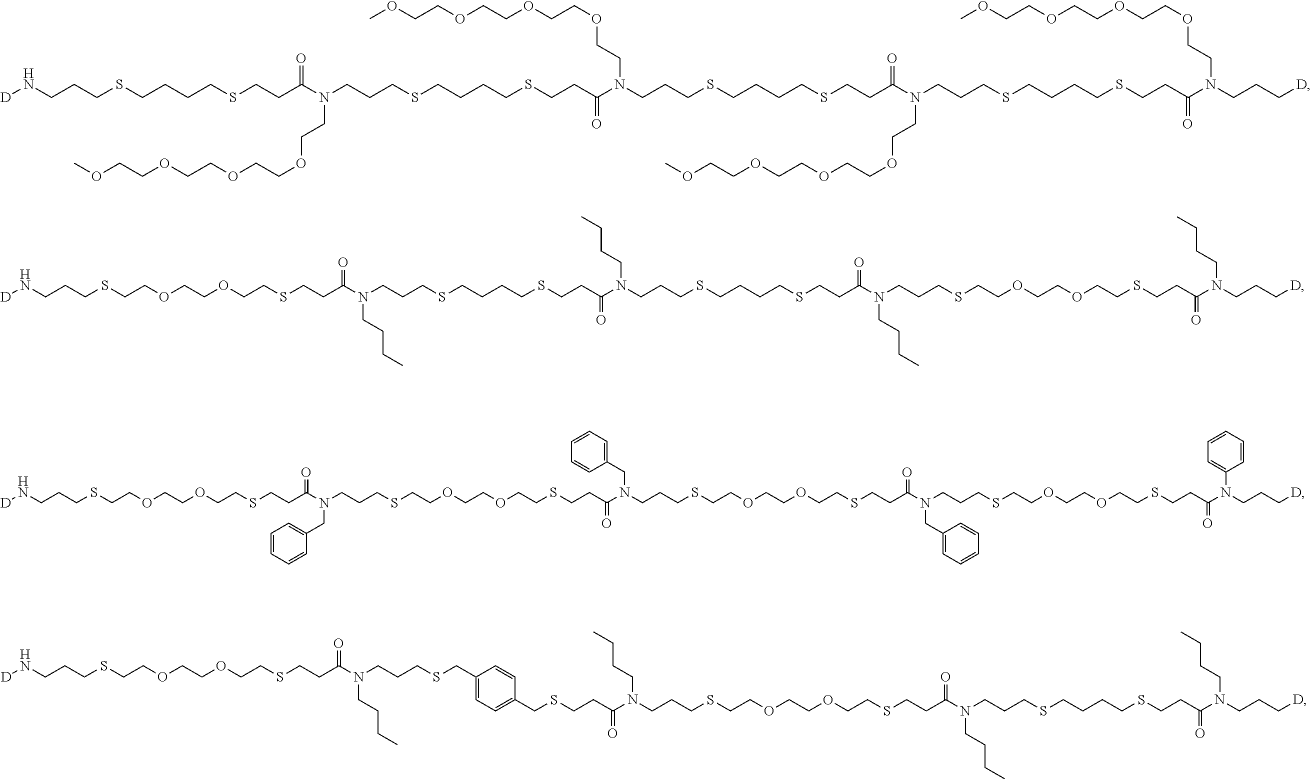

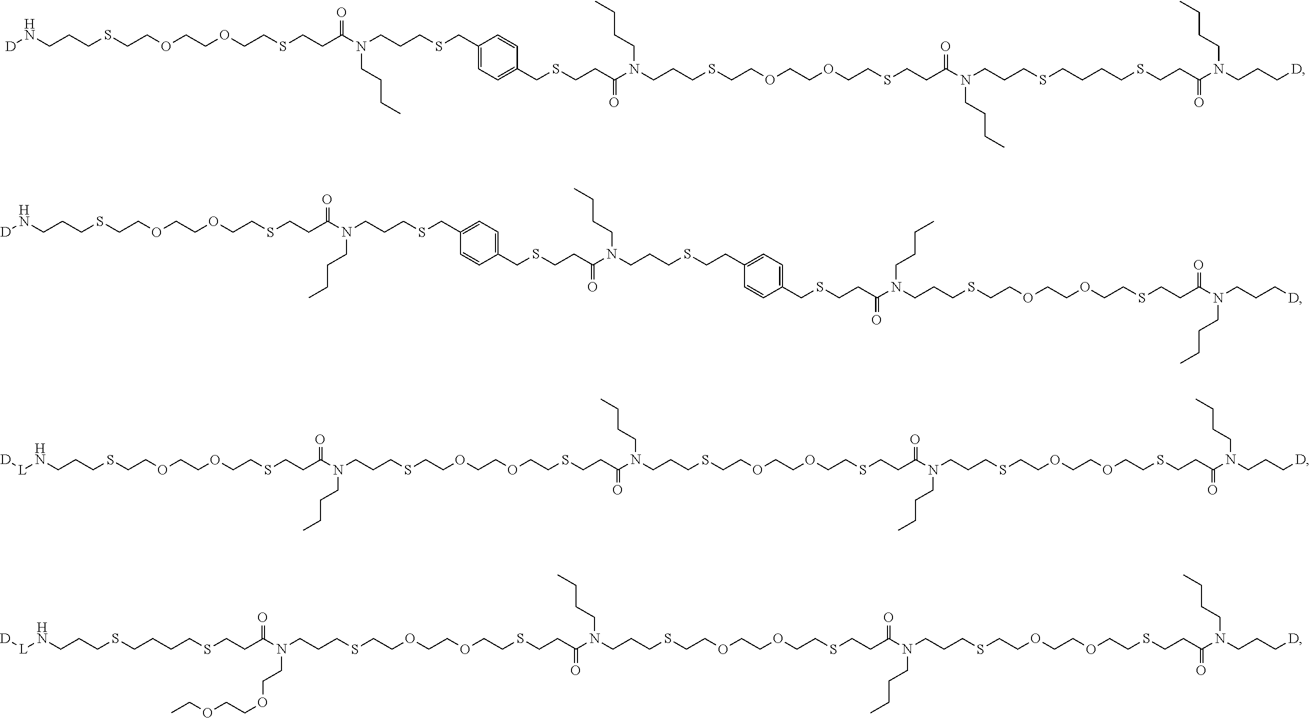

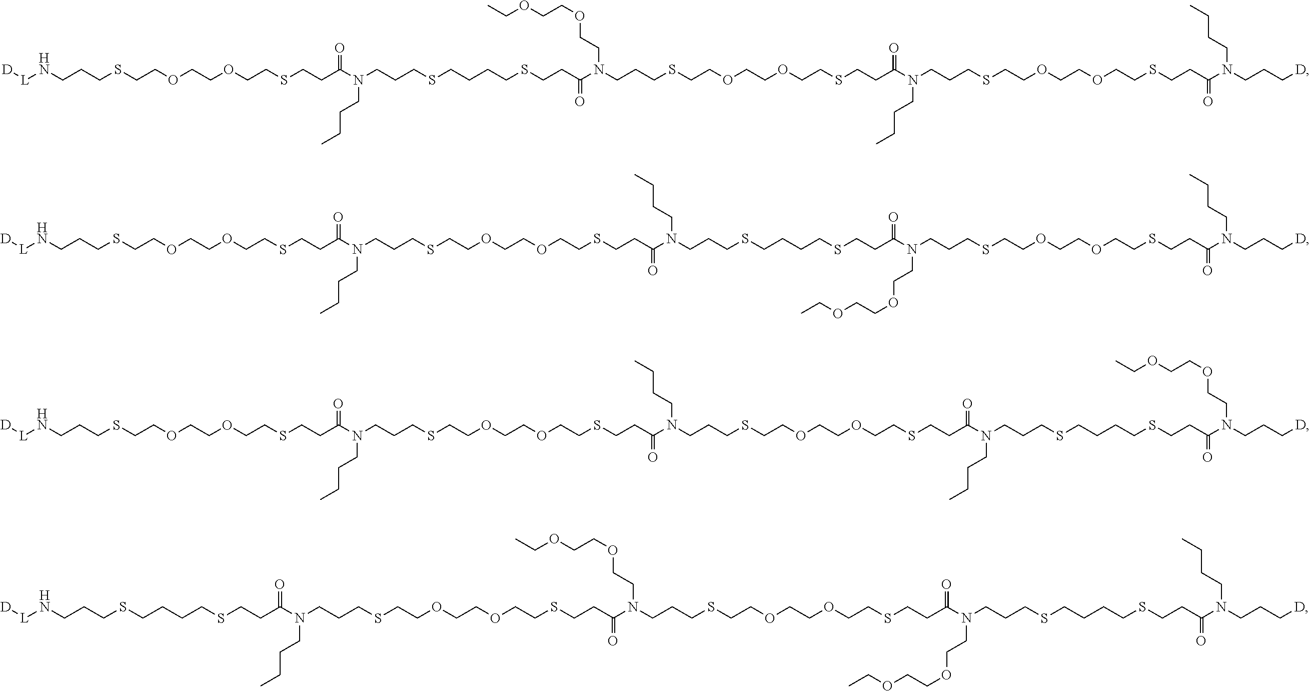

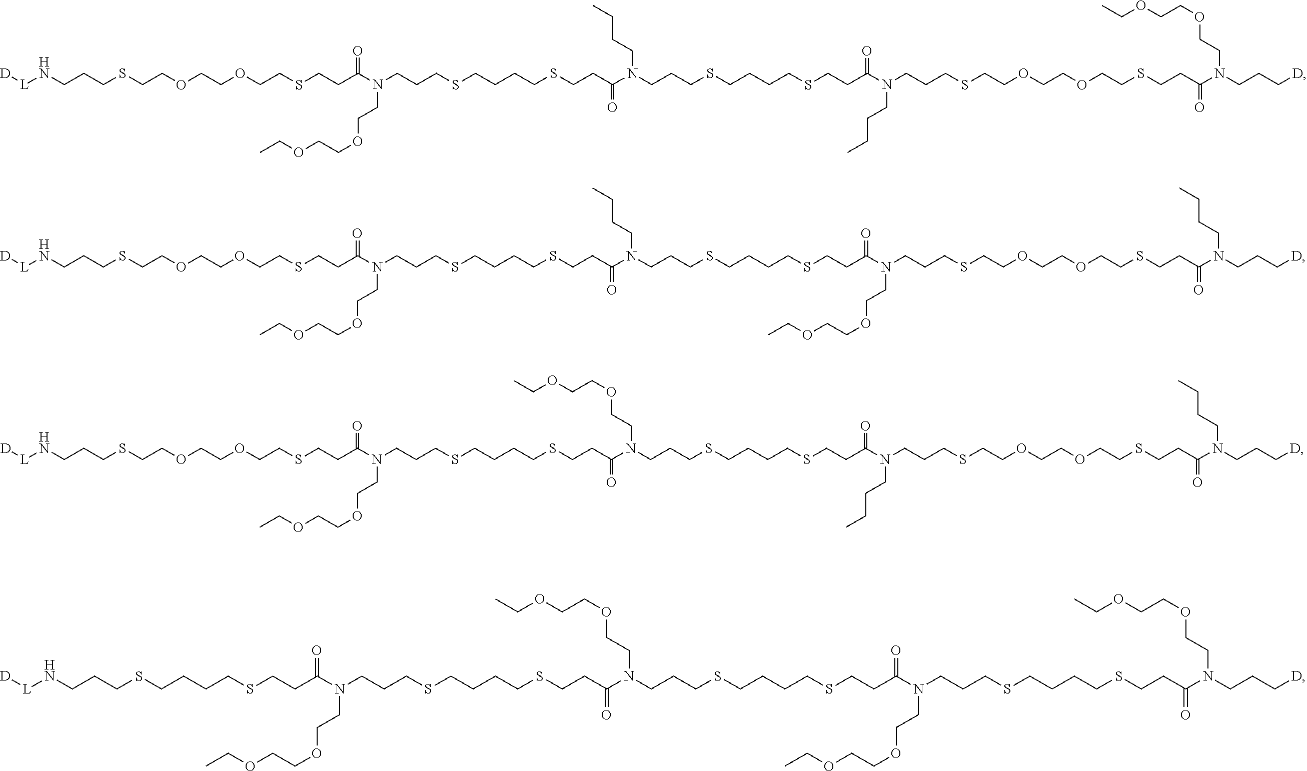

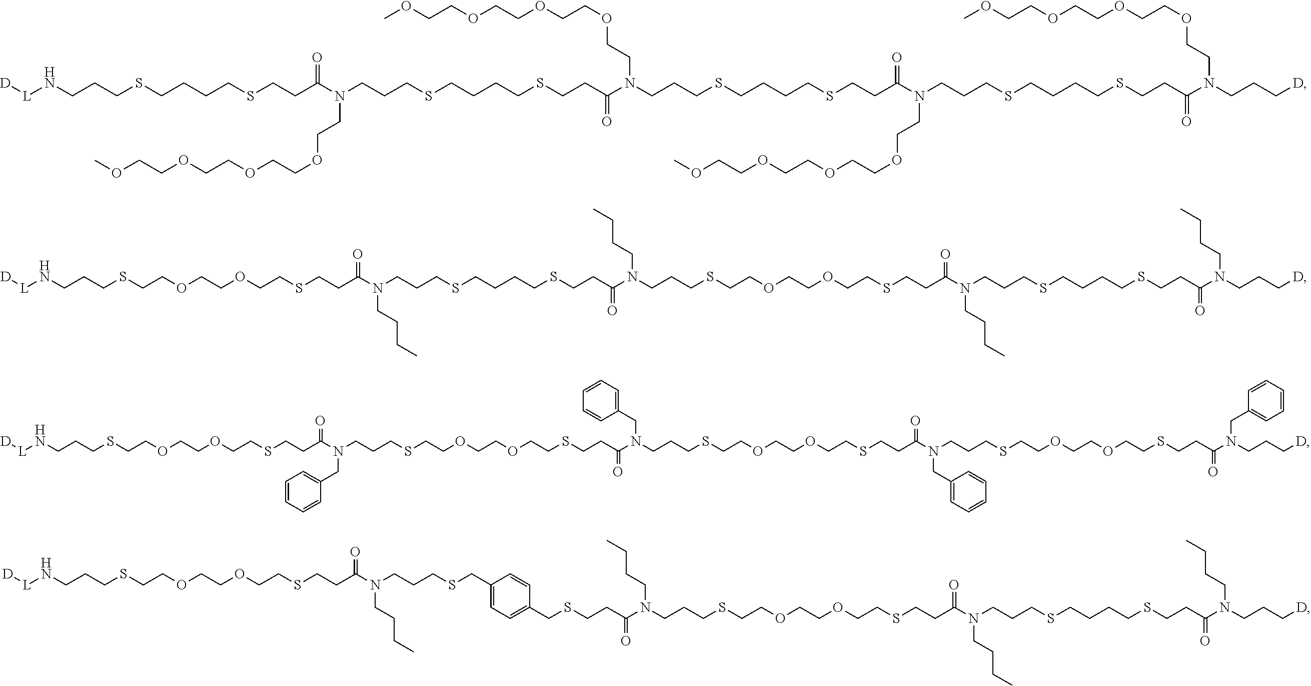

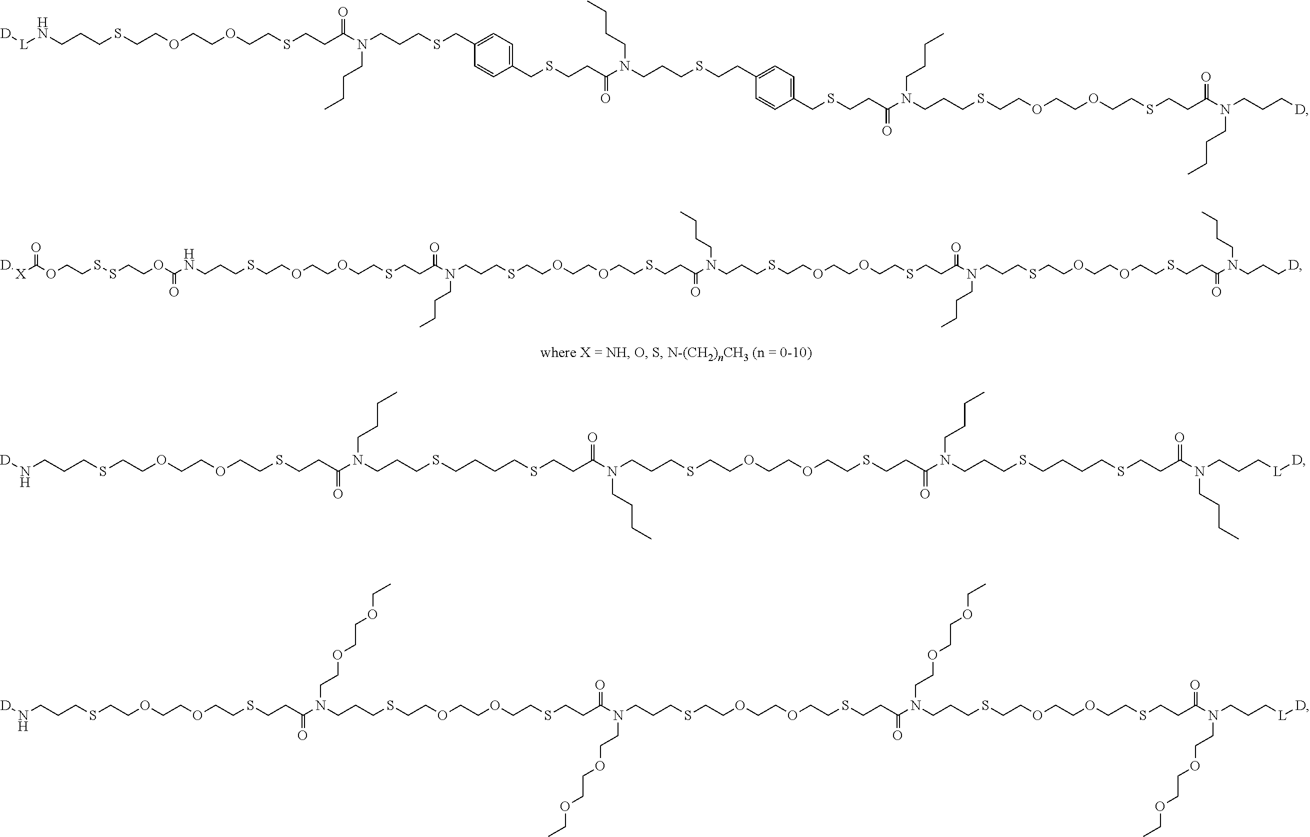

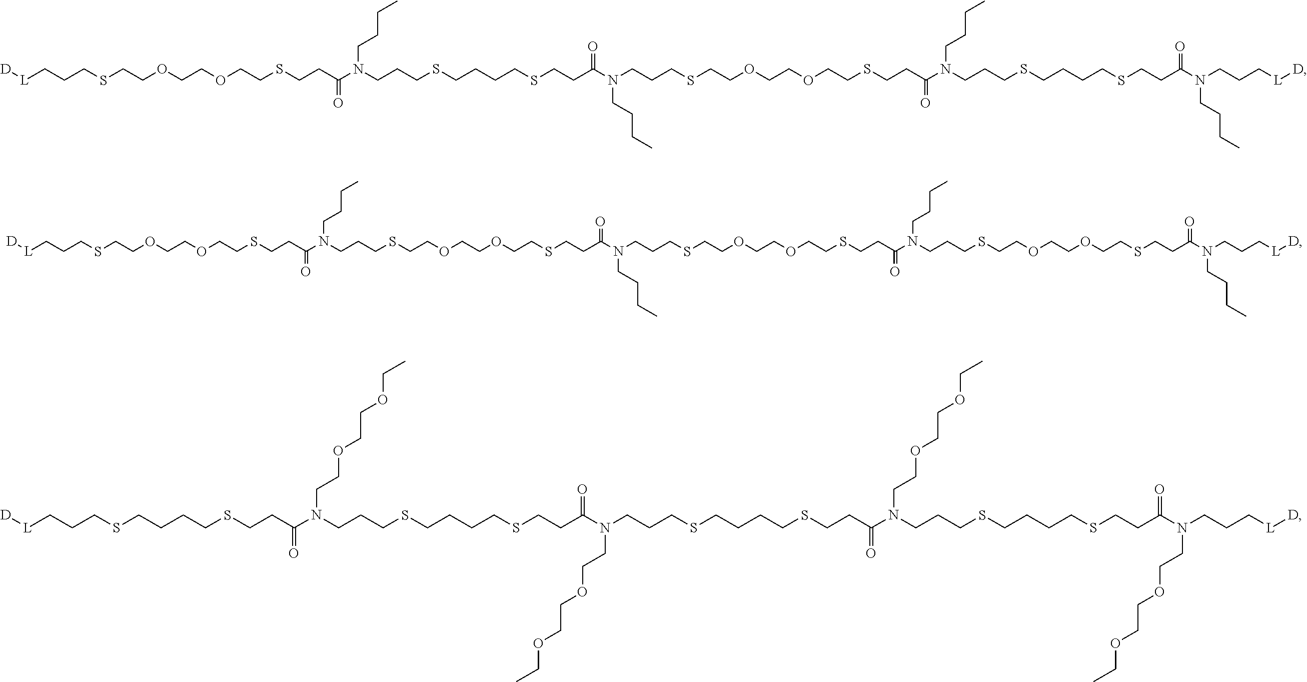

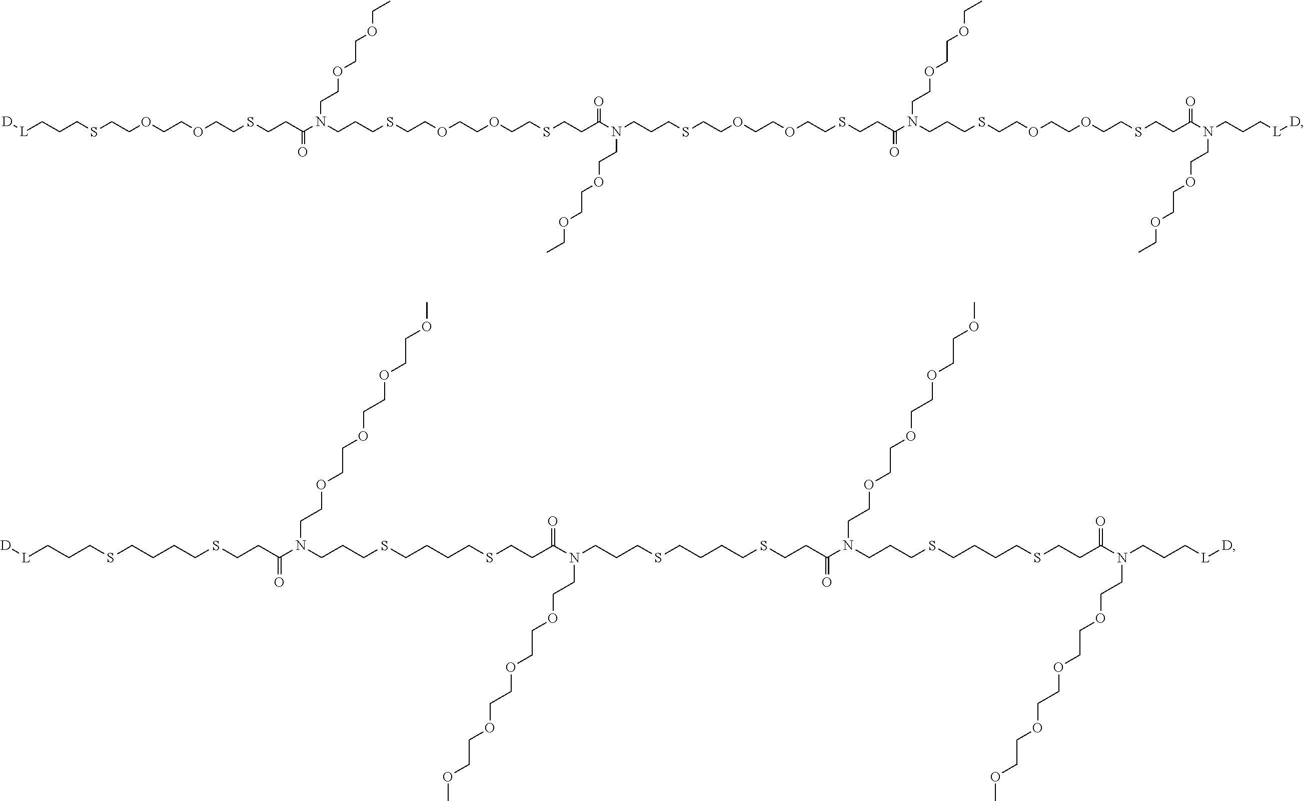

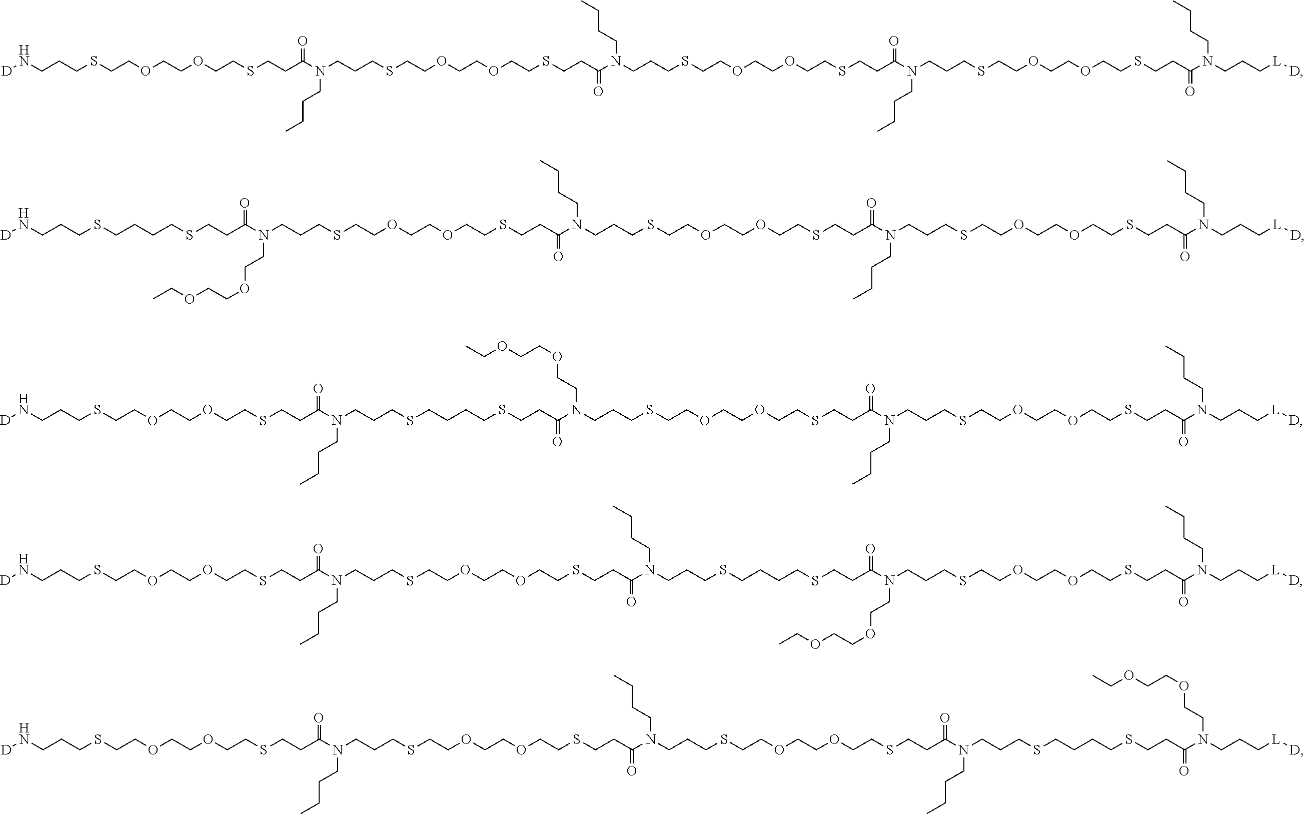

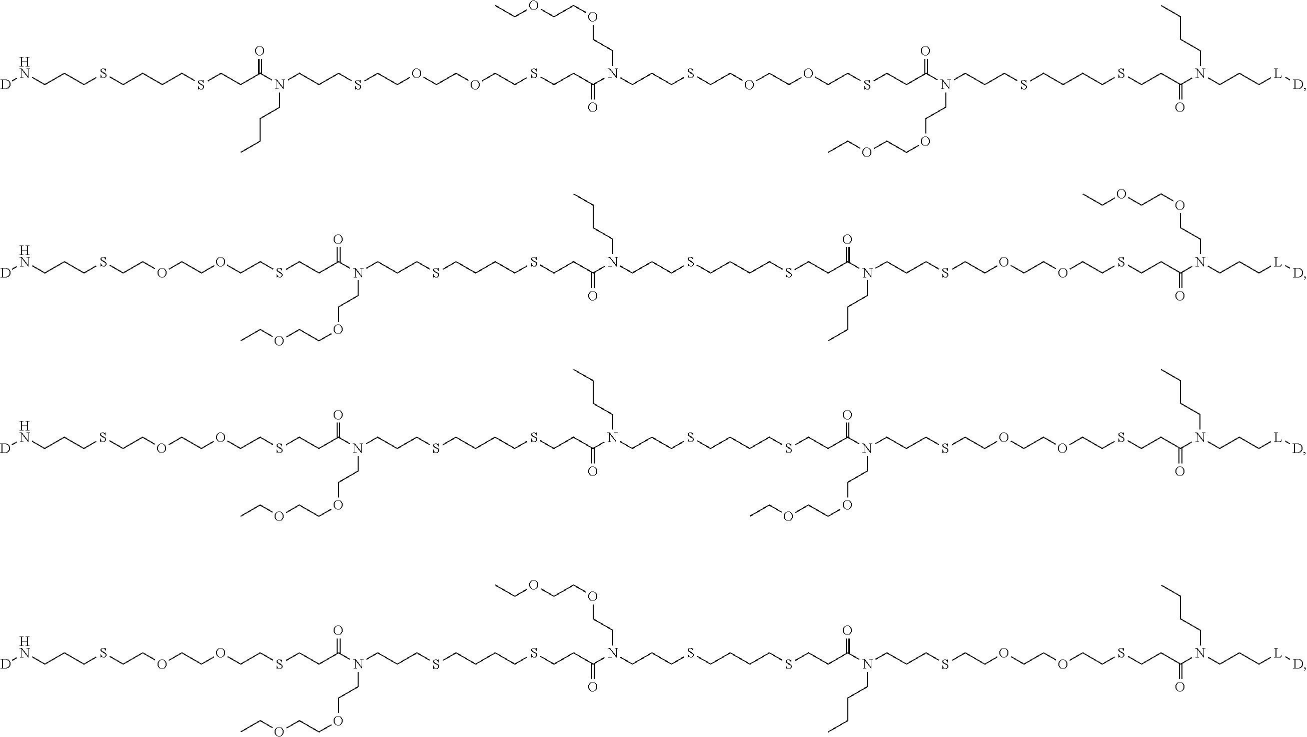

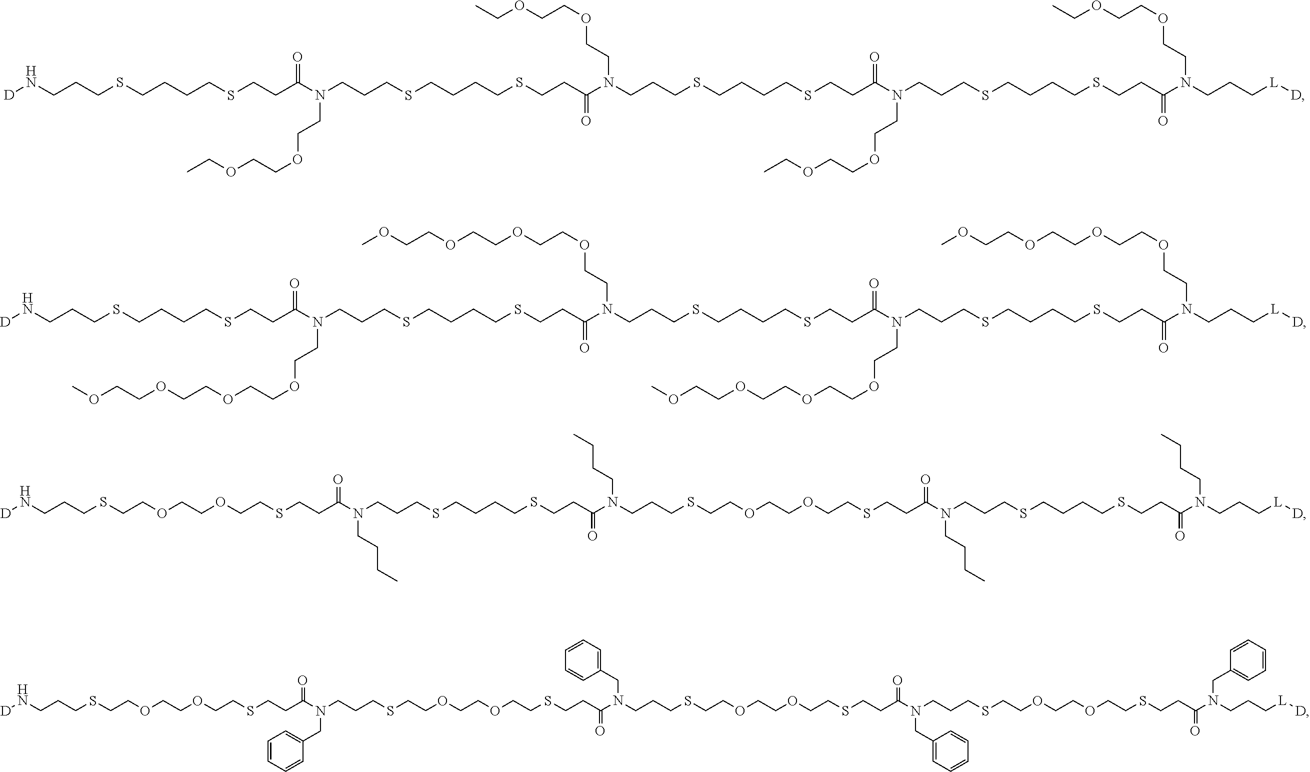

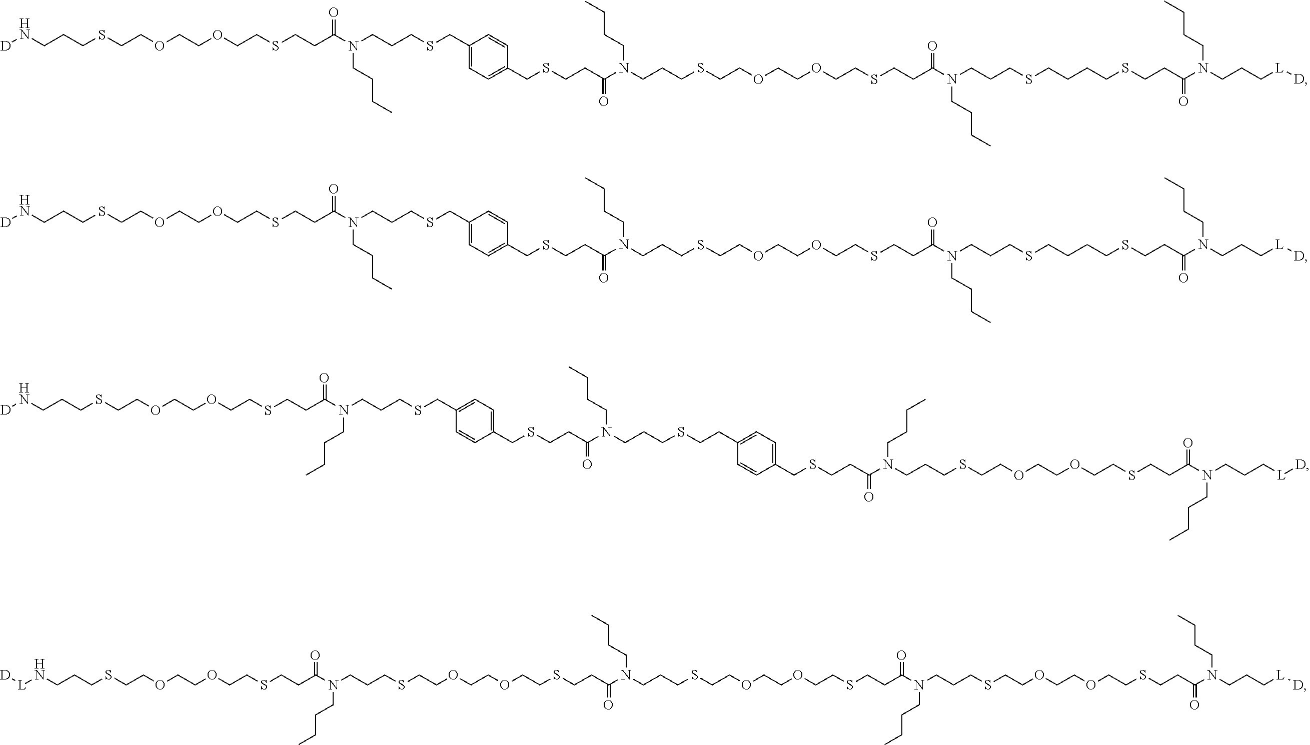

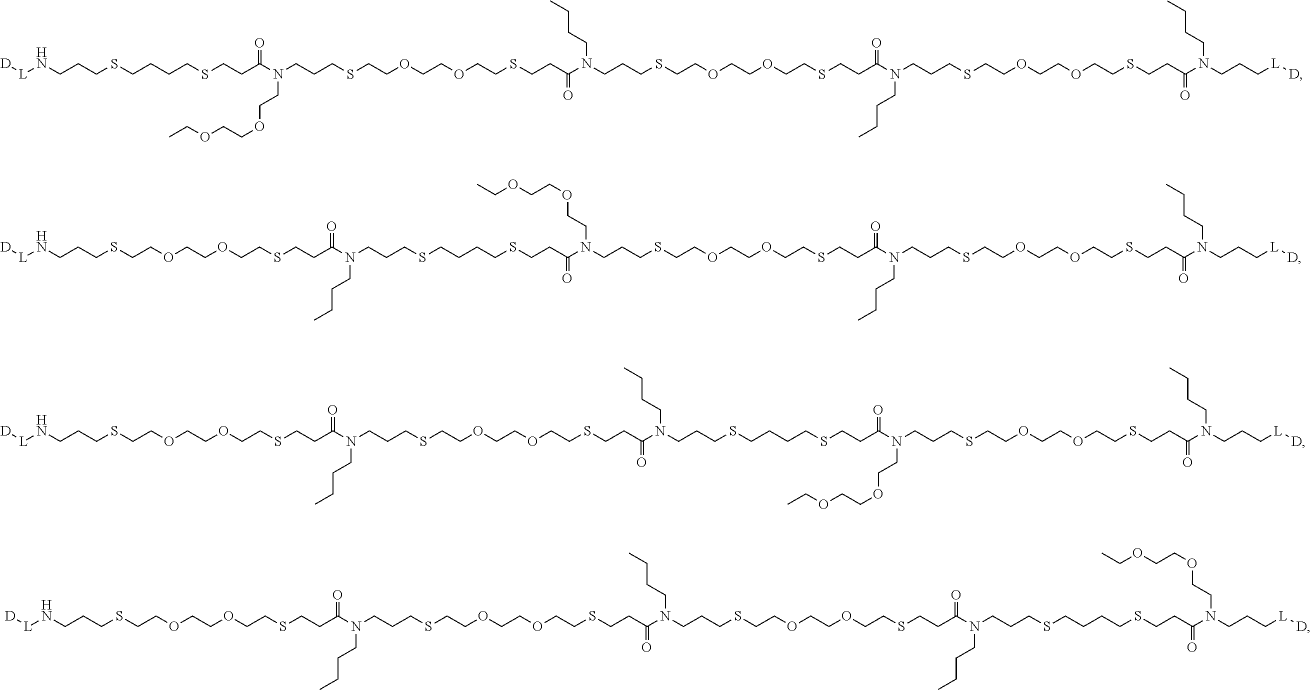

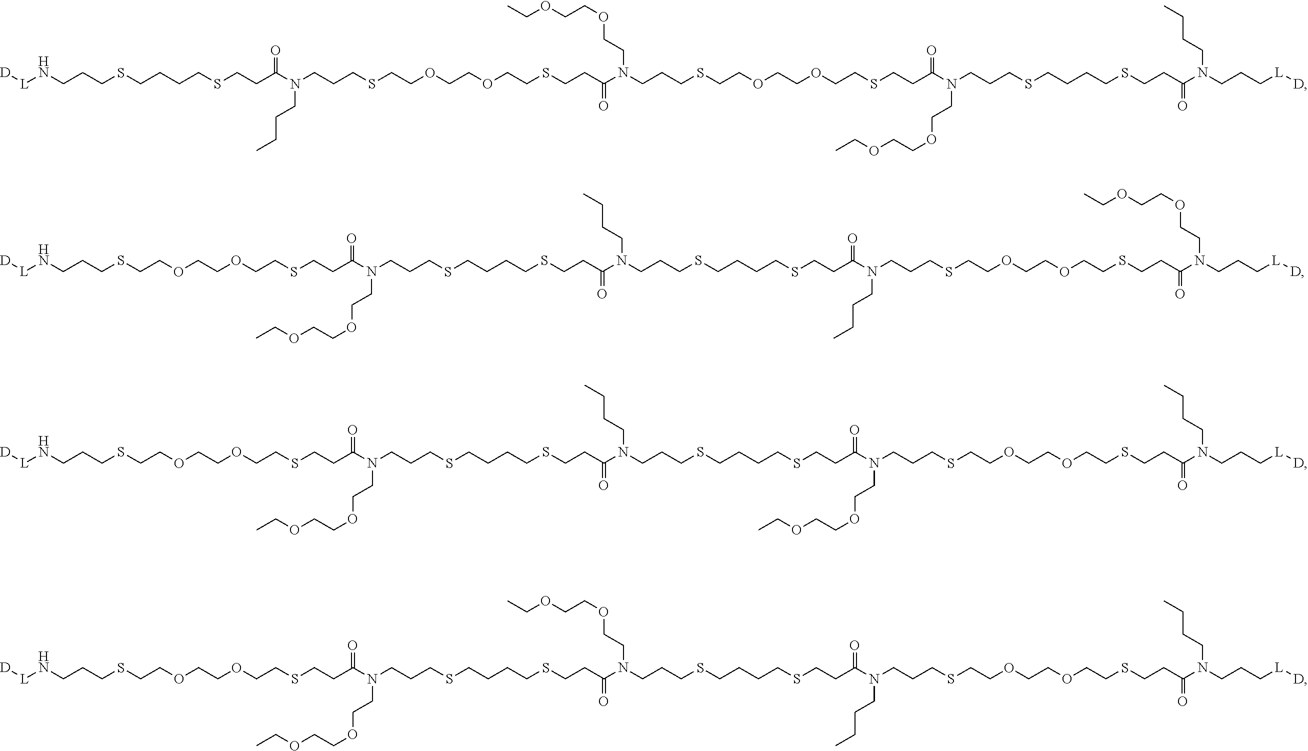

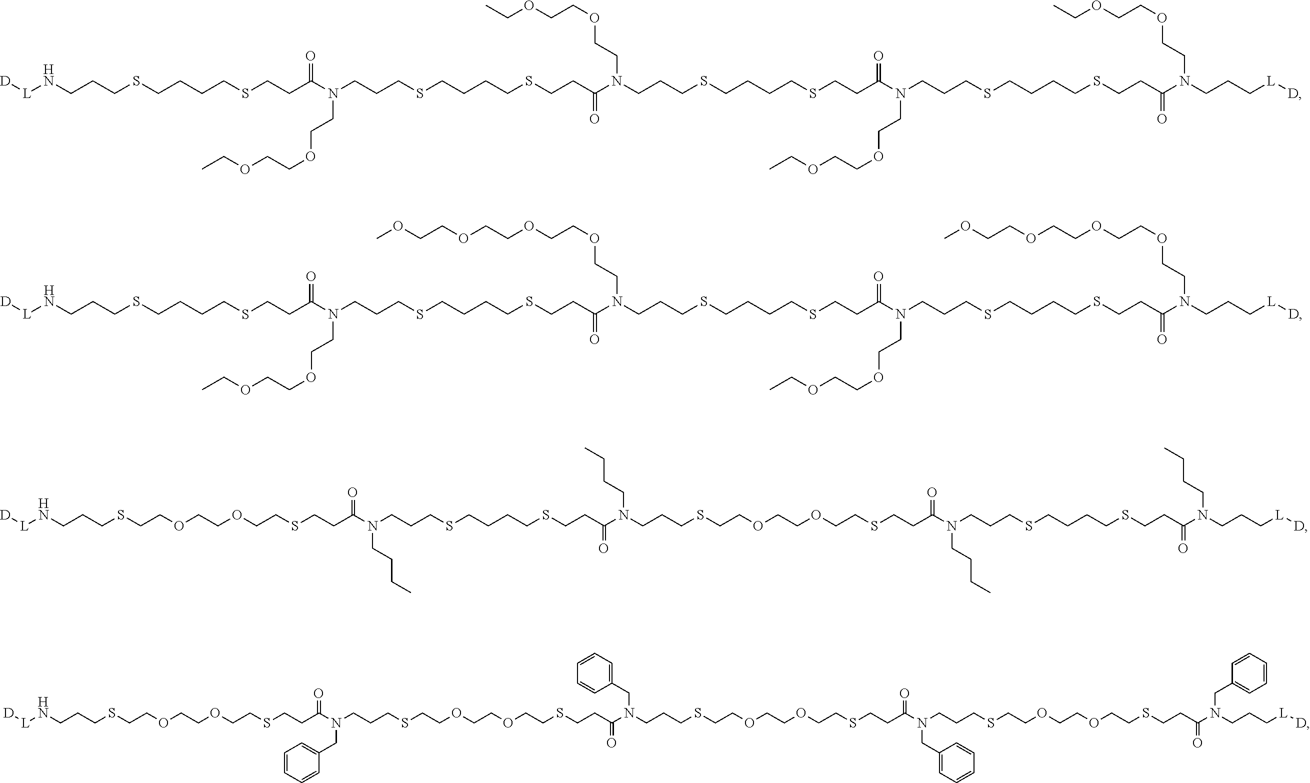

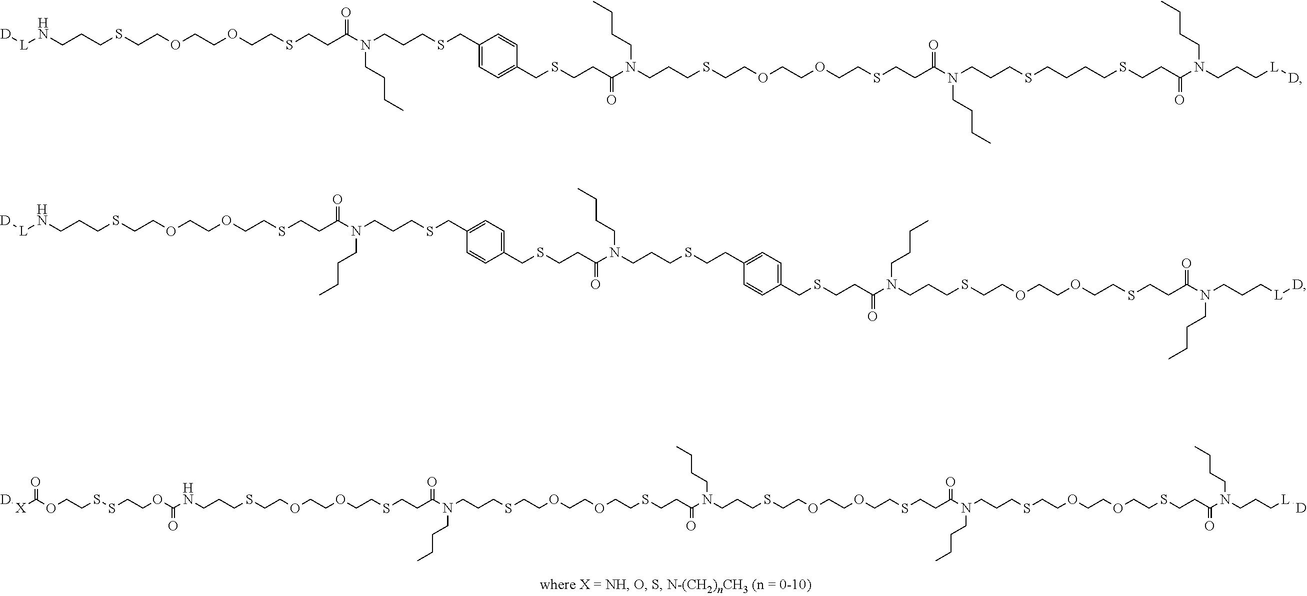

or isomers thereof, where L is chosen from a linking group, NH, N, O, and S, and D is one or more cargo group.
Statement 9. The compound according to any one of Statements 1-8, where the compound has the following structure:

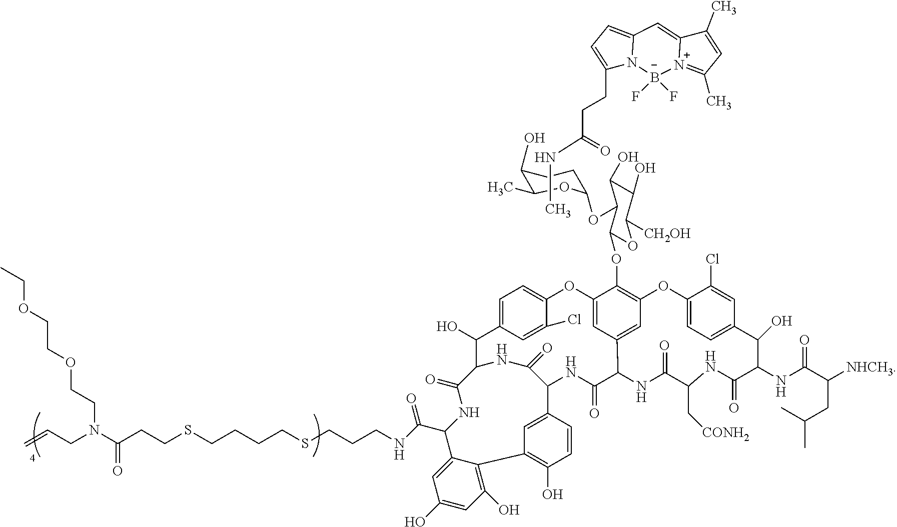

Statement 10. The compound according to any one of Statements 1-8, where the compound has the following structure:

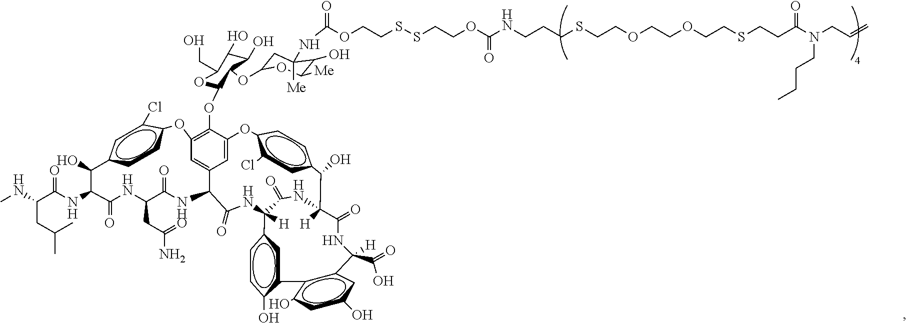

Statement 11. The compound according to any one of Statements 1-8, where the compound has the following structure:

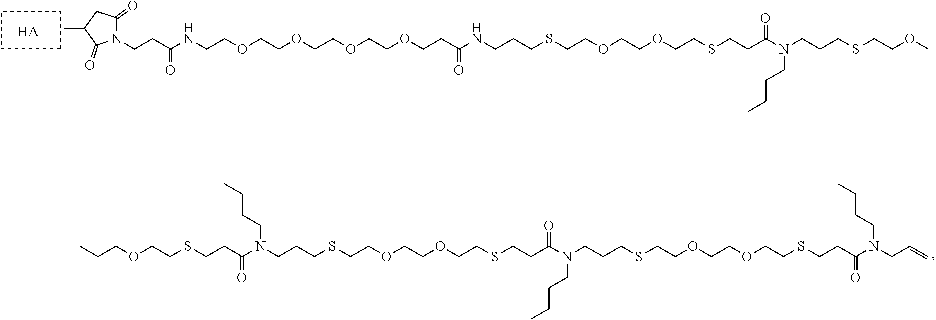

where HA is KADNAAIESIRNGTYDHDVYRDEALNNRFQIKGVELKSGYKDW-S- (SED ID NO:1) and the underlined S is a sulfur atom.
Statement 12. The compound according to any one of Statements 1-8, where the compound has the following structure:

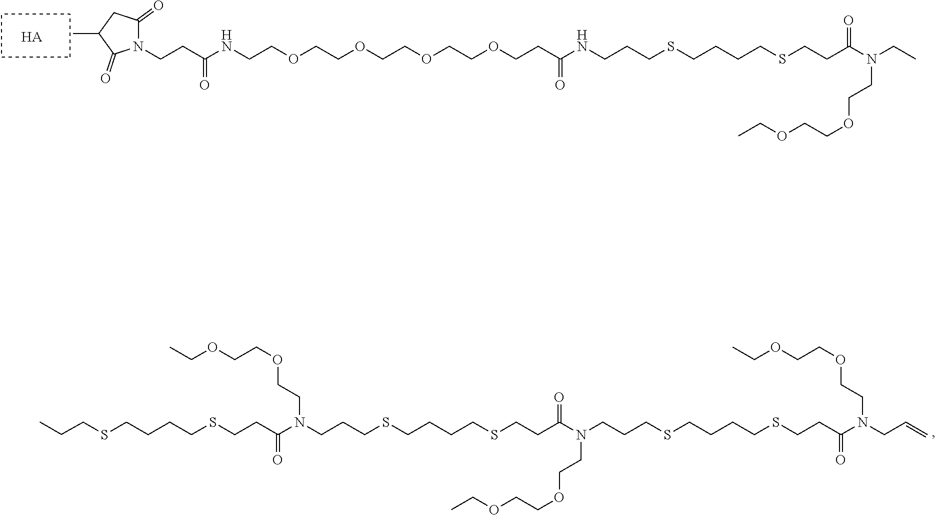

where HA is KADNAAIESIRNGTYDHDVYRDEALNNRFQIKGVELKSGYKDW-S- (SED ID NO:1) and the underlined S is a sulfur atom.
Statement 13. A composition comprising one or more compound according to any one of the preceding Statements and a pharmaceutically acceptable carrier.
Statement 14. A method for intracellular delivery of a compound according to any one of Statements 1-12, comprising administering to a subject in a need of treatment a composition according to Statement 13.
Statement 15. The method according to Statement 14, where the subject in need of treatment has or is suspected of having bacterial infections, cancers, viral infections, urinary tract infections, skin infections, cystic fibrosis, sepsis, fungal infections, or a combination thereof.
Statement 16. The method according to Statement 14 or Statement 15, wherein the bacterial infection is caused by Listeria monocytogenes, Staphylococcus aureus, Pseudomonas aeruginosa, Tuberculosis, Salmonella enterica, Francisella tularensis, and combinations thereof

The following examples are presented to illustrate the present disclosure. They are not intended to limiting in any matter.

### Example 1

The following example describes synthesis and use of compounds of the present disclosure.

Described are cell-penetrating oligothioetheramides (oligoTEAs) (CPOTs) that may undergo, for example, cellular entry across different cell lines with low cytotoxicity. CPOTs may outperform a widely used CPP, R9 peptide. This class of macromolecular transporters, which may be non-charged, are distinct from their cationic counterparts and may be used for intracellular delivery of therapeutics.

Described is the synthesis and biological evaluation of oligoTEAs, which may be non-charged cell-penetrating oligoTEAs (CPOTs). These oligomeric macromolecules may be prepared through fluorous-supported synthesis. Access to the backbone and the use of pendant groups, which may be non-charged, allows tuning of the oligoTEA hydrophobicity and create transporters with uptake across multiple cell lines and enhanced performance over currently and widely-used CPPs. The advantages of these oligoTEAs relative to standard cationic CPPs may be the following: (i) their uptake is not impeded by serum, and their abiotic backbone renders them resistant to proteases, and (ii) their uptake is not affected by heparin sulfate, a common ECM component known to affect the efficiency of cationic CPPs. Without being bound by any particular theory, studies of different cell entry mechanisms suggest that the primary mode of CPOTs cellular uptake may be direct in nature, with a smaller fraction using unknown endocytic mechanisms. Addition of more ethylene oxides to the backbone may improve solubility and decrease cytotoxicity of oligoTEAs without sacrificing uptake efficiency.

Materials and Instrumentation

Precursors for the monomer synthesis (amines and halides) were purchased from Aldrich and Santa Cruz Biotechnology. Fluorous BOC-ON (C₈F₁₇ BOC-ON) and fluorous silica were purchased from Boron Specialties. The peptide R9 (Ac-RRRRRRRRRK-Am) (SEQ ID NO:3) was purchased from GenScript. NHS-fluorescein (5/6-carboxyfluorescein succinimidyl ester, mixed isomer) was purchased from Thermo Fisher®. CellTiter 96® AQueous Non-Radioactive Cell-Proliferation Assay (MTS) solution was purchased from Promega. Single donor human red blood cells (RBC) were acquired from Innovative Research. All other chemicals were purchased from Sigma Aldrich. LCMS experiments were carried out on a Poroshell 120 EC-C18 column (3×100 mm, 2.7 μm) from Agilent Technology. All masses were detected in positive ion mode. LCMS solvents were water with 0.1% acetic acid (solvent A) and acetonitrile with 0.1% acetic acid (solvent B). Compounds were eluted at a flow rate of 0.6 mL/min with a linear gradient of 5% to 100% solvent B over 10 mins, constant at 100% solvent B for 2 min before equilibrating the column back to 5% solvent B over 3 min. HPLC purification was performed on a 1100 Series Agilent HPLC system equipped with a UV diode array detector and a 1100 Infinity analytical scale fraction collector using reverse phase C18 column (9.4×250 mm, 5 μm).

Methods

Cellular uptake protocol: 50,000 cells/well (HeLa, HEK293, or SKOV3) were plated in 24-well plates and incubated at 37° C. for 20-24 hrs. Cells were washed with 1×PBS pH 7.4 and incubated with 250 μL of fluorescein-oligoTEA conjugates at 5 μM in DMEM with 10% FBS at 37° C. for 1 hr; each compound was tested in duplicates. After the incubation, cells were washed with PBS and incubated with 200 μL of Trypsin EDTA at 37° C. for 3-5 mins. 1 mL of DMEM with 10% FBS was then added to quench the trypsin. Each well was transferred to an Eppendorf® tube and centrifuged at 500×g for 5 mins. The supernatant was removed, and cells were then re-suspended in 200-500 μL of PBS. Readings were taken on a FACSCalibur™ flow cytometry analyzer (Becton Dickinson). Results were analyzed by FlowJo software. The data presented is the mean fluorescence from 10,000 gated cells.

Treatment with Trypan Blue: The standard uptake procedure was followed, except that cells were washed 3 times with PBS after 1-hr (hour) incubation with fluorescein conjugates. Cells were then treated with 1:4 0.4% w/v Trypan Blue:PBS at room temperature for 10 mins prior to reading. All other conditions remained the same. Readings were taken on a FACSCalibur™ flow cytometry analyzer (Becton Dickinson). Transferrin-AlexaFluor 488 (Tf-488) (100 nM) was used as the positive control for this assay.

Dose-dependent uptake: The standard uptake procedure was followed, except that cells were treated with the labeled oligoTEAs at 0.5 μM to 5 μM. All other conditions remained the same. Readings were taken on a FACSCalibur™ flow cytometry analyzer (Becton Dickinson).

Uptake kinetics study: The standard uptake procedure was followed, except that cells were treated with fluorescein-PEO²-B at 2.5 μM for 15-120 mins (minutes). After the incubation, the plate was cells were washed with PBS and incubated with 200 μL of Trypsin EDTA at 37° C. for 3-5 mins. All other conditions remained the same. Readings were taken on a FACSCalibur™ flow cytometry analyzer (Becton Dickinson).

Mechanism of uptake: The standard uptake procedure was followed with the following changes for each mechanistic condition. Readings were taken on a FACSAria™ Fusion flow cytometry analyzer (Becton Dickinson).

Uptake with and without serum: Prior to treatment, cells were washed with PBS. Cells were then incubated with fluorescein conjugates in either DMEM with 10% FBS or Opti-MEM at 37° C. for 1 hr. Following treatment, cells were washed with PBS before addition of Trypsin EDTA.

Pretreatment with heparin sulfate: Prior to treatment, fluorescein conjugates were incubated with different concentrations of heparin sulfate and DMEM with 10% FBS in Eppendorf® tubes for 30 mins at 37° C. Cells were washed with PBS, and incubated with the treatment solutions at 37° C. for 1 hr. Following treatment, cells were washed with PBS once more before addition of Trypsin EDTA.

Temperature dependence: Prior to treatment, fluorescein conjugates were incubated with DMEM with 10% FBS in Eppendorf® tubes for 30 mins at 37° C. or 4° C. Cells were washed with warm (37° C.) or cool (4° C.) PBS for 5 mins. Pre-warmed or pre-cooled fluorescein conjugate solution was then added, and cells were incubated at 37° C. or refrigerated at 4° C. for 1 hr. Following treatment, cells were washed with PBS before addition of Trypsin EDTA.

Uptake with and without NaN3 and 2-deoxy-D-glucose (DOG): Prior to treatment, cells were washed with PBS. Cells were then incubated with 10 mM NaN3 and 25 mM DOG in DMEM with 10% FBS at 37° C. for 1 hr. Fluorescein conjugates were added, and cells were incubated at 37° C. for another hour. Following treatment, cells were washed with PBS before addition of Trypsin EDTA.

Uptake with and without chlorpromazine (CPM): Prior to treatment, cells were washed with PBS. Cells were then incubated with 5 μg/mL (or 15.7 μM) CPM in DMEM with 10% FBS at 37° C. for 1 hr. Fluorescein conjugates were added, and cells were incubated at 37° C. for another hour. Following treatment, cells were washed with PBS before addition of Trypsin EDTA.

Uptake with and without cytochalasin D (CCL-D): Prior to treatment, cells were washed with PBS. Cells were then incubated with 5 μg/mL (or 9.9 μM) CCL-D in DMEM with 10% FBS at 37° C. for 1 hr. Fluorescein conjugates were added, and cells were incubated at 37° C. for another hour. Following treatment, cells were washed with PBS before addition of Trypsin EDTA.

Uptake with and without filipin III (FLP-III): Prior to treatment, cells were washed with PBS. Cells were then incubated with 5 μg/mL (or 7.6 μM) FLP-III in DMEM with 10% FBS at 37° C. for 30 mins. Fluorescein conjugates were added, and cells were incubated at 37° C. for another hour. Following treatment, cells were washed with PBS before addition of Trypsin EDTA.

Live-Cell confocal microscopy: 70,000 HeLa cells/chamber were plated in a 4-chamber 35-mm glass-bottom microwell dish (MatTeK) and cultured at 37° C. for 20-24 hrs. Cells were washed with 2×PBS pH 7.4 and incubated with 5 μM fluorescein conjugates for 1 hr. For co-localization experiments, cells were incubated with fluorescein conjugates along with 150 nM Transferrin-Alexa Fluor 647, or 150 μM Dextran-Alexa Fluor 647 for 1 hr at 37° C. Cells were gently washed 3 times with PBS, and FluoroBrite DMEM Media supplemented with 10% FBS was added to each chamber for imaging. Images were taken on a Zeiss LSM880 live-cell confocal/multiphoton inverted microscope, equipped with a 63× oil objective, with 488-nm and 633-nm lasers enabled for fluorescein and Transferrin-Alexa Fluor 647/Dextran-Alexa Fluor 647, respectively. Images were processed using Fiji software.

Time-lapse live-cell imaging: 70,000 HeLa cells/chamber were plated in a 4-chamber 35-mm glass-bottom microwell dish (MatTeK) and cultured at 37° C. for 20-24 hrs. Cells were washed twice with PBS pH 7.4. 2.5 μM fluorescein conjugates and trypan blue (a 0.4 wt/v % trypan blue solution was diluted 1:10 with FluoroBrite DMEM with 10% FBS) was added to cells, and time-lapse imaging was started immediately with pictures taken every 1 min over 60 mins. Images were taken on a Zeiss LSM880 live-cell confocal/multiphoton inverted microscope, equipped with a 63× oil objective, with 488-nm laser enabled for fluorescein. Images were processed using Fiji software.

MTS cell proliferation assay: 15,000 HeLa cells/well were plated in 96-well plates and incubated at 37° C. for 20-24 hrs. Cells were washed with 1×PBS pH 7.4 and incubated with 100 μL of 5 μM to 40 μM of oligoTEAs in DMEM with 10% FBS at 37° C. for 1 hr. After the incubation, cells were washed 3 times with PBS. 100 μL of clear DMEM with 10% FBS and 10 μL of MTS solution (Promega) were added, and the plate was incubated for 1 hr. Absorbance measurements were taken at 490 nm on a TECAN Infinite M1000 PRO Microplate reader and normalized to untreated cells (100%) or no cells (0%). All experiments were performed in triplicates.

Hemolysis assay: Single donor human red blood cells were acquired from Innovative Research. A total of 200 μL of red blood cells was washed 2× with 1×PBS at pH 7.4 by centrifugation (5 mins at 500×g) and re-suspended in 5 mL of the same buffer for a 4% v/v RBC solution. OligoTEA solutions (diluted in PBS) or controls were mixed 1:1 with the RBC solution in a V-bottom, 96-well plate to reach a final volume of 100 μL. The resulting mixture was incubated on a shaker at 37° C. for 1 hr and then centrifuged (5 mins at 2120×g) at 4° C. A total of 75 μL of supernatant was transferred to a flat-bottom, 96-well plate. Hemolysis was measured via absorbance of released hemoglobin at 540 nm on a TECAN Infinite M1000 PRO Microplate reader and normalized to 0.1% Triton-X (100%) or PBS buffer (0%). All experiments were performed in triplicates.

GP Value Measurements with Laurdan

Sample preparation: 70,000 HeLa cells/well were plated in 4-well glass-bottomed microscope dish and cultured for 24 hrs at 37° C. Prior to imaging, cells were washed twice with 1×PBS pH 7.4 and incubated with 10 μM of Laurdan in DMEM with 10% FBS for 30 mins at 37° C. Cells were then washed 2× with pre-warmed PBS for imaging.

Equipment set-up: Laurdan GP images were collected on a two-photon fluorescence microscope with a two-channel detection system. A mode-locked titanium sapphire laser set to 780 nm was used as the two-photon excitation source. A 40× water objective was used. Two-channel acquisition was conducted in the emission ranges of 410-470 nm and 471-530 nm.

Data acquisition and analysis: Laurdan data were processed and displayed as pseudo-colored GP images using Fiji with a custom-written macro previously described. The GP values were calculated according to the following equation.

$$GP = \frac{{I}_{410 - 470} - {GI}_{471 - 530}}{{I}_{410 - 470} + {GI}_{471 - 530}}$$

The G factor is used in the GP calculation to compensate for the differences in the collection efficiency of the two channels caused by the use of different PMT gains between experiments. The G factor is calculated as follows:

$$G = \frac{{GP}_{ref} + {GP}_{ref} {GP}_{mes} - {GP}_{mes} - 1}{{GP}_{mes} + {GP}_{ref} {GP}_{mes} - {GP}_{ref} - 1}$$

GPₘₑₛ is the GP value of Laurdan in pure DMSO (25 μM) measured with the same microscope set-up as for real samples. GP_{ref} is the reference value for the dye in DMSO and is chosen to be 0.207 by convention so that the GP values for model membranes with liquid-ordered and -disordered phases are separated at around GP=0.

Synthesis of Monomers

Boc-Guanidine Monomer Synthesis:

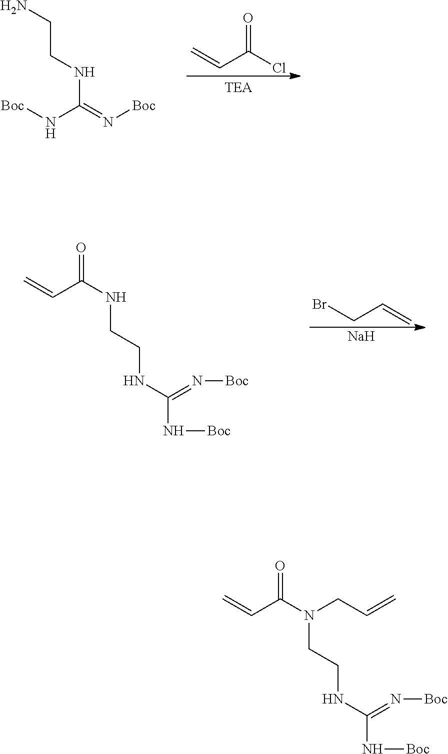

One equivalent of 2-(2-aminoethyl)-1,3-di-Boc-guanidine was dissolved in dry dichloromethane (DCM) to a final concentration of 150 mM. 1.2 equivalents of triethylamine was added and the mixture was stirred on ice for 15 mins. 1.1 equivalents of acryloyl chloride diluted in dry DCM was added dropwise over 1 hr, then the reaction was stirred for an additional 1 hr on ice and 1 hr at room temperature. The reaction mixture was washed twice with water and once with a saturated brine solution. The organic layer was then dried over anhydrous Na₂SO₄ and concentrated under reduced pressure. The crude product was used without additional purification.

The acylation product was dissolved in dry N,N-dimethylformamide (DMF) to a final concentration of 200 mM. 4 equivalents of sodium hydride was added and the mixture was stirred at room temperature for 15 mins. 2.5 equivalents of allyl bromide diluted in dry DMF was added dropwise over 15 mins, and the reaction mixture was stirred for 45 mins at room temperature. The reaction was quenched with water and extracted with diethyl ether. The combined organic layers were washed with water and a saturated brine solution and dried over anhydrous Na₂SO₄. Solvent was removed under reduced pressure, and the product was purified by silica column flash chromatography. The product was eluted with 25% ethyl acetate in hexanes.

Butyl Monomer Synthesis:

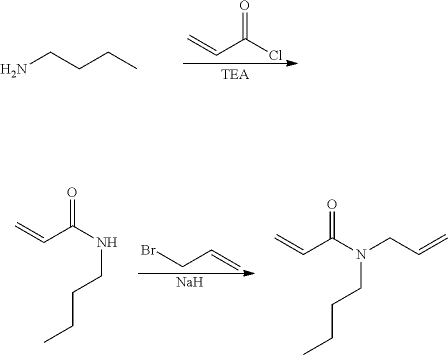

One equivalent of butyl amine was dissolved in dry DCM to a final concentration of 150 mM. 1.2 equivalents of triethylamine was added and the mixture was stirred on ice for 15 mins. 1.1 equivalents of acryloyl chloride diluted in dry DCM was added dropwise over 1 hr, then the reaction was stirred for an additional 1 hr on ice and 1 hr at room temperature. The reaction mixture was washed twice with water and once with a saturated brine solution. The organic layer was then dried over anhydrous Na₂SO₄ and concentrated under reduced pressure. The crude product was used without additional purification.

The acylation product was dissolved in dry N,N-dimethylformamide (DMF) to a final concentration of 200 mM. 1.5 equivalents of sodium hydride was added and the mixture was stirred at room temperature for 15 mins. 1.5 equivalents of allyl bromide diluted in dry DMF was added dropwise over 15 mins, and the reaction mixture was stirred for 45 mins at room temperature. The reaction was quenched with water and extracted with diethyl ether. The combined organic layers were washed with water and a saturated brine solution and dried over anhydrous Na₂SO₄. Solvent was removed under reduced pressure, and the product was purified by silica column flash chromatography. The product was eluted with 2% methanol in DCM. Purity was confirmed as previously reported by ¹H NMR and LC-MS.

OligoTEA Synthesis

OligoTEAs were synthesized using alternating thiol-ene and thiol-Michael addition reactions, followed by cleavage of the fluorous tag. Completed oligoTEAs were purified using reverse-phase HPLC and verified using LC-MS and ¹H NMR.

Thiol-ene reaction: Three equivalents of dithiol and 2,2-dimethoxy-2-phenylacetophenone (DMPA, 5 mol % of dithiol) were added to a solution of corresponding fluorous-olefin (100 mM) in methanol. The reaction mixture was subjected to UV irradiation for 270 s at 20 mW/cm². The product (fluorous-thiol) was purified by fluorous solid-phase extraction (FSPE).

Thiol-Michael addition: Two equivalents of corresponding monomer and dimethyl phenyl phosphine (Me₂PhP, 5 mol % of monomer) were added to the fluorous-thiol (100 mM) in methanol eluted from the purification of last thiol-ene reaction. Methanol was removed by reduced pressure in 1-1.5 hours. The time required for the evaporation of methanol was enough for the quantitative conversion of Michael addition. The reaction mixture was purified by FSPE.

FSPE: The fluorous organic mixture was loaded onto a cartridge pre-packed with 2 g of fluorous silica. A fluorophobic wash (4:1 methanol:water) was used to elute the non-fluorous molecules whereas the fluorous molecules were retained on the fluorous silica gel. A fluorophilic wash with methanol was then used to elute the fluorous molecules from the fluorous stationary phase.

Fluorous tag cleavage reaction: Fluorous-assembled oligoTEAs were dissolved in a 5 mM 1:1 trifluoroacetic acid (TFA):DCM mixture and stirred for 1 hr at room temperature. TFA and DCM was removed under nitrogen, and the oligoTEAs were purified using reverse-phase HPLC.

HPLC purification: OligoTEAs were purified on a 1100 Series Agilent HPLC system equipped with a UV diode array detector and a 1100 Infinity analytical scale fraction collector using reverse phase C18 column (9.4×250 mm, 5 μm). The column compartment was kept at 30° C. during fractionation. Solvents for HPLC were water with 0.1% TFA (solvent A) and acetonitrile with 0.1% TFA (solvent B). On a standard gradient, oligoTEAs were eluted at a flow rate of 4 mL/min with 5% solvent B, followed by a linear gradient of 5% to 100% solvent B over 30 mins, and finally 100% solvent B for 10 mins before equilibrating the column back to 5% solvent B over 3 mins. OligoTEAs were collected based on their absorption at 230 nm. The fractionated oligoTEA was transferred to a vial, dried and stored under argon until further analysis.

Fluorescein-OligoTEA Conjugate Synthesis

Pure, cleaved oligomers (10 mg/mL in DMSO) were reacted with 6.5 equivalents of NHS-fluorescein (mixed isomers of 5- and 6-carboxyfluorescein succinimidyl ester at 7.5 mg/mL in DMSO) and 10 equivalents of triethylamine for 1 hr at room temperature. The reaction mixture was then purified via HPLC. Fluorescein-oligoTEA conjugates were collected based on their absorption at 230 and 460 nm. The fractionated oligoTEA was transferred to a vial, dried and stored until further analysis. The conjugates were quantified by their fluorescence signals (Ex./Em. 493/515 nm) using a standard curve of NHS-fluorescein.

Polyethylene glycol monomer synthesis:

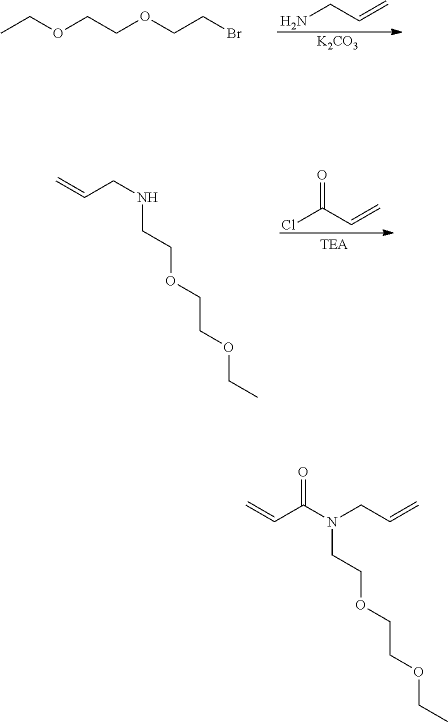

Allyl amine was mixed with 1.2 equivalents of K₂CO₃, and 0.2 equivalent of 2-(2-ethoxyethoxy)ethyl bromide was added at room temperature and stirred overnight. The reaction mixture was then filtered through celite and washed with CH₂Cl₂. The filtrate was concentrated at reduced pressure. Allyl amine was evaporated under high vacuum. The resulting products containing the secondary amine (desired product) and tertiary amine (side product) and 1.2 equivalents of triethylamine were dissolved in CH₂Cl₂. The reaction mixture was cooled to 0° C. for 15 mins while being stirred. 1.1 equivalents of acryloyl chloride (diluted in CH₂Cl₂) was added drop wise over a period of 1 hr at 0° C. and stirred for another hour at room temperature. The reaction mixture was washed twice with water and once with brine solution. The organic layer was dried over anhydrous Na₂SO₄, filtered, and concentrated at reduced pressure. The crude reaction mixture was purified by silica gel column chromatography. The product was eluted with 2% MeOH in CH₂Cl₂. Purity was confirmed by ¹H NMR and LCMS.

A new synthetic approach for the assembly of sequence-defined oligothioetheramides (oligoTEAs) via orthogonal N-allylacrylamide building blocks and a liquid-phase fluorous support was developed. OligoTEAs have three distinct advantages over native peptides that make them a promising scaffold for the design of cell penetrating agents. First, sequence-defined oligoTEAs are abiotic and not susceptible to protease degradation. Second, access to direct modification of the oligoTEA backbone enables direct control over the backbone flexibility and pendant group spacing to ultimately tune the interactions between the binding motifs and the cell membrane. Last, the use of a synthetic scaffold will prevent first pass immune recognition and clearance. The activity of CPPs was improved by incorporating CPP-like functionalities into the oligoTEA scaffold while benefiting from the stability, flexibility and diverse composition of oligoTEAs. The design and synthesis of a new class of non-charged oligoTEA molecular transporters is presented herein.

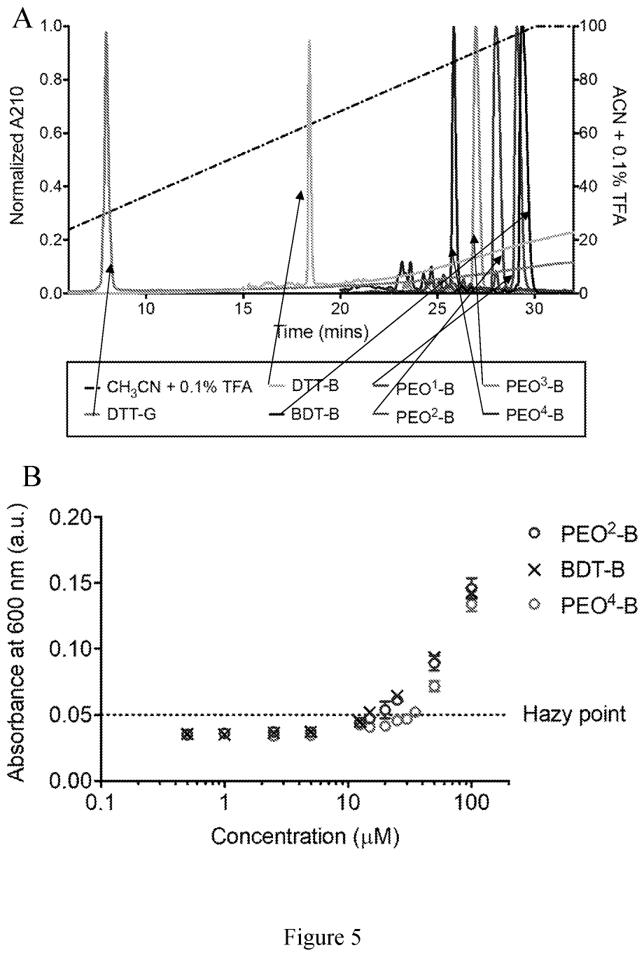

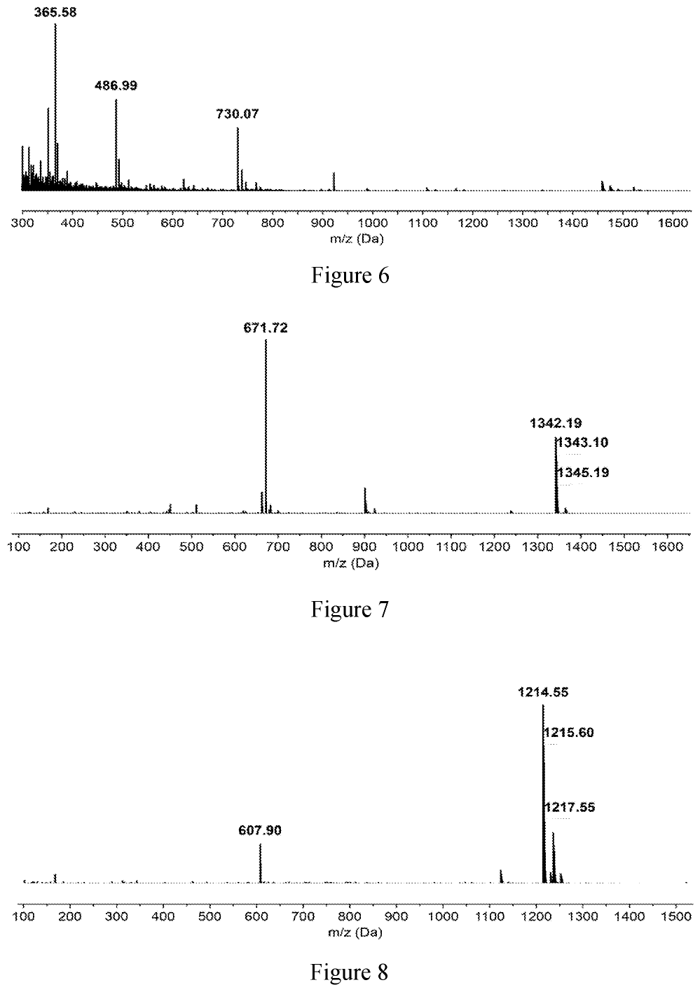

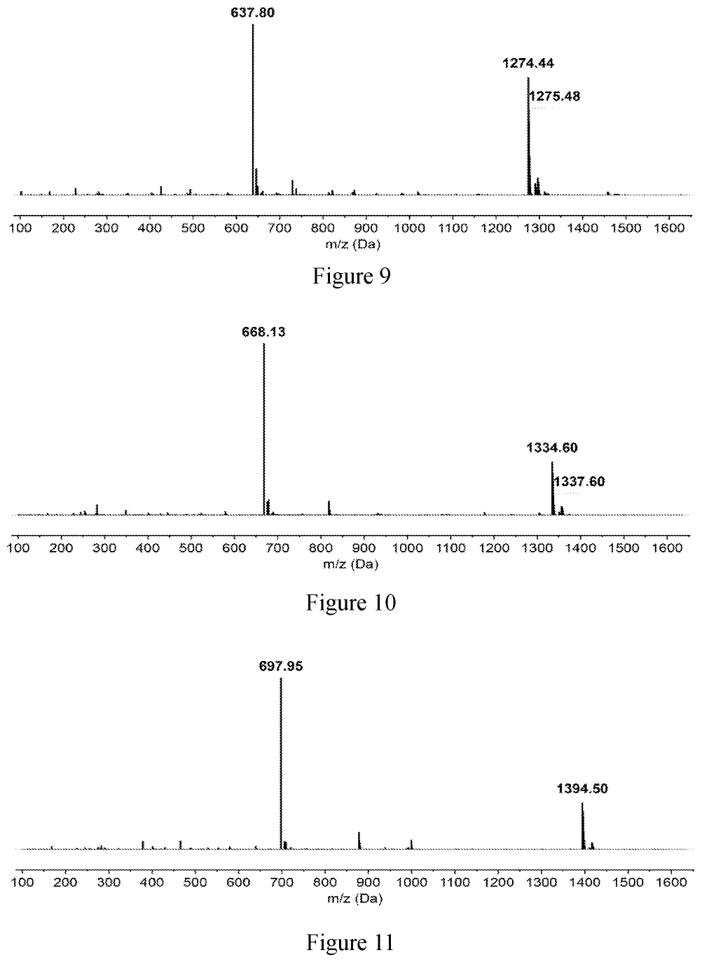

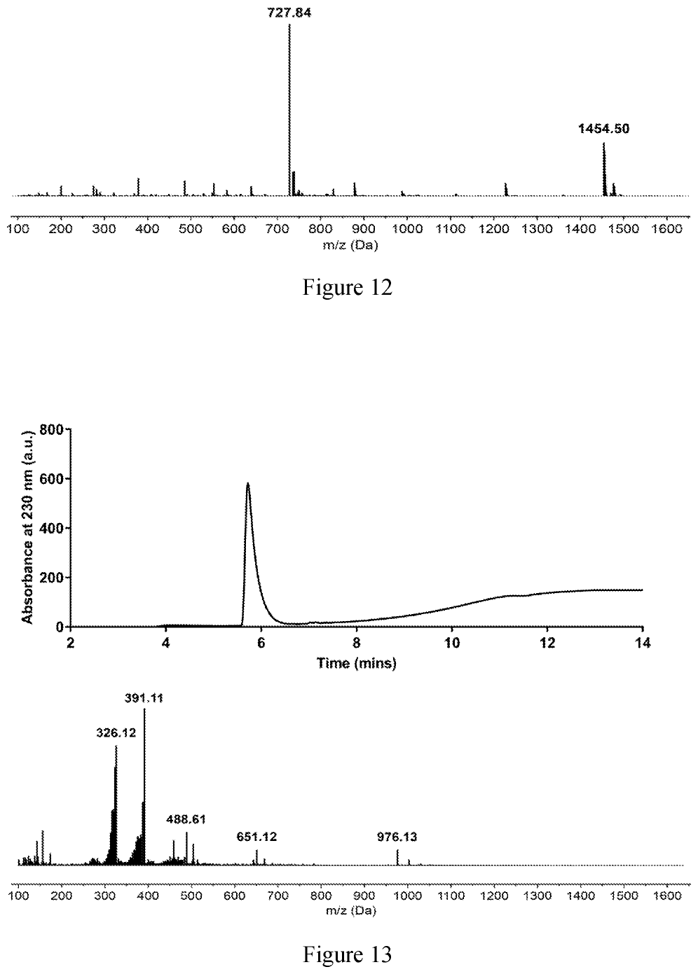

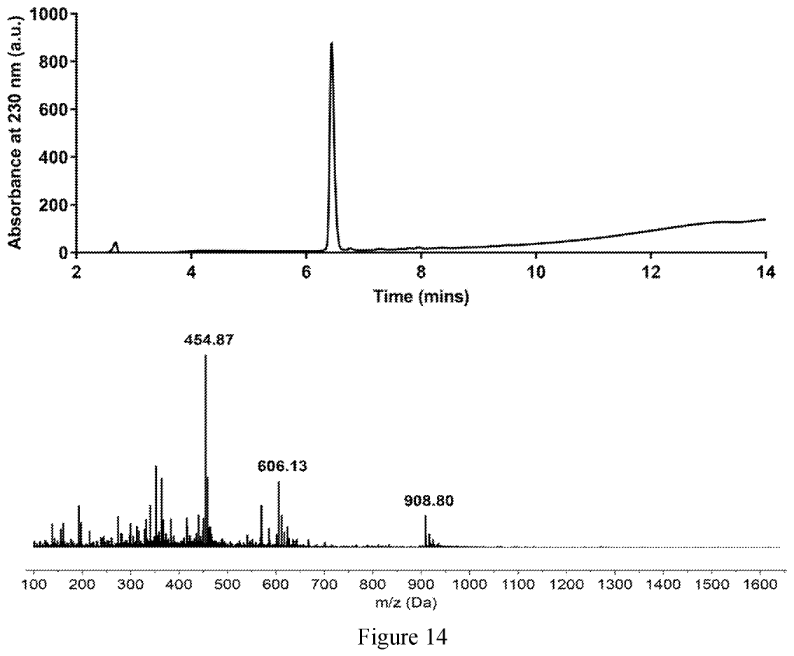

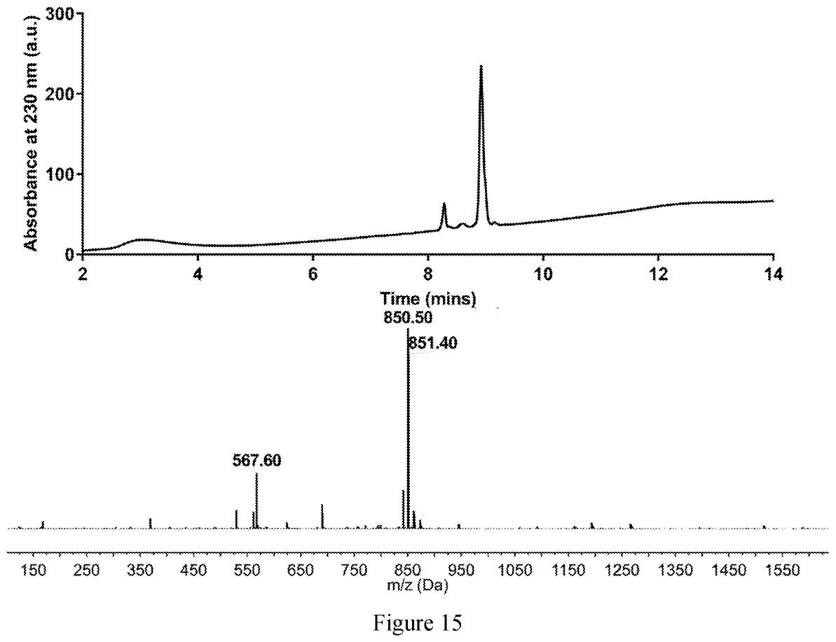

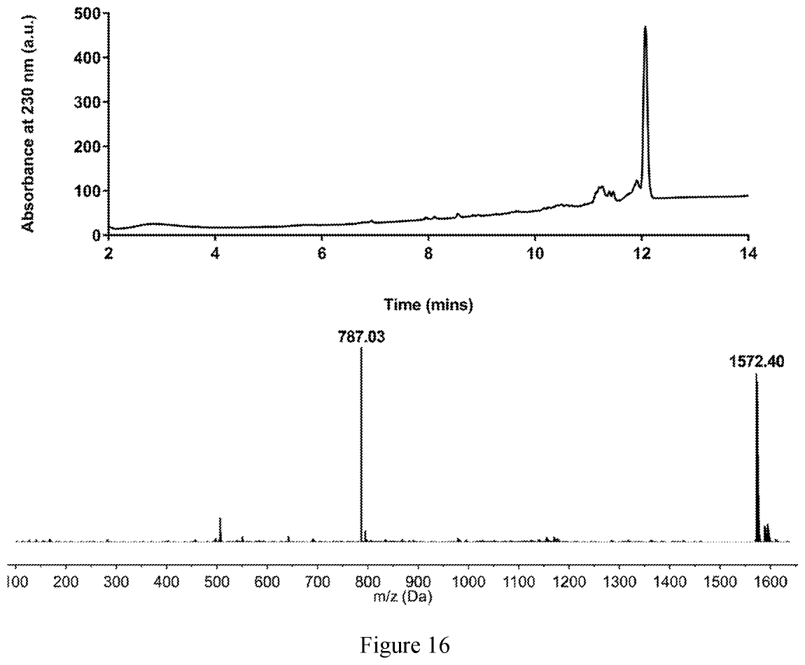

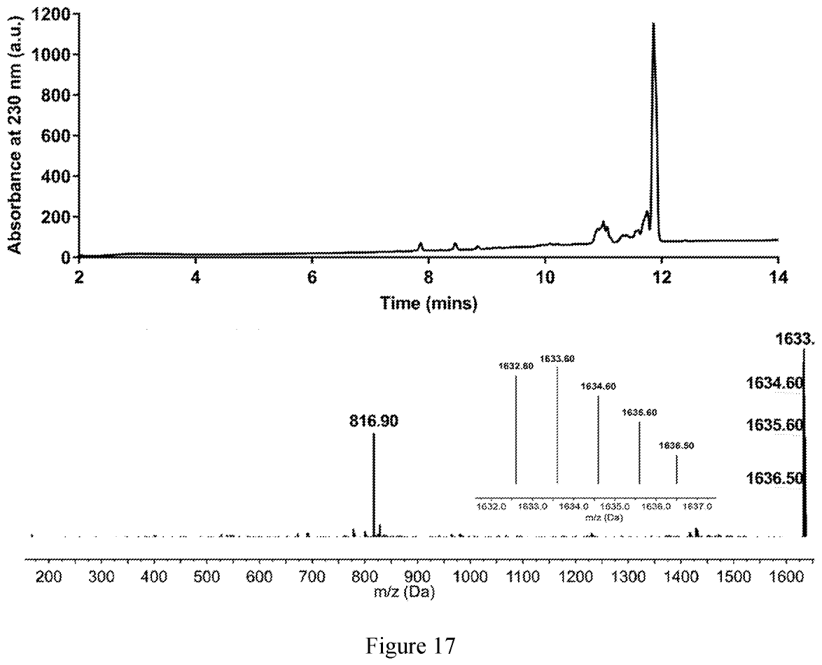

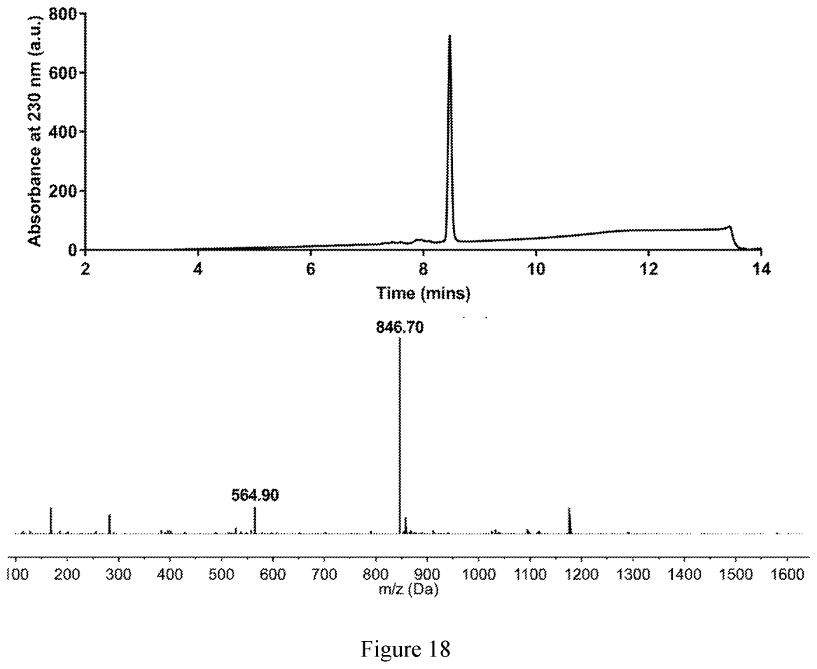

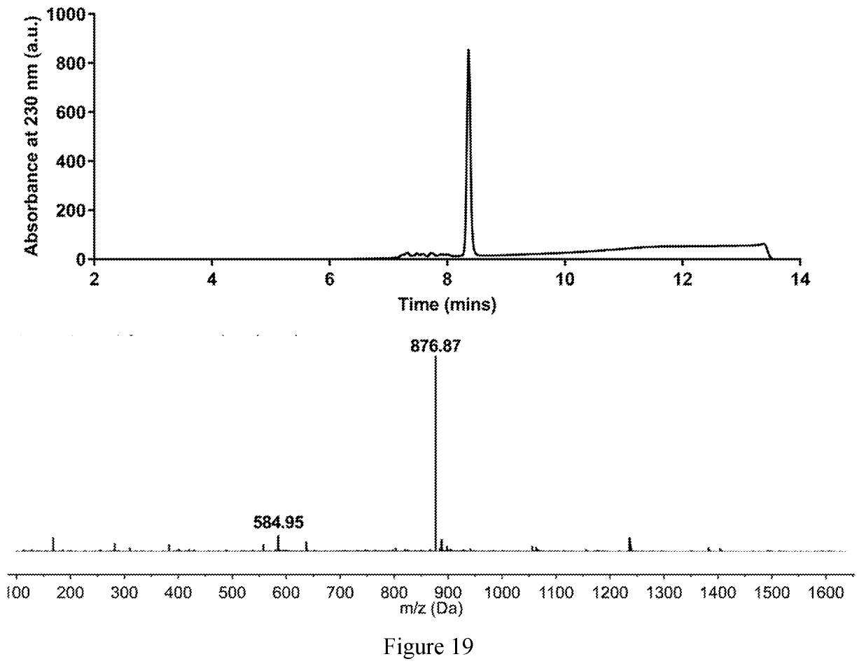

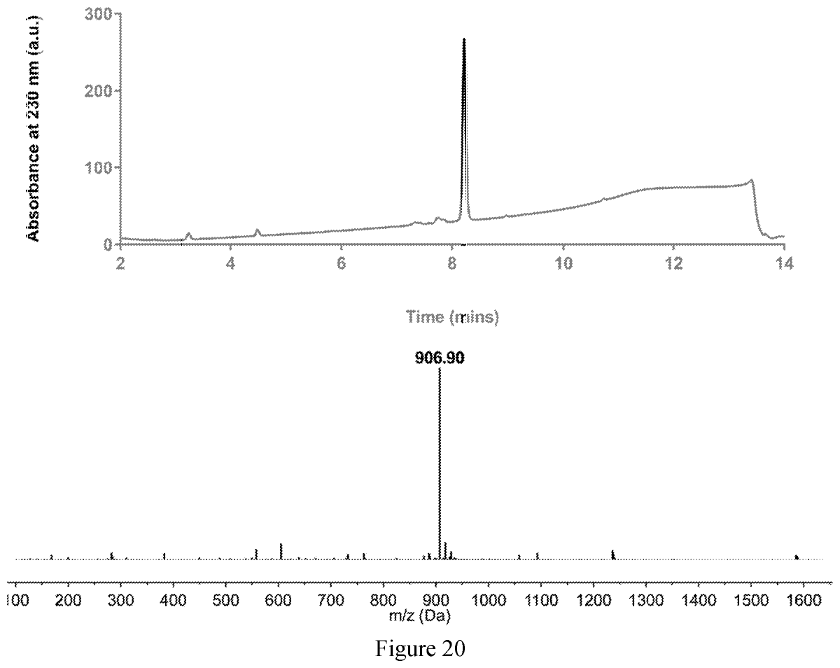

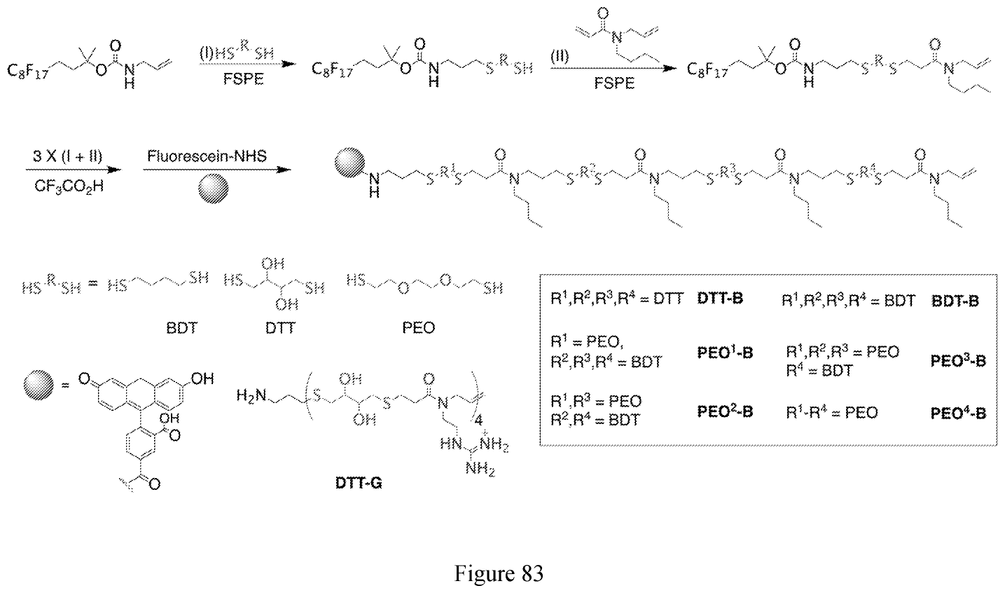

The assembly of oligoTEAs begins with a liquid-phase fluorous tag bearing a functional terminal allyl group. The first dithiol monomer is attached via a thiolene-click reaction under UV light in the presence of a photoinitiator, followed by a fluorous solid-phase extraction (FSPE) to isolate the resulting fluorous thiol (FIG. 83). An N-allylacrylamide monomer is then attached to the thiol group via a thiol-Michael addition of the acrylamide group in the presence of a phosphine catalyst. The resulting product is purified by FSPE, and this iterative process is continued until the desired oligomer length is obtained (Scheme 1). Next, the Boc functionality connecting the fluorous tag to the oligoTEA is cleaved off with a Brønsted acid, revealing a primary amine available for conjugation to the selected cargo. Following cleavage, oligoTEAs are purified by reverse-phase HPLC and confirmed by LCMS (FIGS. 5-12). In this work, oligoTEAs with 4 pendant groups (8 total monomers) were employed. The first oligomer, (Scheme 1, DTT-G), was synthesized using a hydrophilic backbone (DL-1,4-dithiothreitol) and a cationic guanidine monomer to mimic guanidinium-rich CPPs. Six non-charged oligoTEAs were synthesized (FIG. 83) using a hydrophobic (butyl) N-allylacrylamide monomer and either a hydrophobic butane dithiol backbone, an amphipathic polyethylene oxide (PEO) backbone, or a combination of both. The PEO backbone was employed to improve overall hydrophilicity and solubility (FIGS. 5A and 5B), but was later found to also improve molecular transport. All oligoTEAs synthesized in Scheme 1 were labeled with fluorescein, purified and confirmed via LCMS (FIGS. 14-20).

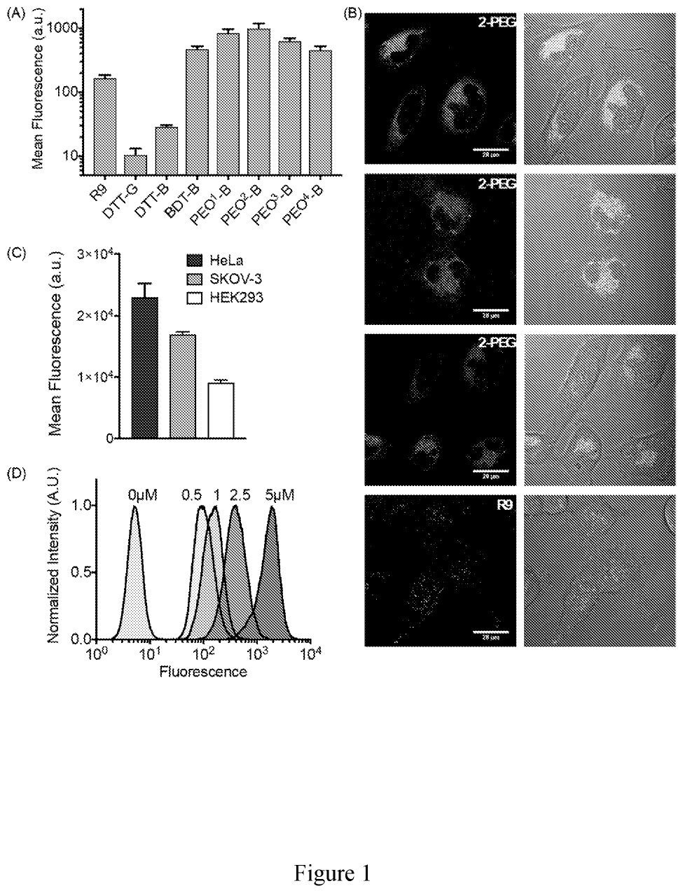

Two oligoTEAs with a hydrophilic glycol DTT backbone (DTT-G and DTT-B) were initially created to confirm the importance of the guanidinium group for cellular uptake. When fluorescently labeled DTT-G and DTT-B were evaluated for uptake in HeLa cells by flow cytometry, we were surprised to observe that the non-charged DTT-B was two-fold better than DTT-G at transporting fluorescein into cells (FIG. 1A). This result was counterintuitive but reproducible and suggests that for this class of macromolecules cationic charges are not a requirement for uptake, suggesting that hydrophobicity and amphiphilicity play a greater role in their cellular uptake. To further investigate this phenomenon, the hydrophilic DTT backbone of DTT-B was substituted to a hydrophobic butane backbone in BDT-B. Consistent with our initial result, the hydrophobic BDT-B outperformed both DTT-B and DTT-G with a 45-fold increase in fluorescence over DTT-G and about a 3-fold increase over the cationic CPP, R9. Although BDT-B showed excellent uptake capabilities, its hydrophobicity was a concern, especially since most drugs in need of a delivery vehicle are hydrophobic. To improve its water solubility without introducing a cationic charge, the BDT backbone was replaced with one or more amphiphilic water-soluble PEO dithiol monomers (FIG. 83). All oligoTEAs with PEO in their backbone were more hydrophilic than BDT-B (FIG. 5) and showed similar or in some cases better uptake than BDT-B (FIG. 1A). Overall, these non-charged oligoTEAs appeared to be efficient molecular transporters.

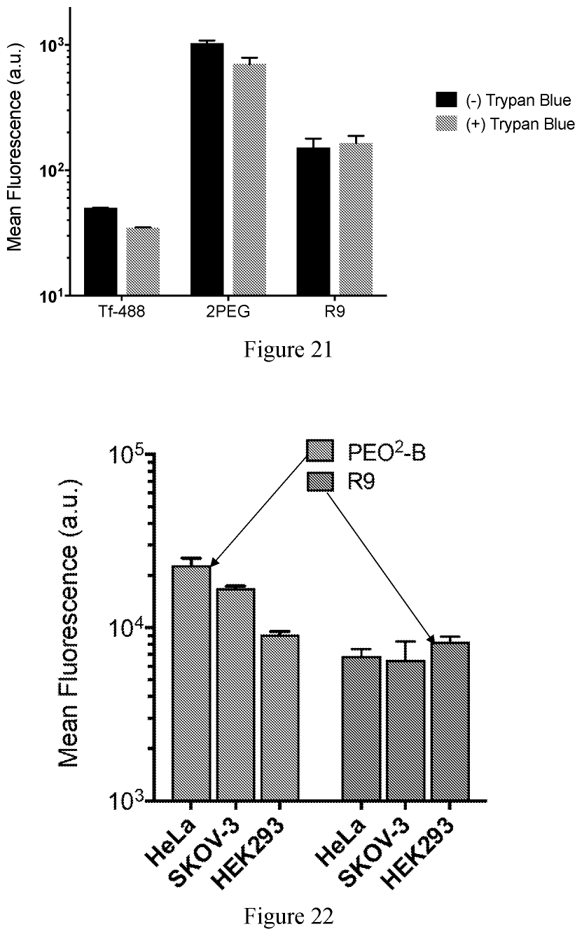

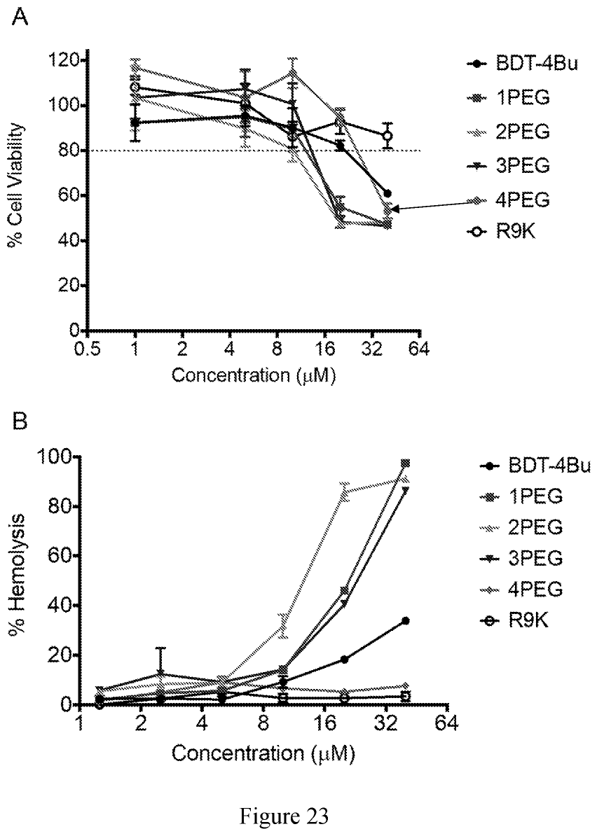

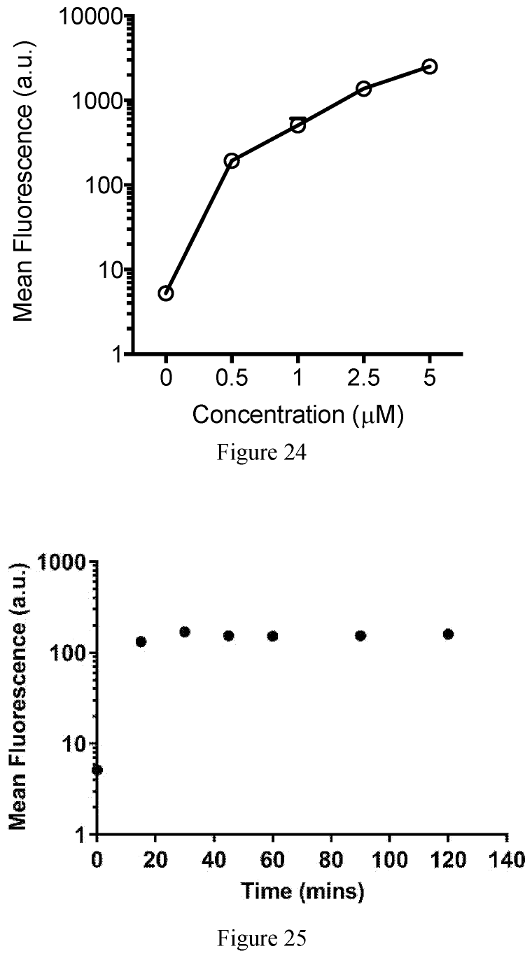

One of the best CPOTs, PEO²-B, exhibited a 6-fold increase in internalization relative to the fully charged R9 standard. To verify that the fluorescein-oligoTEA conjugates were internalized and not just adsorbing to the cell membrane, a trypan blue quenching experiment was performed to eliminate extracellular fluorescence. The data (FIG. 21) shows that only about 30% of the signal is membrane bound, similar to a transferrin control. The majority of the fluorescence signal (about 70%) is due to internalized conjugate. PEO²-B was selected for further studies. The subcellular distribution of PEO²-B was imaged by live-cell confocal microscopy (FIG. 1B). The R9 conjugate appeared primarily as punctate spots while the PEO²-B fluorescein conjugate was mostly diffuse and spread throughout the cell interior. The live-cell confocal images corroborated the cytosolic uptake seen in the flow cytometry data and also confirmed that the fluorescein-oligoTEA conjugates were primarily internalized and distributed broadly in the cytosolic space. Time-lapse live cell imaging further confirmed rapid intracellular localization of the PEO²-B fluorescein conjugates and no internalization of fluorescein acid alone. To evaluate the scope of its uptake, we explored the uptake capability of PEO²-B across a variety of cell lines. Uptake was evaluated in HeLa cells (human cervical cancer) along with SKOV-3 (human ovarian cancer) and HEK293 (healthy human embryonic kidney) cells. Significantly, all cell lines showed robust uptake of PEO²-B (FIG. 1C), and PEO²-B showed similar or better uptake than R9 in all cell lines (FIG. 22). A dose-dependent uptake study performed on HeLa cells shows that PEO²-B facilitates intracellular delivery at concentrations as low as 500 nM (FIG. 1D) with negligible cytotoxicity (FIG. 23). The same efficient uptake is observed with PEO⁴-B (FIG. 24). The uptake of PEO²-B was rapid, with maximum uptake achieved in 15 minutes (FIG. 25).

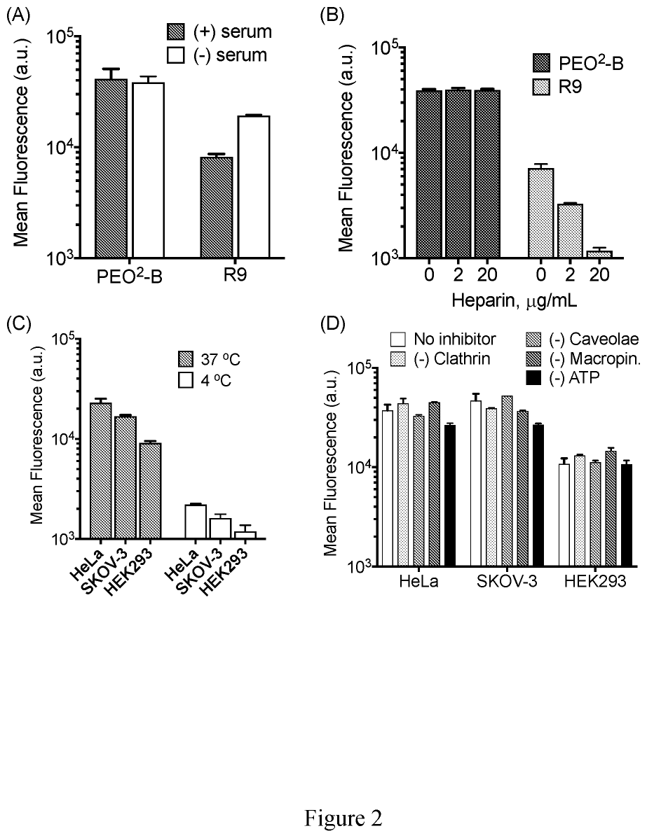

To gather insight into the uptake mechanism of PEO²-B, several well-established cellular uptake pathways were examined. First, the effects of serum and heparin sulfate on the uptake of PEO²-B were explored. Since this non-charged oligoTEA is moderately hydrophobic, and all uptake studies were performed in the presence of 10% serum, it was hypothesized that PEO²-B and other PEO-based oligoTEAs may interact with serum proteins and undergo protein-mediated uptake. This hypothesis was tested by evaluating uptake in the presence and absence of serum. The results in FIG. 2A show that the uptake of PEO²-B is not affected or mediated by serum proteins in HeLa cells. On the contrary, the uptake of cationic R9 increased by 2-fold in the absence of serum, indicating that serum proteins adversely affect the uptake of R9, presumably via non-specific binding or proteolytic degradation. The effect of heparan sulfate proteoglycans (HSPG) on the uptake efficiency of PEO²-B was also examined. HSPGs present on cell membranes as the glycocalyx have been reported to be involved in the initial step of membrane-CPP interactions. Due to their high anionic character, free heparin sulfate in solution should compete with HSPGs on the cell surface for CPP binding, and thus hinder the cellular internalization of cationic CPPs. This is what was observed when R9 was treated with heparin sulfate prior to exposure to cells. The uptake of R9 decreased by 6-fold in the presence of heparin sulfate (FIG. 2B). However, intracellular delivery with non-charged PEO²-B was unaffected by heparin sulfate, suggesting that the uptake mechanism of PEO²-B is different than that of traditional cationic CPPs. Lack of non-specific interactions of PEO²-B with serum proteins and HSPGs bodes well for potential systemic applications in vivo.

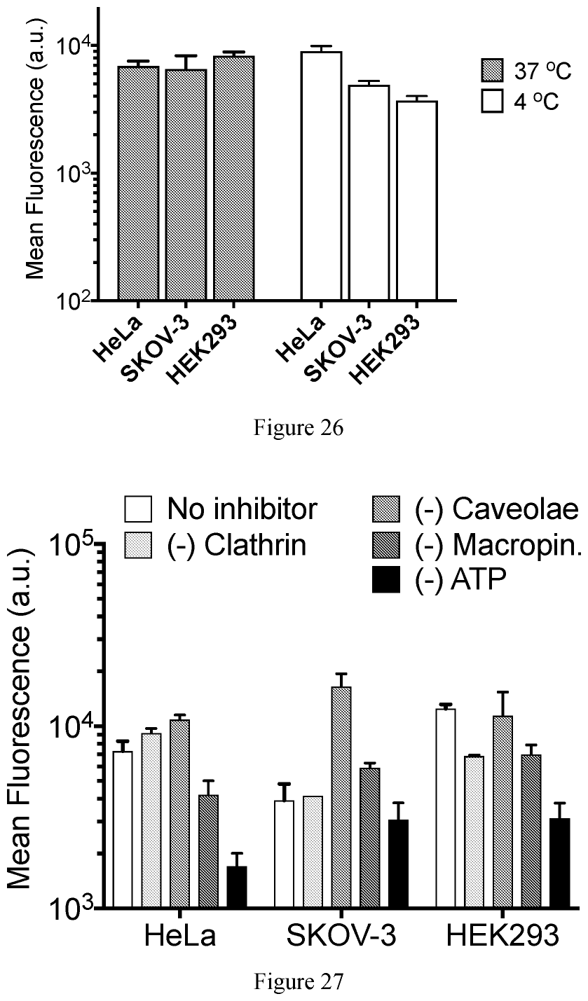

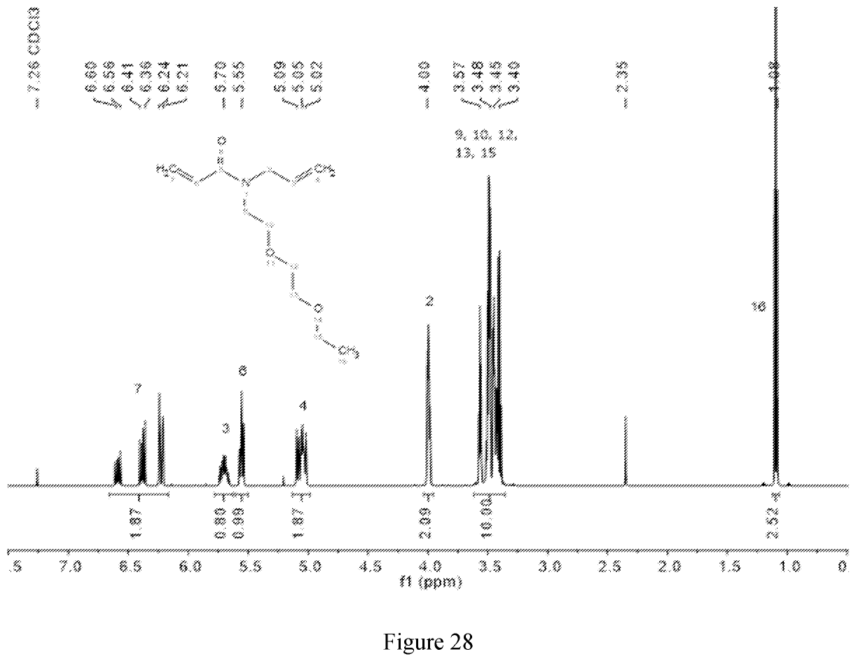

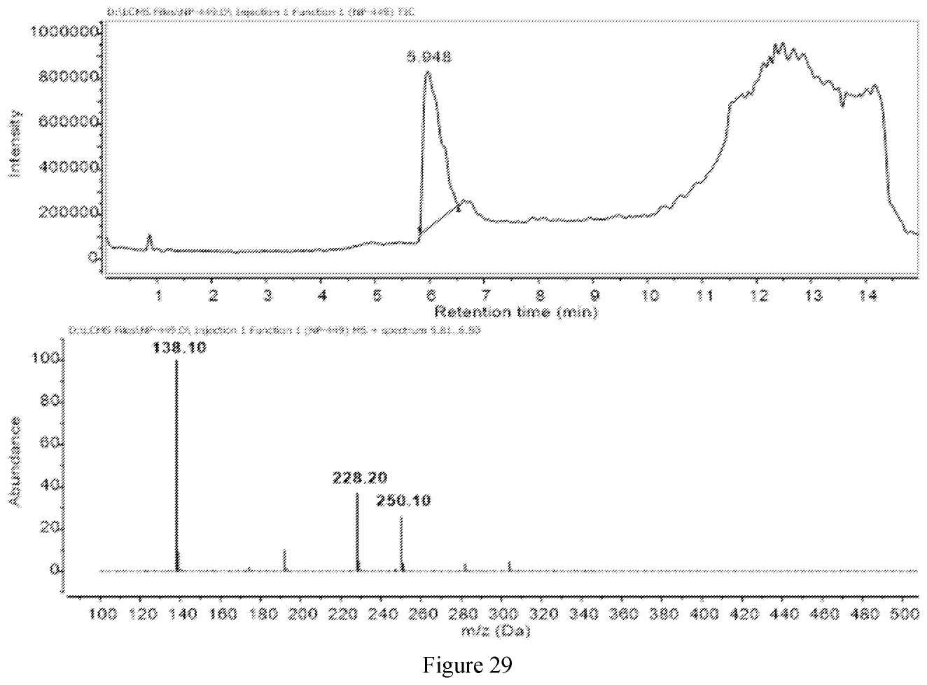

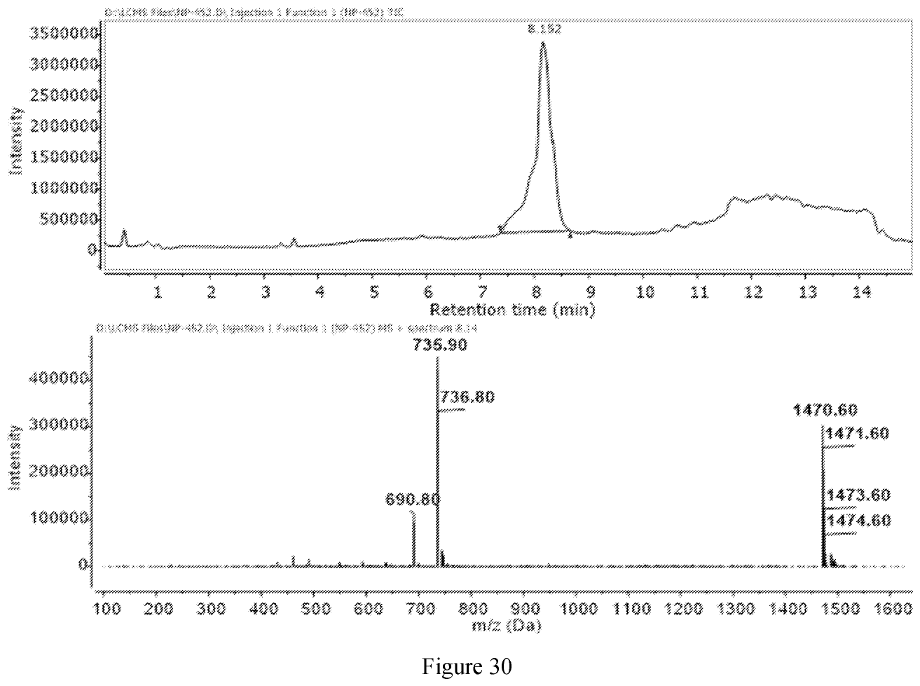

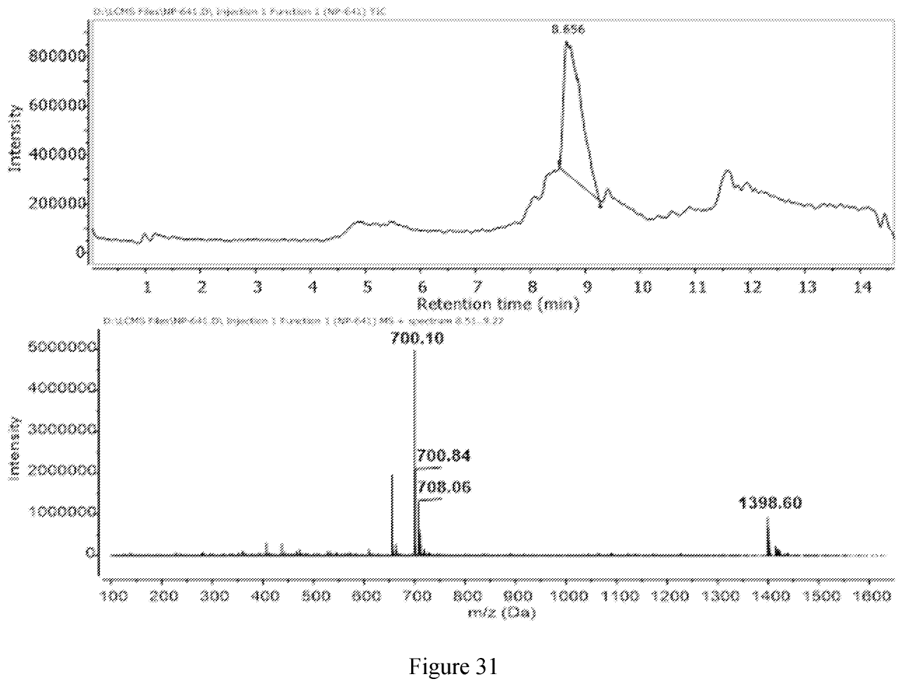

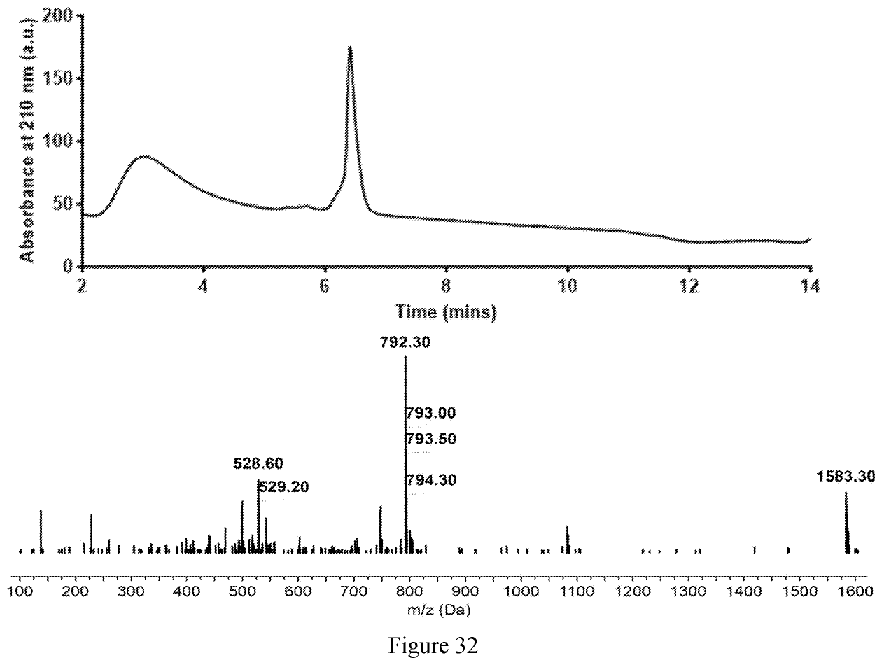

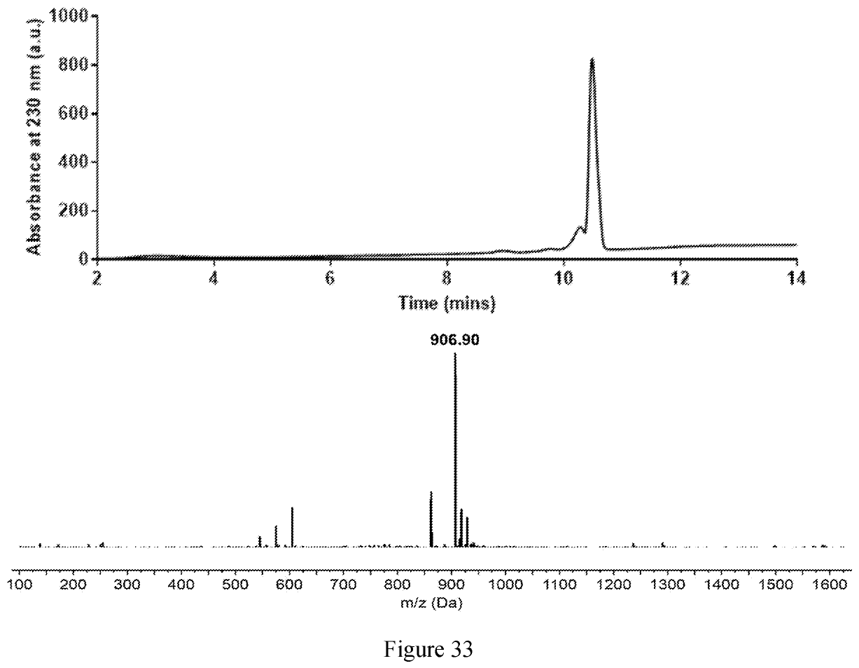

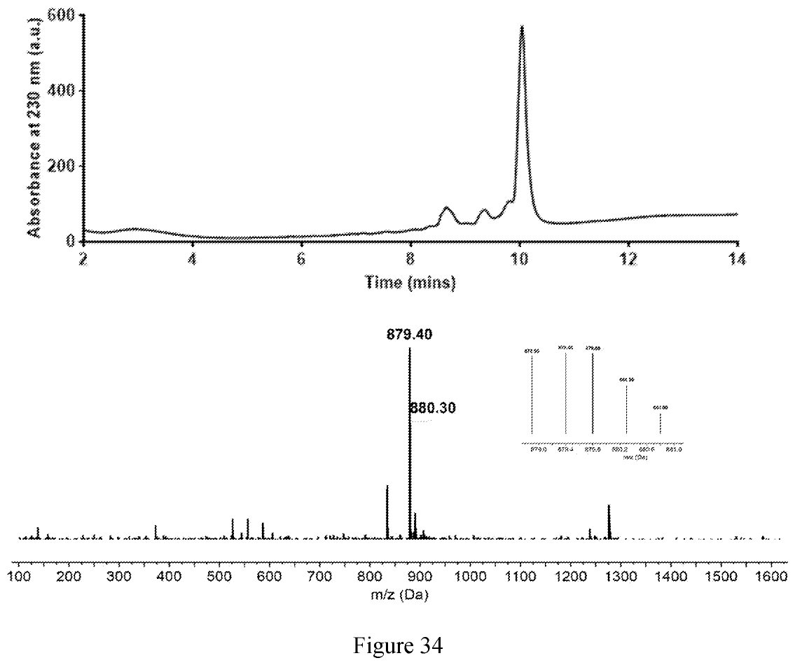

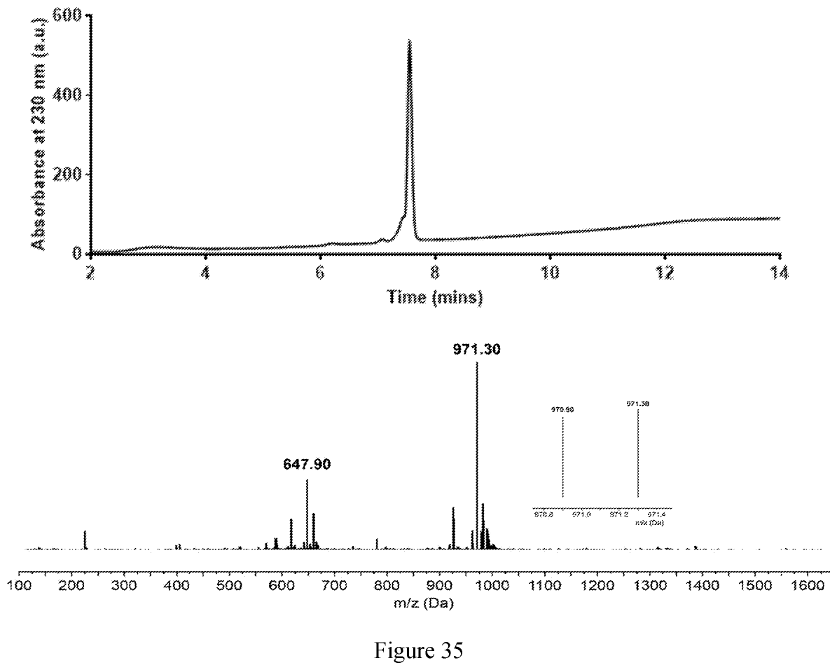

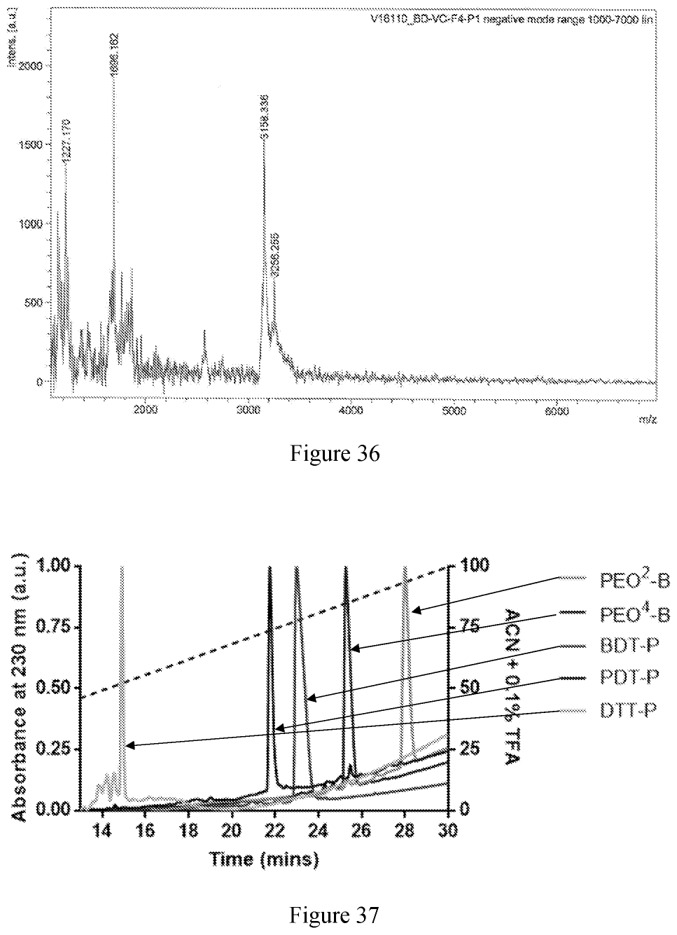

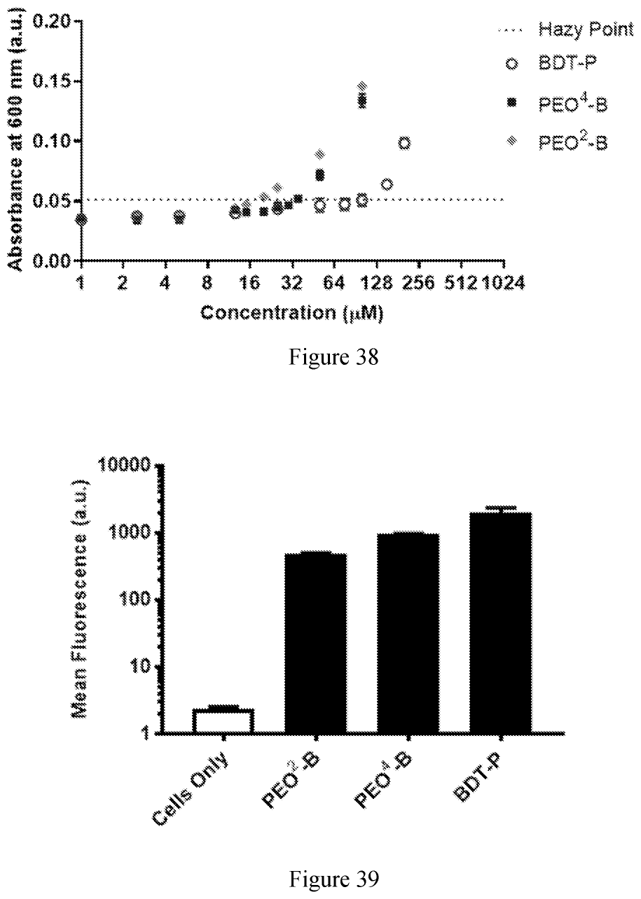

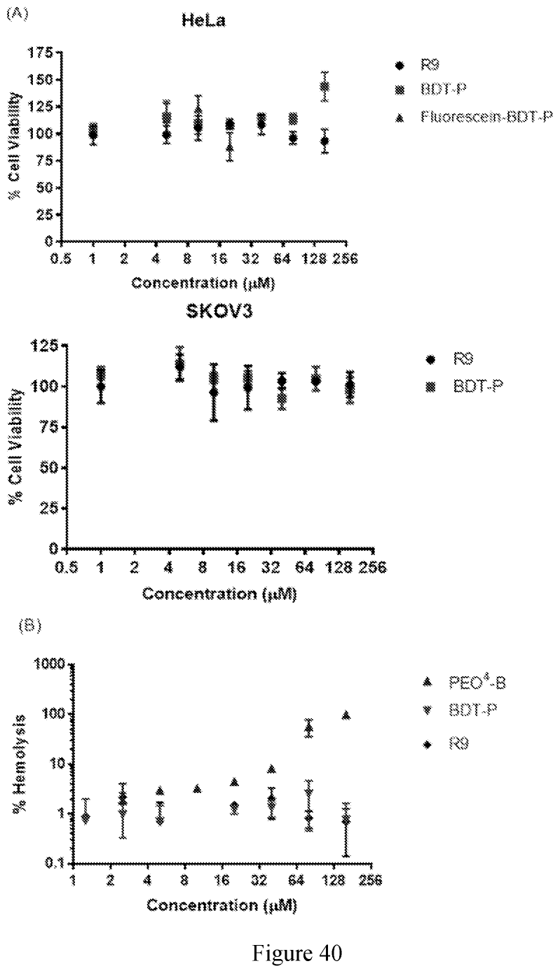

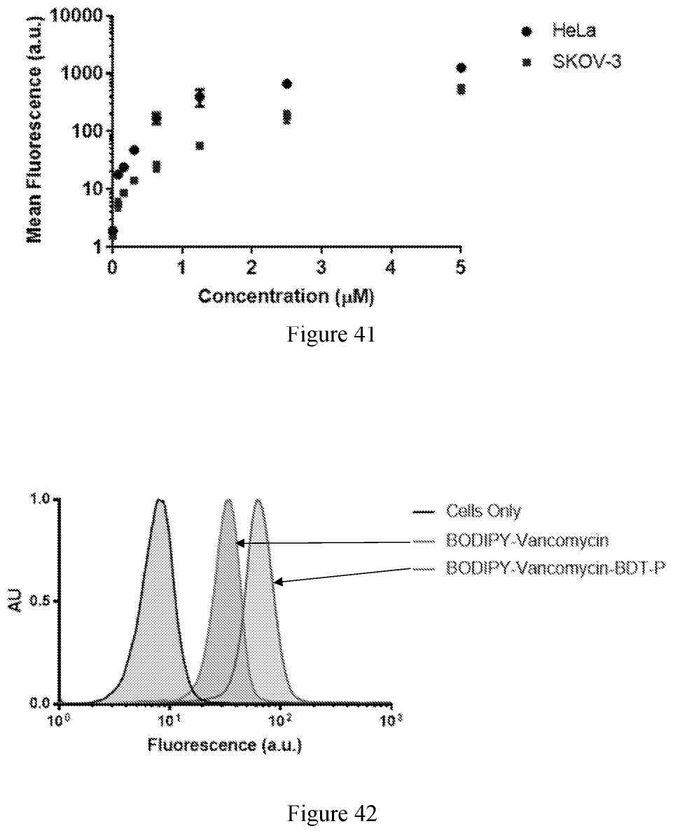

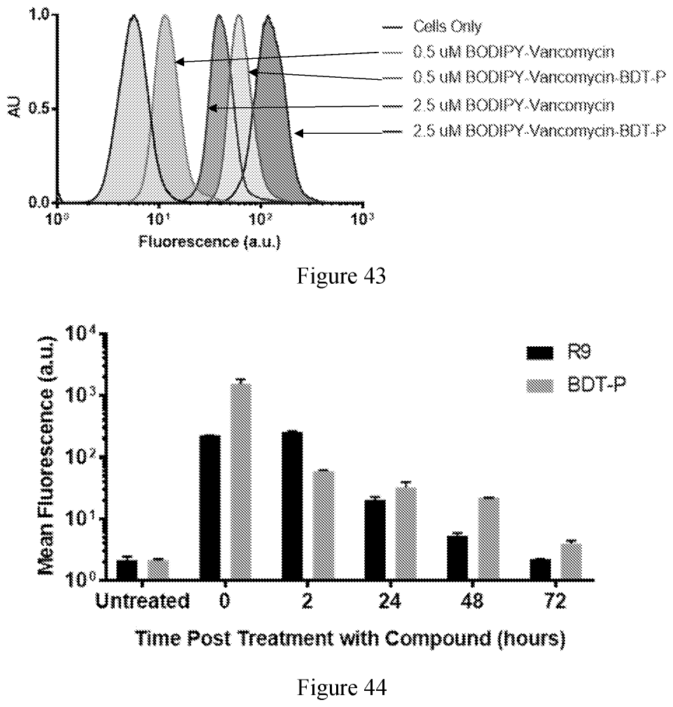

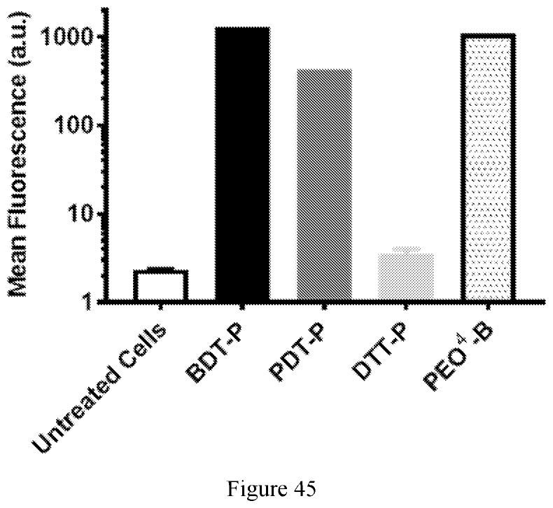

Having ruled out serum protein and HSPG interactions, it was hypothesized that the PEO²-B uptake involved one or more of the endocytosis pathways. Since endocytosis is an energy-dependent process, lowering the temperature should attenuate cellular entry. To evaluate the dependence of cellular uptake on temperature, cells were treated with PEO²-B at 4° C. Across all cell lines tested, the cellular uptake of PEO²-B at 4° C. was an order of magnitude lower than that at 37° C. (FIG. 2C), indicating a strong dependence on temperature and likely energy dependence as well. In contrast, R9 showed a weaker dependence on temperature across the three cell lines tested (FIG. 26). To examine which endocytic pathway was being used by PEO²-B, cells were pre-incubated with inhibitors of distinct endocytic pathways prior to treatment with PEO²-B. For example, chlorpromazine is known to inhibit clathrin-mediated endocytosis by halting the formation of clathrin-coated pits. Caveolae-mediated endocytosis is dependent on lipid rafts and can be inhibited by cholesterol depletion using filipin III. Finally, cytochalasin D induces de-polymerization of F-actin, which is known to attenuate micropinocytosis. Surprisingly, the uptake of PEO²-B was largely unaffected in all three cell lines after pre-treatment with these inhibitors (FIG. 2D), indicating that the primary mode of PEO²-B cellular uptake is not via these three major endocytic pathways. This is in contrast to R9 that showed a dependence on clathrin in HeLa cells and macropinocytosis in both HEK293 and HeLa cells (FIG. 27).

These results led us to question whether the uptake of PEO²-B is indeed energy-dependent. Although energy-dependent processes should depend on temperature, temperature-dependent processes are not necessarily energy-dependent. To directly probe energy dependence, sodium azide (NaN3) and 2-deoxy-D-glucose (DOG) were used to inhibit ATP-dependent processes, including endocytosis, by blocking ATP production from oxidative phosphorylation and glycolysis, respectively. Treatment with NaN3 and DOG resulted in a slight reduction of PEO²-B uptake in HeLa and SKOV-3 cells and no reduction in HEK293 cells (FIG. 2D). This result explains why none of the endocytosis inhibitors (which depend on ATP) led to a strong inhibition of PEO²-B uptake. Although other endocytic pathways beyond those tested here cannot be ruled out, these data suggest that alternative modes of cell entry maybe at play.

Based on the collective information in FIG. 2, it was hypothesized that uptake could occur via a physical translocation through the cell membrane. This mode of entry does not require cellular energy, i.e., ATP, but is temperature-dependent. In the context of the cell, temperature affects several chemical and biophysical properties, including cell membrane fluidity. At high temperatures, the lipids in the cell membrane have more kinetic energy, thus making the membrane more fluid and receptive to direct translocation of macromolecules with the right physical properties. At low temperatures, the membrane is more rigid and in a gel-like phase, potentially reducing molecular transport across the membrane. This mode of direct transport would be consistent with all the current data and would render PEO²-B dependent on membrane fluidity.

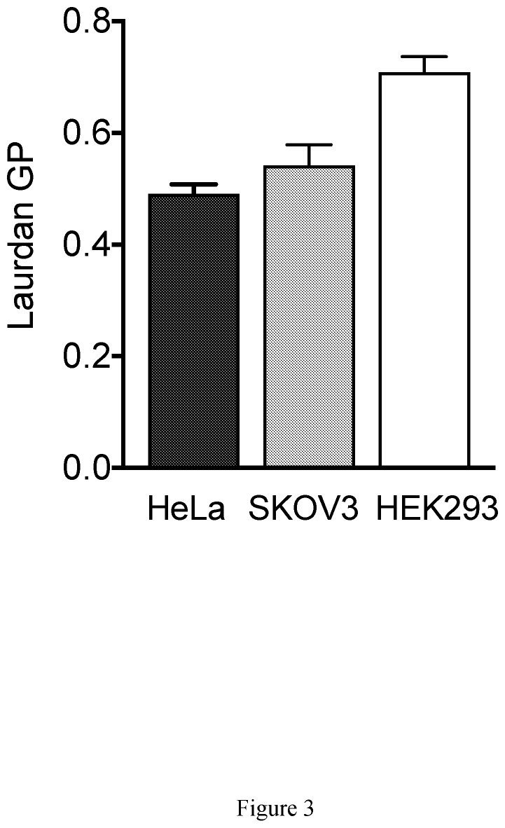

To test this hypothesis, the lipophilic fluorescent probe Laurdan (6-lauryl-2-dimethylamino-napthalene) was used. The Laurdan dye is sensitive to the polarity of its immediate environment and has been used to measure cellular membrane fluidity. This measurement, which is done via two-photon laser microscopy, reports back a generalized polarization (GP) that ranges from −1 to +1 with lower values indicating greater membrane fluidity. The GP value was measured in all three cells lines and the data shows that HeLa cells have the most fluid membrane, followed by SKOV-3 cells, then HEK293 cells (FIG. 3). This data is in very good agreement with the flow cytometry uptake data in FIG. 1C, which shows greater uptake of PEO²-B in HeLa than SKOV-3 and HEK293 cells. These data, coupled with earlier results, suggests that one of PEO²-B's primary modes of cellular uptake is via direct translocation through the cell membrane.

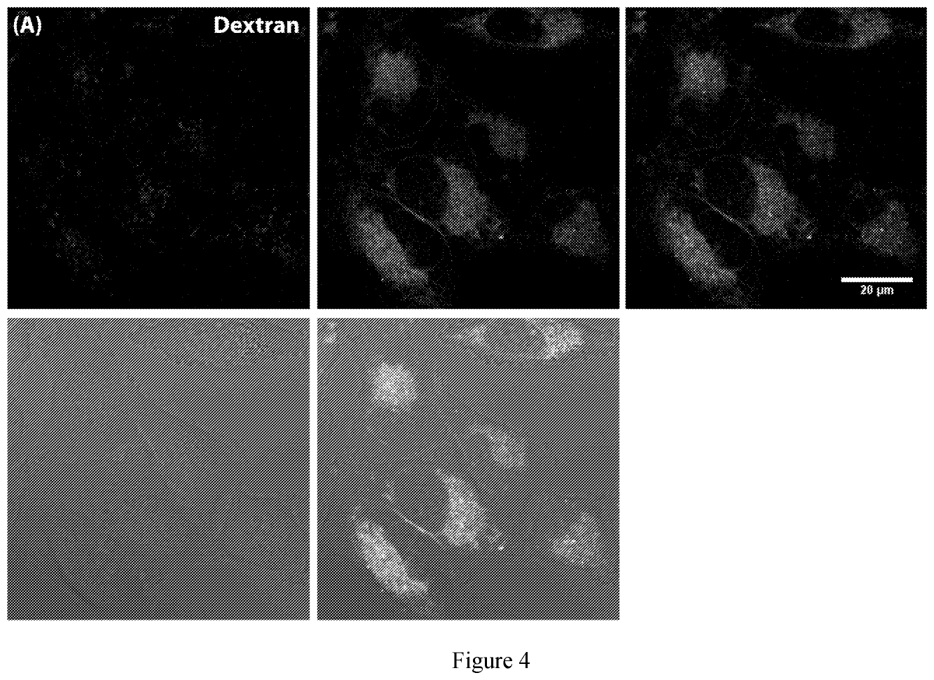

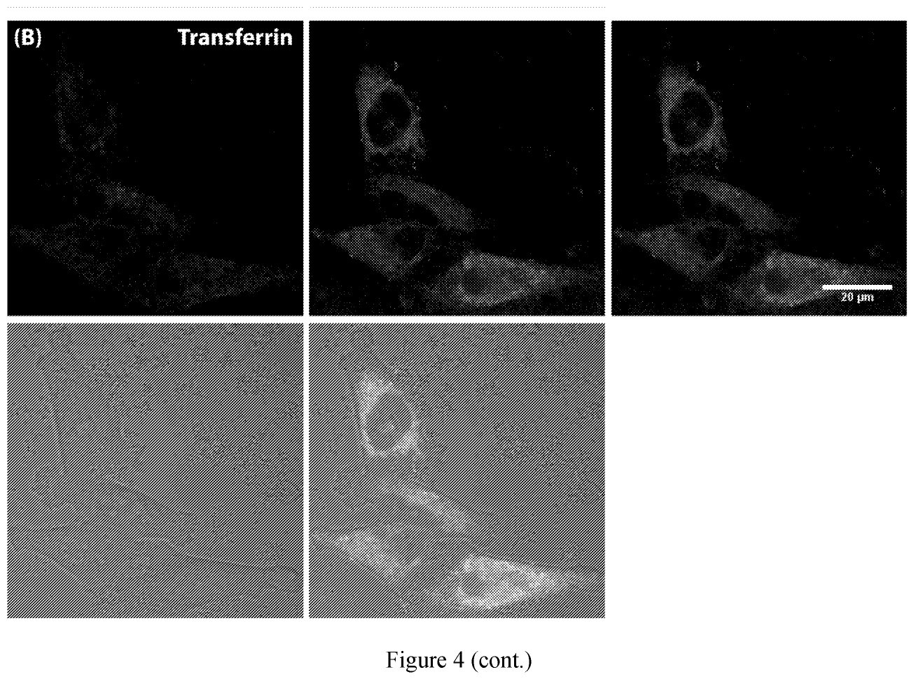

It was earlier confirmed via confocal microscopy that PEO²-B was undergoing cellular internalization as opposed to cell surface binding. Access to the cells via direct membrane translocation could lead to a variety of intracellular locations. To determine the range of compartments where PEO²-B could reside, HeLa cells were treated with fluorescein-labeled PEO²-B and key markers of different compartments-transferrin for early and recycling endosomes, dextran for macropinosomes, and lysotracker for acidic compartments such as lysosomes. Overall, co-delivery with these compartment-specific fluorescent probes showed that PEO²-B is predominantly dispersed in the cytoplasm (FIG. 4). A small fraction of co-localization with transferrin and dextran is observed but the majority of the signal appears diffuse in the cytosol (FIGS. 4A and 4B). These results are consistent with prior experiments suggesting that a large fraction of PEO²-B's uptake is due to direct membrane translocation. A smaller fraction may go in via unknown endocytic mechanisms or cytoplasmic PEO²-B is able to access membrane bound intracellular compartments also via direct membrane diffusion. Collectively, these experiments indicate that non-charged cell-penetrating oligoTEAs provide a general strategy for the intracellular delivery of small molecules.

TABLE 1
List of allylacrylamide monomers and dithiols used in this disclosure.

| Letter code | Sequence |
|---|---|
| G | 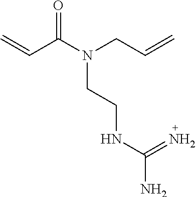 |
| B | 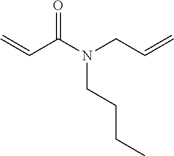 |
| P | 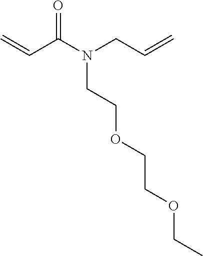 [SMILES: C=CCN(CCOCCOCC)C(=O)C=C] |
| DTT | 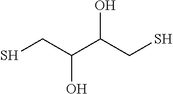 |
| BDT | |
| PDT | |
| PEO | |
| 3,6-dioxa-1,8- | |
| octanedithiol (as shown) | |

DTT: dithiothreitol
BDT: 1,4-butane-dithiol
PDT: 1,3-propane-dithiol
PEO: polyethylene oxide/polyethylene glycol.

TABLE 2
List of oligoTEAs used in this disclosure.

| Name | Sequence |
|---|---|
| DTT-G | DTT-G-DTT-G-DTT-G-DTT-G |
| DTT-B | DTT-B-DTT-B-DTT-B-DTT-B |
| BDT-B | BDT-B-BDT-B-BDT-B-BDT-B |
| PEO¹-B | PEO-B-BDT-B-BDT-B-BDT-B |
| PEO²-B | PEO-B-BDT-B-PEO-B-BDT-B |
| PEO³-B | PEO-B-PEO-B-PEO-B-BDT-B |
| PEO⁴-B | PEO-B-PEO-B-PEO-B-PEO-B |
| BDT-P | BDT-P-BDT-P-BDT-P-BDT-P |
| PDT-P | PDT-P-PDT-P-PDT-P-PDT-P |
| DTT-P | DTT-P-DTT-P-DTT-P-DTT-P |

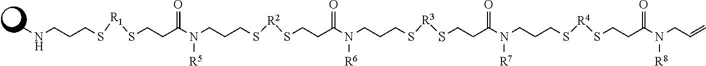

DTT-G: polymer with DTT incorporated into R¹-R⁴ group with R⁵-R⁸ being guanidinium side chains.

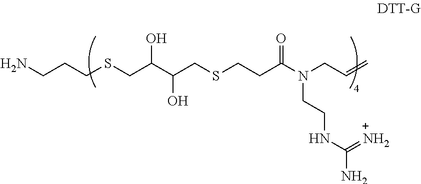

DTT-B: polymer with DTT incorporated into R¹-R⁴ group with R⁵-R⁸ being butane side chains.

BDT-B: polymer with butane incorporated into R¹-R⁴ group with R⁵-R⁸ being butane side chains.

PEO¹-B: polymer with PEO incorporated into R¹, butane into R²-R⁴ group with R⁵-R⁸ being butane side chains.

PEO²-B: polymer with PEO incorporated into R¹ and R³, butane into R² and R⁴ group with R⁵-R⁸ being butane side chains.

PEO³-B: polymer with PEO incorporated into R¹-R³, butane into R⁴ group with R⁵-R⁸ being butane side chains.

PEO⁴-B: polymer with PEO incorporated into R¹-R⁴ with R⁵-R⁸ being butane side chains.

BDT-P: polymer with butane incorporated into R¹-R⁴ group with R⁵-R⁸ being PEO side chains.

PDT-P: polymer with propane incorporated into R¹-R⁴ group with R⁵-R⁸ being PEO side chains.

DTT-P: polymer with DTT incorporated into R¹-R⁴ group with R⁵-R⁸ being PEO side chains.

### Example 2

This example describes oligoTEAs and methods of making oligoTEAs the present disclosure.

Recently, a unique approach for the rapid assembly of sequence-defined oligomers was developed, referred to as oligothioetheramides (oligoTEAs). OligoTEAs have three distinct advantages over native peptides. First, sequence-defined oligomers are abiotic and not susceptible to protease degradation, thus increasing their bioavailability. Second, access to direct modification of the oligoTEA backbone enables direct control over their conformation, rigidity, and pendant group spacing to ultimately tune interactions between the binding motifs and the cell membrane. Finally, the use of synthetic monomers will allow for massive compositional diversity.

Studies conducted on several CPPs thus far indicate that a combination of cationic and hydrophobic residues are critical for translocation across cellular membrane. Based on these and many other studies, an 8-residue oligoTEA library was assembled composed of a hydrophilic backbone (DL-dithiothreitol), a cationic (guanidinium) monomer, and a hydrophobic (benzyl) monomer. Probing this initial library of 16 oligomeric structures for cellular uptake led to the conclusion that for this class of macromolecules, cationic residues are not a requirement for efficient uptake. Expanding this library with other functional group led to the discovery of non-charged cell-penetrating oligoTEAs (CPOTs) that undergo efficient cellular uptake with low cytotoxicity, and outperform R9, a well-known and widely used CPP.

This new class of highly efficient non-charged macromolecular transporters are distinct from their cationic counterparts and show strong promise for the intracellular delivery of therapeutics such as small and medium molecule antibiotics, e.g. vancomycin, to treat intracellular infections as well as peptide and protein therapeutics. As a proof-of-concept, a reducible CPOT-vancomycin conjugate was assembled and demonstrated its efficient transport into host cells towards the treatment of intracellular bacteria. CPOT-vancomycin conjugates were efficient at clearing intracellular Listeria monocytogenes within macrophages in less than six hours, thus showing promise as viable macromolecular therapeutics. It also demonstrated efficient intracellular transport of a small 43 amino acid peptide (˜5 kDa).

OligoTEA Synthesis

OligoTEAs were synthesized using alternating thiol-ene and thiol-Michael addition reactions, followed by cleavage of the fluorous tag. Completed oligoTEAs were purified using reverse-phase HPLC and verified using LC-MS and ¹H NMR.

Thiol-ene reaction: Three equivalents of dithiol and 2,2-dimethoxy-2-phenylacetophenone (DMPA, 5 mol % of dithiol) were added to a solution of corresponding fluorous-olefin (100 mM) in methanol. The reaction mixture was subjected to UV irradiation for 270 s at 20 mW/cm². The product (fluorous-thiol) was purified by fluorous solid-phase extraction (FSPE).

Thiol-Michael addition: Two equivalents of corresponding monomer and dimethyl phenyl phosphine (Me₂PhP, 5 mol % of monomer) were added to the fluorous-thiol (100 mM) in methanol eluted from the purification of last thiol-ene reaction. Methanol was removed by reduced pressure in 1-1.5 hours. The time required for the evaporation of methanol was enough for the quantitative conversion of Michael addition. The reaction mixture was purified by FSPE.

FSPE: The fluorous organic mixture was loaded onto a cartridge pre-packed with 2 g of fluorous silica. A fluorophobic wash (4:1 methanol:water) was used to elute the non-fluorous molecules whereas the fluorous molecules were retained on the fluorous silica gel. A fluorophilic wash with methanol was then used to elute the fluorous molecules from the fluorous stationary phase.

Fluorous tag cleavage reaction: Fluorous-assembled oligoTEAs were dissolved in a 5 mM 1:1 trifluoroacetic acid (TFA):DCM mixture and stirred for 1 hr at room temperature. TFA and DCM was removed under nitrogen, and the oligoTEAs were purified using reverse-phase HPLC.

HPLC purification: OligoTEAs were purified on a 1100 Series Agilent HPLC system equipped with a UV diode array detector and a 1100 Infinity semi-prep scale fraction collector using reverse phase C18 column (9.4×250 mm, 5 μm). The column compartment was kept at 30° C. during fractionation. Solvents for HPLC were water with 0.1% TFA (solvent A) and acetonitrile with 0.1% TFA (solvent B). On a standard gradient, oligoTEAs were eluted at a flow rate of 4 mL/min with 5% solvent B, followed by a linear gradient of 5% to 100% solvent B over 30 mins, and finally 100% solvent B for 10 mins before equilibrating the column back to 5% solvent B over 3 mins. OligoTEAs were collected based on their absorption at 230 nm. The fractionated oligoTEA was transferred to a vial, dried and stored under argon until further analysis.

Fluorescein-OligoTEA Conjugates Synthesis

Pure, cleaved oligomers (10 mg/mL in DMSO) were reacted with 6.5 equivalents of NHS-fluorescein (mixed isomers of 5- and 6-carboxyfluorescein succinimidyl ester at 7.5 mg/mL in DMSO) and 10 equivalents of triethylamine for 1 hr at room temperature. The reaction mixture was then purified via HPLC. Fluorescein-oligoTEA conjugates were collected based on their absorption at 230 and 460 nm. The fractionated oligoTEA was transferred to a vial, dried and stored until further analysis. The conjugates were quantified by their fluorescence signals (Ex./Em. 493/515 nm) using a standard curve of NHS-fluorescein.

BODIPY-Vancomycin-OligoTEA Conjugates Synthesis

BODIPY-Vancomycin (10 mg/mL) was reacted with 5 equivalents of OligoTEA (50 mg/mL), 30 equivalents of N-methylmorpholine (1 μM), and 20 equivalents of N,N,N′,N′-Tetramethyl-O-(1H-benzotriazol-1-yl)uronium hexafluorophosphate (HBTU, 0.5 M) in 1:1 DMSO:DMF at room temperature for 15 mins. The reaction was then quenched with 1:1 Methanol:Water. The products were purified via HPLC and confirmed by MALDI-MS.

Vancomycin-SS-OligoTEA Conjugates Synthesis

OligoTEA was reacted with 1.5 equivalents of Dinitrophenyl disulfide linker and 3 equivalents of N,N-Diisopropylethylamine in DMF at room temperature overnight. The products were purified via HPLC and confirmed by LC-MS.

Vancomycin hydrochloride was then reacted with 1.2 equivalents of Linker-OligoTEA and 6 equivalents of N,N-Diisopropylethylamine in 1:1 DMSO:DMF at room temperature overnight. The products were purified via HPLC and confirmed by MALDI-MS.

Disulfide Cleavage Kinetics of Vancomycin-SS-oligoTEA Conjugates using DL-DTT

30 μM of vancomycin-SS-oligoTEA conjugates (˜20 ug) were treated with 10 mM of DL-DTT (˜331 ug) in 1×PBS pH 7.4 at 37° C. 20-uL aliquot (˜2 ug) was taken out at each time point (1 to 4 hrs). The aliquots were injected directly into the LC-MS. The LC-MS software was set to extract for the following masses: 1552.40 ([M+H]⁺ for vancomycin-SH), 1448.40 ([M+H]⁺ for vancomycin), and 1036.7 ([M+3H]³⁺ for vancomycin-SS-oligoTEA).

Biological Assays

Flow Cytometry Assay: 50,000 cells/well were plated in 24-well plates and incubated at 37° C. for 20-24 hrs. Cells were washed with 1×PBS pH 7.4 and incubated with fluorescein-oligoTEA conjugates at the desired concentration in normal growth media at 37° C. for 1 hr; each compound was tested in duplicates. After the incubation, cells were washed with PBS. For A549 and MC-3T3-E1 cell lines, cells were incubated with Trypsin EDTA at 37° C. for 3-5 mins. Normal growth media was then added to quench the trypsin. For J774 cell line, normal growth media was added, and cells were de-attached using a cell scraper. Cells from each well were transferred to an Eppendorf® tube and centrifuged at 500×g for 5 mins. The supernatant was removed, and cells were then re-suspended in 500 μL of PBS. Readings were taken on a FACSCalibur™ flow cytometry analyzer (Becton Dickinson). Results were analyzed by FlowJo software. The data presented is the mean fluorescence from 10,000 gated cells.

MTS Cell Proliferation Assay: 15,000 J774 cells/well were plated in 96-well plates and incubated at 37° C. for 20-24 hrs. Cells were washed with 1×PBS pH 7.4 and incubated with 100 μL of 10 μM to 120 μM of compounds in DMEM with 10% FBS at 37° C. for 1 hr. After the incubation, cells were washed 3 times with PBS. 100 μL of clear DMEM with 10% FBS and 10 μL of MTS solution (Promega) were added, and the plate was incubated for 1 hr. Absorbance measurements were taken at 490 nm on a TECAN Infinite M1000 PRO Microplate reader and normalized to untreated cells (100%). All experiments were performed in triplicates.

In vitro Intracellular Infection Assay: 100,000 cells/well J774 cells in DMEM supplemented with 10% FBS without pen-strep, referred to as normal growth media, were plated in a 24-well plate and incubated for 20-24 hrs before use. Listeria monocytogenes DP-L1942 strain was taken from an exponentially growing culture (OD600 of ˜0.5) and washed with 1×PBS pH 7.4. Macrophages were infected with 200,000 bacteria in normal growth media to achieve an initial infection of approximately two bacteria per cell (MOI=2). The cells were washed twice with PBS at 30 mins after infection and supplemented with normal growth media. At 1-hr post infection, gentamicin (50 μg/ml) was added to eliminate all extracellular bacteria. The cells were then washed twice with PBS after 30 mins of incubation and supplemented with normal growth media. At 2-hr post infection, 30 μM of CPOT-vancomycin conjugates were added to the infected cells and incubated for 4 hrs. At the end of the incubation period, macrophages were washed twice with PBS, and the cells were lysed with 0.1% Triton-X in PBS. The cell lysate was diluted 10× in Brain Heart Infusion (BHI) broth and plated in a 96-well plate. The plate was incubated at 37° C. with agitation. The growth curve kinetics were generated from the absorbance measurements at 600 nm taken every 5 mins for 14 hrs.

Peptide-PEG₄-oligoTEA Synthesis

Purified oligomers (10 mg/mL in DMSO) were reacted with 5 equivalents of Mal-PEG₄-NHS and 10 equivalents of triethylamine for 1 hr at room temperature. The reaction mixture was then purified via RP-HPLC. Mal-PEG₄-oligoTEAs were collected and confirmed via LC-MS.

TAT-HA and HA peptides were then reacted with 2 equivalents of Mal-PEG₄-oligoTEAs and 10 equivalents of N,N-Diisopropylethylamine for 24 hr at 37° C. The reaction mixture was then purified via RP-HPLC. Peptide-PEG₄-oligoTEA conjugates were collected and confirmed via LC-MS or MALDI-MS.

Immunofluorescence Staining

70,000 HeLa cells/well were plated in a 4-well chambered coverglass and incubated at 37° C. for 20-24 hrs. Cells were washed with 1×PBS pH 7.4 and incubated with 5 μM TAT-HA-Cholesterol, TAT-HA-oligoTEA and HA-oligoTEA conjugates for 1 hr at 37° C. Cells were washed 3 times with PBS and fixed with 4% formaldehyde for 15 mins at room temperature. Cells were washed twice with PBS and blocked with blocking buffer (5% Normal Goat Serum, 0.3% Triton-X in PBS) overnight at 4° C.

Cells were incubated with 1:500 dilution of rabbit anti-HA peptide-2 primary antibody in blocking buffer for 1 hr at room temperature. Cells were washed with blocking buffer and incubated with 1:500 dilution of AlexaFluor 568 goat anti-rabbit IgG secondary antibody in blocking buffer for 1 hr at room temperature. Cells were washed with blocking buffer and stained with 1:10,000 dilution of Hoechst 33342 in PBS for 15 mins at room temperature. Cells were washed with PBS and stored in PBS for imaging.

Results and Discussion

OligoTEA Synthesis

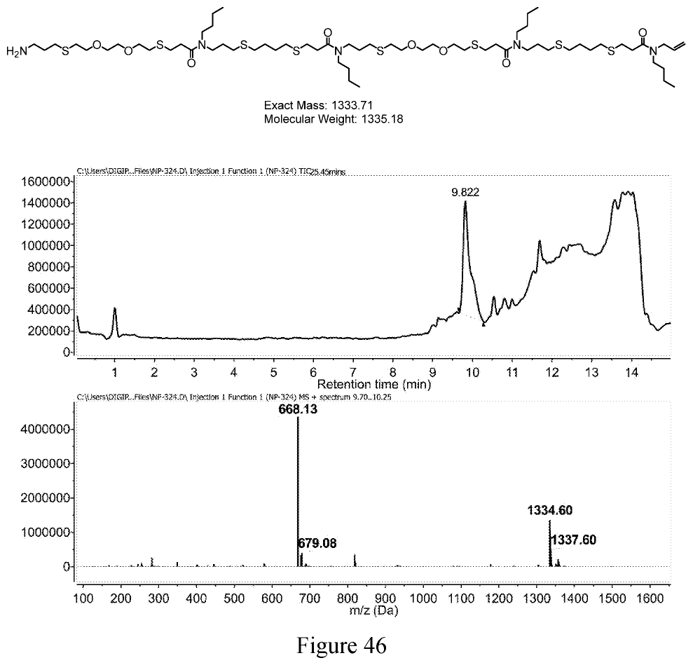

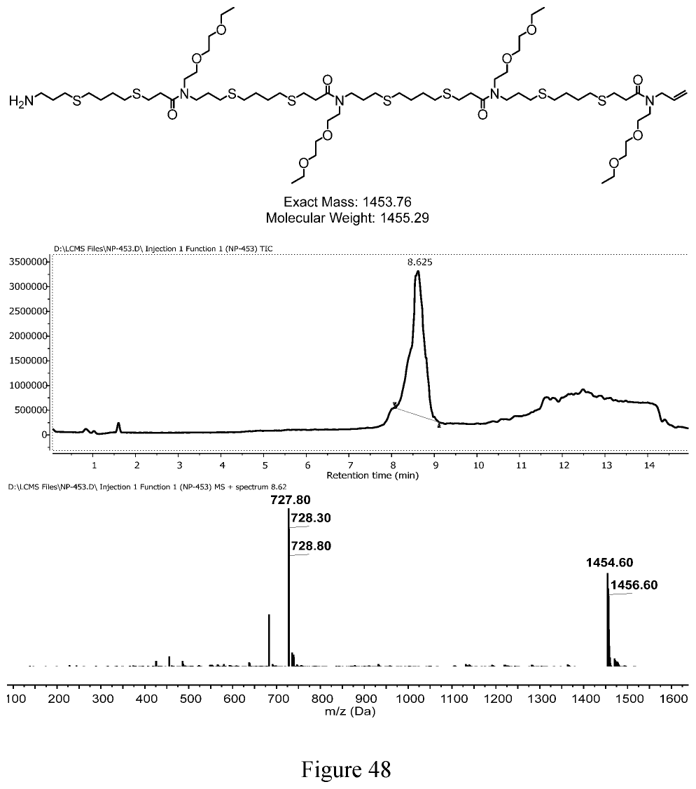

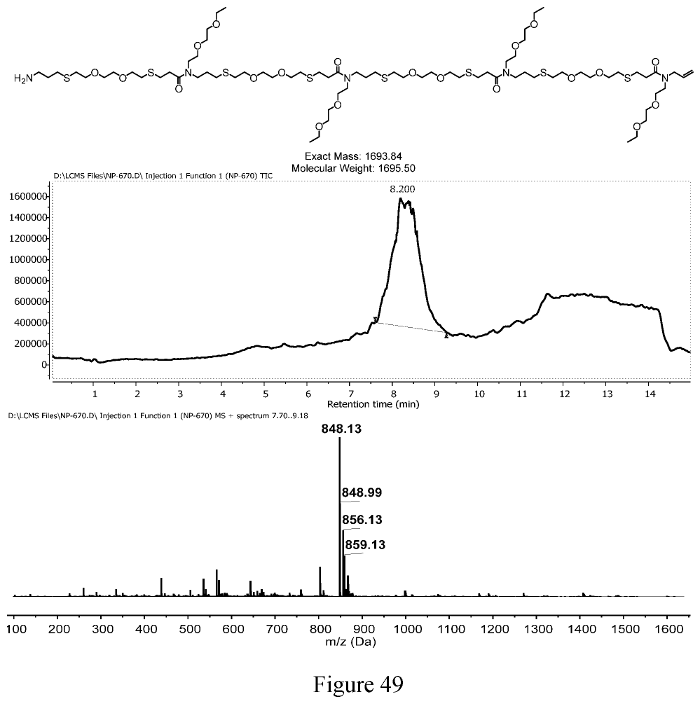

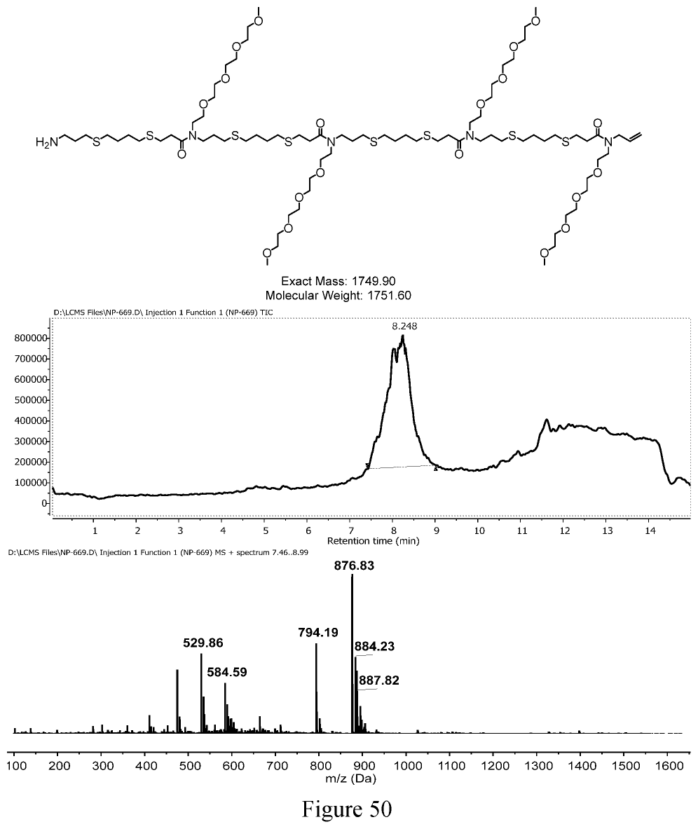

OligoTEAs were synthesized as described above. The products were purified via HPLC and confirmed by LC-MS. See FIGS. 46-50.

HPLC Retention Times and Solubility in Aqueous Solution

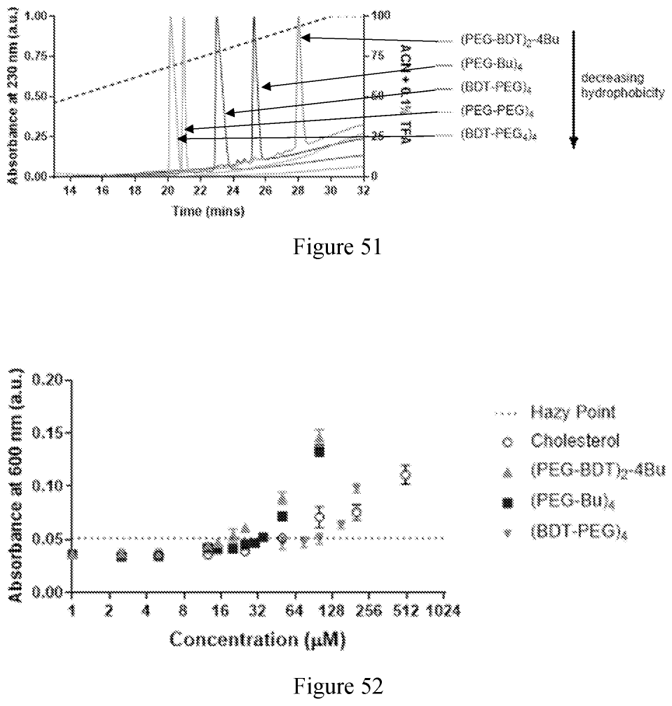

To compare the relative hydrophobicity of oligoTEAs, we ran them on the same HPLC gradient. The earlier the product elutes, the more hydrophilic it is, as seen in FIG. 51.

To evaluate the solubility of oligoTEAs, we measured their absorbance at 600 nm over a wide range of concentrations in 1×PBS at pH 7.4. The hazy point is the point at which a faint cloudiness is observed, which corresponds to an A600 of ˜0.05. FIG. 52 shows the solubility limits of selective oligoTEAs. This solubility trend matches with the hydrophobicity trend that we have seen with the HPLC retention times.

Fluorescein-OligoTEA Conjugates Synthesis

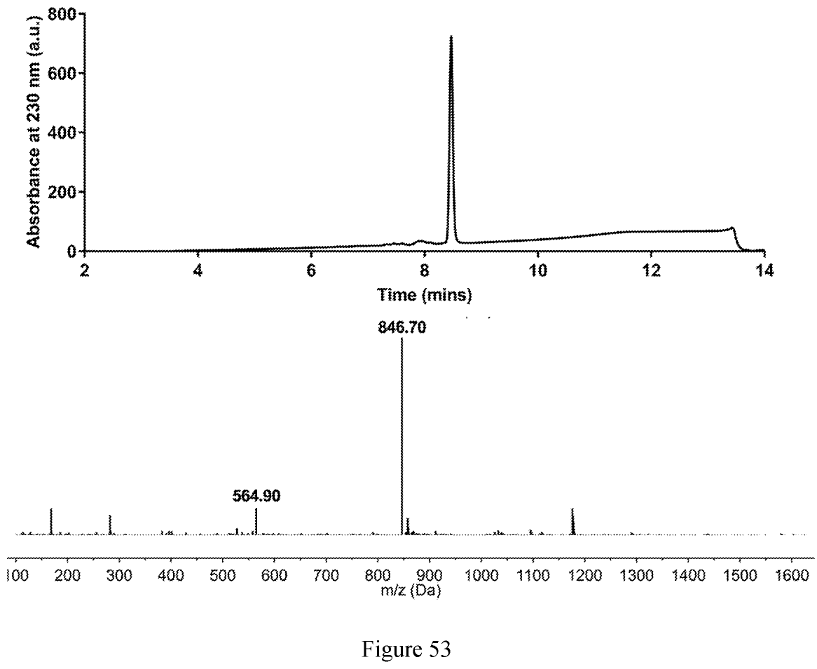

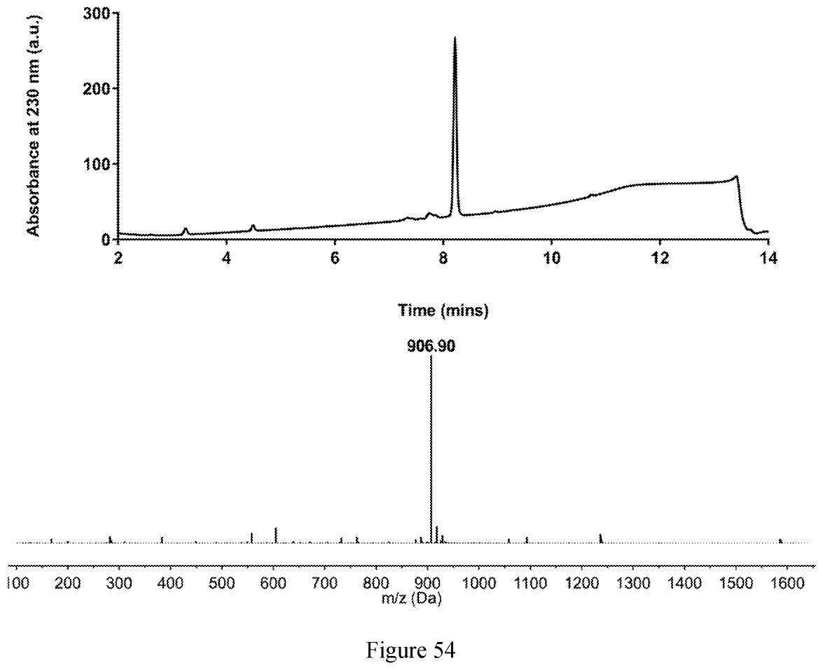

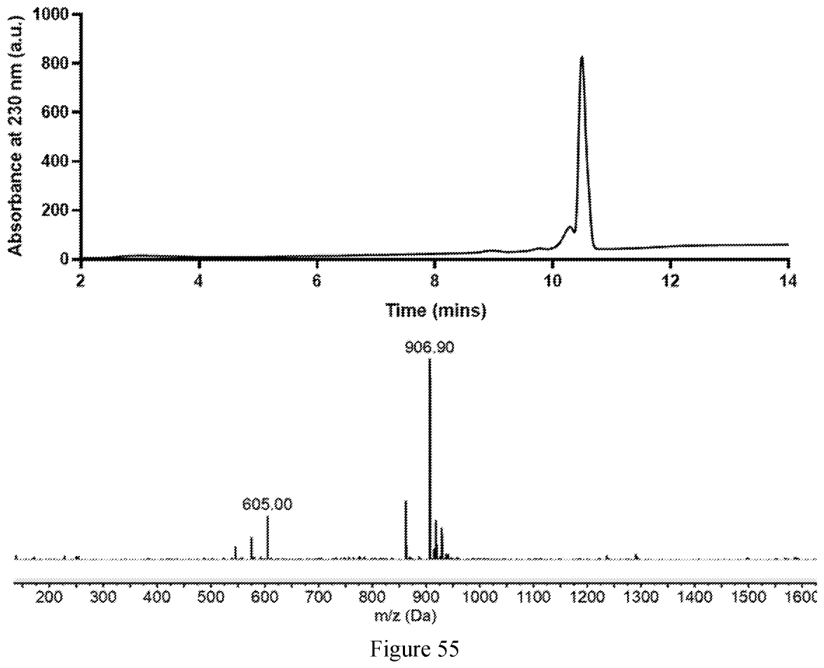

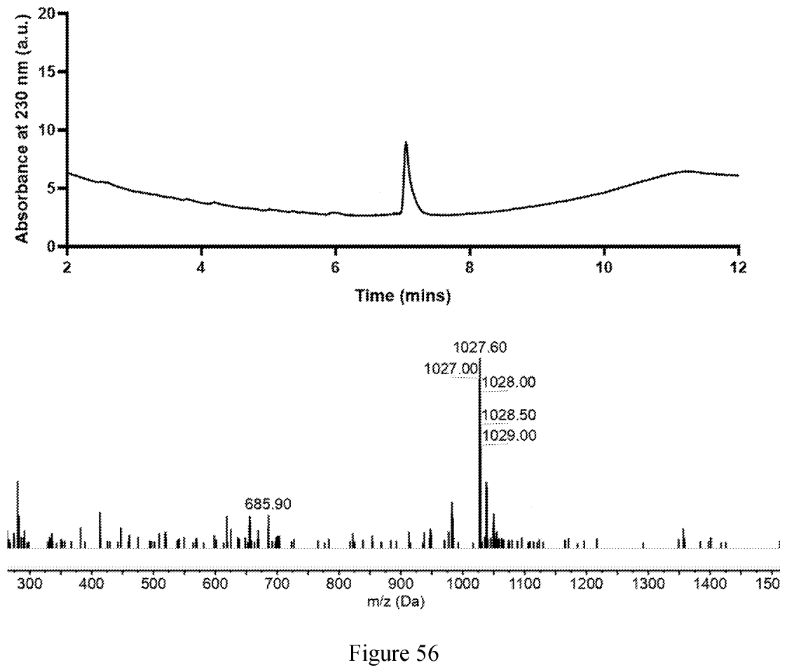

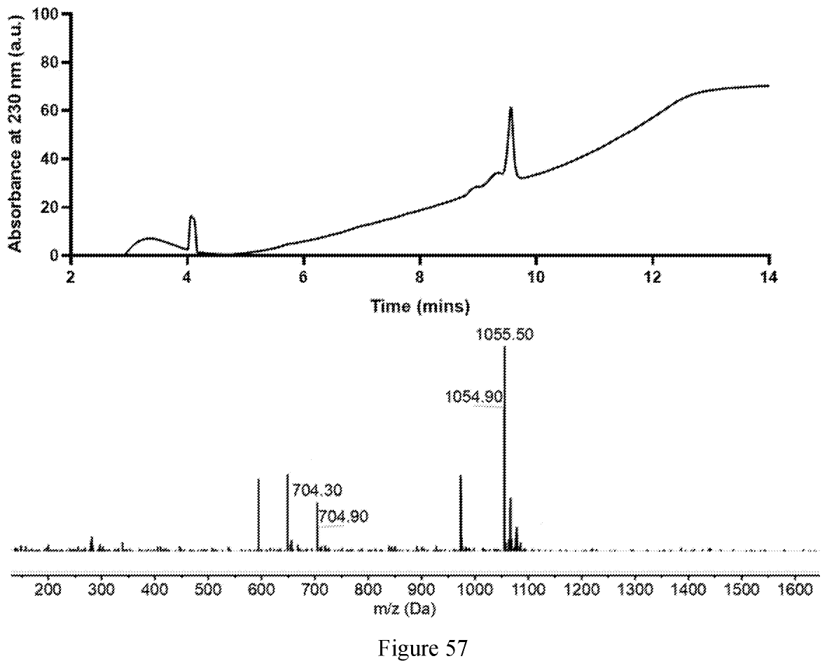

Fluorescein-oligoTEAs were synthesized as described above. The products were purified via HPLC and confirmed by LC-MS. See FIG. 53-57.

BODIPY-Vancomycin-OligoTEA Conjugates Synthesis

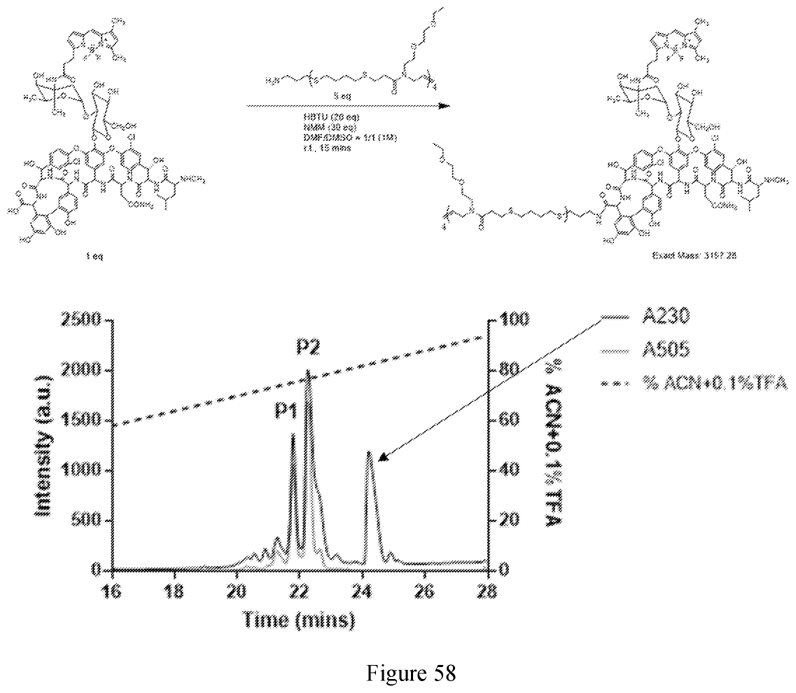

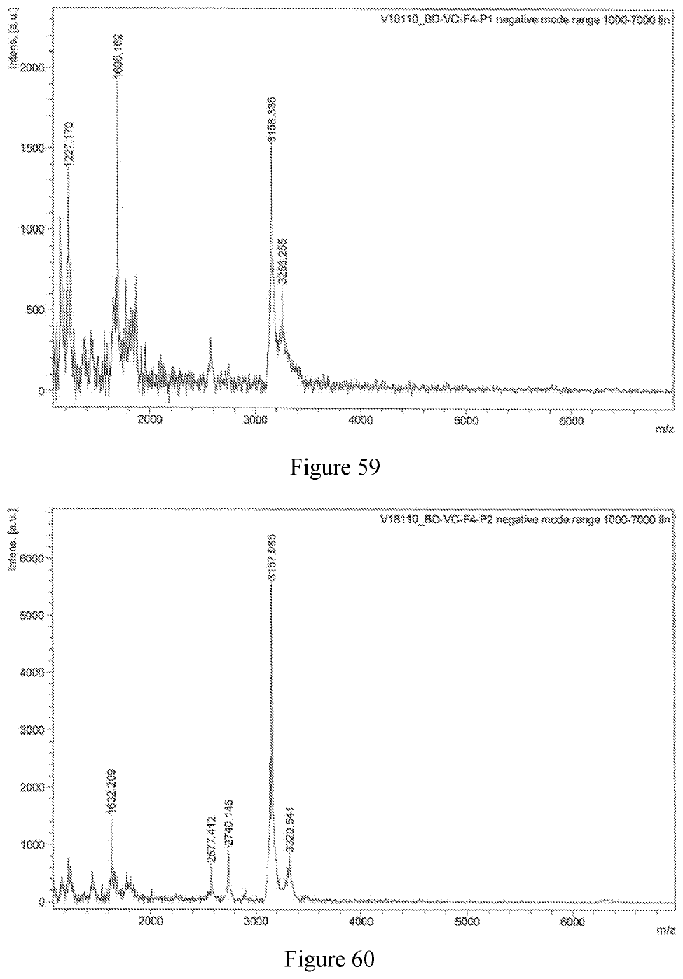

BODIPY-Vancomycin-(BDT-PEG)₄ conjugates were synthesized as described in the experimental section. The reaction mixture was purified via HPLC as shown in FIG. 58. The BODIPY-Vancomycin stock obtained from is a mixture of conjugates in which the BODIPY is attached to either the primary or secondary amine of vancomycin. Thus, when (BDT-PEG)₄ was conjugated to BODIPY-Vancomycin, there were two products, referred to as P1 and P2. The products were collected and confirmed by MALDI-MS (FIGS. 59 and 60).

Vancomycin-SS-OligoTEA Conjugates Synthesis

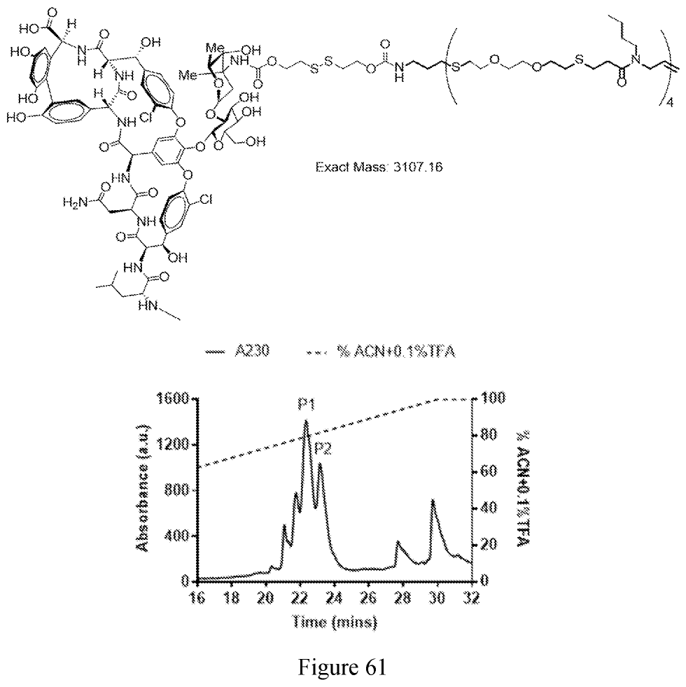

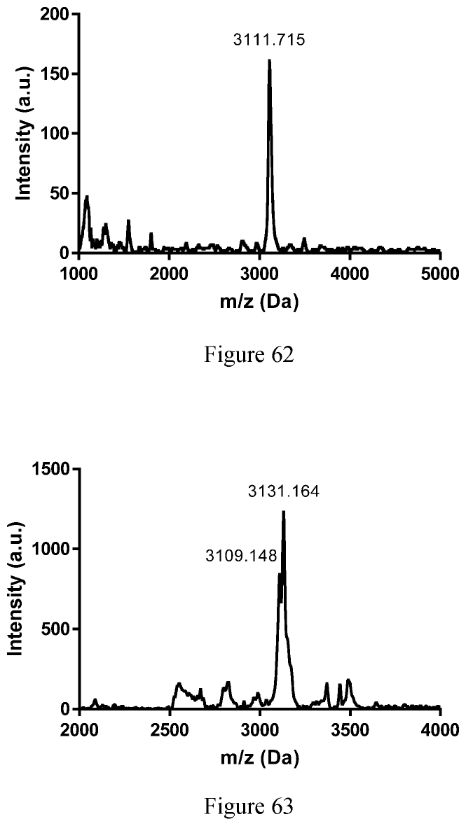

Vancomycin-SS-(PEG-Bu)₄ conjugates were synthesized by conjugating vancomycin hydrochloride to Linker-(PEG-Bu)₄ as described above. The reaction mixture was purified via HPLC as shown in FIG. 61. Since the Linker-(PEG-Bu)₄ can attach to either the primary or the secondary amine on vancomycin, two products were collected and confirmed by MALDI-MS (FIGS. 62 and 63).

Uptake of Fluorescein-OligoTEAs in J774, MC-3T3-E1, and A549 Cells

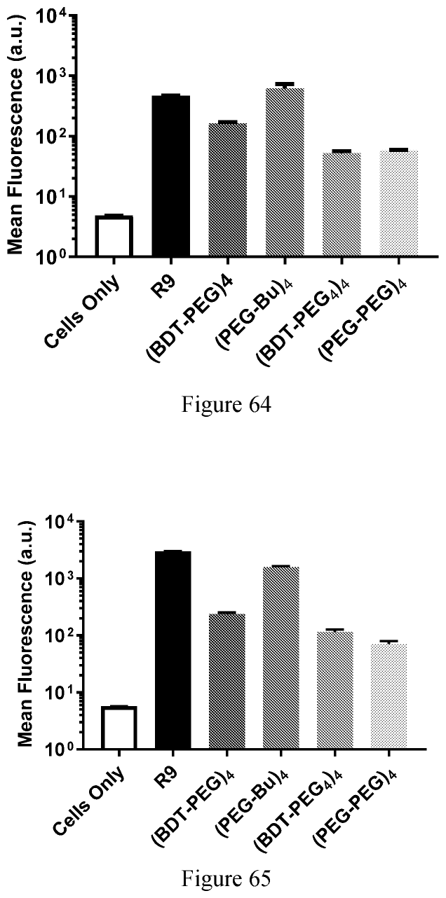

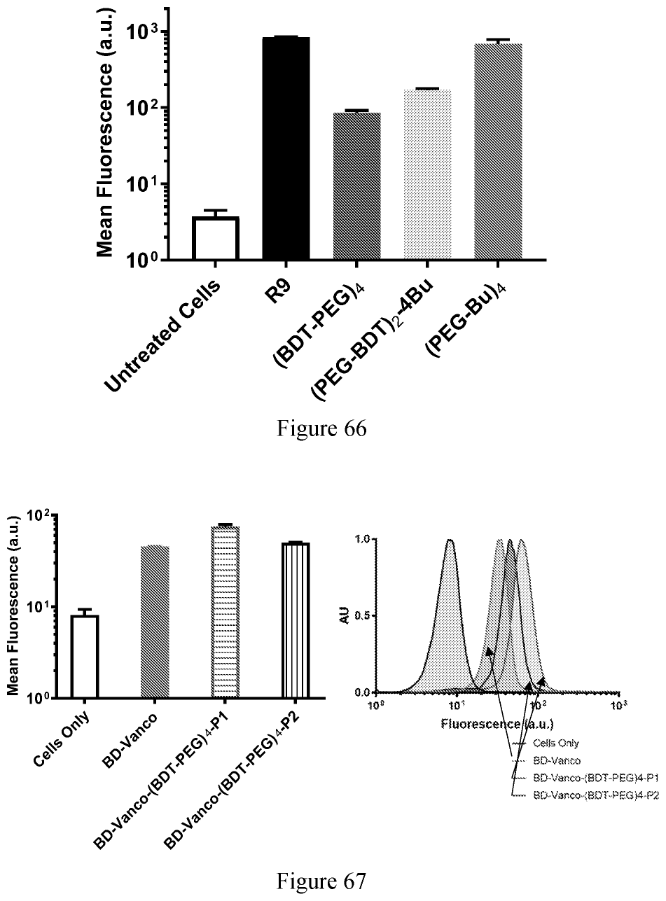

As shown in FIGS. 64 and 65, uptake of fluorescein cargo by oligoTEAs in J774 and MC-3T3-E1 cells seem to follow the same trend: R9 (PEG-Bu)₄>(BDT-PEG)₄>(BDT-PEG₄)₄˜(PEG-PEG)₄. Uptake of some of these oligoTEAs in A549 cells (FIG. 66) also agree with this trend.

Uptake of BODIPY-Vancomycin-(BDT-PEG)₄ in HeLa Cells

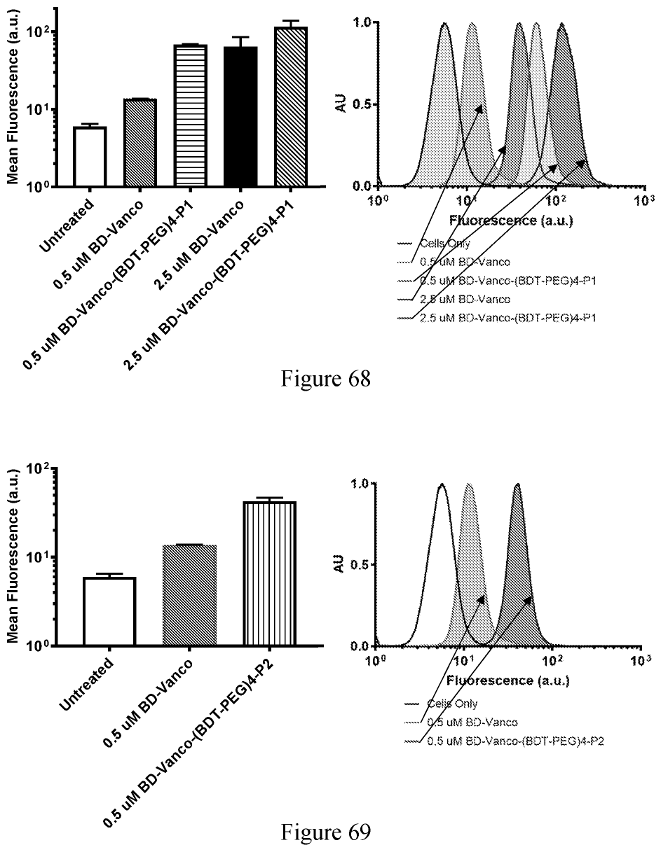

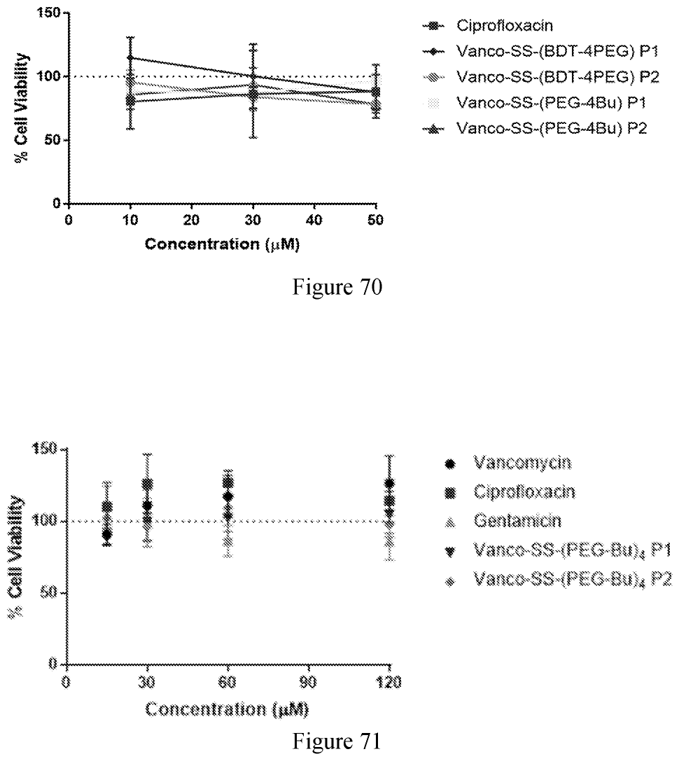

To see if oligoTEAs are able to deliver a different cargo into cells, (BDT-PEG)₄ was conjugated to BODIPY-Vancomycin and measured uptake of the two products P1 and P2 in HeLa cells. As seen in FIGS. 67-69, P1 and P2 at undergo efficient uptake at 0.5 to 2.5 μM. As the concentration was decreased (FIGS. 68 and 69), the difference between the uptake of the conjugates and that of BODIPY-vancomycin became more significant.

Antibacterial Activity of Vancomycin-SS-oligoTEA Conjugates

To evaluate the effectiveness of our vancomycin-SS-oligoTEA conjugates in the treatment of intracellular bacteria, we decided to use Listeria monocytogenes DP-L1942 strain to infect J774 cells. The infection protocol is described above based on published data on Listeria macrophage infections. DP-L1942 is an actA deletion (AActA) strain, a virulence-attenuated mutant that is unable to polymerize actin and spread from cell to cell.

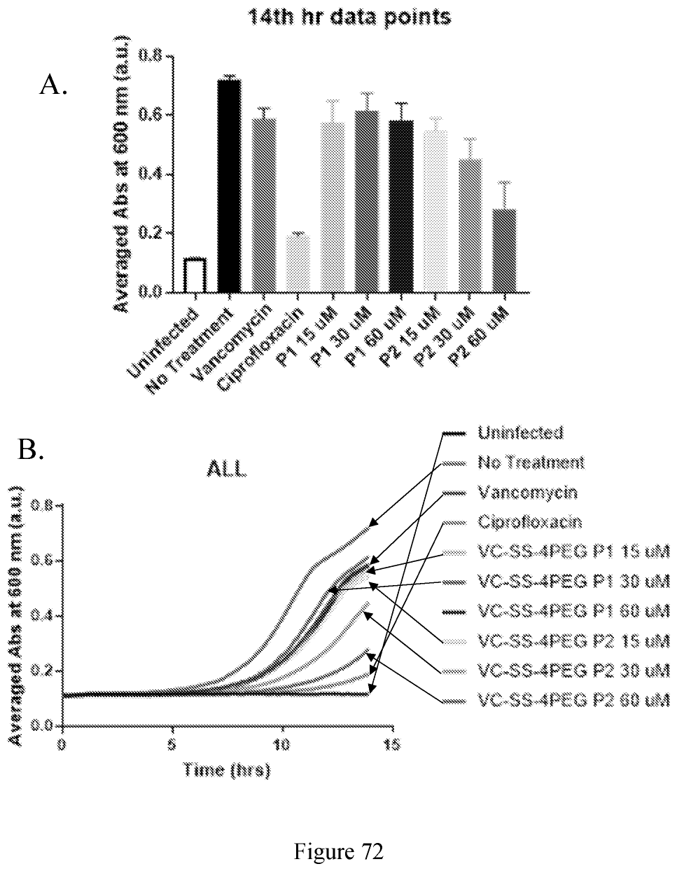

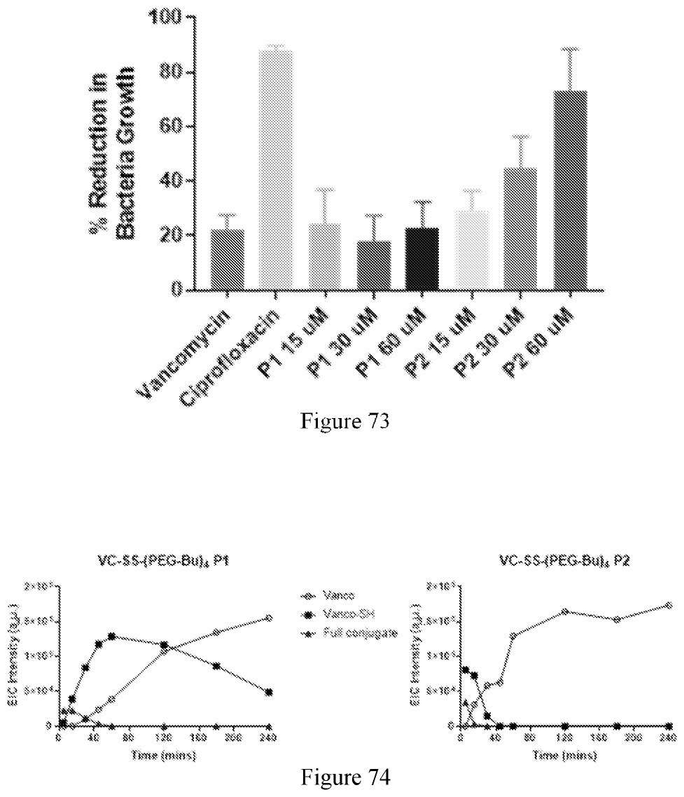

As shown in FIGS. 72 and 73, vancomycin cannot enter the host cells, and thus has little to no effect on bacteria growth. Ciprofloxacin is an antibiotic commonly used to treat intracellular infection. Thus, it is active and able to inhibit more than 80% of bacteria growth. P1 appears to have no activity at all at up to 60 μM. On the other hand, P2 seems to have a dose-dependent effect on intracellular Listeria. P2 can inhibit more than 40% of bacteria growth at 30 μM and more than 70% of bacteria growth at 60 μM, which is almost as active as ciprofloxacin at 30 μM. In addition, since the conjugates appear non-toxic to macrophages at up to 120 μM, concentration can be increased if needed to achieve the desired activity.

Cleavage Studies of Vancomycin-SS-(PEG-Bu)₄ Conjugates with DL-DTT

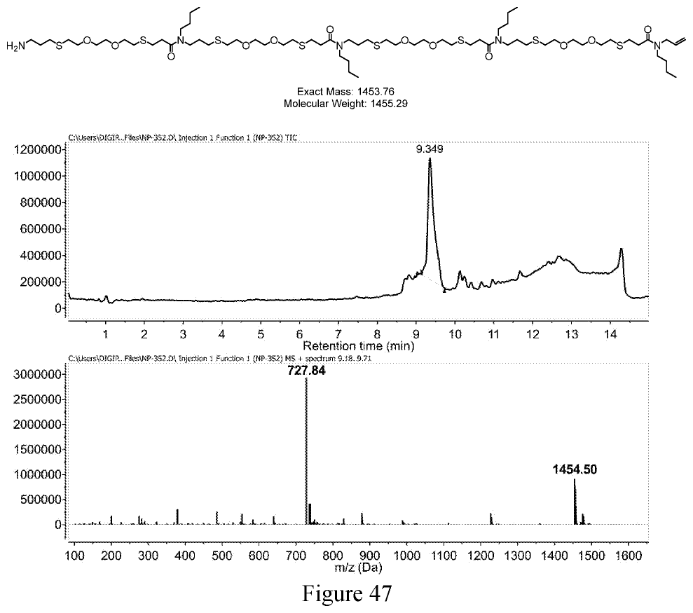

The cleavage of P1 and P2 were monitored by LCMS at 5, 15, 30, 45, 60, 120, 180, and 240 minutes. The reaction mixture was injected directly into the LCMS. As seen in FIG. 74, P2 seems to cleave faster than P1. At 45 mins, some of P1 full conjugate was still present while there was no detection of P2 full conjugate. FIG. 47 also shows that the thiolate on the vancomycin of P1 was completely eliminated at 60 mins, while that process of P1 was still present even at 4 hrs. The faster cleavage kinetics of P2 may explain while it is more potent than P1.

Dynamic Light Scattering (DLS) Measurements of Vancomycin-SS-(PEG-Bu)₄ Conjugates

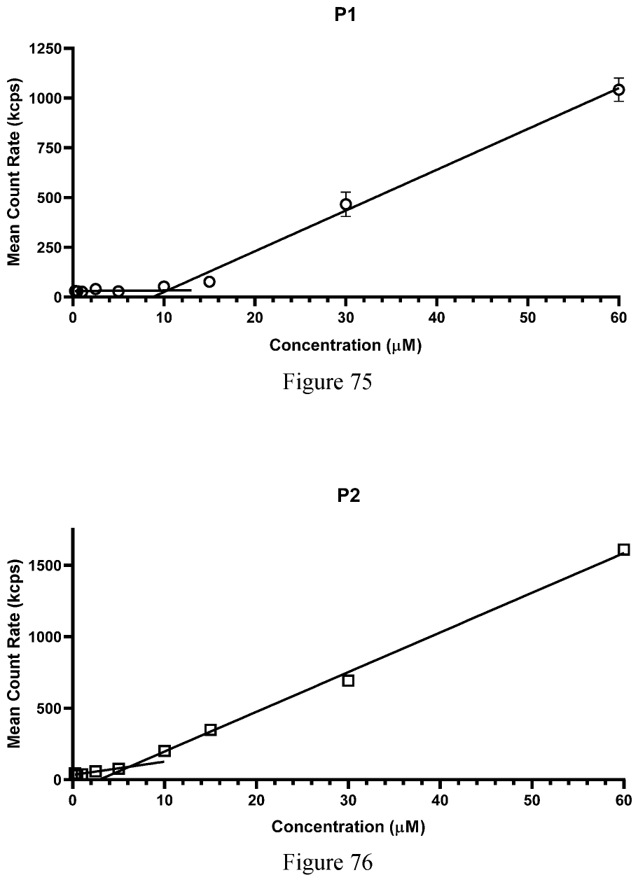

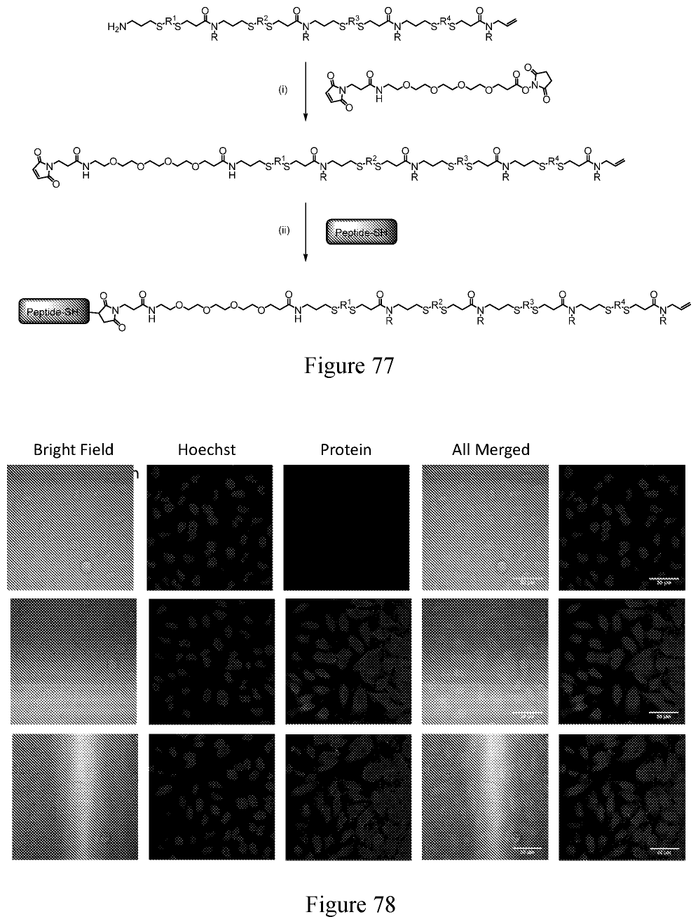

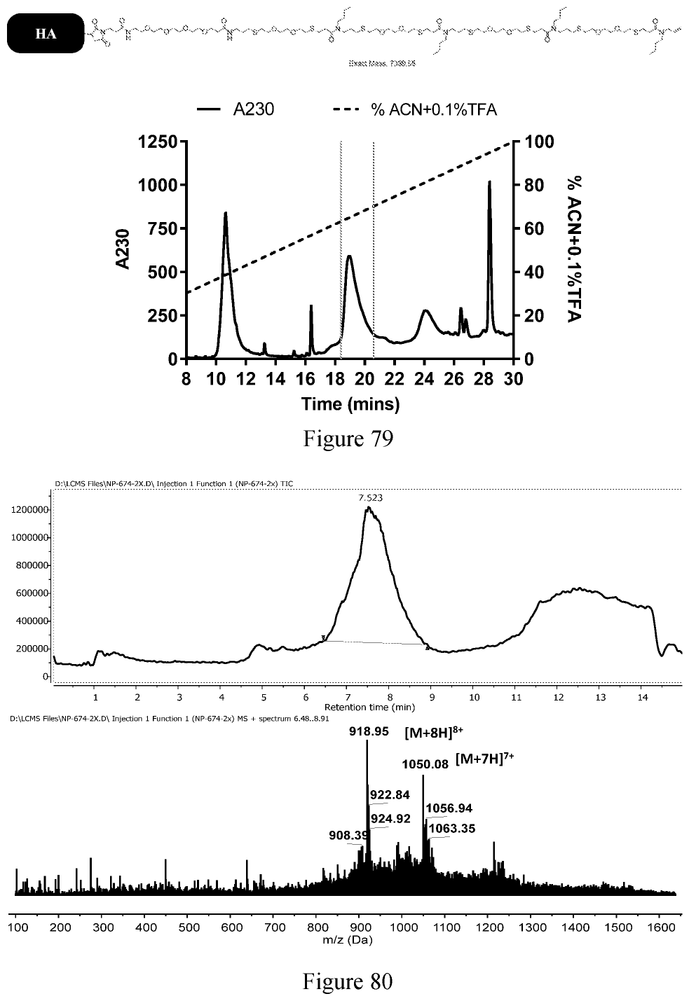

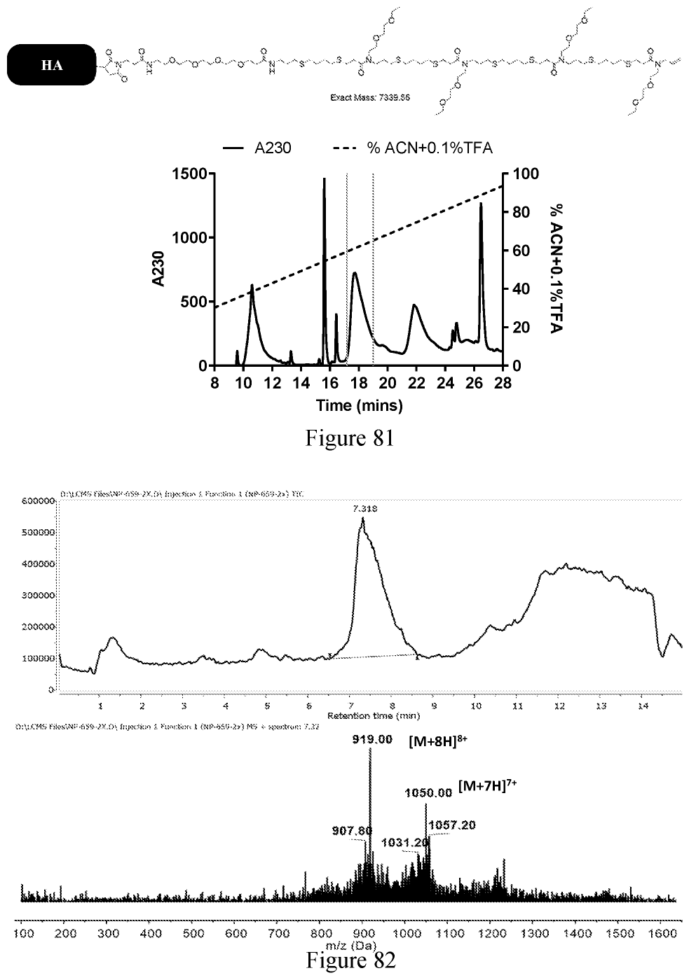

To learn whether the vancomycin-SS-(PEG-Bu)₄ conjugates form any aggregates in aqueous solution, DLS measurements at different concentrations using the Zetasizer was performed. The mean count rates were collected as a function of sample concentration with the attenuator being kept at 8. By fitting two slopes over the data, the critical micelle concentration (CMC) for each conjugate may be obtained. As seen in FIGS. 75 and 76, P1 starts forming aggregates at around 10 μM while P2 starts forming aggregates at around 5 μM.

Results and Discussion of Protein Delivery

The small protein (long peptide) used here is a 43 amino acid ˜5 kDa antiviral peptide. The peptide sequence is: KADNAAIESIRNGTYDHDVYRDEALNNRFQIKGVELKSGYKDW (SEQ ID NO:1). All of the protein-oligoTEA conjugates were successfully synthesized. Their LC-MS and MALDI-MS spectra are shown herein. The transport of these conjugates was evaluated in the cell using immunofluorescence staining with the anti-protein primary antibody and AlexFluor 568 (AF-568) secondary antibody. The confocal microscopic images of protein-(PEG-Bu)₄ and protein-(Bu-PEG)₄ are shown in FIG. 78. In general, all peptide conjugates appear to be predominantly dispersed in the cytoplasm with some images indicating endosomal localization.

Although the present disclosure has been described with respect to one or more particular embodiments and/or examples, it will be understood that other embodiments and/or examples of the present disclosure may be made without departing from the scope of the present disclosure.

1. A compound having the structure: 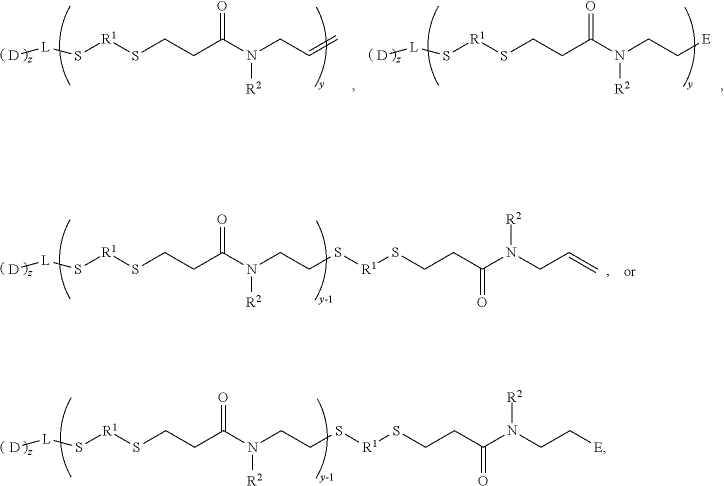
wherein
L is chosen from a linking group, NH, N, O, and S;
D is a cargo group;
R¹ is independently at each occurrence in the compound chosen from straight chain or branched C₂ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)ₐ—, wherein a is 1 to 10, b is 0 to 8, and d is 0 to 8; diol groups having the structure —CH₂—CHOH—(CH₂)ₑ—CHOH—CH₂—, wherein e is 0 to 10 (e.g., 1, 2, 3, 4, 5, 6, 7, 8, 9, 10); substituted or unsubstituted C₅ to C₁₀ aryl groups; and substituted or unsubstituted C₃ to C₈ aliphatic cyclic groups;
R² is independently at each occurrence in the compound chosen from cationic groups, aliphatic electrophilic groups, aliphatic nucleophilic groups, straight chain or branched C₁ to C₂₀ alkyl groups; straight chain or branched C₂ to C₂₀ alkenyl groups; straight chain or branched C₂ to C₂₀ alkynyl groups; polyether groups having the structure —(CH₂)_{b}—[—O—CH₂—CH₂—]ₐ—O—(CH₂)ₐ—, where a is 1 to 10, b is 0 to 8, and d is 0 to 8; diol groups having the structure —CH₂—CHOH—(CH₂)ₑ—CHOH—CH₂—; where e is 0 to 10; substituted or unsubstituted C₅ to C₁₀ aryl groups; and substituted or unsubstituted C₃ to C₈ aliphatic cyclic groups;
E is an end group chosen from ═CH₂, D, and L-(D)_{z};
y is 1 to 12; and
z is 1 to 5.

2. The compound of claim 1, wherein the compound has the following structure: 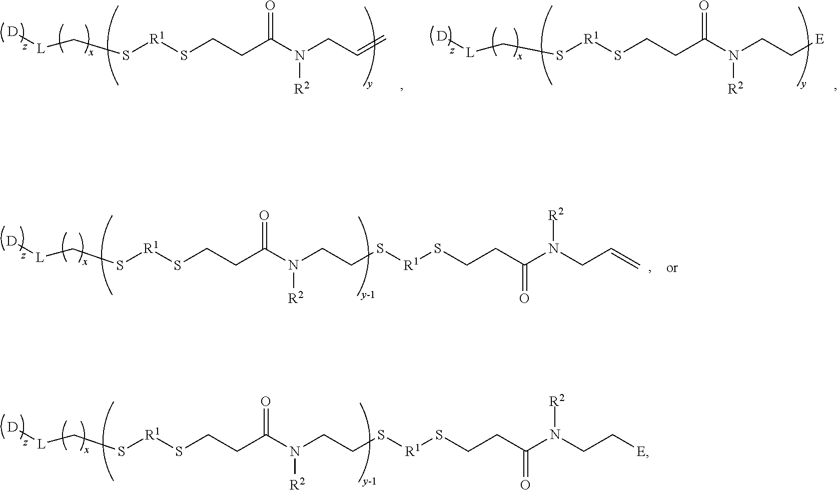
wherein x is 1 to 8.

3. The compound of claim 1, wherein the cargo group is chosen from chemotherapeutic groups, antibiotic groups, fluorophore groups, peptide groups, protein groups, nucleic acid groups, kinase inhibitor groups, antibody groups, enzyme inhibitor groups, small molecule drug groups, sugars/glycan groups, and combinations thereof.

4. The compound of claim 3, wherein the cargo group is chosen from a non-functionalized vancomycin group, a fluorophore-modified vancomycin group, a fluorescein group, an Atto 488 group, and a peptide group having the sequence KADNAAIESIRNGTYDHDVYRDEALNNRFQIKGVELKSGYKDW (SEQ ID NO:1), and combinations thereof.

5. The compound of claim 1, wherein R¹ is independently at each occurrence in the compound chosen from substituted or unsubstituted propyl groups, substituted or unsubstituted butyl groups, 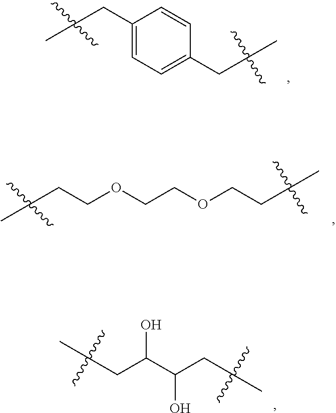 [SMILES: *Cc1ccc(C*)cc1 ; *CCOCCOCC* ; *CC(O)C(O)C*]
and combinations thereof.

6. The compound of claim 1, wherein R² is independently at each occurrence in the compound chosen from substituted or unsubstituted butyl groups, substituted or unsubstituted benzyl groups,

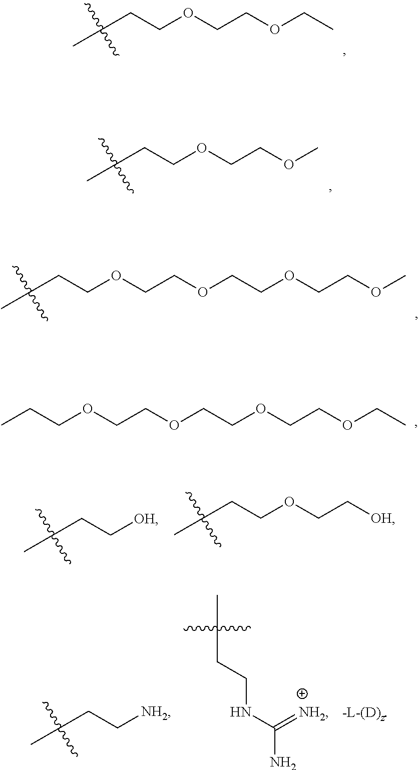

7. The compound of claim 1, wherein the linking group is chosen from 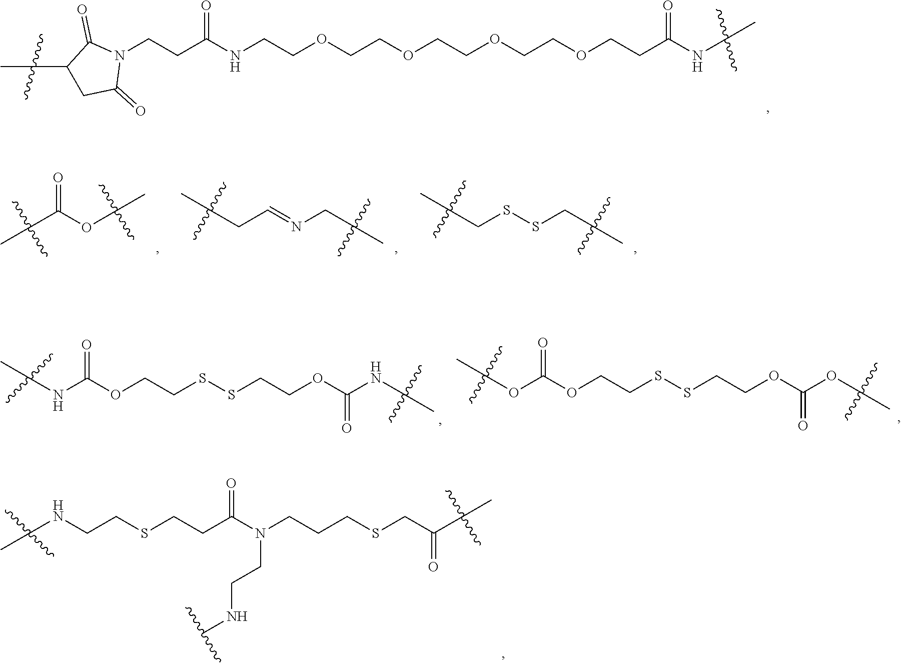
-Val-citrulline-, and combinations thereof.

8. The compound of claim 1, wherein the compound has the following structure: 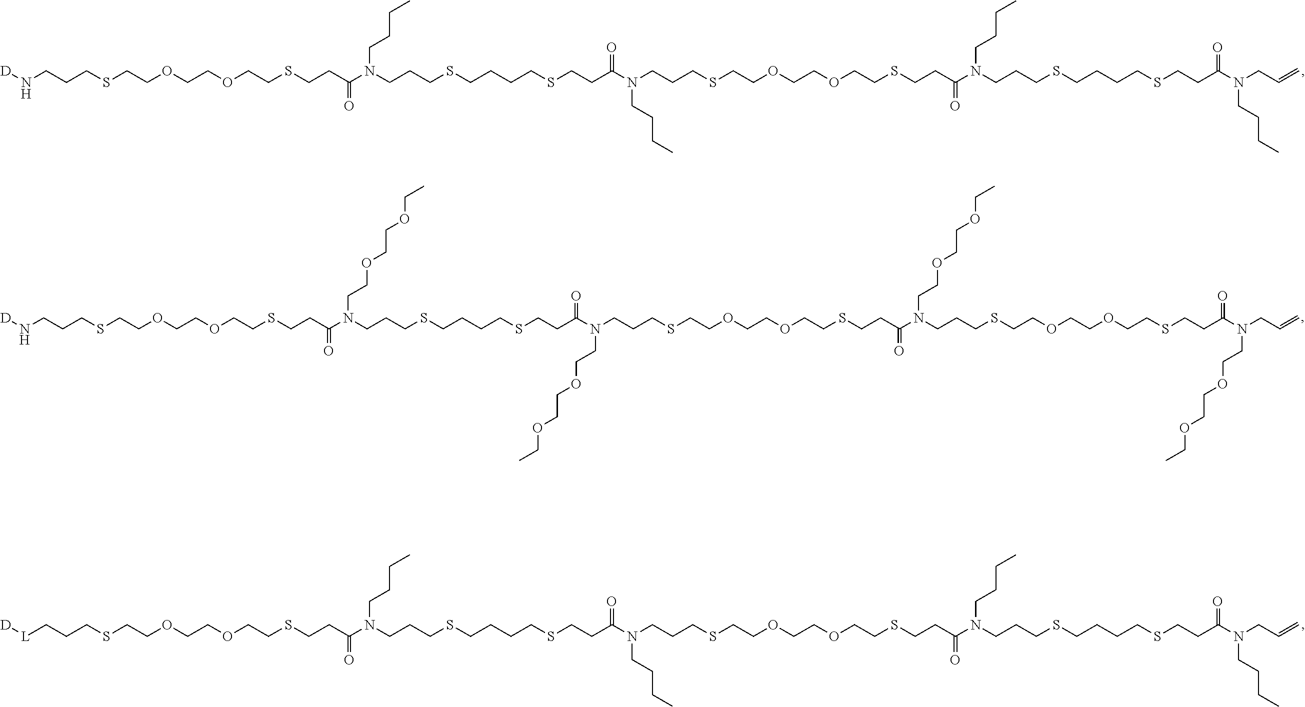 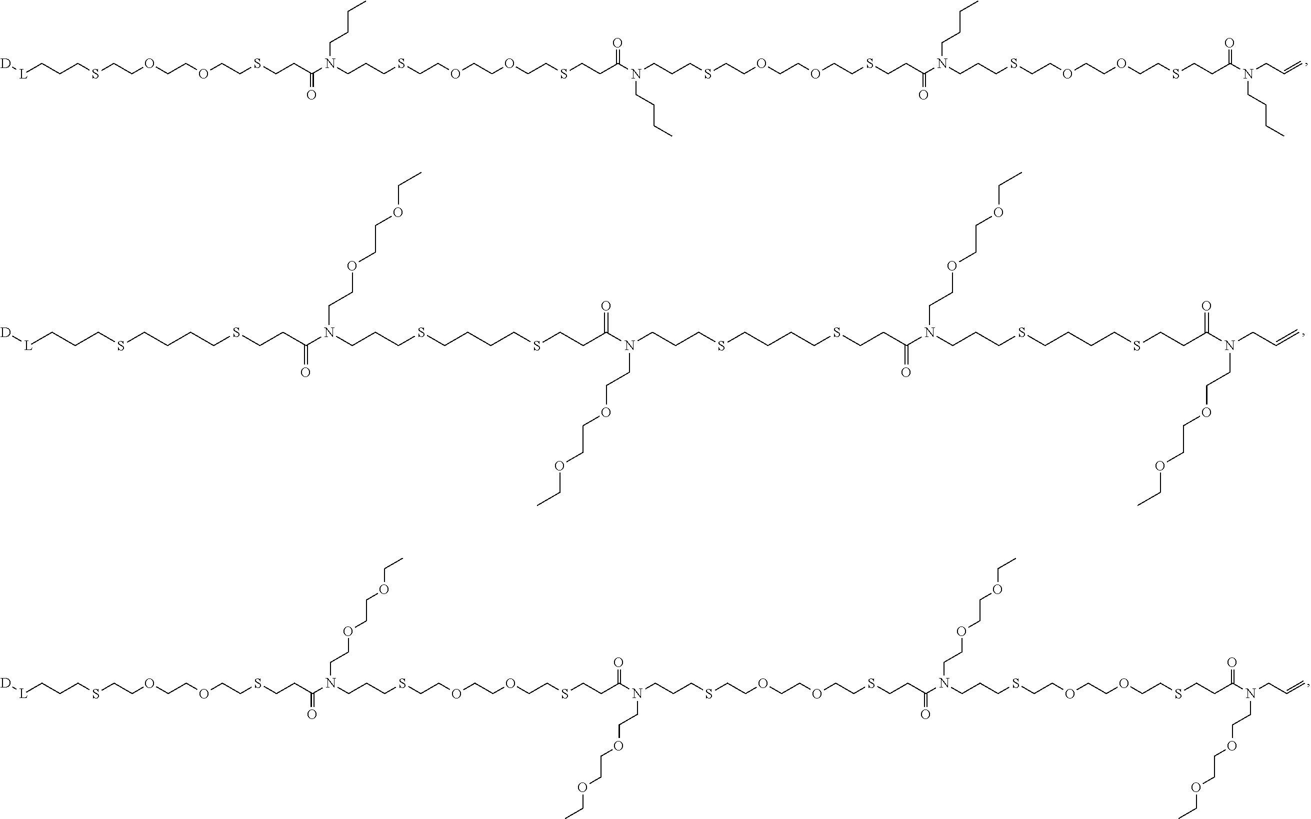 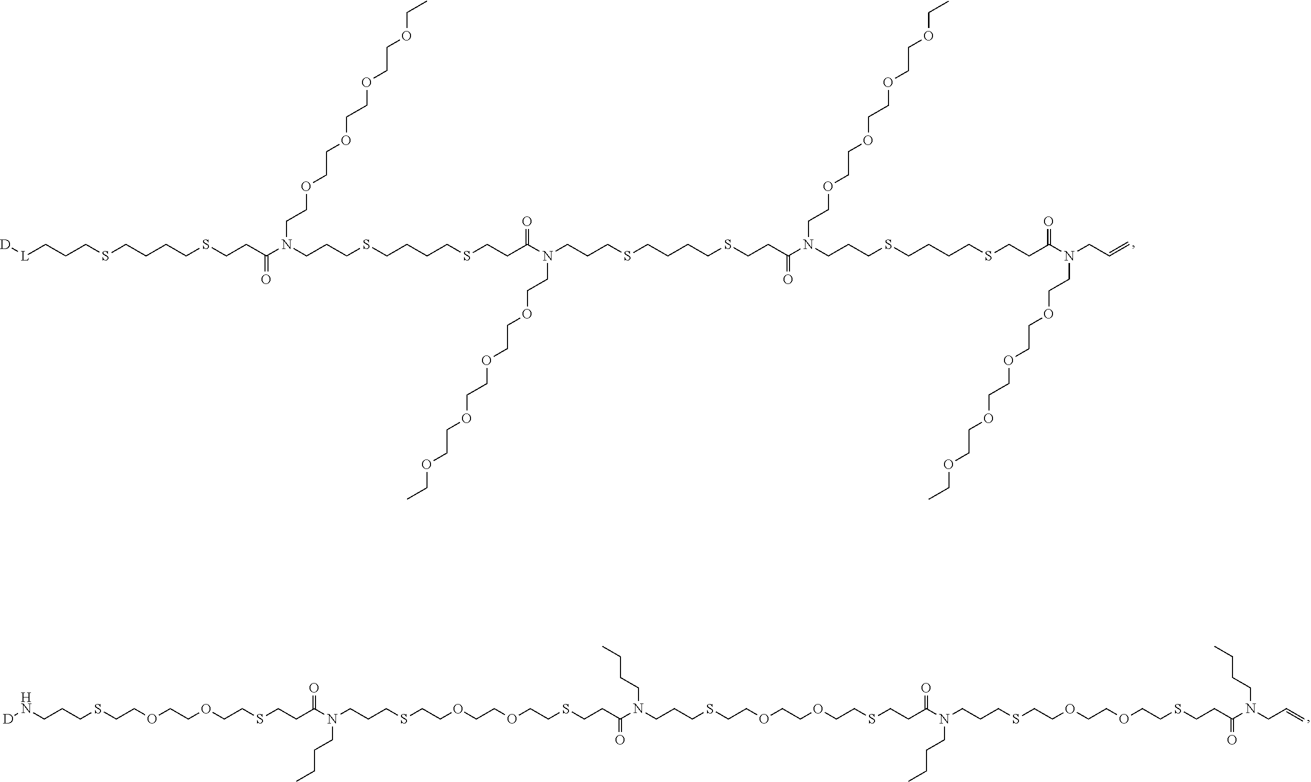 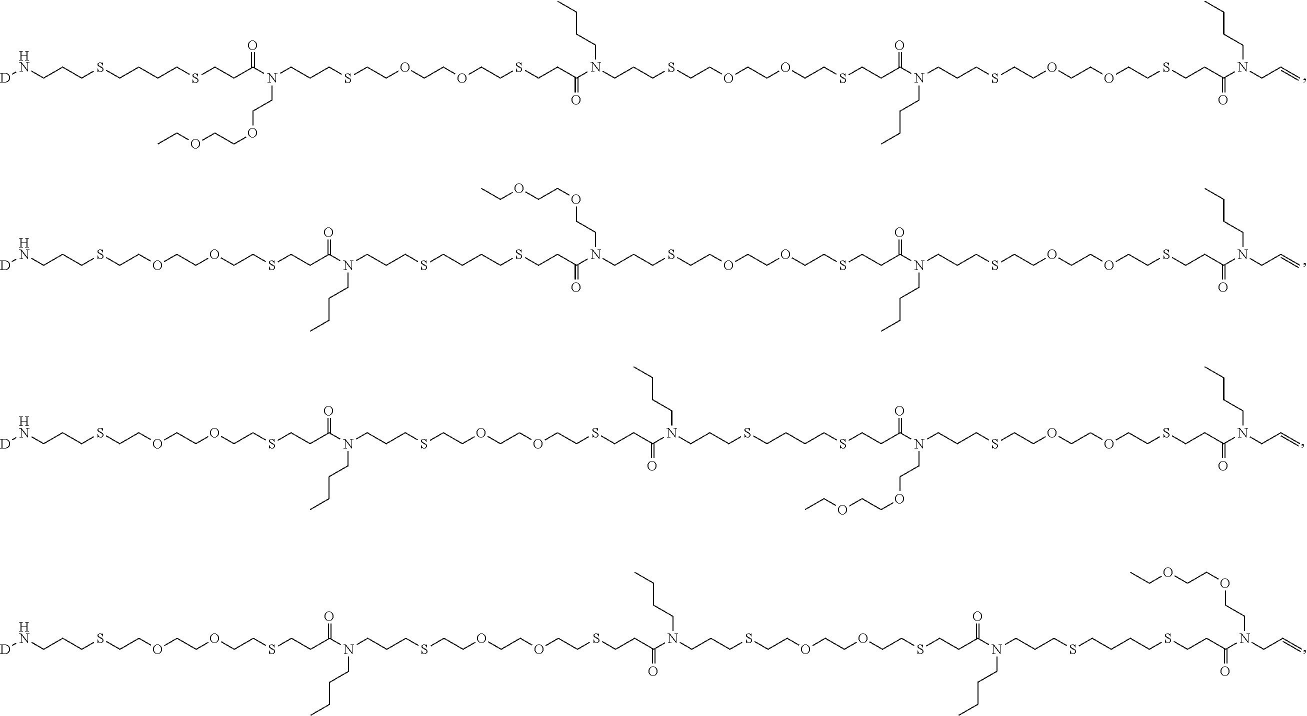 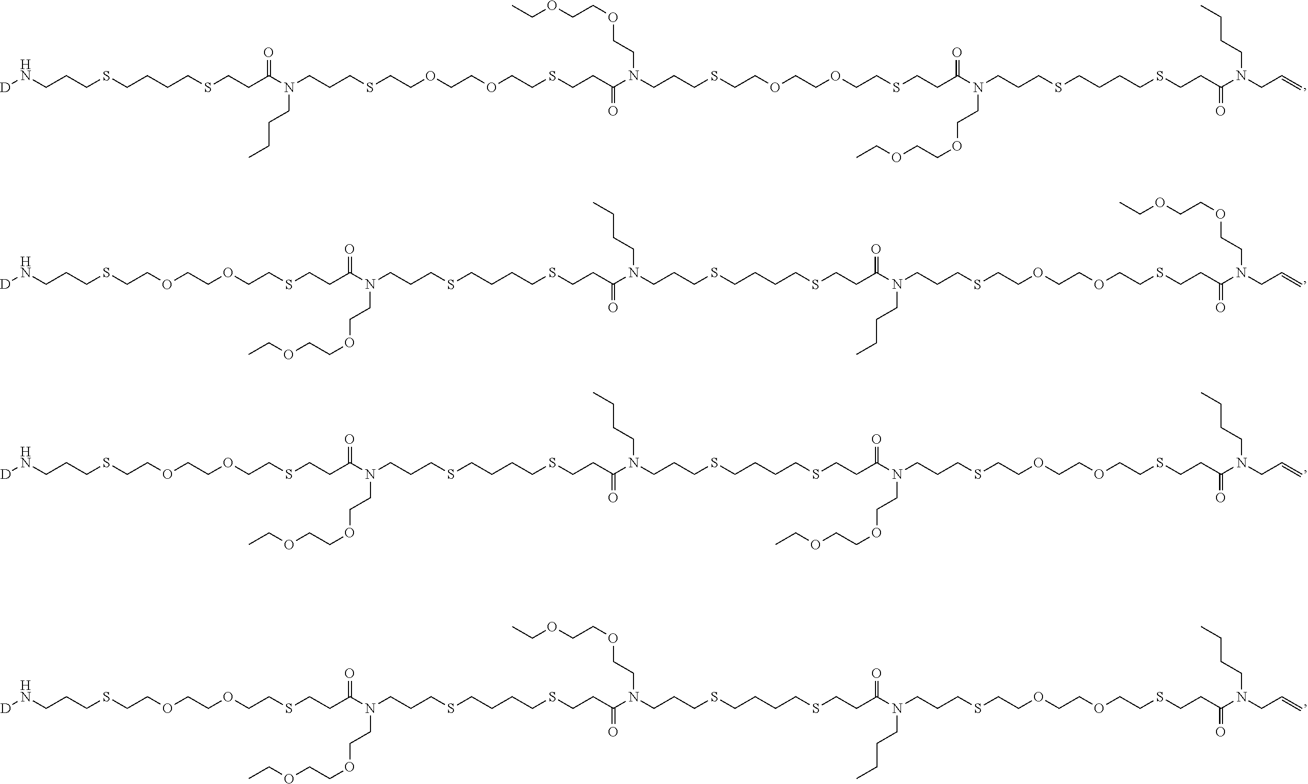 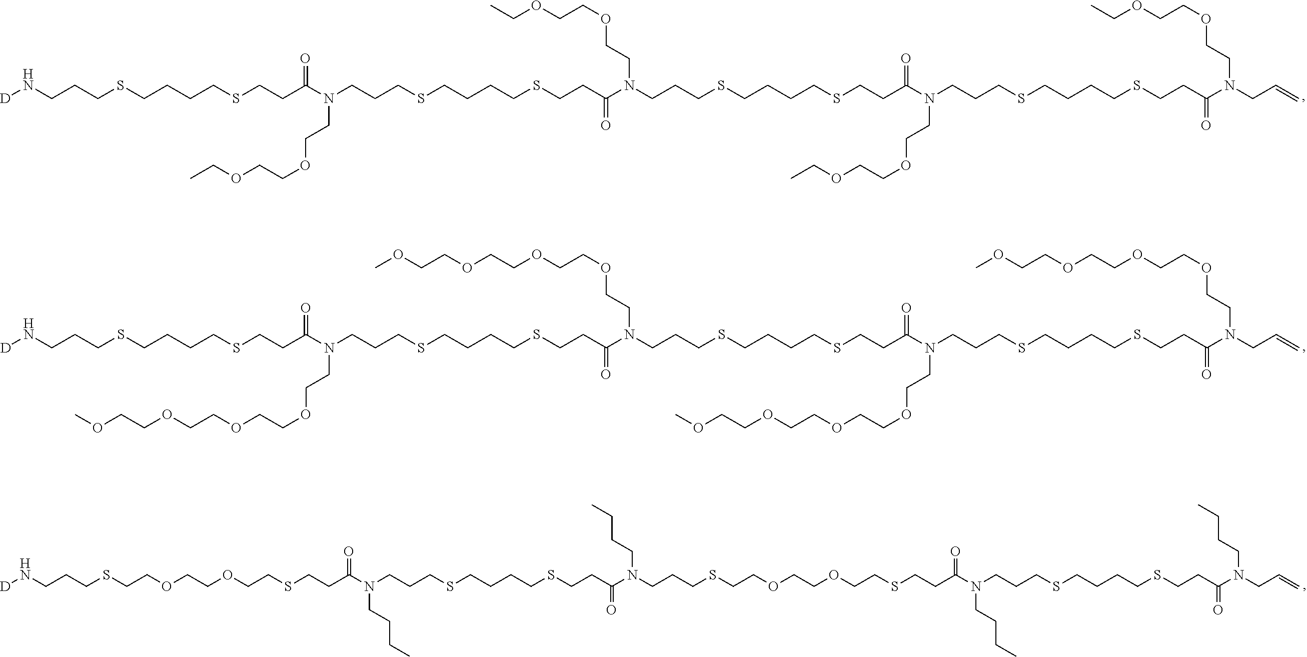 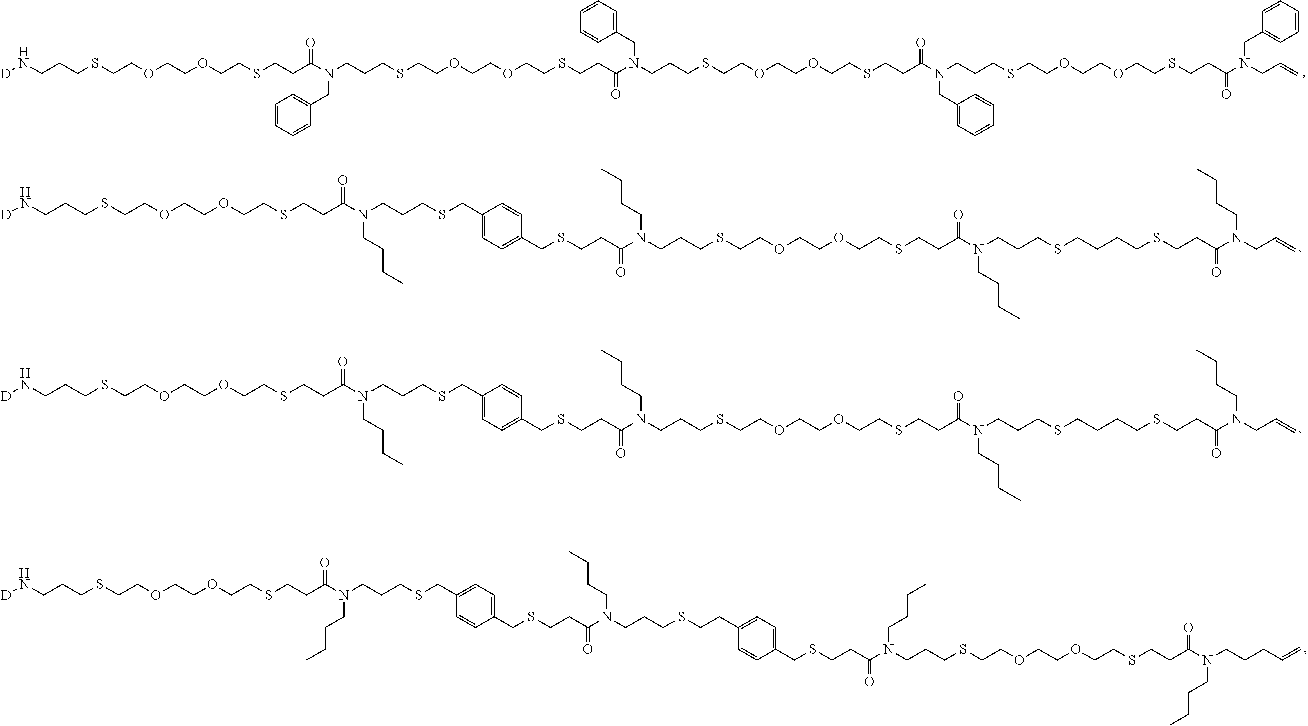 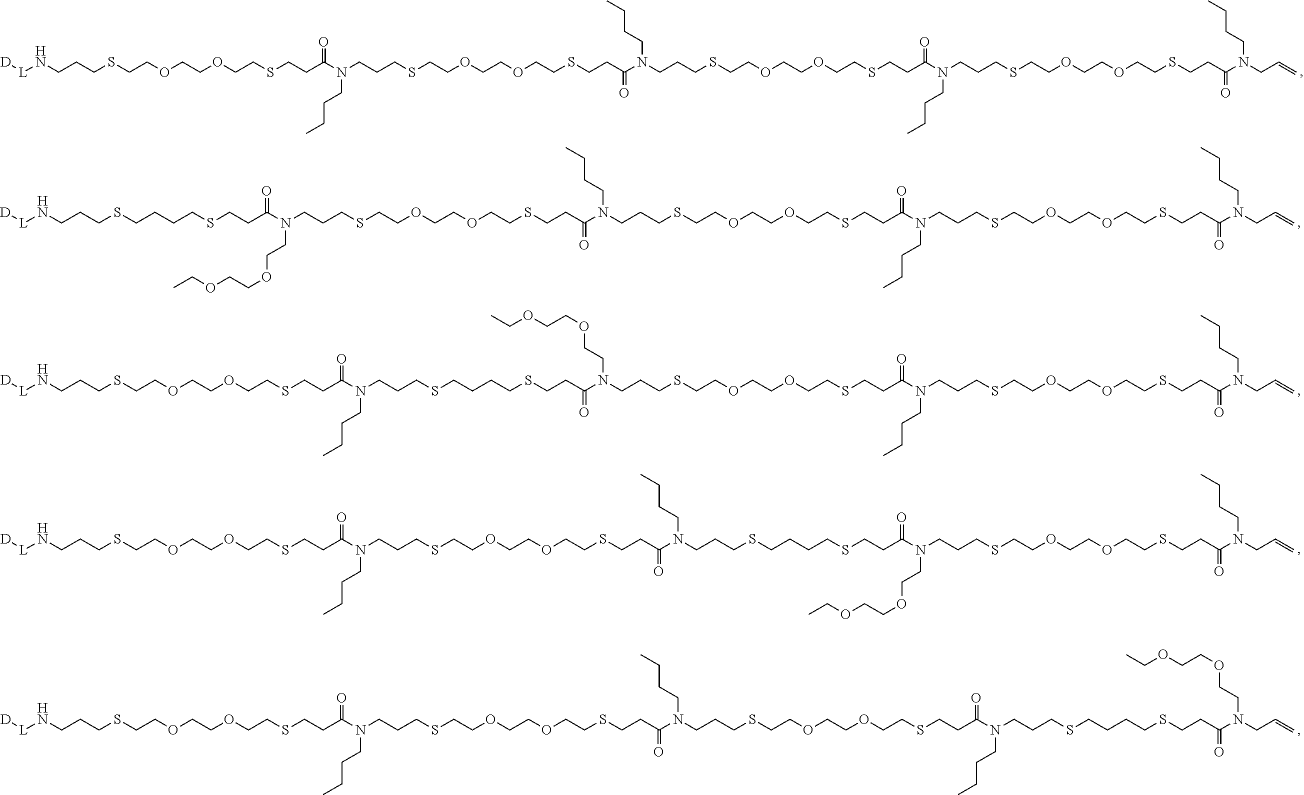 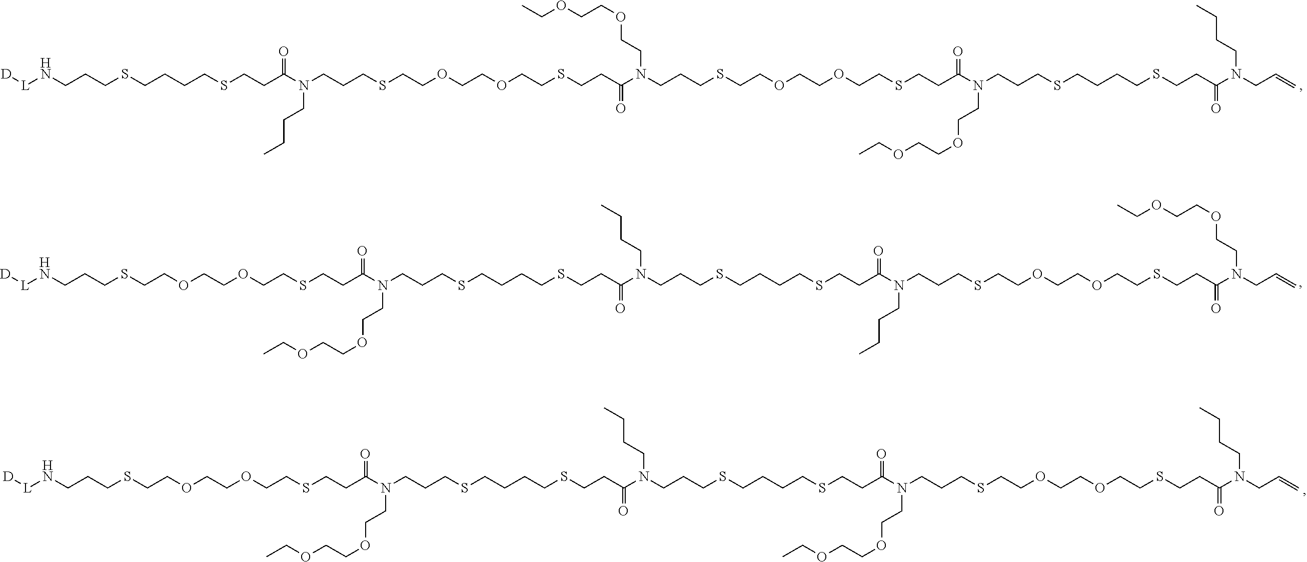 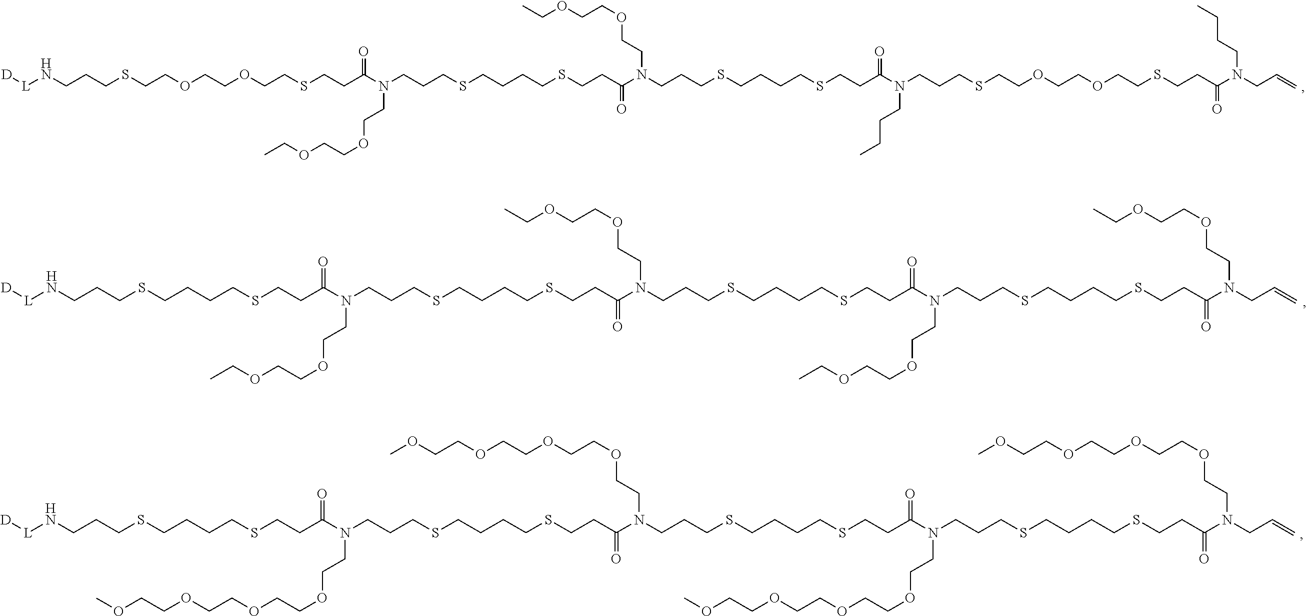 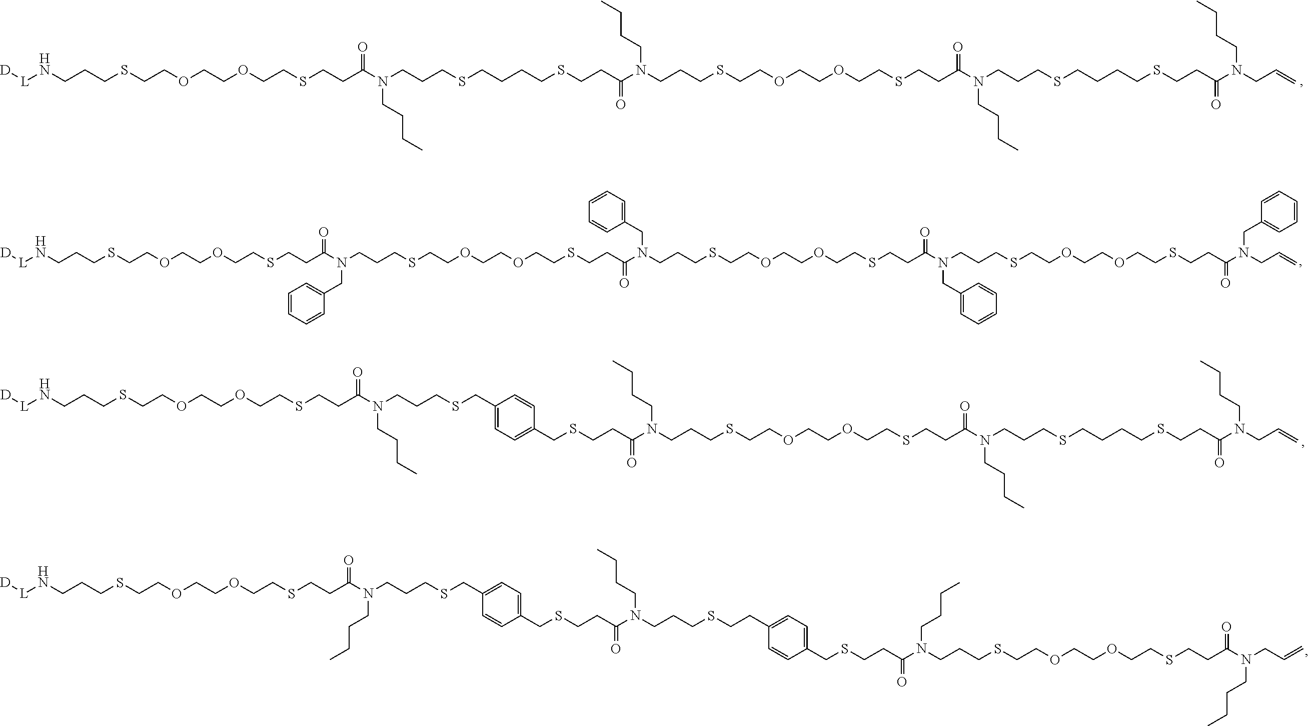 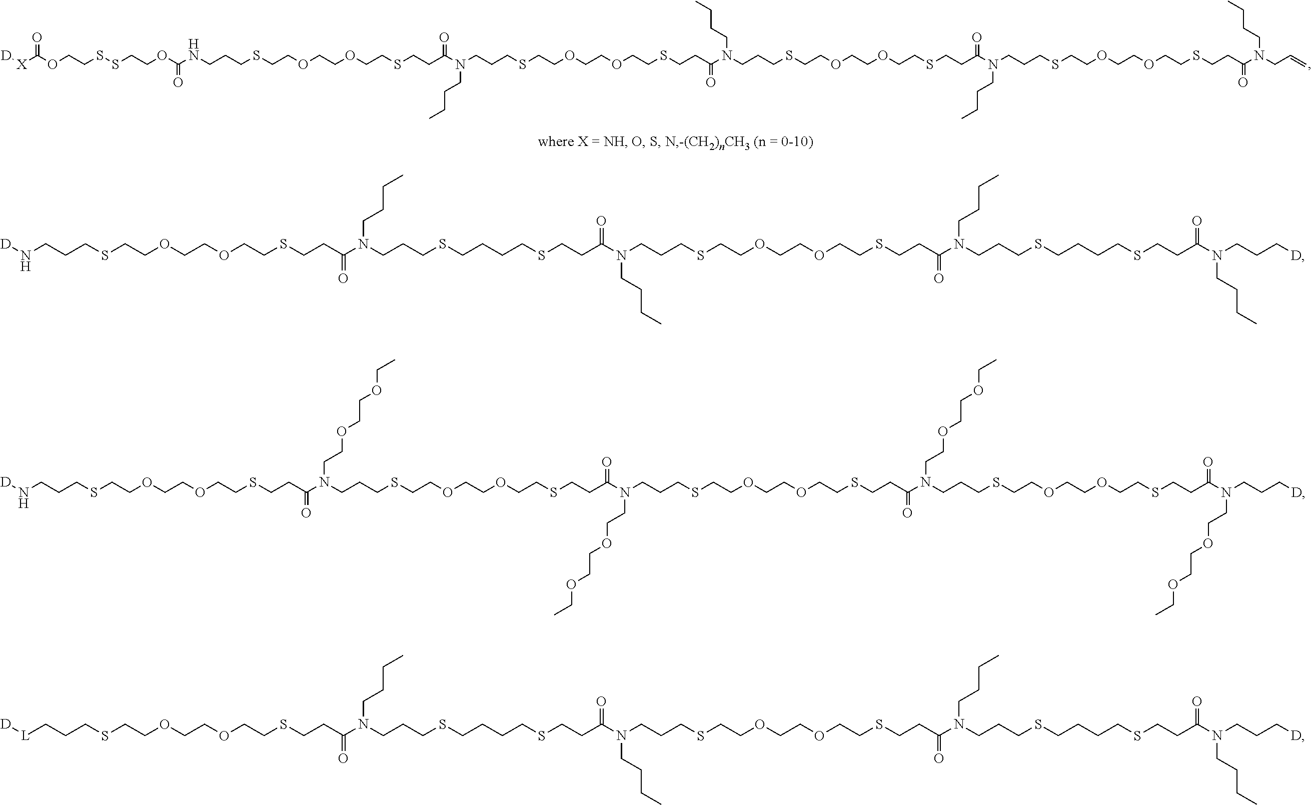 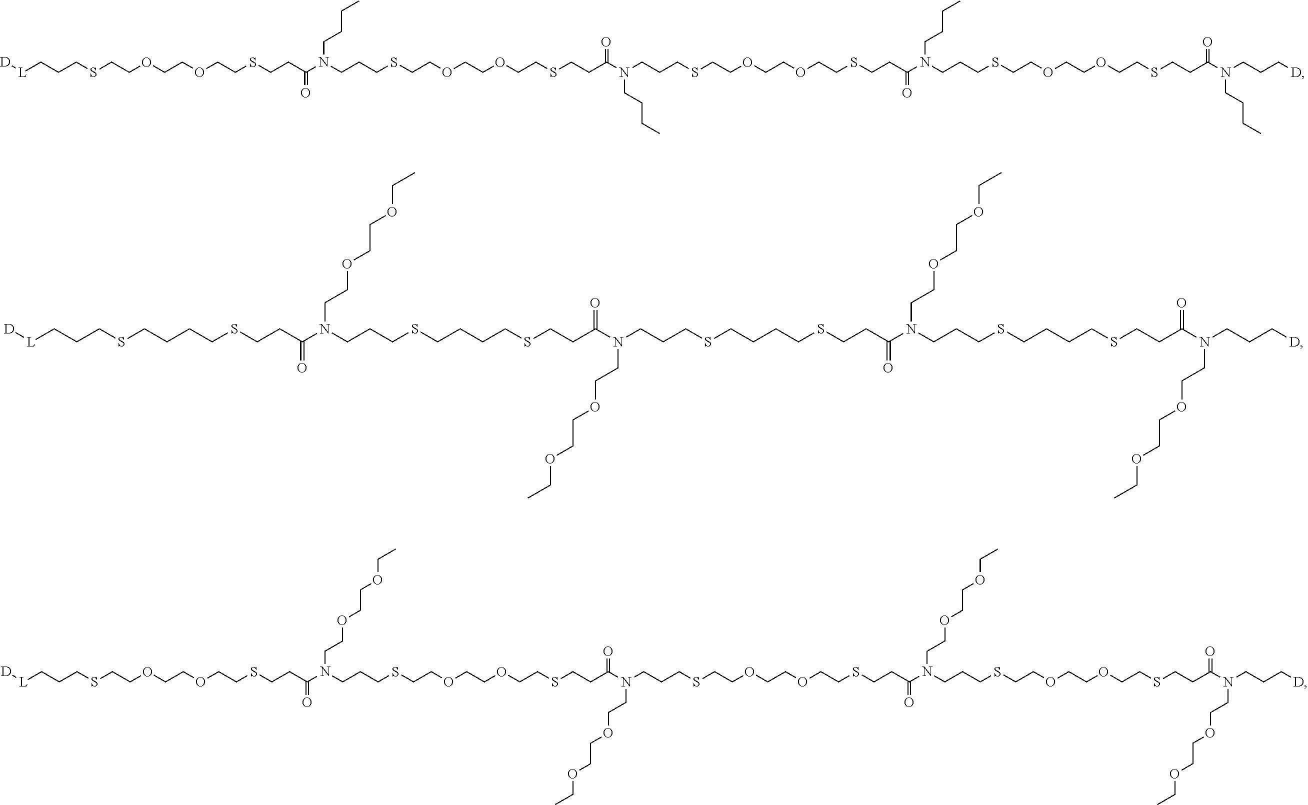 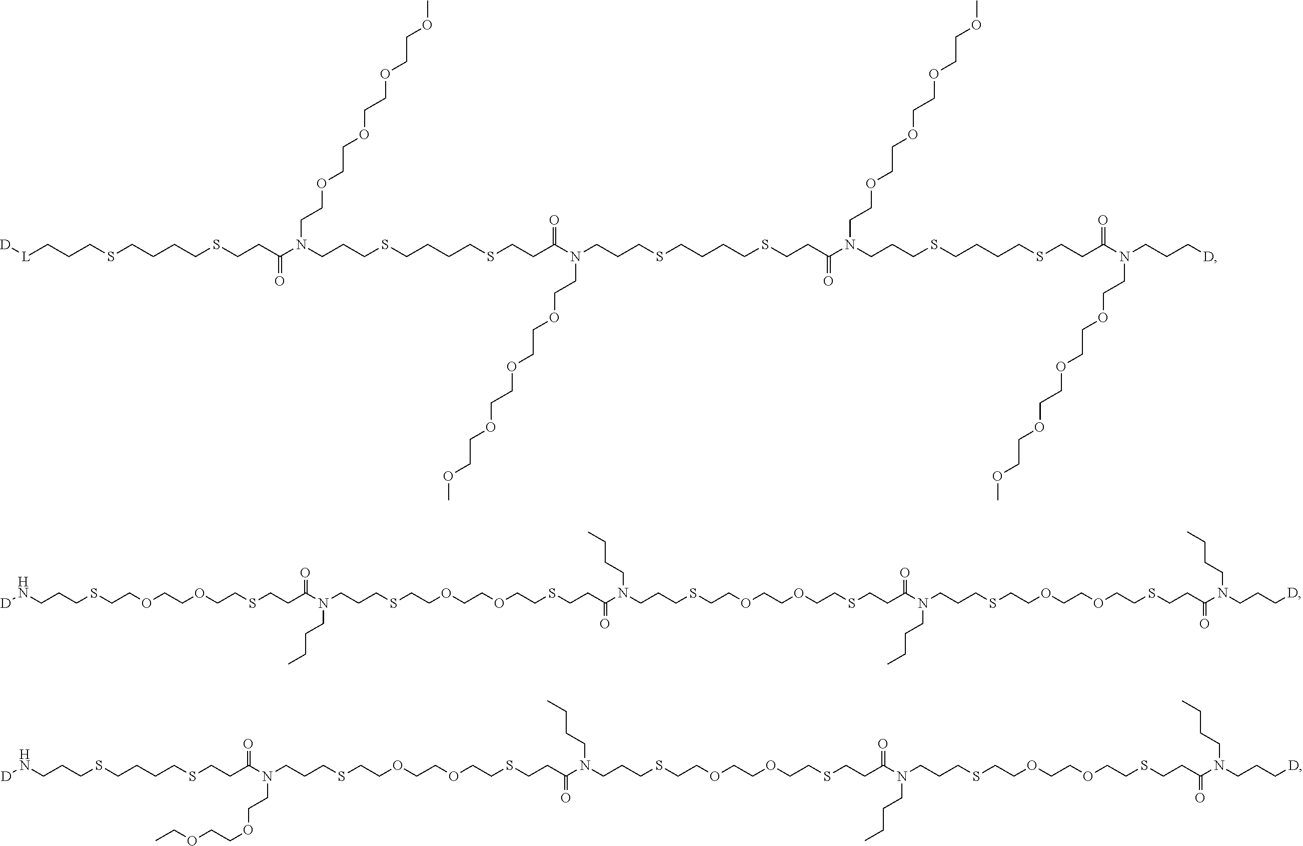 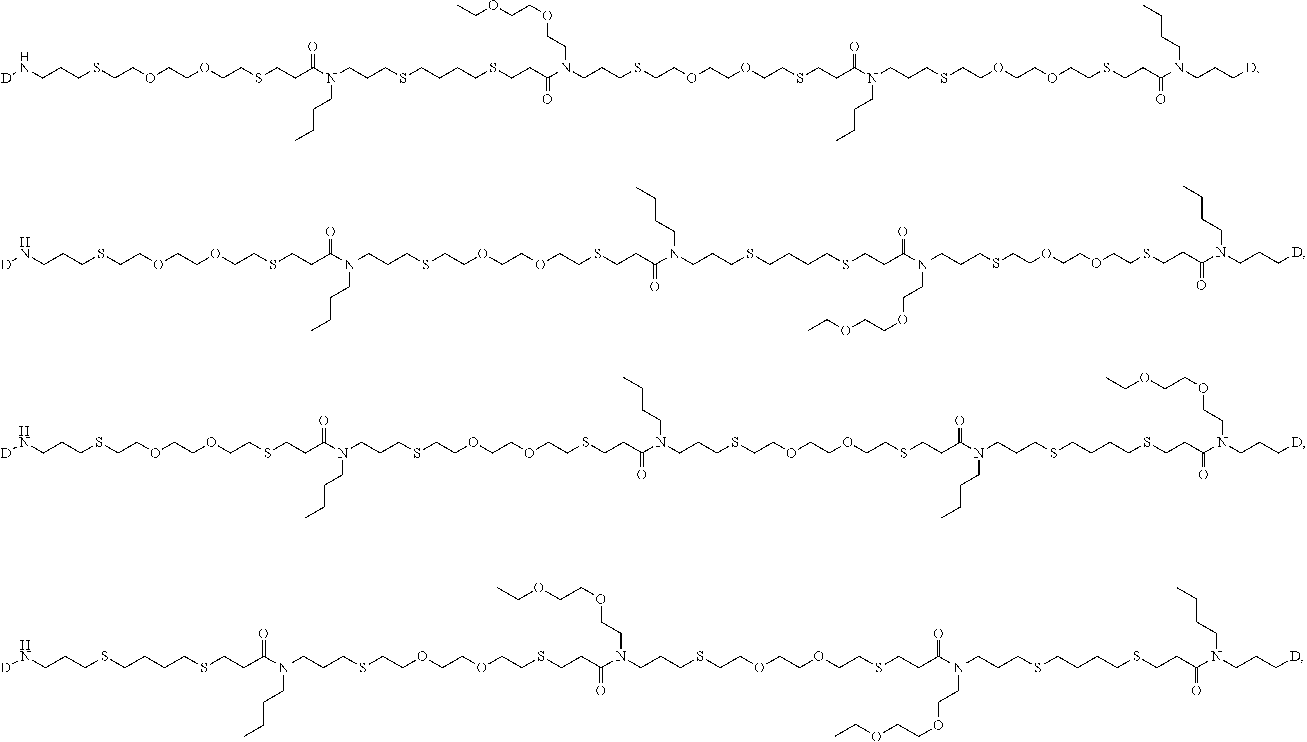 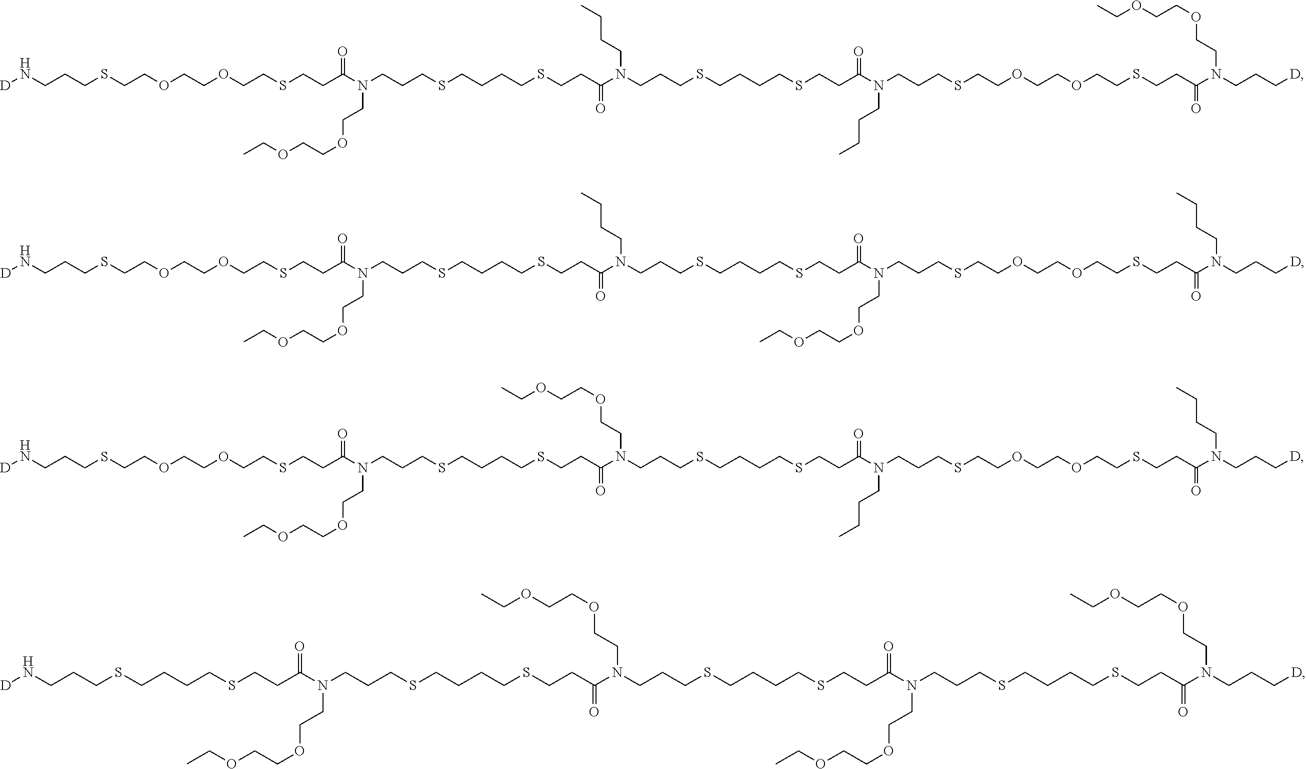 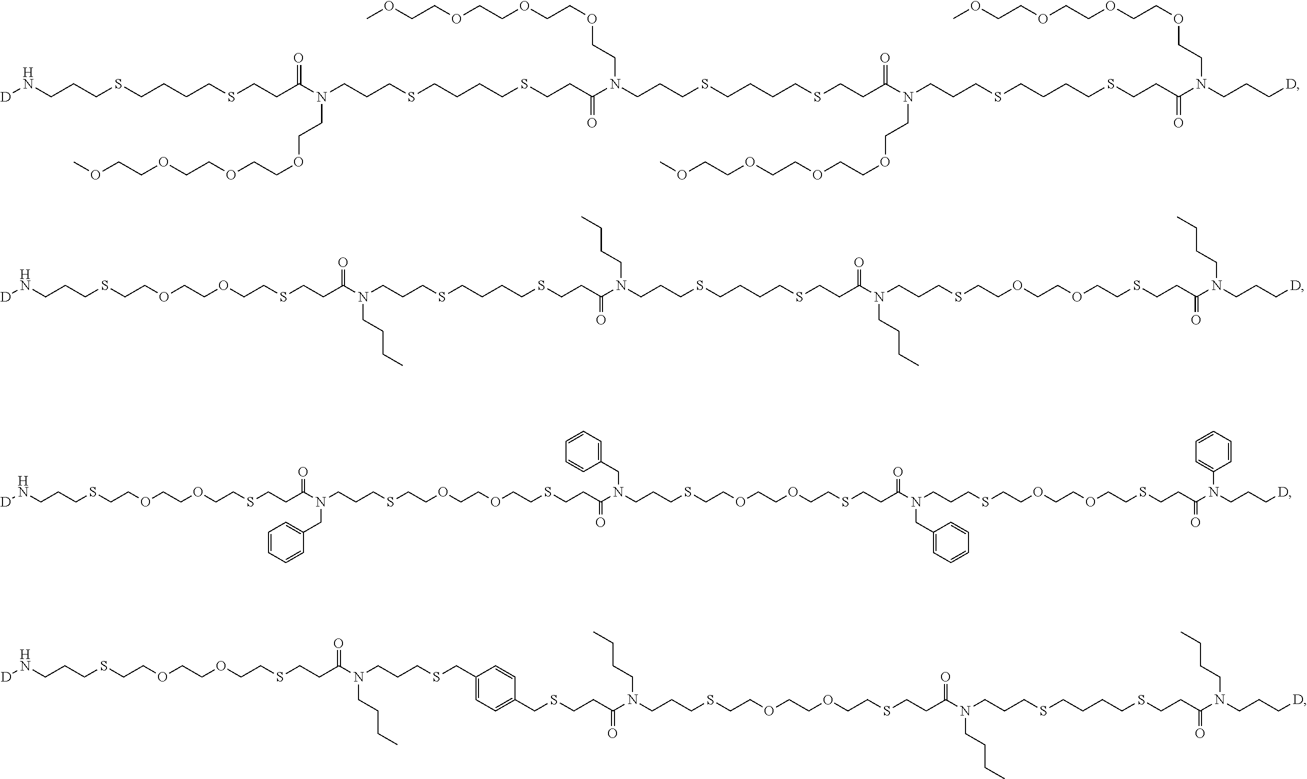 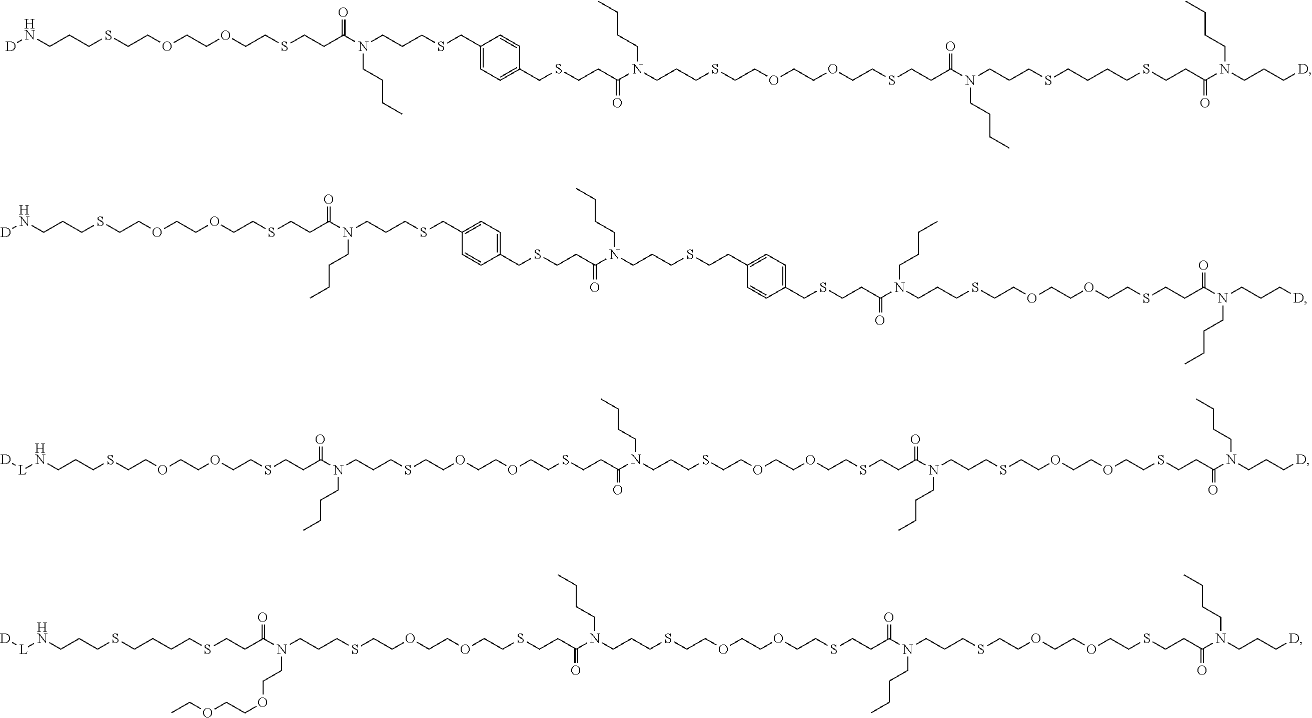 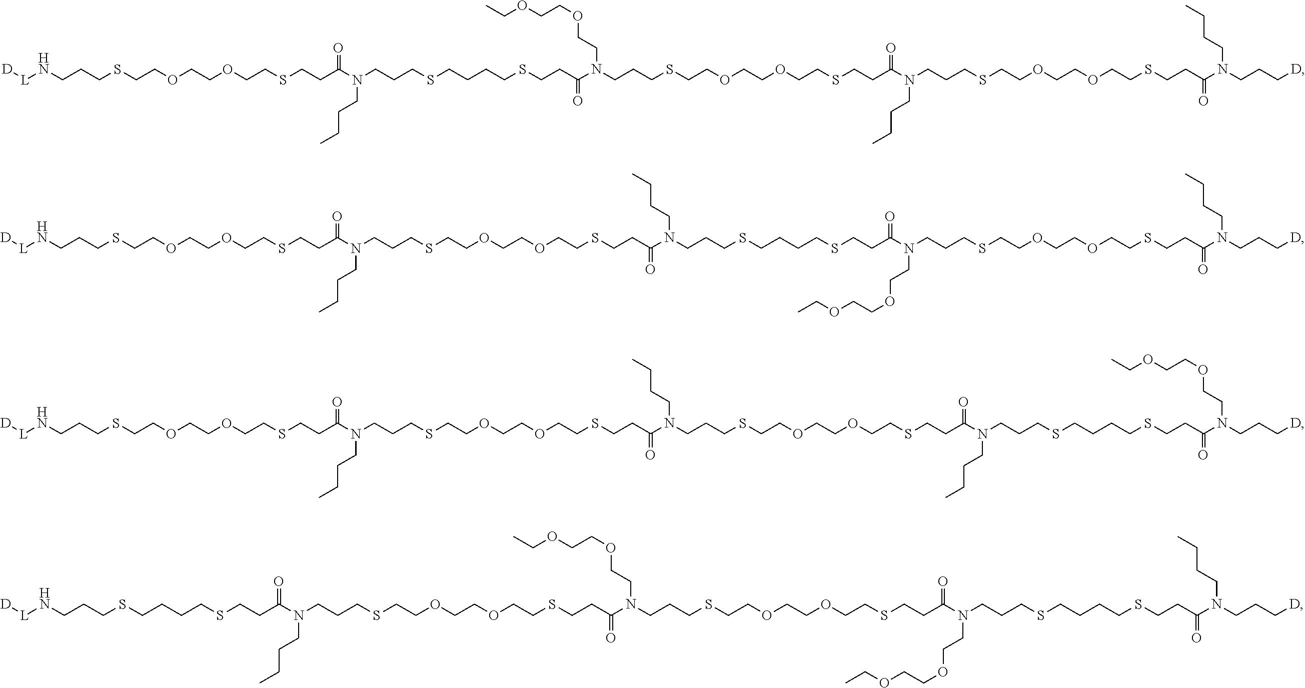 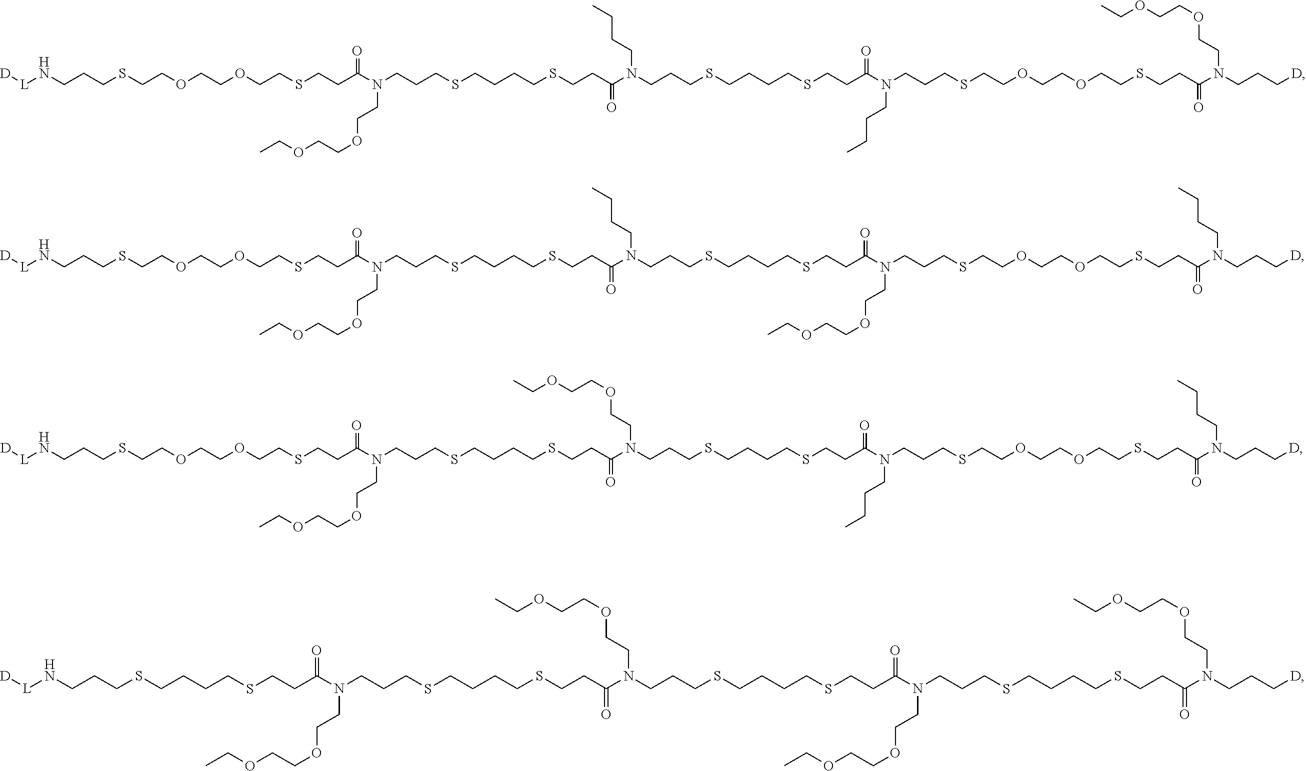 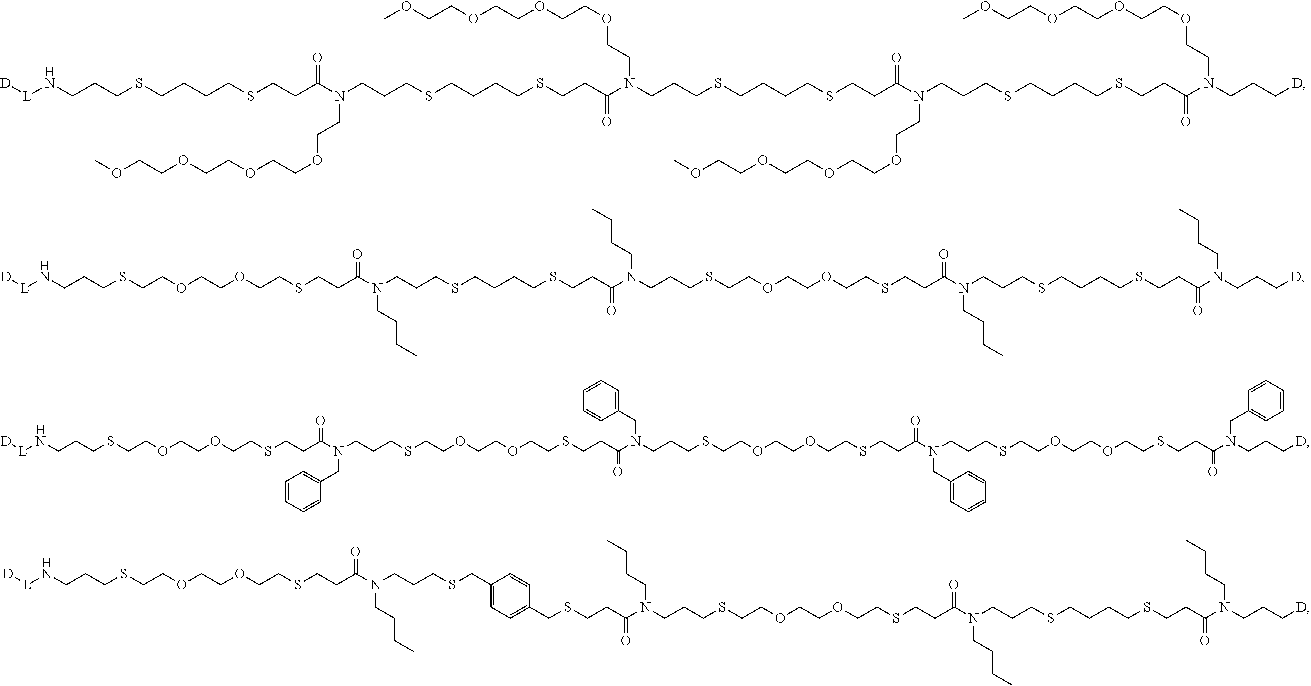 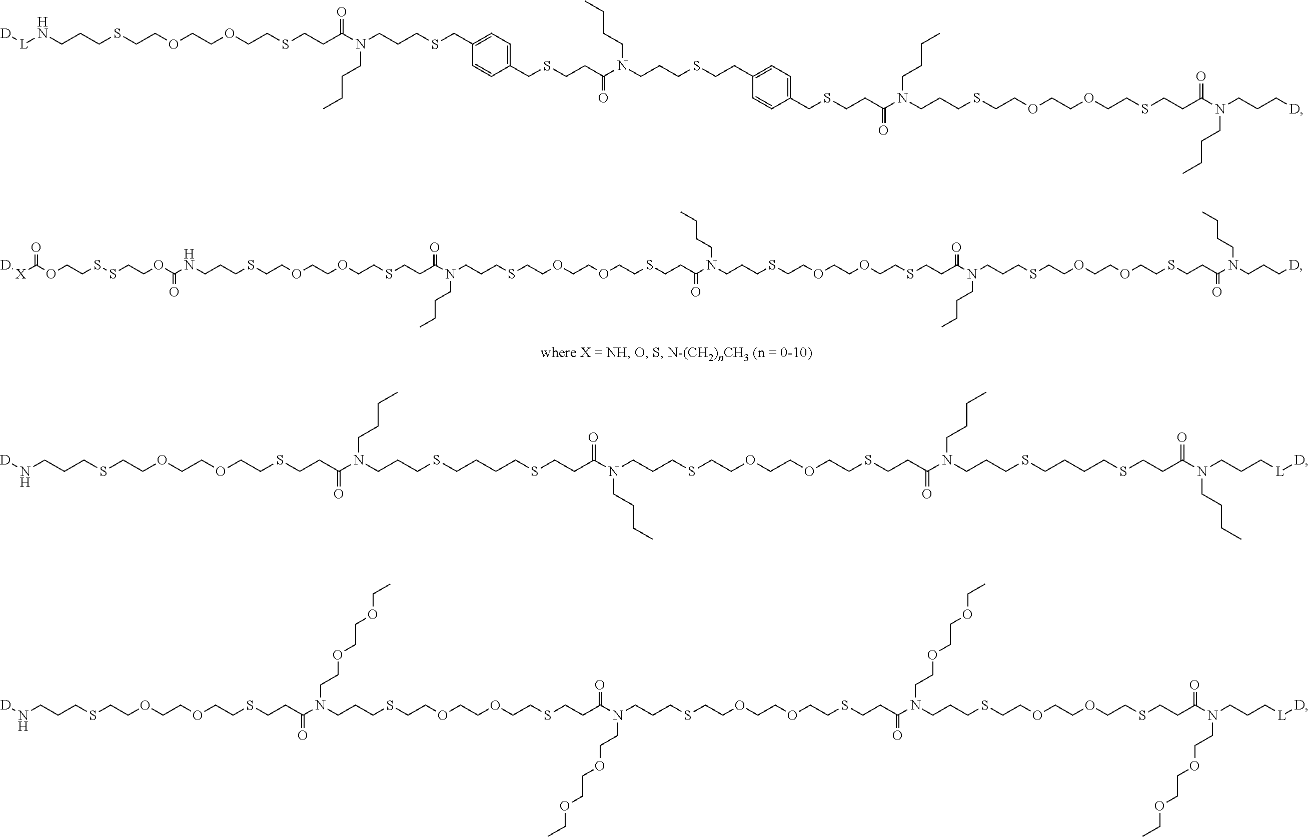 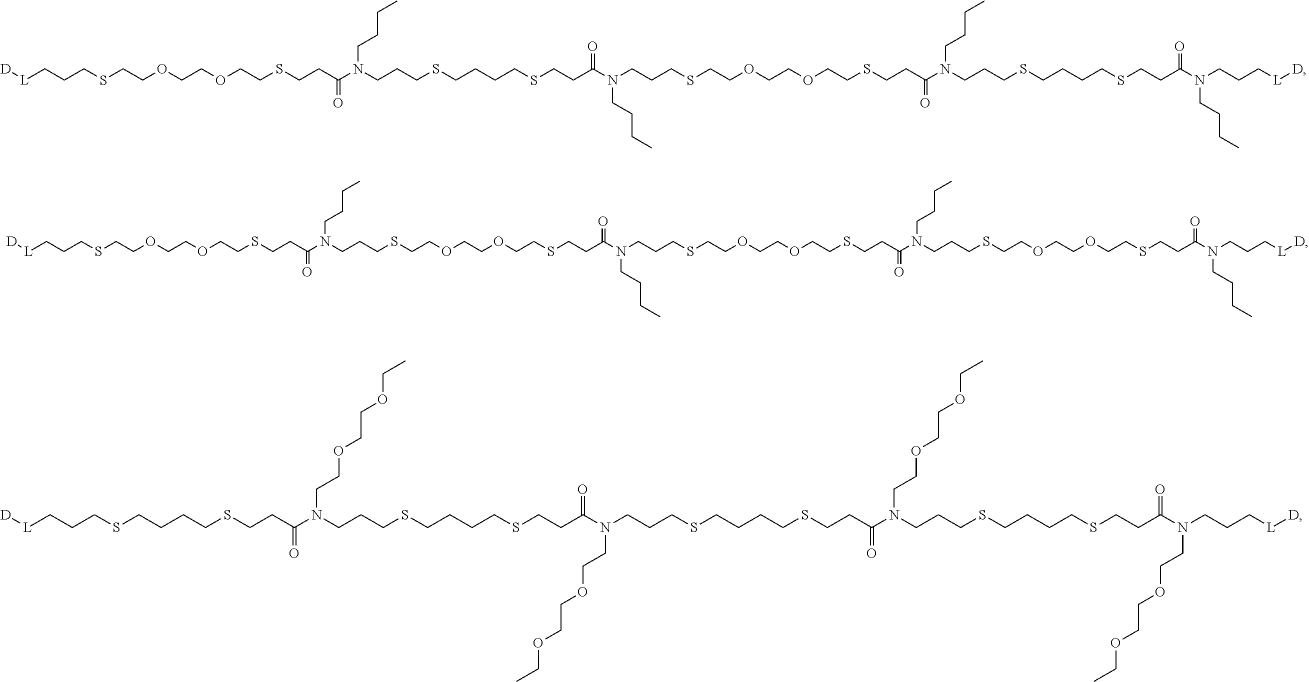 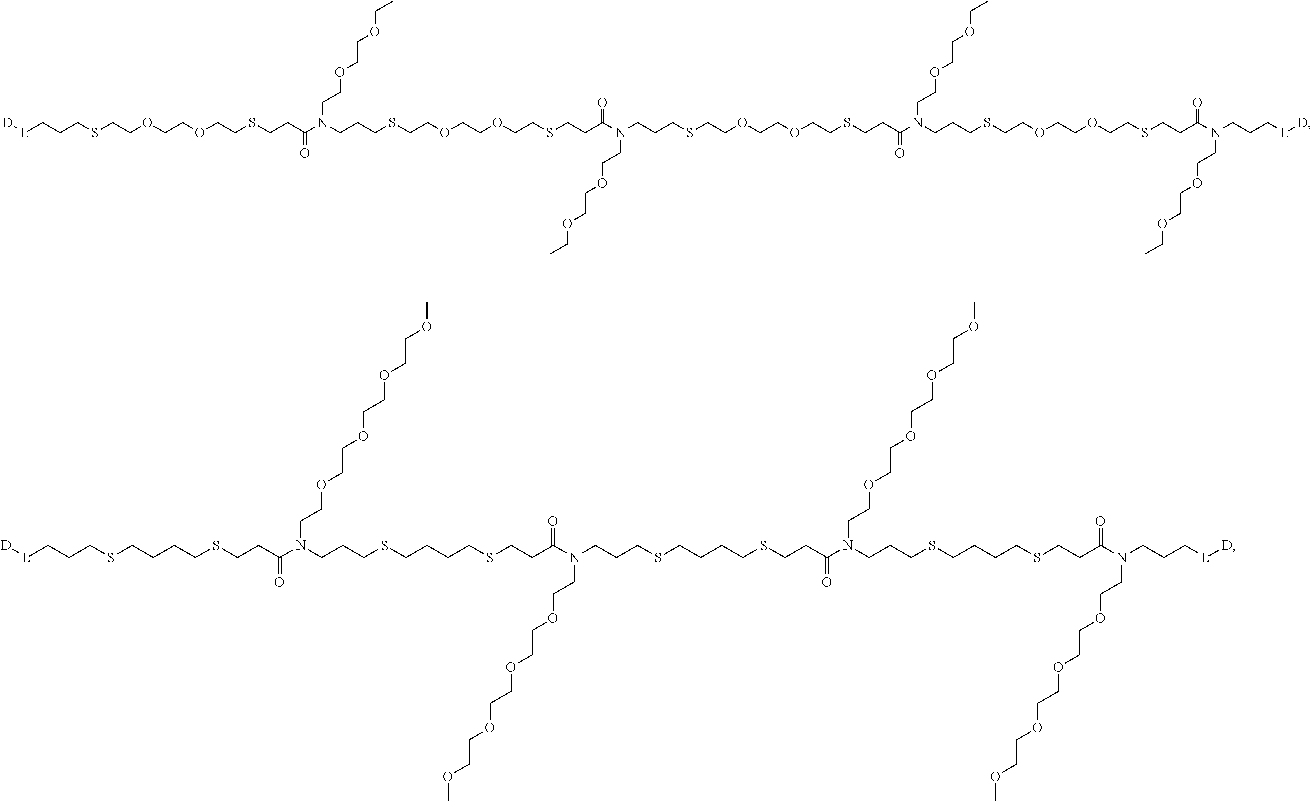 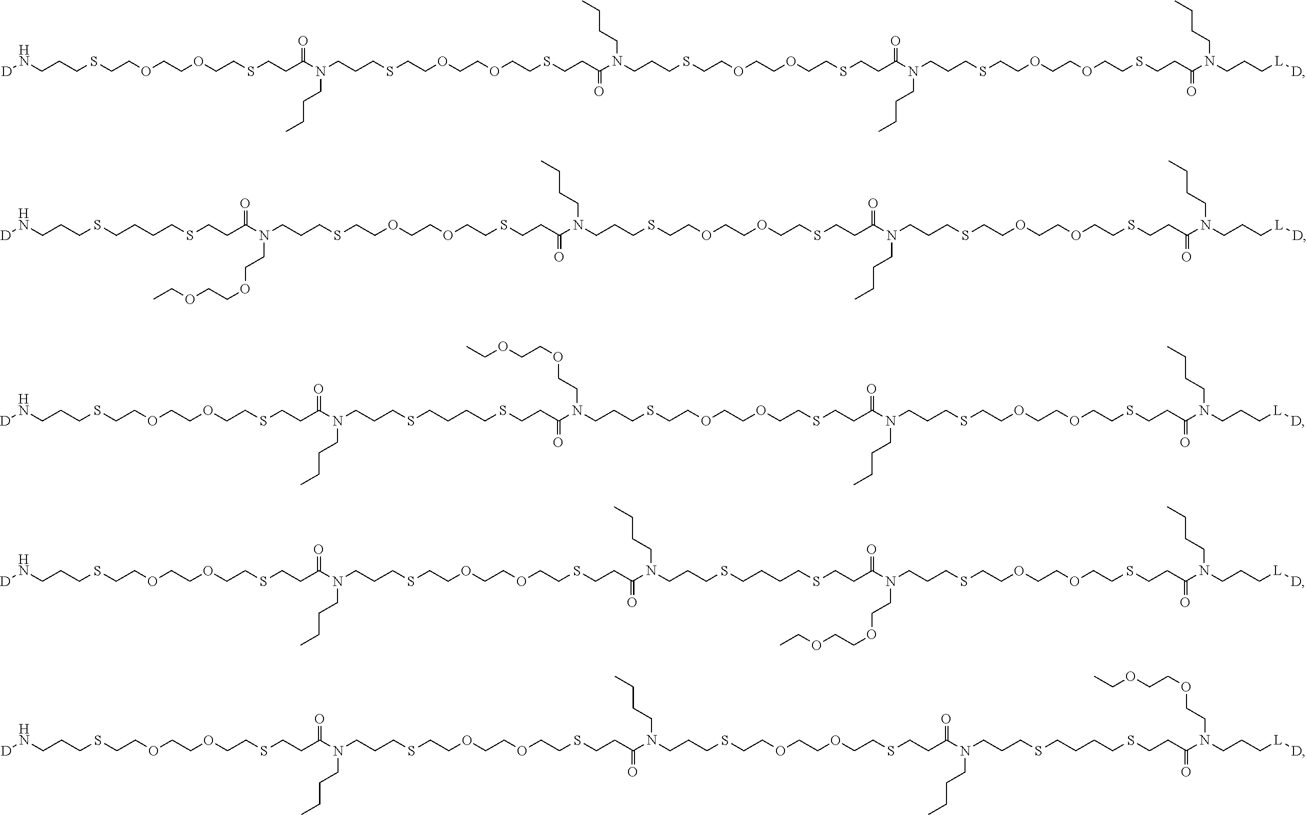 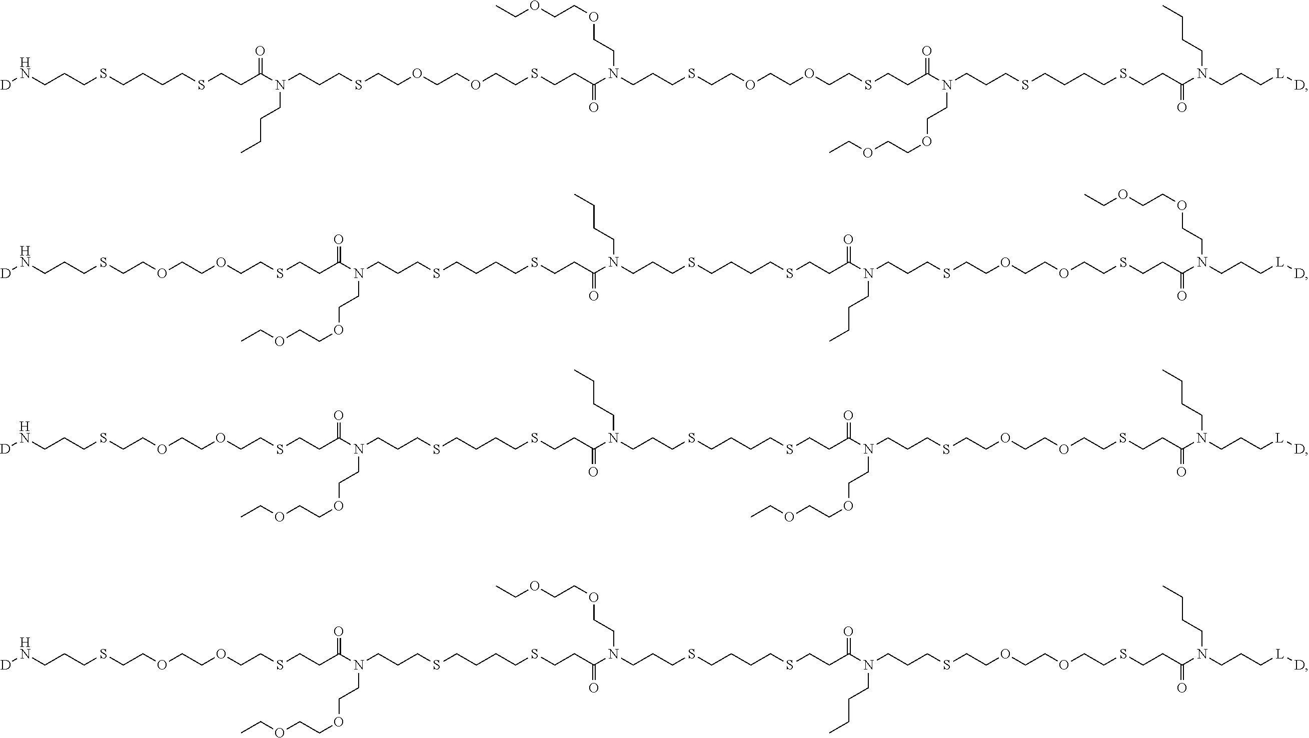 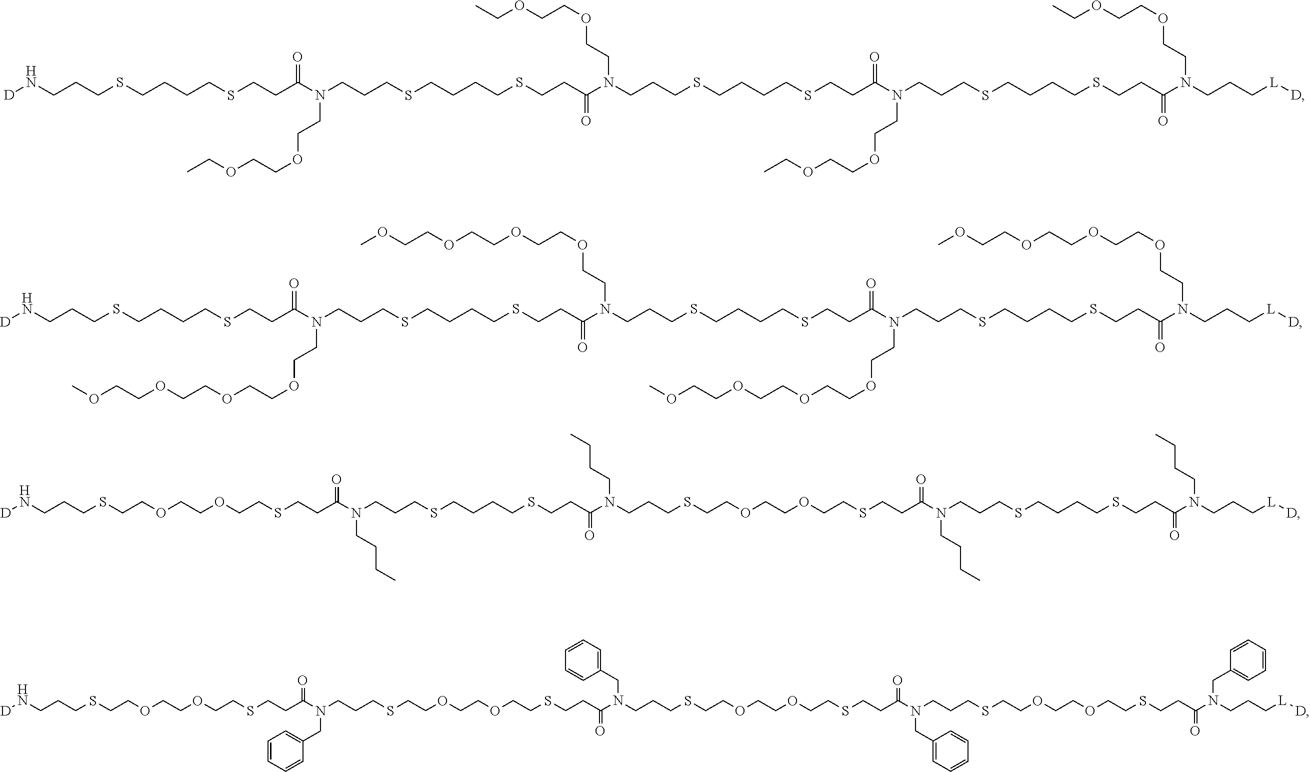 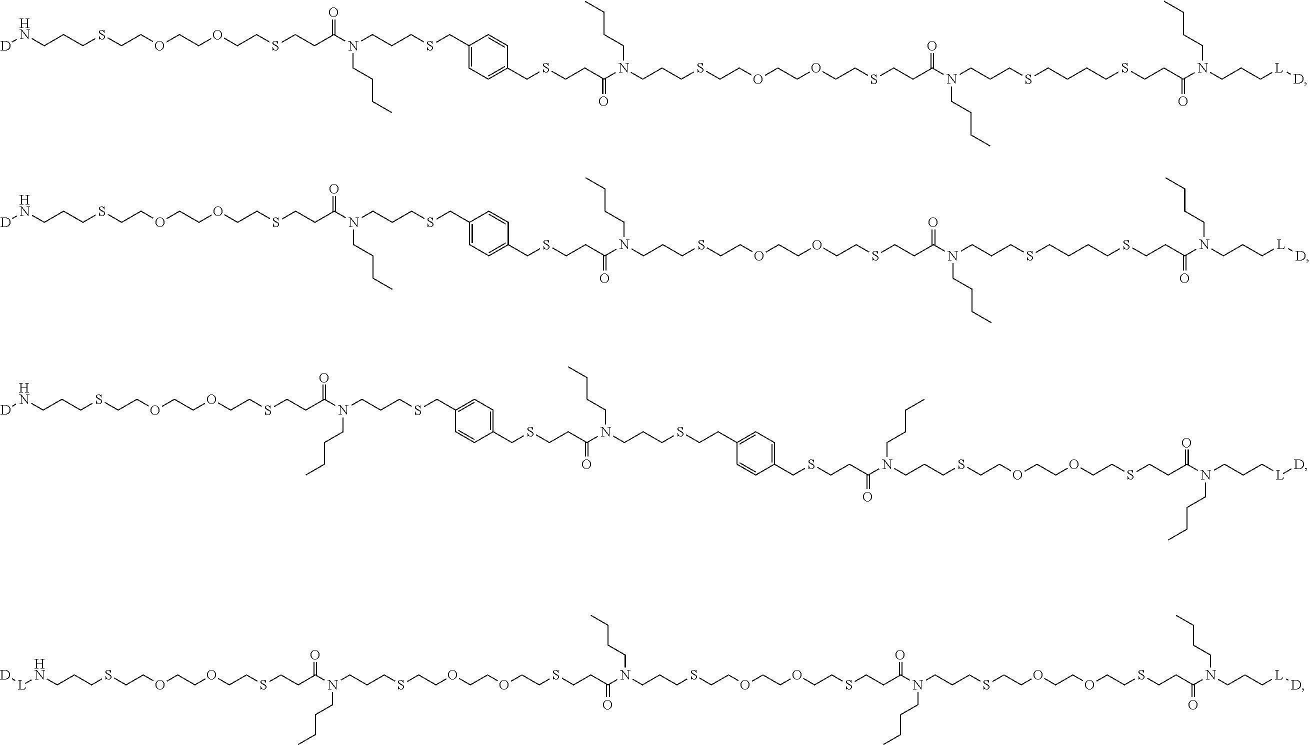 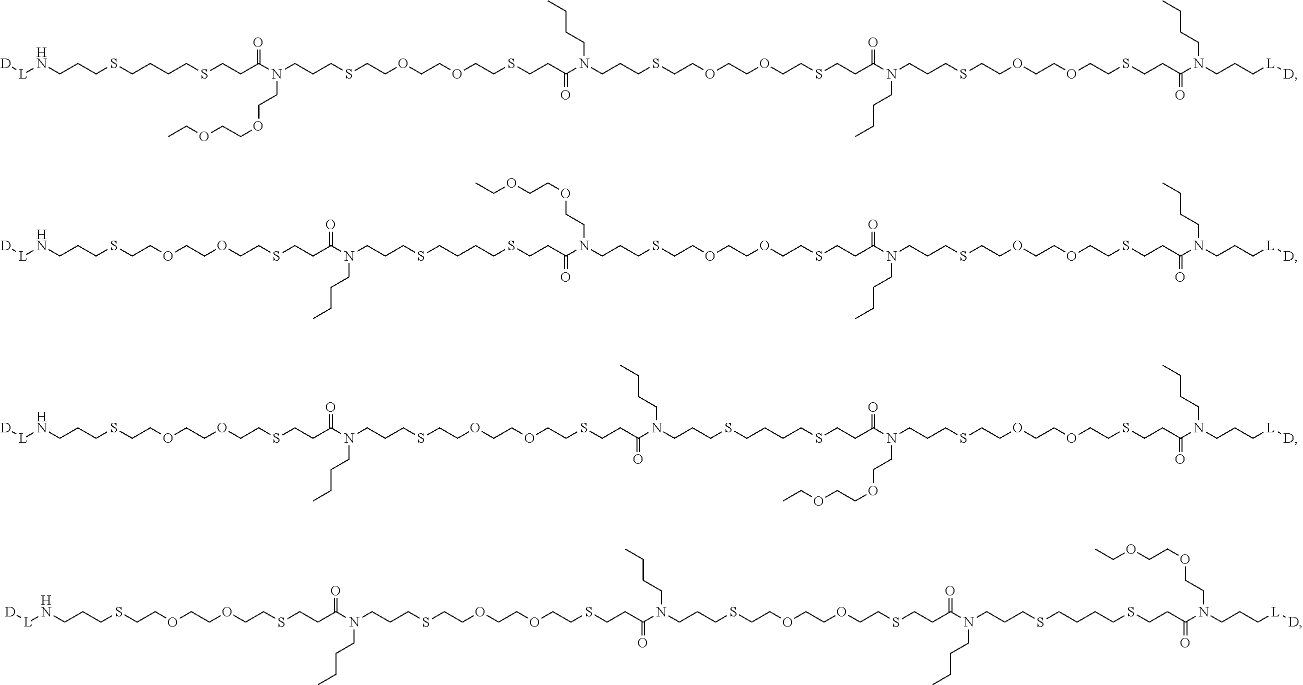 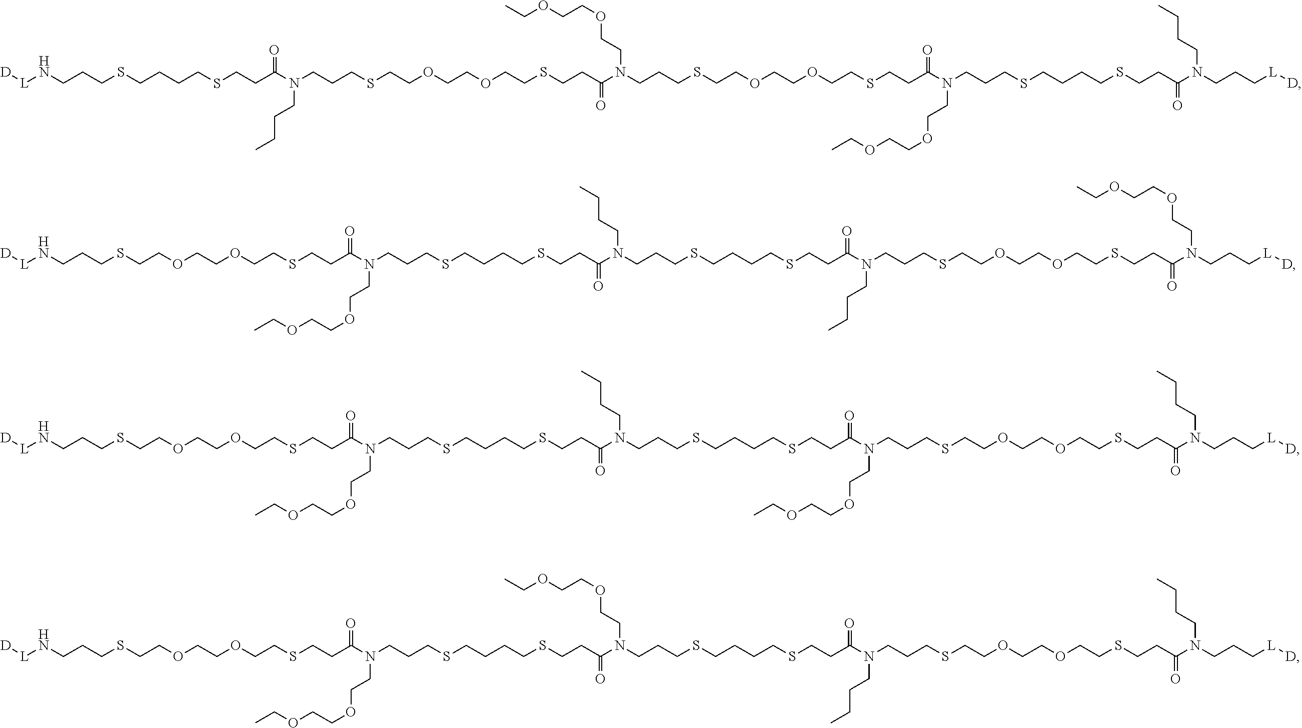 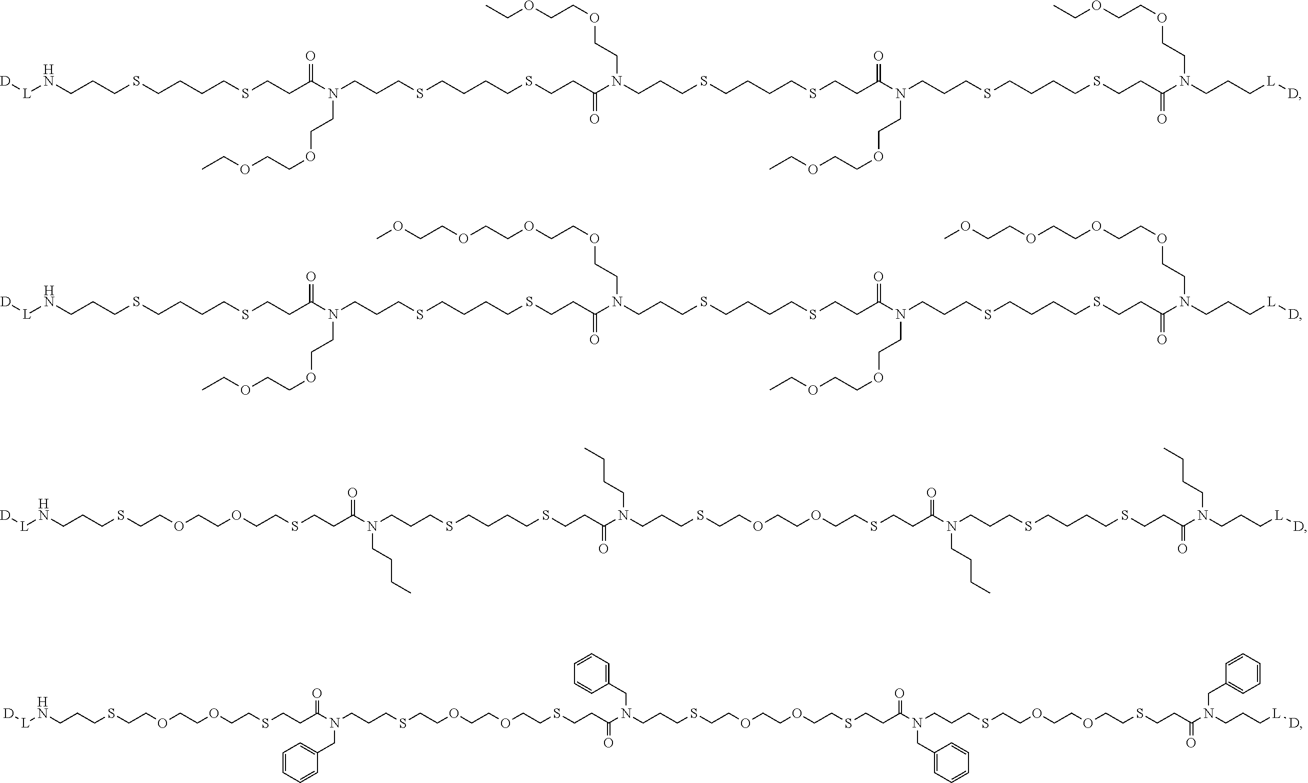 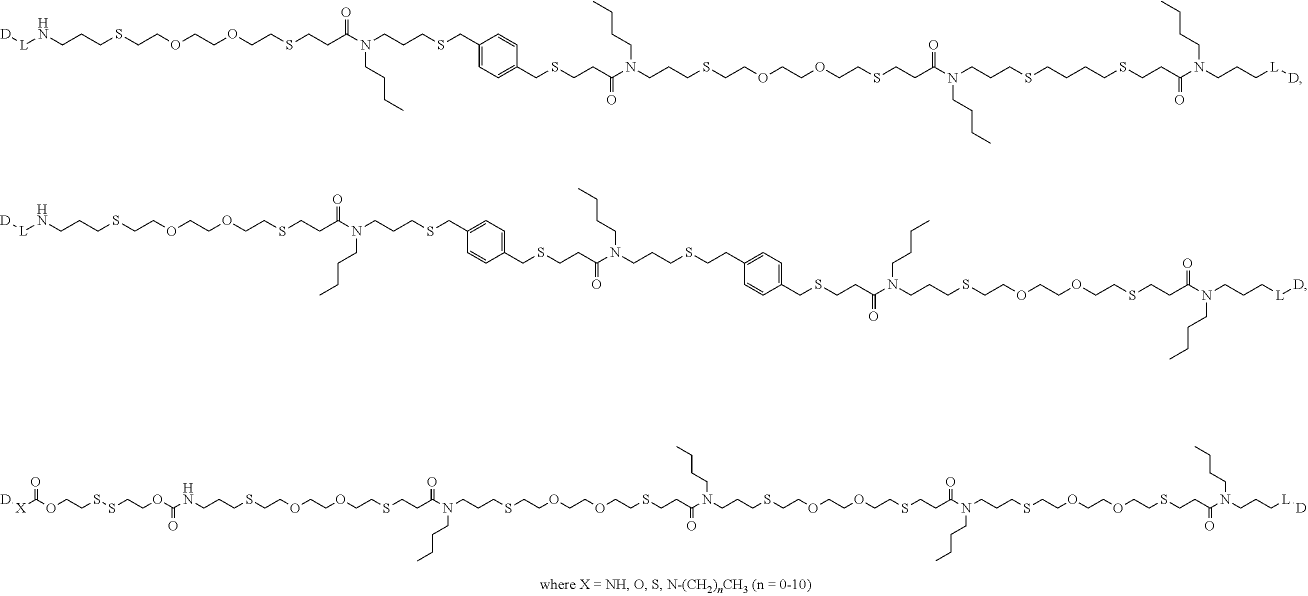
or isomers thereof, wherein L is chosen from a linking group, NH, N, O, and S, and D is one or more cargo group.

9. The compound of claim 1, wherein the compound has the following structure:

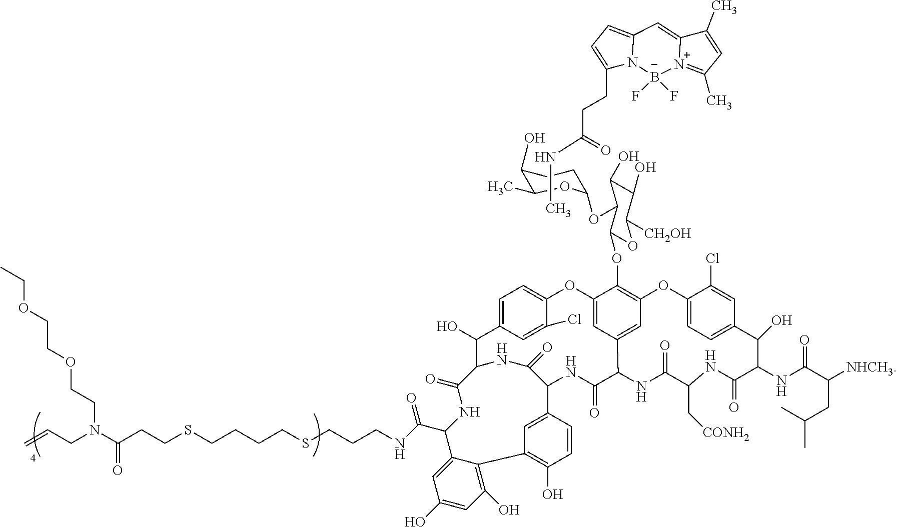

10. The compound of claim 1, wherein the compound has the following structure:

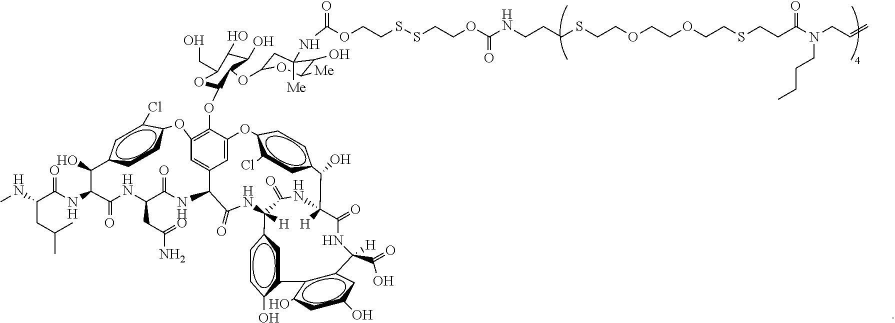

11. The compound of claim 1, wherein the compound has the following structure: 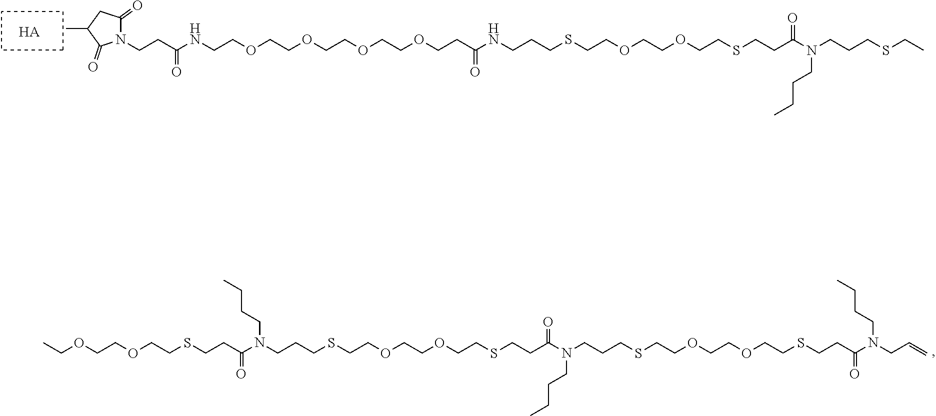
wherein HA is KADNAAIESIRNGTYDHDVYRDEALNNRFQIKGVELKSGYKDW-S- (SEQ ID NO:1) and the underlined S is a sulfur atom.

12. The compound of claim 1, wherein the compound has the following structure: 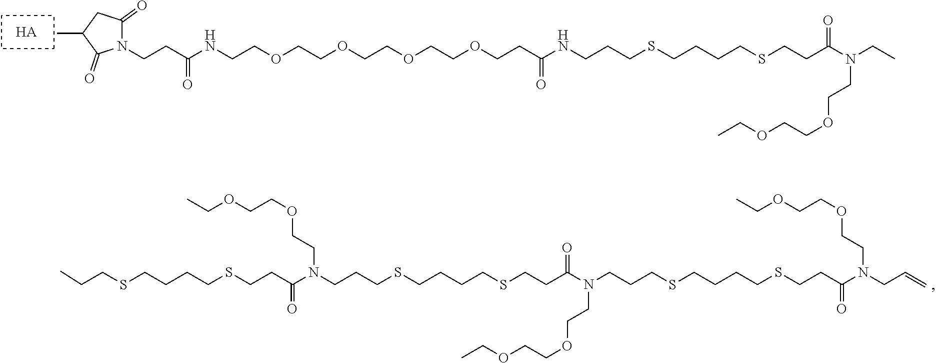
wherein HA is KADNAAIESIRNGTYDHDVYRDEALNNRFQIKGVELKSGYKDW-S- (SEQ ID NO:1) and the underlined S is a sulfur atom.

13. A composition comprising one or more compound of claim 1 and a pharmaceutically acceptable carrier.

14. A method for intracellular delivery of a compound comprising administering to a subject in a need of treatment a composition of claim 13.

15. The method of claim 14, wherein the subject in need of treatment has or is suspected of having bacterial infections, cancers, viral infections, urinary tract infections, skin infections, cystic fibrosis, sepsis, fungal infections, or a combination thereof.

16. The method of claim 15, wherein the bacterial infection is caused by Listeria monocytogenes, Staphylococcus aureus, Pseudomonas aeruginosa, Tuberculosis, Salmonella enterica, Francisella tularensis, and combinations thereof


SEQUENCE LISTING (3 sequences)

SEQ ID NO: 1 - protein, 43 aa, Artificial Sequence
  synthetic peptide
KADNAAIESIRNGTYDHDVYRDEALNNRFQIKGVELKSGYKDW

SEQ ID NO: 2 - protein, 9 aa, Artificial Sequence
  Tat49-57 9-mer
RKKRRQRRR

SEQ ID NO: 3 - protein, 10 aa, Artificial Sequence
  synthetic peptide
RRRRRRRRRK